(19) United States
(12) Patent Application Publication
(10) Pub. No.: US 2025/0346605 A1
(43) Pub. Date: Nov. 13, 2025

(54) NEW DERIVATIVES FOR TREATING TRPM3 MEDIATED DISORDERS

(71) Applicants: Katholieke Universiteit Leuven, Leuven (BE); Biohaven Therapeutics Ltd., New Haven, CT (US)

(72) Inventors: Arnaud MARCHAND, Bierbeek (BE); Jean-Christophe VANHERCK, Heverlee (BE); Patrick CHALTIN, Zétrud-Lemay (BE); Melanie REICH, Aachen (DE); Sebastian KRÜGER, Aachen (DE); Damien MARCHAND, Antwerpen (BE); Thomas VOETS, Kessel-Lo (BE); Joris VRIENS, Wakkerzeel (BE)

(21) Appl. No.: 18/868,758

(22) PCT Filed: May 25, 2023
(86) PCT No.: PCT/EP2023/063997

§ 371 Date: Nov. 24, 2024

(30) Foreign Application Priority Data
May 25, 2022 (EP) 22175523.4

(51) Int. Cl. C07D 491/048; A61K 31/4355; A61K 31/444; A61K 31/4545

(52) U.S. Cl. CPC C07D 491/048; A61K 31/4355; A61K 31/444; A61K 31/4545

(57) ABSTRACT

The invention relates to compounds that are useful for the prevention or treatment of TRPM3 mediated disorders, more in particular disorders selected from pain, epilepsy and inflammatory hypersensitivity. The invention also relates to a method for the prevention or treatment of said TRPM3 mediated disorders.

### FIELD OF THE INVENTION

The invention relates to compounds that are useful for the prevention or treatment of TRPM3 mediated disorders, more in particular disorders selected from pain, epilepsy and inflammatory hypersensitivity. The invention also relates to a method for the prevention or treatment of said TRPM3 mediated disorders.

### BACKGROUND OF THE INVENTION

The TRP superfamily consists of proteins with six transmembrane domains (6TM) that assemble as homo- or heterotetramers to form cation-permeable ion channels. The name TRP originates from the Drosophila trp (transient receptor potential) mutant, which is characterized by a transient receptor potential in the fly photoreceptors in the response to sustained light. In the last 15 years, trp-related channels have been identified in yeast, worms, insects, fish and mammals, including 27 TRPs in humans. Based on sequence homology, TRP channels can be divided into seven subfamilies: TRPC, TRPV, TRPM, TRPA, TRPP, TRPML and TRPN.

Members of the TRP superfamily are expressed in probably all mammalian organs and cell types, and in recent years great progress has been made in the understanding of their physiological role. The tailored selectivity of certain TRP channels enables them to play key roles in the cellular uptake and/or transepithelial transport of Ca²⁺, Mg²⁺ and trace metal ions. Moreover, the sensitivity of TRP channels to a broad array of chemical and physical stimuli, allows them to function as dedicated biological sensors involved in processes ranging from vision to taste, and tactile sensation. In particular, several members of the TRP superfamily exhibit a very high sensitivity to temperature. These so-called thermoTRPs are highly expressed in sensory neurons and/or skin keratinocytes, where they act as primary thermosensors for the detection of innocuous and noxious (painful) temperatures.

It is becoming increasingly clear that TRP channel dysfunction is directly involved in the etiology of various inherited and acquired diseases. Indeed, both loss-of-function and gain-of-function mutations in the TRP channel genes have been identified as the direct cause of inherited diseases, including brachyolmia, hypomagnesemia with secondary hypocalcemia, polycystic kidney disease, mucolipidosis type IV and familial focal segmental glomerulosclerosis. Moreover, TRP channel function/dysfunction has been directly linked to a wide range of pathological conditions, including chronic pain, hypertension, cancer, epilepsy and neurodegenerative disorders.

TRPM3 (Transient receptor potential melastatin 3) represents a promising pharmacological target. TRPM3 is expressed in a large subset of small-diameter sensory neurons from dorsal root and trigeminal ganglia, and is involved in heat sensing. The neurosteroid pregnenolone sulfate is a potent known activator of TRPM3 (Wagner et al., 2008). The neurosteroid pregnenolone sulfate evoked pain in wild type mice but not in knock-out TRPM3 mice. It was also recently shown that CFA induced inflammation and inflammatory pain are eliminated in TRPM3 knock-out mice. Therefore, TRPM3 antagonists could be used as analgesic drugs to counteract pain, such as inflammatory pain (Vriens J. et al. Neuron, May 2011). TRPM3 is also expressed in a number of other tissues, including the brain; reports have shown that two mutations in TRPM3 are associated with a developmental and epileptic encephalopathy (Zhao, s., et al. Channels (Austin). 2021).

A few TRPM3 antagonists are known, but none of them points towards the compounds of the current invention (Straub I et al. Mol Pharmacol, November 2013). For instance, Liquiritigenin, a postulated TRPM3 blocker has been described to decrease mechanical and cold hyperalgesia in a rat pain model (Chen L et al. Scientific reports, July 2014). There is still a great medical need for novel, alternative and/or better therapeutics for the prevention or treatment of TRPM3 mediated disorders such as pain and epilepsy, more in particular for pain such as inflammatory pain, or epilepsy such as epileptic encephalopathies. Therapeutics with good potency on a certain type of pain, low level or no side-effects (such as no possibilities for addiction as with opiates, no toxicity) and/or good or better pharmacokinetic or -dynamic properties are highly needed.

The invention provides a class of novel compounds which are antagonists of TRPM3 and can be used as modulators of TRPM3 mediated disorders.

### SUMMARY OF THE INVENTION

A first aspect of the present invention provides a compound of formula (I), a stereo-isomeric form, a physiologically acceptable salt, solvate and/or polymorph thereof

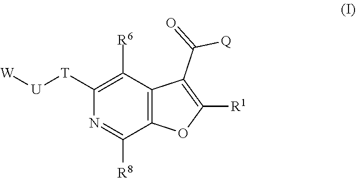

wherein
R¹ represents —F, —Cl, —Br, —I, —CN, —R^{w}, —OR^{w}, —OC(═O)R^{w}, —NR^{w}R^{x}, —NR^{w}C(═O)R^{x}, —SR^{w}, —S(═O)R^{w}, —S(═O)₂R^{w}, —C(═O)R^{w}, —C(═O)OR^{w}, or —C(═O)NR^{w}R^{x};
Q represents —NR³R⁴;
R³ represents —OH or —R^{Y1};
R⁴ represents —R^{Y2} or —S(═O)₂R^{Y3};
   or R³ and R⁴ together form a 4, 5, 6, 7 or 8 membered heterocycle containing 1 to 3 heteroatoms selected from N, O and S, saturated or unsaturated, unsubstituted or mono- or polysubstituted;
T represents —O— and U represents —(CR⁵R^{5′})ₙ—; or T represents —(CR⁵R⁵′)ₙ— and U represents —O—;
n is an integer selected from 1, 2, 3, 4, or 5;
R⁵ and R^{5′} independently of one another represent —R^{Y4};
R⁶ and R⁸ independently of one another represent —F, —Cl, —Br, —I, —CN, —NO₂, —SF₅, —R^{w}, —OR^{w}, —OC(═O)R^{w}, —NR^{w}R^{x}, —NR^{w}C(═O)R^{X}, —SR^{w}, —S(═O)R^{w}, —S(═O)₂R^{w}, —C(═O)R^{w}, —C(═O)OR^{w}, or —C(═O)NR^{w}R^{x};
W represents
3-14-membered cycloalkyl, saturated or unsaturated; unsubstituted, mono- or polysubstituted;
6-14-membered aryl, unsubstituted, mono- or polysubstituted;
3-14-membered heterocycloalkyl, saturated or unsaturated, mono- or polysubstituted;
5-14-membered heteroaryl, mono- or polysubstituted; or
—C₁-C₆-alkyl, —C₂-C₆-alkenyl, —C₂-C₆-alkynyl; in each case unsubstituted, mono- or polysubstituted;
wherein
R^{w} and R^{x} independently of one another in each case independently represent
   —H;
   —C₁-C₆-alkyl, saturated or unsaturated, unsubstituted, mono- or poly substituted;
   —C₁-C₆-heteroalkyl, saturated or unsaturated, unsubstituted, mono- or poly substituted;
   3-14-membered cycloalkyl, saturated or unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted; or
   3-14-membered heterocycloalkyl, saturated or unsaturated, unsubstituted, mono- or polysubstituted; wherein said 3-14-membered heterocycloalkyl is optionally connected through —C₁-C₆-alkylene- or —C₁-C₆-heteroalkylene-, in each case saturated or unsaturated, unsubstituted, mono- or polysubstituted;
R¹, R², R³, and R⁴ independently of one another in each case independently represent
   —H;
   —C₁-C₆-alkyl, saturated or unsaturated, unsubstituted, mono- or poly substituted;
   —C₁-C₆-heteroalkyl, saturated or unsaturated, unsubstituted, mono- or poly substituted;
   3-14-membered cycloalkyl, saturated or unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted;
   3-14-membered heterocycloalkyl, saturated or unsaturated, unsubstituted, mono- or polysubstituted; wherein said 3-14-membered heterocycloalkyl is optionally connected through —C₁-C₆-alkylene- or —C₁-C₆-heteroalkylene-, in each case saturated or unsaturated, unsubstituted, mono- or polysubstituted; 6-14-membered aryl, unsubstituted, mono- or polysubstituted; wherein said 6-14-membered aryl is optionally connected through —C₁-C₆-alkylene- or —C₁-C₆-heteroalkylene-, in each case saturated or unsaturated, unsubstituted, mono- or polysubstituted; or
   5-14-membered heteroaryl, unsubstituted, mono- or polysubstituted; wherein said 5-14-membered heteroaryl is optionally connected through —C₁-C₆-alkylene- or —C₁-C₆-heteroalkylene-, in each case saturated or unsaturated, unsubstituted, mono- or polysubstituted;
and wherein “mono- or polysubstituted” in each case independently means substituted with one or more substituents independently of one another selected from —F, —Cl, —Br, —I, —CN, —C₁₋₆-alkyl, —CF₃, —CF₂H, —CFH₂, —CF₂Cl, —CFCl₂, —C₁₋₆-alkylene-CF₃, —C₁₋₆-alkylene-CF₂H, —C₁₋₆-alkylene-CFH₂, —C₁₋₆-alkylene-NH—C₁₋₆-alkylene-CF₃, —C₁₋₆-alkylene-N(C₁₋₆-alkyl)-C₁₋₆-alkylene-CF₃, —C(═O)—C₁₋₆-alkyl, —C₁₋₆-alkylene-C(═O)—C₁₋₆-alkyl, —C(═O)OH, —C₁₋₆-alkylene-C(═O)—OH, —C(═O)—OC₁₋₆-alkyl, —C₁₋₆-alkylene-C(═O)—OC₁₋₆-alkyl, —C(═O)O—C₁₋₆-alkylene-CF₃, —C(═O)—NH₂, —C₁₋₆-alkylene-C(═O)—NH₂, —C(═O)—NH(C₁₋₆-alkyl), —C₁₋₆-alkylene-C(═O)—NH(C₁₋₆-alkyl), —C(═O)—N(C₁₋₆-alkyl)₂, —C₁₋₆-alkylene-C(═O)—N(C₁₋₆-alkyl)₂, —C(═O)—NH(OH), —C₁₋₆-alkylene-C(═O)—NH(OH), —OH, —C₁₋₆-alkylene-OH, ═O, —OCF₃, —OCF₂H, —OCFH₂, —OCF₂Cl, —OCFCl₂, —O—C₁₋₆-alkyl, —C₁₋₆-alkylene-O—C₁₋₆-alkyl, —O—C₁₋₆-alkylene-O—C₁₋₆-alkyl, —O—C₁₋₆-alkylene-N₁₋₂, —O—C₁₋₆-alkylene-NH—C₁₋₆-alkyl, —O—C₁₋₆-alkylene-N(C₁₋₆-alkyl)₂, —O—C(═O)—C₁₋₆-alkyl, —C₁₋₆-alkylene-O—C(═O)—C₁₋₆-alkyl, —O—C(═O)—O—C₁₋₆-alkyl, —C₁₋₆-alkylene-O—C(═O)—O—C₁₋₆-alkyl, —O—C(═O)—NH(C₁₋₆-alkyl), —C₁₋₆-alkylene-O—C(═O)—NH(C₁₋₆-alkyl), —O—C(═O)—N(C₁₋₆-alkyl)₂, —C₁₋₆-alkylene-O—C(═O)—N(C₁₋₆-alkyl)₂, —O—S(═O)₂—NH₂, —C₁₋₆-alkylene-O—S(═O)₂—NH₂, —O—S(═O)₂—NH(C₁₋₆-alkyl), —C₁₋₆-alkylene-O—S(═O)₂—NH(C₁₋₆-alkyl), —O—S(═O)₂—N(C₁₋₆-alkyl)₂, —C₁₋₆-alkylene-O—S(═O)₂—N(C₁₋₆-alkyl)₂, —NH₂, —NO, —NO₂, —C₁₋₆-alkylene-NH₂, —NH(C₁₋₆-alkyl), —C₁₋₆-alkylene-NH(C₁₋₆-alkyl), —N(C₁₋₆-alkyl)₂, —C₁₋₆-alkylene-N(C₁₋₆-alkyl)₂, —NH—C(═O)—C₁₋₆-alkyl, —C₁₋₆-alkylene-NH—C(═O)—C₁₋₆-alkyl, —NH—C(═O)—O—C₁₋₆-alkyl, —C₁₋₆-alkylene-NH—C(═O)—O—C₁₋₆-alkyl, —NH—C(═O)—NH₂, —C₁₋₆-alkylene-NH—C(═O)—NH₂, —NH—C(═O)—NH(C₁₋₆-alkyl), —C₁₋₆-alkylene-NH—C(═O)—NH(C₁₋₆-alkyl), —NH—C(═O)—N(C₁₋₆-alkyl)₂, —C₁₋₆-alkylene-NH—C(═O)—N(C₁₋₆-alkyl)₂, —N(C₁₋₆-alkyl)-C(═O)—C₁₋₆-alkyl, —C₁₋₆-alkylene-N(C₁₋₆-alkyl)-C(═O)—C₁₋₆-alkyl, —N(C₁₋₆-alkyl)-C(═O)—O—C₁₋₆-alkyl, —C₁₋₆-alkylene-N(C₁₋₆-alkyl)-C(═O)—O—C₁₋₆-alkyl, —N(C₁₋₆-alkyl)-C(═O)—NH₂, —C₁₋₆-alkylene-N(C₁₋₆-alkyl)-C(═O)—NH₂, —N(C₁₋₆-alkyl)-C(═O)—NH(C₁₋₆-alkyl), —C₁₋₆-alkylene-N(C₁₋₆-alkyl)-C(═O)—NH(C₁₋₆-alkyl), —N(C₁₋₆-alkyl)-C(═O)—N(C₁₋₆-alkyl)₂, —C₁₋₆-alkylene-N(C₁₋₆-alkyl)-C(═O)—N(C₁₋₆-alkyl)₂, —NH—S(═O)₂OH, —C₁₋₆-alkylene-NH—S(═O)₂OH, —NH—S(═O)₂—C₁₋₆-alkyl, —C₁₋₆-alkylene-NH—S(═O)₂—C₁₋₆-alkyl, —NH—S(═O)₂—O—C₁₋₆-alkyl, —C₁₋₆-alkylene-NH—S(═O)₂—O—C₁₋₆-alkyl, —NH—S(═O)₂—NH₂, —C₁₋₆-alkylene-NH—S(═O)₂—NH₂, —NH—S(═O)₂—NH(C₁₋₆-alkyl), —C₁₋₆-alkylene-NH—S(═O)₂—NH(C₁₋₆-alkyl), —NH—S(═O)₂N(C₁₋₆-alkyl)₂, —C₁₋₆-alkylene-NH—S(═O)₂N(C₁₋₆-alkyl)₂, —N(C₁₋₆-alkyl)-S(═O)₂—OH, —C₁₋₆-alkylene-N(C₁₋₆-alkyl)-S(═O)₂—OH, —N(C₁₋₆-alkyl)-S(═O)₂—C₁₋₆-alkyl, —C₁₋₆-alkylene-N(C₁₋₆-alkyl)-S(═O)₂—C₁₋₆-alkyl, —N(C₁₋₆-alkyl)-S(═O)₂—O—C₁₋₆-alkyl, —C₁₋₆-alkylene-N(C₁₋₆-alkyl)-S(═O)₂—O—C₁₋₆-alkyl, —N(C₁₋₆-alkyl)-S(═O)₂—NH₂, —C₁₋₆-alkylene-N(C₁₋₆-alkyl)-S(═O)₂—NH₂, —N(C₁₋₆-alkyl)-S(═O)₂—NH(C₁₋₆-alkyl), —C₁₋₆-alkylene-N(C₁₋₆-alkyl)-S(═O)₂—NH(C₁₋₆-alkyl), —N(C₁₋₆-alkyl)-S(═O)₂—N(C₁₋₆-alkyl)₂, —C₁₋₆-alkylene-N(C₁₋₆-alkyl)-S(═O)₂—N(C₁₋₆-alkyl)₂, —SH, ═S, —SF₅, —SCF₃, —SCF₂H, —SCFH₂, —S—C₁₋₆-alkyl, —C₁₋₆-alkylene-S—C₁₋₆-alkyl, —S(═O)—C₁₋₆-alkyl, —C₁₋₆-alkylene-S(═O)—C₁₋₆-alkyl, —S(═O)₂-C₁₋₆-alkyl, —C₁₋₆-alkylene-S(═O)₂—C₁₋₆-alkyl, —S(═O)₂—OH, —C₁₋₆-alkylene-S(═O)₂—OH, —S(═O)₂—O—C₁₋₆-alkyl, —C₁₋₆-alkylene-S(═O)₂—O—C₁₋₆-alkyl, —S(═O)₂—NH₂, —C₁₋₆-alkylene-S(═O)₂—NH₂, —S(═O)₂—NH(C₁₋₆-alkyl), —C₁₋₆-alkylene-S(═O)₂—NH(C₁₋₆-alkyl), —S(═O)₂—N(C₁₋₆-alkyl)₂, —C₁₋₆-alkylene-S(═O)₂—N(C₁₋₆-alkyl)₂, 3-14-membered cycloalkyl, —C₁₋₆-alkylene-(3-14-membered cycloalkyl), 3 to 14-membered heterocycloalkyl, —C₁₋₆-alkylene-(3 to 14-membered heterocycloalkyl), -phenyl, —C₁₋₆-alkylene-phenyl, 5 to 14-membered heteroaryl, —C₁₋₆-alkylene-(5 to 14-membered heteroaryl), —O-(3-14-membered cycloalkyl), —O-(3 to 14-membered heterocycloalkyl), —O-phenyl, —O-(5 to 14-membered heteroaryl), —C(═O)-(3-14-membered cycloalkyl), —C(═O)-(3 to 14-membered heterocycloalkyl), —C(═O)-phenyl, —C(═O)-(5 to 14-membered heteroaryl), —S(═O)₂-(3-14-membered cycloalkyl), —S(═O)₂-(3 to 14-membered heterocycloalkyl), —S(═O)₂-phenyl, or —S(═O)₂-(5 to 14-membered heteroaryl); or the compound is 5-(benzyloxy)-2-methylfuro[2,3-c]pyridine-3-carboxylate.

In particular, the present invention provides a compound of formula (I), a stereo-isomeric form, a physiologically acceptable salt, solvate and/or polymorph thereof

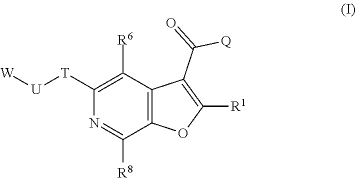

wherein
R¹ represents —F, —Cl, —Br, —I, —CN, —R^{w}, —OR^{w}, —OC(═O)R^{w}, —NR^{w}R^{x}, —NR^{w}C(═O)R^{x}, —SR^{w}, —S(═O)R^{w}, —S(═O)₂R^{w}, —C(═O)R^{w}, —C(═O)OR^{w}, or —C(═O)NR^{w}R^{x};
Q represents —NR³R⁴;
R³ represents —OH or —R^{Y1};
R⁴ represents —R² or —S(═O)₂R^{Y3};
or R³ and R⁴ together form a 4, 5, 6, 7 or 8 membered heterocycle containing 1 to 3 heteroatoms selected from N, O and S, saturated or unsaturated, unsubstituted or mono- or polysubstituted;
T represents —O— and U represents —(CR⁵R^{5′})ₙ—; or T represents —(CR⁵R⁵′)ₙ— and U represents —O—;
n is an integer selected from 1, 2, 3, 4, or 5;
R⁵ and R⁵′ independently of one another represent —R^{Y4};
R⁶, and R⁸ independently of one another represent —F, —Cl, —Br, —I, —CN, —NO₂, —SF₅, —R^{w}, —OR^{w}, —OC(═O)R^{w}, —NR^{w}R^{x}, —NR^{w}C(═O)R^{x}, —SR^{w}, —S(═O)R^{w}, —S(═O)₂R^{w}, —C(═O)R^{w}, —C(═O)OR^{w}, or —C(═O)NR^{w}R^{x};
W represents
3-14-membered cycloalkyl, saturated or non-aromatic unsaturated; unsubstituted, mono- or polysubstituted;
6-14-membered aryl, unsubstituted, mono- or polysubstituted;
3-14-membered heterocycloalkyl, saturated or unsaturated, mono- or polysubstituted;
5-14-membered heteroaryl, mono- or polysubstituted; or
—C₁-C₆-alkyl, —C₂-C₆-alkenyl, —C₂-C₆-alkynyl; in each case unsubstituted, mono- or polysubstituted;
wherein
R^{w} and R^{x} independently of one another in each case independently represent
—H;
—C₁-C₆-alkyl, saturated or unsaturated, unsubstituted, mono- or polysubstituted;
—C₁-C₆-heteroalkyl, saturated or unsaturated, unsubstituted, mono- or polysubstituted;
3-14-membered cycloalkyl, saturated or non-aromatic unsaturated, unsubstituted, mono- or polysubstituted;
wherein said 3-14-membered cycloalkyl is optionally connected through —C₁-C₆-alkylene- or —C₁-C₆-heteroalkylene-, in each case saturated or unsaturated, unsubstituted, mono- or polysubstituted; or
3-14-membered heterocycloalkyl, saturated or unsaturated, unsubstituted, mono- or polysubstituted; wherein said 3-14-membered heterocycloalkyl is optionally connected through —C₁-C₆-alkylene- or —C₁-C₆-heteroalkylene-, in each case saturated or unsaturated, unsubstituted, mono- or polysubstituted;
R^{Y1}, R^{Y2}, R^{Y3}, and R^{Y4} independently of one another in each case independently represent
—H;
—C₁-C₆-alkyl, saturated or unsaturated, unsubstituted, mono- or polysubstituted;
—C₁-C₆-heteroalkyl, saturated or unsaturated, unsubstituted, mono- or polysubstituted;
3-14-membered cycloalkyl, saturated or non-aromatic unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted;
3-14-membered heterocycloalkyl, saturated or unsaturated, unsubstituted, mono- or polysubstituted; wherein said 3-14-membered heterocycloalkyl is optionally connected through —C₁-C₆-alkylene- or —C₁-C₆-heteroalkylene-, in each case saturated or unsaturated, unsubstituted, mono- or polysubstituted;
6-14-membered aryl, unsubstituted, mono- or polysubstituted; wherein said 6-14-membered aryl is optionally connected through —C₁-C₆-alkylene- or —C₁-C₆-heteroalkylene-, in each case saturated or unsaturated, unsubstituted, mono- or polysubstituted; or
5-14-membered heteroaryl, unsubstituted, mono- or polysubstituted; wherein said 5-14-membered heteroaryl is optionally connected through —C₁-C₆-alkylene- or —C₁-C₆-heteroalkylene-, in each case saturated or unsaturated, unsubstituted, mono- or polysubstituted;
and wherein “mono- or polysubstituted” in each case independently means substituted with one or more substituents independently of one another selected from —F, —Cl, —Br, —I, —CN, —C₁₋₆-alkyl, —CF₃, —CF₂H, —CFH₂, —CF₂Cl, —CFCl₂, —C₁₋₆-alkylene-CF₃, —C₁₋₆-alkylene-CF₂H, —C₁₋₆-alkylene-CFH₂, —C₁₋₆-alkylene-NH—C₁₋₆-alkylene-CF₃, —C₁₋₆-alkylene-N(C₁₋₆-alkyl)-C₁₋₆-alkylene-CF₃, —C(═O)—C₁₋₆-alkyl, —C₁₋₆-alkylene-C(═O)—C₁₋₆-alkyl, —C(═O)OH, —C₁₋₆-alkylene-C(═O)—OH, —C(═O)—OC₁₋₆-alkyl, —C₁₋₆-alkylene-C(═O)—OC₁₋₆-alkyl, —C(═O)O—C₁₋₆-alkylene-CF₃, —C(═O)—NH₂, —C₁₋₆-alkylene-C(═O)—NH₂, —C(═O)—NH(C₁₋₆-alkyl), —C₁₋₆-alkylene-C(═O)—NH(C₁₋₆-alkyl), —C(═O)—N(C₁₋₆-alkyl)₂, —C₁₋₆-alkylene-C(═O)—N(C₁₋₆-alkyl)₂, —C(═O)—NH(OH), —C₁₋₆-alkylene-C(═O)—NH(OH), —OH, —C₁₋₆-alkylene-OH, ═O, —OCF₃, —OCF₂H, —OCFH₂, —OCF₂Cl, —OCFCl₂, —O—C₁₋₆-alkyl, —C₁₋₆-alkylene-O—C₁₋₆-alkyl, —O—C₁₋₆-alkylene-O—C₁₋₆-alkyl, —O—C₁₋₆-alkylene-NH₂, —O—C₁₋₆-alkylene-NH—C₁₋₆-alkyl, —O—C₁₋₆-alkylene-N(C₁₋₆-alkyl)₂, —O—C(═O)—C₁₋₆-alkyl, —C₁₋₆-alkylene-O—C(═O)—C₁₋₆-alkyl, —O—C(═O)—O—C₁₋₆-alkyl, —C₁₋₆-alkylene-O—C(═O)—O—C₁₋₆-alkyl, —O—C(═O)—NH(C₁₋₆-alkyl), —C₁₋₆-alkylene-O—C(═O)—NH(C₁₋₆-alkyl), —O—C(═O)—N(C₁₋₆-alkyl)₂, —C₁₋₆-alkylene-O—C(═O)—N(C₁₋₆-alkyl)₂, —O—S(═O)₂—NH₂, —C₁₋₆-alkylene-O—S(═O)₂—NH₂, —O—S(═O)₂—NH(C₁₋₆-alkyl), —C₁₋₆-alkylene-O—S(═O)₂—NH(C₁₋₆-alkyl), —O—S(═O)₂—N(C₁₋₆-alkyl)₂, —C₁₋₆-alkylene-O—S(═O)₂—N(C₁₋₆-alkyl)₂, —NH₂, —NO, —NO₂, —C₁₋₆-alkylene-NH₂, —NH(C₁₋₆-alkyl), —C₁₋₆-alkylene-NH(C₁₋₆-alkyl), —N(C₁₋₆-alkyl)₂, —C₁₋₆-alkylene-N(C₁₋₆-alkyl)₂, —NH—C(═O)—C₁₋₆-alkyl, —C₁₋₆-alkylene-NH—C(═O)—C₁₋₆-alkyl, —NH—C(═O)—O—C₁₋₆-alkyl, —C₁₋₆-alkylene-NH—C(═O)—O—C₁₋₆-alkyl, —NH—C(═O)—NH₂, —C₁₋₆-alkylene-NH—C(═O)—NH₂, —NH—C(═O)—NH(C₁₋₆-alkyl), —C₁₋₆-alkylene-NH—C(═O)—NH(C₁₋₆-alkyl), —NH—C(═O)—N(C₁₋₆-alkyl)₂, —C₁₋₆-alkylene-NH—C(═O)—N(C₁₋₆-alkyl)₂, —N(C₁₋₆-alkyl)-C(═O)—C₁₋₆-alkyl, —C₁₋₆-alkylene-N(C₁₋₆-alkyl)-C(═O)—C₁₋₆-alkyl, —N(C₁₋₆-alkyl)-C(═O)—O—C₁₋₆-alkyl, —C₁₋₆-alkylene-N(C₁₋₆-alkyl)-C(═O)—O—C₁₋₆-alkyl, —N(C₁₋₆-alkyl)-C(═O)—NH₂, —C₁₋₆-alkylene-N(C₁₋₆-alkyl)-C(═O)—NH₂, —N(C₁₋₆-alkyl)-C(═O)—NH(C₁₋₆-alkyl), —C₁₋₆-alkylene-N(C₁₋₆-alkyl)-C(═O)—NH(C₁₋₆-alkyl), —N(C₁₋₆-alkyl)-C(═O)—N(C₁₋₆-alkyl)₂, —C₁₋₆-alkylene-N(C₁₋₆-alkyl)-C(═O)—N(C₁₋₆-alkyl)₂, —NH—S(═O)₂OH, —C₁₋₆-alkylene-NH—S(═O)₂OH, —NH—S(═O)₂—C₁₋₆-alkyl, —C₁₋₆-alkylene-NH—S(═O)₂—C₁₋₆-alkyl, —NH—S(═O)₂—O—C₁₋₆-alkyl, —C₁₋₆-alkylene-NH—S(═O)₂—O—C₁₋₆-alkyl, —NH—S(═O)₂—NH₂, —C₁₋₆-alkylene-NH—S(═O)₂—NH₂, —NH—S(═O)₂—NH(C₁₋₆-alkyl), —C₁₋₆-alkylene-NH—S(═O)₂—NH(C₁₋₆-alkyl), —NH—S(═O)₂N(C₁₋₆-alkyl)₂, —C₁₋₆-alkylene-NH—S(═O)₂N(C₁₋₆-alkyl)₂, —N(C₁₋₆-alkyl)-S(═O)₂—OH, —C₁₋₆-alkylene-N(C₁₋₆-alkyl)-S(═O)₂—OH, —N(C₁₋₆-alkyl)-S(═O)₂—C₁₋₆-alkyl, —C₁₋₆-alkylene-N(C₁₋₆-alkyl)-S(═O)₂—C₁₋₆-alkyl, —N(C₁₋₆-alkyl)-S(═O)₂—O—C₁₋₆-alkyl, —C₁₋₆-alkylene-N(C₁₋₆-alkyl)-S(═O)₂—O—C₁₋₆-alkyl, —N(C₁₋₆-alkyl)-S(═O)₂—NH₂, —C₁₋₆-alkylene-N(C₁₋₆-alkyl)-S(═O)₂—NH₂, —N(C₁₋₆-alkyl)-S(═O)₂—NH(C₁₋₆-alkyl), —C₁₋₆-alkylene-N(C₁₋₆-alkyl)-S(═O)₂—NH(C₁₋₆-alkyl), —N(C₁₋₆-alkyl)-S(═O)₂—N(C₁₋₆-alkyl)₂, —C₁₋₆-alkylene-N(C₁₋₆-alkyl)-S(═O)₂—N(C₁₋₆-alkyl)₂, —SH, ═S, —SF₅, —SCF₃, —SCF₂H, —SCFH₂, —S—C₁₋₆-alkyl, —C₁₋₆-alkylene-S—C₁₋₆-alkyl, —S(═O)—C₁₋₆-alkyl, —C₁₋₆-alkylene-S(═O)—C₁₋₆-alkyl, —S(═O)₂—C₁₋₆-alkyl, —C₁₋₆-alkylene-S(═O)₂—C₁₋₆-alkyl, —S(═O)₂—OH, —C₁₋₆-alkylene-S(═O)₂—OH, —S(═O)₂—O—C₁₋₆-alkyl, —C₁₋₆-alkylene-S(═O)₂—O—C₁₋₆-alkyl, —S(═O)₂—NH₂, —C₁₋₆-alkylene-S(═O)₂—NH₂, —S(═O)₂—NH(C₁₋₆-alkyl), —C₁₋₆-alkylene-S(═O)₂—NH(C₁₋₆-alkyl), —S(═O)₂—N(C₁₋₆-alkyl)₂, —C₁₋₆-alkylene-S(═O)₂—N(C₁₋₆-alkyl)₂, 3-14-membered cycloalkyl, —C₁₋₆-alkylene-(3-14-membered cycloalkyl), 3 to 14-membered heterocycloalkyl, —C₁₋₆-alkylene-(3 to 14-membered heterocycloalkyl), -phenyl, —C₁₋₆-alkylene-phenyl, 5 to 14-membered heteroaryl, —C₁₋₆-alkylene-(5 to 14-membered heteroaryl), —O-(3-14-membered cycloalkyl), —O-(3 to 14-membered heterocycloalkyl), —O-phenyl, —O-(5 to 14-membered heteroaryl), —C(═O)-(3-14-membered cycloalkyl), —C(═O)-(3 to 14-membered heterocycloalkyl), —C(═O)-phenyl, —C(═O)-(5 to 14-membered heteroaryl), —S(═O)₂-(3-14-membered cycloalkyl), —S(═O)₂-(3 to 14-membered heterocycloalkyl), —S(═O)₂-phenyl, or —S(═O)₂-(5 to 14-membered heteroaryl).

A second aspect of the present invention encompasses a pharmaceutical composition comprising a compound of formula (I) as described in the first aspect.

According to a third aspect the invention also provides a compound of formula (I) as described in the first aspect or a pharmaceutical composition as described in the second aspect for use as a medicament.

According to a fourth aspect, the present invention also encompasses a compound of formula (I) as described in the first aspect or a pharmaceutical composition as described in the second aspect for use in the prevention and/or treatment of TRPM3 mediated disorders, especially for use in the prevention and/or treatment of pain, epilepsy and/or inflammatory hypersensitivity; and/or for counteracting pain, epilepsy and/or inflammatory hypersensitivity. In particular for use in the in the prevention and/or treatment of pain, epileptic encephalopathies and/or inflammatory hypersensitivity; and/or for counteracting pain, epileptic encephalopathies and/or inflammatory hypersensitivity

The present invention also provides a method for the prevention or treatment of a TRPM3 mediated disorder by administering a compound of formula (I) as described in the first aspect to a subject in need thereof. More in particular, the invention relates to such method for the prevention and/or treatment of pain, epilepsy and/or inflammatory hypersensitivity; and/or for counteracting pain, epilepsy and/or inflammatory hypersensitivity. More in particular the invention relates to such method for the prevention and/or treatment of pain, epileptic encephalopathies and/or inflammatory hypersensitivity; and/or for counteracting pain, epileptic encephalopathies and/or inflammatory hypersensitivity.

The invention further provides a method for the preparation of a compound of formula (I) as described in the first aspect, comprising the steps of:
converting 6-(Benzyloxy)-4-iodopyridin-3-ol derivative into a 5-benzyloxyfuro[2,3-c]pyridine-3-carboxylate ester derivative,
substituting previously obtained 5-benzyloxyfuro[2,3-c]pyridine-3-carboxylate ester derivatives with suitable derivatives bearing a leaving group or alcohol derivatives under Mitsunobu or alkylation conditions to obtain 5-O-substituted-furo[2,3-c]pyridine-3-carboxylate ester derivatives
converting the previously obtained 5-O-substituted- furo[2,3-c]pyridine-3-carboxylate ester derivatives in carboxylic acid to obtain 5-O-substituted- furo[2,3-c]pyridine-3-carboxylic acid derivatives, and
coupling the previously obtained 5-O-substituted-furo[2,3-c]pyridine-3-carboxylic acid derivatives with a suitable amine to obtain the desired derivatives of the invention.

### DETAILED DESCRIPTION OF THE INVENTION

Before the present invention is described, it is to be understood that this invention is not limited to particular processes, methods, and compounds described, as such processes, methods, and compounds may, of course, vary. It is also to be understood that the terminology used herein is not intended to be limiting, since the scope of the present invention will be limited only by the appended claims.

When describing the compounds and processes of the invention, the terms used are to be construed in accordance with the following definitions, unless a context dictates otherwise.

As used in the specification and the appended claims, the singular forms “a”, “an,” and “the” include both singular and plural referents unless the context clearly dictates otherwise. By way of example, “a compound” means one compound or more than one compound.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The term “about” as used herein when referring to a measurable value such as a parameter, an amount, a temporal duration, and the like, is meant to encompass variations of +/−10% or less, preferably +1-5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

As used herein, the term “and/or,” when used in a list of two or more items, means that any one of the listed items can be employed by itself or any combination of two or more of the listed items can be employed. For example, if a list is described as comprising group A, B, and/or C, the list can comprise A alone; B alone; C alone; A and B in combination; A and C in combination, B and C in combination; or A, B, and C in combination.

The recitation of numerical ranges by endpoints includes all integer numbers and, where appropriate, fractions subsumed within that range (e.g., 1 to 5 can include 1, 2, 3, 4 when referring to, for example, a number of elements, and can also include 1.5, 2, 2.75 and 3.80, when referring to, for example, measurements). The recitation of end points also includes the end point values themselves (e.g., from 1.0 to 5.0 includes both 1.0 and 5.0). Any numerical range recited herein is intended to include all sub-ranges subsumed therein.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

Similarly, it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects.

Unless otherwise defined, all terms used in disclosing the invention, including technical and scientific terms, have the meaning as commonly understood by one of ordinary skill in the art to which this invention belongs. By means of further guidance, definitions for the terms used in the description are included to better appreciate the teaching of the present invention.

When describing the present invention, the terms used are to be construed in accordance with the following definitions, unless a context dictates otherwise.

The terms described above and others used in the specification are well understood to those in the art.

Whenever the term “substituted” is used herein, it is meant to indicate that one or more hydrogen atoms on the atom indicated in the expression using “substituted” is replaced with a selection from the indicated group, provided that the indicated atom's normal valence is not exceeded, and that the substitution results in a chemically stable compound, i.e., a compound that is sufficiently robust to survive isolation from a reaction mixture.

In each of the following definitions, the number of carbon atoms represents the maximum number of carbon atoms generally optimally present in the substituent or linker; it is understood that where otherwise indicated in the present application, the number of carbon atoms represents the optimal maximum number of carbon atoms for that particular substituent or linker.

The term “leaving group” or “LG” as used herein means a chemical group which is susceptible to be displaced by a nucleophile or cleaved off or hydrolyzed in basic or acidic conditions. In a particular embodiment, a leaving group is selected from a halogen atom (e.g., Cl, Br, I) or a sulfonate (e.g., mesylate, tosylate, triflate).

The term “protecting group” refers to a moiety of a compound that masks or alters the properties of a functional group or the properties of the compound as a whole. The chemical substructure of a protecting group varies widely. One function of a protecting group is to serve as intermediates in the synthesis of the parental drug substance. Chemical protecting groups and strategies for protection/deprotection are well known in the art. See: “Protective Groups in Organic Chemistry”, Theodora W. Greene (John Wiley & Sons, Inc., New York, 1991. Protecting groups are often utilized to mask the reactivity of certain functional groups, to assist in the efficiency of desired chemical reactions, e.g., making and breaking chemical bonds in an ordered and planned fashion.

Protection of functional groups of a compound alters other physical properties besides the reactivity of the protected functional group, such as the polarity, lipophilicity (hydrophobicity), and other properties which can be measured by common analytical tools. Chemically protected intermediates may themselves be biologically active or inactive.

Protected compounds may also exhibit altered, and in some cases, optimized properties in vitro and in vivo, such as passage through cellular membranes and resistance to enzymatic degradation or sequestration. In this role, protected compounds with intended therapeutic effects may be referred to as prodrugs. Another function of a protecting group is to convert the parental drug into a prodrug, whereby the parental drug is released upon conversion of the prodrug in vivo. Because active prodrugs may be absorbed more effectively than the parental drug, prodrugs may possess greater potency in vivo than the parental drug. Protecting groups are removed either in vitro, in the instance of chemical intermediates, or in vivo, in the case of prodrugs. With chemical intermediates, it is not particularly important that the resulting products after deprotection, e.g., alcohols, be physiologically acceptable, although in general it is more desirable if the products are pharmacologically innocuous.

The term “heteroatom(s)” as used herein means an atom selected from nitrogen, which can be quaternized; oxygen; and sulfur, including sulfoxide and sulfone.

The term “alkyl, saturated or unsaturated” as used herein encompasses saturated alkyl as well as unsaturated alkyl such as alkenyl, alkynyl, and the like. Suitable examples of alkyl, saturated or unsaturated, include, but are not limited to methyl, ethyl, 1-propyl (n-propyl), 2-propyl (iPr), 1-butyl, 2-methyl-1-propyl(i-Bu), 2-butyl (s-Bu), 2-dimethyl-2-propyl (t-Bu), 1-pentyl (n-pentyl), 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, and 3,3-dimethyl-2-butyl. “C₁₋₆alkyl” includes all linear or branched alkyl groups with between 1 and 6 carbon atoms, and thus includes methyl, ethyl, n-propyl, i-propyl, butyl and its isomers (e.g., n-butyl, i-butyl and t-butyl); pentyl and its isomers, hexyl and its isomers. For example, “C₁₋₅alkyl” includes all includes all linear or branched alkyl groups with between 1 and 5 carbon atoms, and thus includes methyl, ethyl, n-propyl, i-propyl, butyl and its isomers (e.g., n-butyl, i-butyl and t-butyl); pentyl and its isomers; ethylene or vinyl (—CH═CH₂), allyl (—CH₂CH═CH₂), 5-hexenyl (—CH₂CH₂CH₂CH₂CH═CH₂); 2-butenyl, 3-butenyl, 2-pentenyl and its isomers, 2-hexenyl and its isomers, 2,4-pentadienyl, ethynyl, 2-propynyl, 2-butynyl, 3-butynyl, 2-pentynyl and its chain isomers, 2-hexynyl and its chain isomers.

The term “alkyl” as used herein means normal, secondary, or tertiary, linear or branched hydrocarbon with no site of unsaturation. Examples are methyl, ethyl, 1-propyl (n-propyl), 2-propyl (iPr), 1-butyl, 2-methyl-1-propyl(i-Bu), 2-butyl (s-Bu), 2-dimethyl-2-propyl (t-Bu), 1-pentyl (n-pentyl), 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, and 3,3-dimethyl-2-butyl. “C₁₋₆alkyl” includes all linear or branched alkyl groups with between 1 and 6 carbon atoms, and thus includes methyl, ethyl, n-propyl, i-propyl, butyl and its isomers (e.g., n-butyl, i-butyl and t-butyl); pentyl and its isomers, hexyl and its isomers. For example, “C₁₋₅alkyl” includes all includes all linear or branched alkyl groups with between 1 and 5 carbon atoms, and thus includes methyl, ethyl, n-propyl, i-propyl, butyl and its isomers (e.g., n-butyl, i-butyl and t-butyl); pentyl and its isomers. For example, “C₁₋₄alkyl” includes all linear or branched alkyl groups with between 1 and 4 carbon atoms, and thus includes methyl, ethyl, n-propyl, i-propyl, butyl and its isomers (e.g., n-butyl, i-butyl and t-butyl). For example, “C₁₋₃alkyl” includes all linear or branched alkyl groups with between 1 and 3 carbon atoms, and thus includes methyl, ethyl, n-propyl, i-propyl. A “substituted C₁₋₆alkyl” refers to a C₁₋₆alkyl group substituted with one or more substituent(s) (for example 1 to 3 substituent(s), for example 1, 2, or 3 substituent(s)) at any available point of attachment.

The term “alkenyl” as used herein means normal, secondary or tertiary, linear or branched hydrocarbon with at least one site (usually 1 to 3, preferably 1) of unsaturation, namely a carbon-carbon, sp2 double bond. Examples include, but are not limited to: ethylene or vinyl (—CH═CH₂), allyl (—CH₂CH═CH₂), and 5-hexenyl (—CH₂CH₂CH₂CH₂CH═CH₂). The double bond may be in the cis or trans configuration. When a subscript is used herein following a carbon atom, the subscript refers to the number of carbon atoms that the named group may contain. For example, the term “C₂₋₆alkenyl” refers to an unsaturated hydrocarbyl group, which may be linear, or branched comprising one or more carbon-carbon double bonds and comprising from 2 to 6 carbon atoms. For example, C₂₋₄alkenyl includes all linear, or branched alkenyl groups having 2 to 4 carbon atoms. Examples of C₂₋₆alkenyl groups are ethenyl, 2-propenyl, 2-butenyl, 3-butenyl, 2-pentenyl and its isomers, 2-hexenyl and its isomers, 2,4-pentadienyl, and the like.

The term “alkynyl” as used herein means normal, secondary, tertiary, linear or branched hydrocarbon with at least one site (usually 1 to 3, preferably 1) of unsaturation, namely a carbon-carbon, sp triple bond. Examples include, but are not limited to: ethynyl (—C═CH), and 1-propynyl (propargyl, —CH₂C═CH). When a subscript is used herein following a carbon atom, the subscript refers to the number of carbon atoms that the named group may contain. For example, the term “C₂₋₆alkynyl” refers to an unsaturated hydrocarbyl group, which may be linear, or branched comprising one or more carbon-carbon triple bonds and comprising from 2 to 6 carbon atoms. For example, C₂₋₄alkynyl includes all linear, or branched alkynyl groups having 2 to 4 carbon atoms. Non limiting examples of C₂₋₆alkynyl groups include ethynyl, 2-propynyl, 2-butynyl, 3-butynyl, 2-pentynyl and its chain isomers, 2-hexynyl and its chain isomers, and the like.

The term “alkylene, saturated or unsaturated” as used herein encompasses saturated alkylene as well as unsaturated alkylene such as alkenylene, alkynylene, and the like. The term “alkylene” as used herein means saturated, linear or branched chain hydrocarbon radical having two monovalent radical centers derived by the removal of two hydrogen atoms from the same or two different carbon atoms of a parent alkane. Typical alkylene radicals include, but are not limited to: methylene (—CH₂—), ethylene (—CH₂—CH₂—), methylmethylene (—CH(CH₃)—), 1-methyl-ethylene (—CH(CH₃)—CH₂—), n-propylene (—CH₂—CH₂—CH₂—), 2-methylpropylene (—CH₂—CH(CH₃)—CH₂—), 3-methylpropylene (—CH₂—CH₂—CH(CH₃)—), n-butylene (—CH₂—CH₂—CH₂—CH₂—), 2-methylbutylene (—CH₂—CH(CH₃)—CH₂—CH₂—), 4-methylbutylene (—CH₂—CH₂—CH₂—CH(CH₃)—), pentylene and its chain isomers, hexylene and its chain isomers.

The term “alkenylene” as used herein means linear or branched chain hydrocarbon radical with at least one site (usually 1 to 3, preferably 1) of unsaturation, namely a carbon-carbon, sp2 double bond, and having two monovalent radical centers derived by the removal of two hydrogen atoms from the same or two different carbon atoms of a parent alkene. “C₂₋₆alkenylene”, by itself or as part of another substituent, refers to C₂₋₆alkenyl groups that are divalent, i.e., with two single bonds for attachment to two other groups.

The term “alkynylene” as used herein means linear or branched chain hydrocarbon radical with at least one site (usually 1 to 3, preferably 1) of unsaturation, namely a carbon-carbon, sp triple bond, and having two monovalent radical centers derived by the removal of two hydrogen atoms from the same or two different carbon atoms of a parent alkyne. As used herein, the term “C₂₋₆alkynylene”, by itself or as part of another substituent, refers to C₂₋₆alkynyl groups that are divalent, i.e., with two single bonds for attachment to two other groups.

The term “alkenyl” as a group or part of a group, refers to an unsaturated hydrocarbyl group, which may be linear, or branched, comprising one or more carbon-carbon double bonds. When a subscript is used herein following a carbon atom, the subscript refers to the number of carbon atoms that the named group may contain. For example, the term “C₂₋₆alkenyl” refers to an unsaturated hydrocarbyl group, which may be linear, or branched comprising one or more carbon-carbon double bonds and comprising from 2 to 6 carbon atoms. For example, C₂₋₄alkenyl includes all linear, or branched alkenyl groups having 2 to 4 carbon atoms. Examples of C₂₋₆alkenyl groups are ethenyl, 2-propenyl, 2-butenyl, 3-butenyl, 2-pentenyl and its isomers, 2-hexenyl and its isomers, 2,4-pentadienyl, and the like.

The term “alkynyl” by itself or as part of another substituent, refers to an unsaturated hydrocarbyl group, which may be linear, or branched, comprising one or more carbon-carbon triple bonds. When a subscript is used herein following a carbon atom, the subscript refers to the number of carbon atoms that the named group may contain. For example, the term “C₂₋₆alkynyl” refers to an unsaturated hydrocarbyl group, which may be linear, or branched comprising one or more carbon-carbon triple bonds and comprising from 2 to 6 carbon atoms. For example, C₂₋₄alkynyl includes all linear, or branched alkynyl groups having 2 to 4 carbon atoms. Non limiting examples of C₂₋₆alkynyl groups include ethynyl, 2-propynyl, 2-butynyl, 3-butynyl, 2-pentynyl and its chain isomers, 2-hexynyl and its chain isomers, and the like.

The term “halo C₁₋₆alkyl” as a group or part of a group, refers to a C₁₋₆alkyl group having the meaning as defined above wherein one, two, or three hydrogen atoms are each replaced with a halogen as defined herein. Non-limiting examples of such halo C₁₋₆alkyl groups include chloromethyl, 1-bromoethyl, fluoromethyl, difluoromethyl, trifluoromethyl, 1,1,1-trifluoroethyl and the like.

The term “heteroalkyl, saturated or unsaturated” as used herein encompasses saturated heteroalkyl as well as unsaturated heteroalkyl such as heteroalkenyl, heteroalkynyl, and the like. The term “heteroalkyl” as used herein means linear or branched chain alkyl wherein one or more carbon atoms (usually 1, 2 or 3) are replaced by a heteroatom, i.e., an oxygen, nitrogen or sulfur atom, with the proviso that said chain may not contain two adjacent O atoms or two adjacent S atoms. This means that one or more —CH₃ of said alkyl can be replaced by —NH₂ and/or that one or more —CH₂— of said alkyl can be replaced by —NH—, —O— or —S—. The S atoms in said chains may be optionally oxidized with one or two oxygen atoms, to afford sulfoxides and sulfones, respectively. Furthermore, the heteroalkyl groups in the compounds of the invention can contain an oxo or thio group at any carbon or heteroatom that will result in a stable compound. Exemplary heteroalkyl groups include, but are not limited to, alcohols, alkyl ethers (such as for example -methoxy, -ethoxy, -butoxy, . . . ), primary, secondary, and tertiary alkyl amines, amides, ketones, esters, alkyl sulfides, and alkyl sulfones. The term “heteroalkenyl” means linear or branched chain alkenyl wherein one or more carbon atoms (usually 1, 2 or 3) are replaced by an oxygen, nitrogen or sulfur atom, with the proviso that said chain may not contain two adjacent O atoms or two adjacent S atoms.

The term heteroalkenyl thus comprises imines, —O-alkenyl, —NH-alkenyl, —N(alkenyl)₂, —N(alkyl)(alkenyl), and —S— alkenyl. The term “heteroalkynyl” as used herein means linear or branched chain alkynyl wherein one or more carbon atoms (usually 1, 2 or 3) are replaced by an oxygen, nitrogen or sulfur atom, with the proviso that said chain may not contain two adjacent O atoms or two adjacent S atoms. The term heteroalkynyl thus comprises -cyano, —O-alkynyl, —NH-alkynyl, —N(alkynyl)₂, —N(alkyl)(alkynyl), —N(alkenyl)(alkynyl), and —S-alkynyl.

The term “heteroalkylene, saturated or unsaturated” as used herein encompasses saturated heteroalkylene as well as unsaturated heteroalkylene such as heteroalkenylene, heteroalkynylene, and the like. The term “heteroalkylene” as used herein means linear or branched chain alkylene wherein one or more carbon atoms (usually 1, 2 or 3) are replaced by a heteroatom, i.e., an oxygen, nitrogen or sulfur atom, with the proviso that said chain may not contain two adjacent O atoms or two adjacent S atoms. The term “heteroalkenylene” as used herein means linear or branched chain alkenylene wherein one or more carbon atoms (usually 1, 2 or 3) are replaced by an oxygen, nitrogen or sulfur atom, with the proviso that said chain may not contain two adjacent O atoms or two adjacent S atoms. The term “heteroalkynylene” as used herein means linear or branched chain alkynylene wherein one or more carbon atoms (usually 1, 2 or 3) are replaced by an oxygen, nitrogen or sulfur atom, with the proviso that said chain may not contain two adjacent O atoms or two adjacent S atoms.

The term “cycloalkyl, saturated or unsaturated” as used herein encompasses saturated cycloalkyl as well as unsaturated cycloalkyl such as cycloalkenyl, cycloalkynyl and the like. Particularly, the term “cycloalkyl, saturated or unsaturated” as used herein encompasses saturated cycloalkyl as well as unsaturated non-aromatic cycloalkyl such as cycloalkenyl, and cycloalkynyl. In particular, the terms “cycloalkyl, saturated or unsaturated” and “cycloalkyl, saturated or non-aromatic unsaturated” are synonymous. The term “cycloalkyl, saturated or unsaturated” also includes all saturated and unsaturated hydrocarbon groups containing 1 or more rings, including monocyclic or bicyclic groups. The further rings of multi-ring cycloalkyls may be either fused, bridged and/or joined through one or more spiro atoms. Suitable examples of cycloalkyl, saturated or unsaturated, include, but are not limited to cyclopropyl, cyclopropanyl, cyclobutyl, cyclobutenyl, cyclopentyl, cyclopentenyl, cyclohexyl, cyclohexenyl cycloheptyl, cycloheptenyl, cyclooctyl, cyclooctenyl, 1,3-cyclohexadienyl, 1,4-cyclohexadienyl, 1,5-cyclooctadienyl, bicycle[2.2.1]heptan-2yl, (1S,4R)-norbornan-2-yl, (1R,4R)-norbornan-3-yl, (1S,4S)-norbornan-2-yl, (1R,4S)-norbornan-2-yl, decalinyl, adamantyl, spiro[3.3]heptan-2-yl, 3-bicyclo[3.1.0]hexanyl The term “cycloalkyl” as used herein and unless otherwise stated means a saturated cyclic hydrocarbon radical, including monocyclic or bicyclic groups; the further rings of multi-ring cycloalkyls may be either fused, bridged and/or joined through one or more spiro atoms. Examples of cycloalkyl include for instance cyclopropyl, cyclobutyl, cyclopentyl, cyclohexyl, cycloheptyl, cyclooctyl, norbornyl, fenchyl, decalinyl, adamantyl, spiro[3.3]heptan-2-yl, 3-bicyclo[3.1.0]hexanyl and the like. The term “cycloalkenyl” as used herein means a non-aromatic cyclic hydrocarbon radical with at least one site (usually 1 to 3, preferably 1) of unsaturation, namely a carbon-carbon, sp2 double bond. The term “cycloalkenyl” also includes monocyclic or bicyclic groups; the further rings of multi-ring cycloalkenyls may be either fused, bridged and/or joined through one or more spiro atoms. Examples include, but are not limited to cyclopentenyl and cyclohexenyl. The double bond may be in the cis or trans configuration. The term “cycloalkynyl” as used herein means a non-aromatic cyclic hydrocarbon radical with at least one site (usually 1 to 3, preferably 1) of unsaturation, namely a carbon-carbon, sp triple. An example is cyclohept-1-yne. Fused systems of a cycloalkyl ring with a heterocycloalkyl ring are considered as heterocycloalkyl irrespective of the ring that is bound to the core structure. Fused systems of a cycloalkyl ring with an aryl ring are considered as aryl irrespective of the ring that is bound to the core structure. Fused systems of a cycloalkyl ring with a heteroaryl ring are considered as heteroaryl irrespective of the ring that is bound to the core structure.

The term “heterocycloalkyl, saturated or unsaturated” as used herein encompasses saturated heterocycloalkyl as well as unsaturated non-aromatic heterocycloalkyl including at least one heteroatom, i.e., an N, O, or S as ring member. In particular, the terms “heterocycloalkyl, saturated or unsaturated” and “heterocycloalkyl, saturated or non-aromatic unsaturated” are synonymous. The term “heterocycloalkyl” as used herein and unless otherwise stated means “cycloalkyl” wherein one or more carbon atoms (usually 1, 2 or 3) are replaced by an oxygen, nitrogen or sulfur atom, with the proviso that said chain may not contain two adjacent O atoms or two adjacent S atoms. The term “heterocycloalkenyl” as used herein and unless otherwise stated means “cycloalkenyl” wherein one or more carbon atoms (usually 1, 2 or 3) are replaced by an oxygen, nitrogen or sulfur atom, with the proviso that said chain may not contain two adjacent O atoms or two adjacent S atoms. The term “heterocycloalkynyl” as used herein and unless otherwise stated means “cycloalkynyl” wherein one or more carbon atoms (usually 1, 2 or 3) are replaced by an oxygen, nitrogen or sulfur atom, with the proviso that said chain may not contain two adjacent O atoms or two adjacent S atoms. Examples of saturated and unsaturated heterocycloalkyl include but are not limited to azepane, 1,4-oxazepane, azetane, azetidine, aziridine, azocane, diazepane, dioxane, dioxolane, dithiane, dithiolane, imidazolidine, isothiazolidine, isoxalidine, morpholine, oxazolidine, oxepane, oxetane, oxirane, piperazine, piperidine, pyrazolidine, pyrrolidine, quinuclidine, tetrahydrofuran, tetrahydropyran, tetrahydrothiopyran, thiazolidine, thietane, thiirane, thiolane, thiomorpholine, indoline, dihydrobenzofuran, dihydrobenzothiophene, 1,1-dioxothiacyclohexane, 2-azaspiro[3.3]heptane, 2-oxaspiro[3.3]heptane, 7-azaspiro[3.5]nonane, 8-azabicyclo[3.2.1]octane, 9-azabicyclo[3.3.1]nonane, hexahydro-1H-pyrrolizine, hexahydrocyclopenta[c]pyrrole, octahydrocyclopenta[c]pyrrole, and octahydropyrrolo[1,2-a]pyrazin. Further heterocycloalkyls in the meaning of the invention are described in Paquette, Leo A. “Principles of Modern Heterocyclic Chemistry” (W.A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; Katritzky, Alan R., Rees, C. W. and Scriven, E. “Comprehensive Heterocyclic Chemistry” (Pergamon Press, 1996); and J. Am. Chem. Soc. (1960) 82:5566. When the heterocycloalkyl contains no nitrogen as ring member, it is typically bonded through carbon. When the heterocycloalkyl contains nitrogen as ring member, it may be bonded through nitrogen or carbon. Fused systems of heterocycloalkyl ring with a cycloalkyl ring are considered as heterocycloalkyl irrespective of the ring that is bound to the core structure. Fused systems of a heterocycloalkyl ring with an aryl ring are considered as heterocycloalkyl irrespective of the ring that is bound to the core structure. Fused systems of a heterocycloalkyl ring with a heteroaryl ring are considered as heteroaryl irrespective of the ring that is bound to the core structure.

The term “aryl” as used herein means an aromatic hydrocarbon. Typical aryl groups include, but are not limited to 1 ring, or 2 or 3 rings fused together, radicals derived from benzene, naphthalene, anthracene, biphenyl, and the like. Fused systems of an aryl ring with a cycloalkyl ring are considered as aryl irrespective of the ring that is bound to the core structure. Fused systems of an aryl ring with a heterocycloalkyl ring are considered as heterocycloalkyl irrespective of the ring that is bound to the core structure. Thus, indoline, dihydrobenzofuran, dihydrobenzothiophene and the like are considered as heterocycloalkyl according to the invention. Fused systems of an aryl ring with a heteroaryl ring are considered as heteroaryl irrespective of the ring that is bound to the core structure.

The term “heteroaryl” as used herein means an aromatic ring system including at least one heteroatom, i.e., N, O, or S as ring member of the aromatic ring system. Examples of heteroaryl include but are not limited to benzimidazole, benzisoxazole, benzoxazole, benzodioxole, benzofuran, benzothiadiazole, benzothiazole, benzothiophene, carbazole, cinnoline, dibenzofuran, furan, furazan, imidazole, imidazopyridine, indazole, indole, indolizine, isobenzofuran, isoindole, isoquinoline, isothiazole, isoxazole, naphthyridine, oxadiazole, oxazole, oxindole, phthalazine, purine, pyrazine, pyrazole, pyridazine, pyridine, pyrimidine, pyrrole, quinazoline, quinoline, quinoxaline, tetrazole, thiadiazole, thiazole, thiophene, triazine, triazole, and [1,2,4]triazolo[4,3-a]pyrimidine.

By further way of example, carbon bonded heterocyclic rings are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline.

Preferred carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl. By way of example, nitrogen bonded heterocyclic rings are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of an isoindole, or isoindoline, position 4 of a morpholine, and position 9 of a carbazole, or β-carboline. Preferred nitrogen bonded heterocycles include 1-aziridyl, 1-azetedyl, 1-pyrrolyl, 1-imidazolyl, 1-pyrazolyl, and 1-piperidinyl. Further heteroaryls in the meaning of the invention are described in Paquette, Leo A. “Principles of Modern Heterocyclic Chemistry” (W.A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; Katritzky, Alan R., Rees, C. W. and Scriven, E. “Comprehensive Heterocyclic Chemistry” (Pergamon Press, 1996); and J. Am. Chem. Soc. (1960) 82:5566.

As used herein with respect to a substituting group, and unless otherwise stated, the terms “monosubstituted” “disubstituted”, “trisubstituted”, “polysubstituted” and the like means chemical structures defined herein, wherein the respective moiety is substituted with one or more substituents, meaning that one or more hydrogen atoms of said moiety are each independently replaced with a substituent. For example, —C₁₋₆-alkyl that may be polysubstituted with —F covers —CH₂F, —CHF₂, —CF₃, —CH₂CF₃, CF₂CF₃, and the like. Likewise, —C₁₋₆-alkyl that may be polysubstituted with substituents independently of one another selected from —F and —Cl covers —CH₂F, —CHF₂, —CF₃, —CH₂CF₃, CF₂CF₃, —CH₂Cl, —CHC₂, —CC₃, —CH₂CCl₃, CCl₂CCl₃, —CHClF, —CClF₂, —CCl₂CF₃, —CF₂CCl₃, —CClFCCl₂F, and the like. Any substituent designation that is found in more than one site in a compound of this invention shall be independently selected.

As used herein and unless otherwise stated, the term “solvate” includes any combination which may be formed by a derivative of this invention with a suitable inorganic solvent (e.g., hydrates) or organic solvent, such as but not limited to alcohols, ketones, esters, ethers, nitriles and the like.

The term “subject” as used herein, refers to an animal including humans, preferably a mammal, most preferably a human, who has been the object of treatment, observation or experiment.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation or partial alleviation of the symptoms of the disease or disorder being treated.

The term “composition” as used herein is intended to encompass a product comprising the specified ingredients in the therapeutically effective amounts, as well as any product which results, directly or indirectly, from combinations of the specified ingredients in the specified amounts.

The term “antagonist” or “inhibitor” as used herein refers to a compound capable of producing, depending on the circumstance, a functional antagonism of the TRPM3 ion channel, including competitive antagonists, non-competitive antagonists, desensitizing agonists, and partial agonists.

For purposes of the invention, the term “TRPM3-modulated” is used to refer to the condition of being affected by the modulation of the TRPM3 ion channel, including the state of being mediated by the TRPM3 ion channel.

The term “TRPM3 mediated disorder” as used herein refers to disorders or diseases for which the use of an antagonist of TRPM3 would prevent, treat, (partially) alleviate or improve the symptoms and consist of pain and inflammatory hypersensitivity condition. According to the International Association for the Study of Pain and for the purpose of the invention, pain is an unpleasant sensory and emotional experience associated with actual or potential tissue damage, or described in terms of such damage. Preferably, the TRPM3 mediated disorder is pain or epilepsy. Preferably, the TRPM3 mediated disorder is epilepsy which is preferably selected from epileptic encephalopathies. For the purpose of the invention, the term “epileptic encephalopathies” refers to a group of severe epilepsies that are characterized both by seizures, as well as encephalopathy. Preferably, the TRPM3 mediated disorder is pain which is preferably selected from nociceptive pain, inflammatory pain, and neuropathic pain. More preferably, the pain is post-operative pain. For the purpose of the invention, the term “inflammatory hypersensitivity” is used to refer to a condition that is characterized by one or more hallmarks of inflammation, including edema, erythema, hyperthermia and pain, and/or by an exaggerated physiologic or pathophysiologic response to one or more than one type of stimulation, including thermal, mechanical and/or chemical stimulation

The invention will be further described and in some instances with respect to particular embodiments, but the invention is not limited thereto.

Preferred statements (features) and embodiments of the compounds and processes of this invention are now set forth. Each statements and embodiments of the invention so defined may be combined with any other statement and/or embodiments unless clearly indicated to the contrary. In particular, any feature indicated as being preferred or advantageous may be combined with any other feature or features indicated as being preferred or advantageous.

Numbered statements of this invention are:
1. A compound of formula (I), a stereo-isomeric form, a physiologically acceptable salt, solvate and/or polymorph thereof

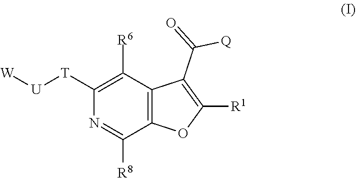

wherein
R¹ represent —F, —Cl, —Br, —I, —CN, —R^{w}, —OR^{w}, —OC(═O)R^{w}, —NR^{w}R^{x}, —NR^{w}C(═O)R^{x}, —SR^{w}, —S(═O)R^{w}, —S(═O)₂R^{w}, —C(═O)R^{w}, —C(═O)OR^{w}, or —C(═O)NR^{w}R^{x};
Q represents —NR³R⁴;
R³ represents —OH or —R^{Y1};
R⁴ represents —R² or —S(═O)₂R^{Y3};
or R³ and R⁴ together form a 4, 5, 6, 7 or 8 membered heterocycle containing 1 to 3 heteroatoms selected from N, O and S, saturated or unsaturated, unsubstituted or mono- or polysubstituted;
T represents —O— and U represents —(CR⁵R^{5′})ₙ—; or T represents —(CR⁵R^{5′})ₙ— and U represents —O—;
n is an integer selected from 1, 2, 3, 4, or 5;
R⁵ and R^{5′} independently of one another represent —R^{Y4};
R⁶, and R⁸ independently of one another represent —F, —Cl, —Br, —I, —CN, —NO₂, —SF₅, —R^{w}, —OR^{w}, —OC(═O)R^{w}, —NR^{w}R^{x}, —NR^{w}C(═O)R^{x}, —SR^{w}, —S(═O)R^{w}, —S(═O)₂R^{w}, —C(═O)R^{w}, —C(═O)OR^{w}, or —C(═O)NR^{w}R^{x};
W represents
3-14-membered cycloalkyl, saturated or unsaturated; unsubstituted, mono- or polysubstituted; in particular 3-14-membered cycloalkyl, saturated or non-aromatic unsaturated; unsubstituted, mono- or polysubstituted;
6-14-membered aryl, unsubstituted, mono- or polysubstituted;
3-14-membered heterocycloalkyl, saturated or unsaturated, mono- or polysubstituted;
5-14-membered heteroaryl, mono- or polysubstituted; or
—C₁-C₆-alkyl, —C₂-C₆-alkenyl, —C₂-C₆-alkynyl; in each case unsubstituted, mono- or polysubstituted;
wherein
R^{w} and R^{x} independently of one another in each case independently represent
—H;
—C₁-C₆-alkyl, saturated or unsaturated, unsubstituted, mono- or polysubstituted;
—C₁-C₆-heteroalkyl, saturated or unsaturated, unsubstituted, mono- or polysubstituted;
3-14-membered cycloalkyl, saturated or unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted, in particular 3-14-membered cycloalkyl, saturated or non-aromatic unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted; or
3-14-membered heterocycloalkyl, saturated or unsaturated, unsubstituted, mono- or polysubstituted; wherein said 3-14-membered heterocycloalkyl is optionally connected through —C₁-C₆-alkylene- or —C₁-C₆-heteroalkylene-, in each case saturated or unsaturated, unsubstituted, mono- or polysubstituted;
R^{Y1}, R^{Y2}, R^{Y3}, and R^{Y4} independently of one another in each case independently represent
—H;
—C₁-C₆-alkyl, saturated or unsaturated, unsubstituted, mono- or polysubstituted;
—C₁-C₆-heteroalkyl, saturated or unsaturated, unsubstituted, mono- or polysubstituted;
3-14-membered cycloalkyl, saturated or unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted, in particular 3-14-membered cycloalkyl, saturated or non-aromatic unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted;
3-14-membered heterocycloalkyl, saturated or unsaturated, unsubstituted, mono- or polysubstituted; wherein said 3-14-membered heterocycloalkyl is optionally connected through —C₁-C₆-alkylene- or —C₁-C₆-heteroalkylene-, in each case saturated or unsaturated, unsubstituted, mono- or polysubstituted; 6-14-membered aryl, unsubstituted, mono- or polysubstituted; wherein said 6-14-membered aryl is optionally connected through —C₁-C₆-alkylene- or —C₁-C₆-heteroalkylene-, in each case saturated or unsaturated, unsubstituted, mono- or polysubstituted; or
5-14-membered heteroaryl, unsubstituted, mono- or polysubstituted; wherein said 5-14-membered heteroaryl is optionally connected through —C₁-C₆-alkylene- or —C₁-C₆-heteroalkylene-, in each case saturated or unsaturated, unsubstituted, mono- or polysubstituted;
and wherein “mono- or polysubstituted” in each case independently means substituted with one or more substituents independently of one another selected from —F, —Cl, —Br, —I, —CN, —C₁₋₆-alkyl, —CF₃, —CF₂H, —CFH₂, —CF₂Cl, —CFCl₂, —C₁₋₆-alkylene-CF₃, —C₁₋₆-alkylene-CF₂H, —C₁₋₆-alkylene-CFH₂, —C₁₋₆-alkylene-NH—C₁₋₆-alkylene-CF₃, —C₁₋₆-alkylene-N(C₁₋₆-alkyl)-C₁₋₆-alkylene-CF₃, —C(═O)—C₁₋₆-alkyl, —C₁₋₆-alkylene-C(═O)—C₁₋₆-alkyl, —C(═O)OH, —C₁₋₆-alkylene-C(═O)—OH, —C(═O)—OC₁₋₆-alkyl, —C₁₋₆-alkylene-C(═O)—OC₁₋₆-alkyl, —C(═O)O—C₁₋₆-alkylene-CF₃, —C(═O)—NH₂, —C₁₋₆-alkylene-C(═O)—NH₂, —C(═O)—NH(C₁₋₆-alkyl), —C₁₋₆-alkylene-C(═O)—NH(C₁₋₆-alkyl), —C(═O)—N(C₁₋₆-alkyl)₂, —C₁₋₆-alkylene-C(═O)—N(C₁₋₆-alkyl)₂, —C(═O)—NH(OH), —C₁₋₆-alkylene-C(═O)—NH(OH), —OH, —C₁₋₆-alkylene-OH, ═O, —OCF₃, —OCF₂H, —OCFH₂, —OCF₂Cl, —OCFCl₂, —O—C₁₋₆-alkyl, —C₁₋₆-alkylene-O—C₁₋₆-alkyl, —O—C₁₋₆-alkylene-O—C₁₋₆-alkyl, —O—C₁₋₆-alkylene-NH₂, —O—C₁₋₆-alkylene-NH—C₁₋₆-alkyl, —O—C₁₋₆-alkylene-N(C₁₋₆-alkyl)₂, —O—C(═O)—C₁₋₆-alkyl, —C₁₋₆-alkylene-O—C(═O)—C₁₋₆-alkyl, —O—C(═O)—O—C₁₋₆-alkyl, —C₁₋₆-alkylene-O—C(═O)—O—C₁₋₆-alkyl, —O—C(═O)—NH(C₁₋₆-alkyl), —C₁₋₆-alkylene-O—C(═O)—NH(C₁₋₆-alkyl), —O—C(═O)—N(C₁₋₆-alkyl)₂, —C₁₋₆-alkylene-O—C(═O)—N(C₁₋₆-alkyl)₂, —O—S(═O)₂—NH₂, —C₁₋₆-alkylene-O—S(═O)₂—NH₂, —O—S(═O)₂—NH(C₁₋₆-alkyl), —C₁₋₆-alkylene-O—S(═O)₂—NH(C₁₋₆-alkyl), —O—S(═O)₂—N(C₁₋₆-alkyl)₂, —C₁₋₆-alkylene-O—S(═O)₂—N(C₁₋₆-alkyl)₂, —NH₂, —NO, —NO₂, —C₁₋₆-alkylene-NH₂, —NH(C₁₋₆-alkyl), —C₁₋₆-alkylene-NH(C₁₋₆-alkyl), —N(C₁₋₆-alkyl)₂, —C₁₋₆-alkylene-N(C₁₋₆-alkyl)₂, —NH—C(═O)—C₁₋₆-alkyl, —C₁₋₆-alkylene-NH—C(═O)—C₁₋₆-alkyl, —NH—C(═O)—O—C₁₋₆-alkyl, —C₁₋₆-alkylene-NH—C(═O)—O—C₁₋₆-alkyl, —NH—C(═O)—NH₂, —C₁₋₆-alkylene-NH—C(═O)—NH₂, —NH—C(═O)—NH(C₁₋₆-alkyl), —C₁₋₆-alkylene-NH—C(═O)—NH(C₁₋₆-alkyl), —NH—C(═O)—N(C₁₋₆-alkyl)₂, —C₁₋₆-alkylene-NH—C(═O)—N(C₁₋₆-alkyl)₂, —N(C₁₋₆-alkyl)-C(═O)—C₁₋₆-alkyl, —C₁₋₆-alkylene-N(C₁₋₆-alkyl)-C(═O)—C₁₋₆-alkyl, —N(C₁₋₆-alkyl)-C(═O)—O—C₁₋₆-alkyl, —C₁₋₆-alkylene-N(C₁₋₆-alkyl)-C(═O)—O—C₁₋₆-alkyl, —N(C₁₋₆-alkyl)-C(═O)—NH₂, —C₁₋₆-alkylene-N(C₁₋₆-alkyl)-C(═O)—NH₂, —N(C₁₋₆-alkyl)-C(═O)—NH(C₁₋₆-alkyl), —C₁₋₆-alkylene-N(C₁₋₆-alkyl)-C(═O)—NH(C₁₋₆-alkyl), —N(C₁₋₆-alkyl)-C(═O)—N(C₁₋₆-alkyl)₂, —C₁₋₆-alkylene-N(C₁₋₆-alkyl)-C(═O)—N(C₁₋₆-alkyl)₂, —NH—S(═O)₂OH, —C₁₋₆-alkylene-NH—S(═O)₂OH, —NH—S(═O)₂—C₁₋₆-alkyl, —C₁₋₆-alkylene-NH—S(═O)₂—C₁₋₆-alkyl, —NH—S(═O)₂—O—C₁₋₆-alkyl, —C₁₋₆-alkylene-NH—S(═O)₂—O—C₁₋₆-alkyl, —NH—S(═O)₂—NH₂, —C₁₋₆-alkylene-NH—S(═O)₂—NH₂, —NH—S(═O)₂—NH(C₁₋₆-alkyl), —C₁₋₆-alkylene-NH—S(═O)₂—NH(C₁₋₆-alkyl), —NH—S(═O)₂N(C₁₋₆-alkyl)₂, —C₁₋₆-alkylene-NH—S(═O)₂N(C₁₋₆-alkyl)₂, —N(C₁₋₆-alkyl)-S(═O)₂—OH, —C₁₋₆-alkylene-N(C₁₋₆-alkyl)-S(═O)₂—OH, —N(C₁₋₆-alkyl)-S(═O)₂—C₁₋₆-alkyl, —C₁₋₆-alkylene-N(C₁₋₆-alkyl)-S(═O)₂—C₁₋₆-alkyl, —N(C₁₋₆-alkyl)-S(═O)₂—O—C₁₋₆-alkyl, —C₁₋₆-alkylene-N(C₁₋₆-alkyl)-S(═O)₂—O—C₁₋₆-alkyl, —N(C₁₋₆-alkyl)-S(═O)₂—NH₂, —C₁₋₆-alkylene-N(C₁₋₆-alkyl)-S(═O)₂—NH₂, —N(C₁₋₆-alkyl)-S(═O)₂—NH(C₁₋₆-alkyl), —C₁₋₆-alkylene-N(C₁₋₆-alkyl)-S(═O)₂—NH(C₁₋₆-alkyl), —N(C₁₋₆-alkyl)-S(═O)₂—N(C₁₋₆-alkyl)₂, —C₁₋₆-alkylene-N(C₁₋₆-alkyl)-S(═O)₂—N(C₁₋₆-alkyl)₂, —SH, ═S, —SF₅, —SCF₃, —SCF₂H, —SCFH₂, —S—C₁₋₆-alkyl, —C₁₋₆-alkylene-S—C₁₋₆-alkyl, —S(═O)—C₁₋₆-alkyl, —C₁₋₆-alkylene-S(═O)—C₁₋₆-alkyl, —S(═O)₂—C₁₋₆-alkyl, —C₁₋₆-alkylene-S(═O)₂—C₁₋₆-alkyl, —S(═O)₂—OH, —C₁₋₆-alkylene-S(═O)₂—OH, —S(═O)₂—O—C₁₋₆-alkyl, —C₁₋₆-alkylene-S(═O)₂—O—C₁₋₆-alkyl, —S(═O)₂—NH₂, —C₁₋₆-alkylene-S(═O)₂—NH₂, —S(═O)₂—NH(C₁₋₆-alkyl), —C₁₋₆-alkylene-S(═O)₂—NH(C₁₋₆-alkyl), —S(═O)₂—N(C₁₋₆-alkyl)₂, —C₁₋₆-alkylene-S(═O)₂—N(C₁₋₆-alkyl)₂, 3-14-membered cycloalkyl, —C₁₋₆-alkylene-(3-14-membered cycloalkyl), 3 to 14-membered heterocycloalkyl, —C₁₋₆-alkylene-(3 to 14-membered heterocycloalkyl), -phenyl, —C₁₋₆-alkylene-phenyl, 5 to 14-membered heteroaryl, —C₁₋₆-alkylene-(5 to 14-membered heteroaryl), —O-(3-14-membered cycloalkyl), —O-(3 to 14-membered heterocycloalkyl), —O-phenyl, —O-(5 to 14-membered heteroaryl), —C(═O)-(3-14-membered cycloalkyl), —C(═O)-(3 to 14-membered heterocycloalkyl), —C(═O)-phenyl, —C(═O)-(5 to 14-membered heteroaryl), —S(═O)₂-(3-14-membered cycloalkyl), —S(═O)₂-(3 to 14-membered heterocycloalkyl), —S(═O)₂-phenyl, or —S(═O)₂-(5 to 14-membered heteroaryl);
or the compound is 5-(benzyloxy)-2-methylfuro[2,3-c]pyridine-3-carboxylate.
2. The compound according to statement 1, wherein T represents —O— and U represents —(CR⁵R⁵′)ₙ—.
3. The compound according to any one of statements 1 or 2, wherein R¹ represents
   —H, —F, —Cl, —Br, —I;
   —C₁₋₆-alkyl, saturated or unsaturated, unsubstituted, mono- or polysubstituted;
   —O—C₁₋₆-alkyl, saturated or unsaturated, unsubstituted, mono- or polysubstituted;
   —C(═O)C₁₋₆-alkyl, saturated or unsaturated, unsubstituted, mono- or polysubstituted;
   —C(═O)OC₁₋₆-alkyl, saturated or unsaturated, unsubstituted, mono- or polysubstituted;
   —C(═O)NH₂;
   —C(═O)NHC₁₋₆-alkyl, saturated or unsaturated, unsubstituted, mono- or polysubstituted;
   —C(═O)N(C₁₋₆-alkyl)₂, saturated or unsaturated, unsubstituted, mono- or polysubstituted;
   —S(═O)C₁₋₆-alkyl, saturated or unsaturated, unsubstituted, mono- or polysubstituted;
   —S(═O)₂—C₁₋₆-alkyl, saturated or unsaturated, unsubstituted, mono- or polysubstituted;
   —C₁-C₆-heteroalkyl, saturated or unsaturated, unsubstituted, mono- or polysubstituted; or
   3-14-membered cycloalkyl, saturated or unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted, in particular 3-14-membered cycloalkyl, saturated or non-aromatic unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted.
4. The compound according to any one of the preceding statements, wherein R¹ represents
   —H, —F, —Cl, —Br, —I;
   —C₁₋₆-alkyl, saturated or unsaturated, unsubstituted, mono- or polysubstituted;
   —O—C₁₋₆-alkyl, saturated or unsaturated, unsubstituted, mono- or polysubstituted;
   —C(═O)C₁₋₆-alkyl, saturated or unsaturated, unsubstituted, mono- or polysubstituted;
   —C(═O)OC₁₋₆-alkyl, saturated or unsaturated, unsubstituted, mono- or polysubstituted;
   —C(═O)NH₂;
   —C(═O)NHC₁₋₆-alkyl, saturated or unsaturated, unsubstituted, mono- or polysubstituted;
   —C(═O)N(C₁₋₆-alkyl)₂, saturated or unsaturated, unsubstituted, mono- or polysubstituted;
   —S(═O)C₁₋₆-alkyl, saturated or unsaturated, unsubstituted, mono- or polysubstituted;
   —S(═O)₂—C₁₋₆-alkyl, saturated or unsaturated, unsubstituted, mono- or polysubstituted;
   —C₁-C₆-heteroalkyl, saturated or unsaturated, unsubstituted, mono- or polysubstituted; or
   3-14-membered saturated cycloalkyl, 5-14-membered cycloalkenyl, 8-14-membered cycloalkynyl, unsubstituted, mono- or polysubstituted; wherein said 3-14-membered cycloalkyl, 5-14-membered cycloalkenyl or 8-14-membered cycloalkynyl, is optionally connected through —C₁-C₆-alkylene- or —C₁-C₆-heteroalkylene-, in each case saturated or unsaturated, unsubstituted, mono- or polysubstituted; preferably 3-12-membered saturated cycloalkyl, 5-12-membered cycloalkenyl or 8-12-membered cycloalkynyl unsubstituted, mono or polysubstituted, wherein said 3-12-membered cycloalkyl, 5-12-membered cycloalkenyl or 8-12-membered cycloalkynyl is optionally connected through —C₁-C₆-alkylene- or —C₁-C₆-heteroalkylene-, in each case saturated or unsaturated, unsubstituted, mono- or polysubstituted; preferably 3-10-membered saturated cycloalkyl, 5-10-membered cycloalkenyl or 8-10-membered cycloalkynyl, unsubstituted, mono or polysubstituted, wherein said 3-10-membered cycloalkyl, 5-10-membered cycloalkenyl or 8-10-membered cycloalkynyl is optionally connected through —C₁-C₆-alkylene- or —C₁-C₆-heteroalkylene-, in each case saturated or unsaturated, unsubstituted, mono- or polysubstituted; preferably 3-8-membered saturated cycloalkyl, 5-8-membered cycloalkenyl or 8-membered cycloalkynyl, unsubstituted, mono or polysubstituted, wherein said 3-8-membered cycloalkyl, 5-8-membered cycloalkenyl or 8-membered cycloalkynyl is optionally connected through —C₁-C₆-alkylene- or —C₁-C₆-heteroalkylene-, in each case saturated or unsaturated, unsubstituted, mono- or polysubstituted.
5. The compound according to any one of the preceding statements, wherein R¹ represents —H, —F, —Cl, —Br, —I, —CN, —C₁₋₆-alkyl, —O—C₁₋₆-alkyl, —C₁₋₆-alkylene-O—C₁₋₆-alkyl, —C₁₋₆-alkylene-NH(C₁₋₆-alkyl), —C₁₋₆-alkylene-N(C₁₋₆-alkyl)₂, —CF₃, —CF₂H, —CFH₂, —CF₂Cl, —CFCl₂, —C₁₋₆-alkylene-CF₃, —C₁₋₆-alkylene-CF₂H, —C₁₋₆-alkylene-CFH₂, —C₁₋₆-alkylene-NH—C₁₋₆-alkylene-CF₃, —C₁₋₆-alkylene-N(C₁₋₆-alkyl)-C₁₋₆-alkylene-CF₃, —C(═O)C₁₋₆-alkyl, —C(═O)OC₁₋₆-alkyl, —C(═O)NH₂; —C(═O)NHC₁₋₆-alkyl, —C(═O)N(C₁₋₆-alkyl)₂, —S(═O)—C₁₋₆-alkyl, —S(═O)₂—C₁₋₆-alkyl, —O—C₁₋₆-alkyl, -cyclopropyl unsubstituted, cyclobutyl unsubstituted, cyclopentyl unsubstituted or cyclohexyl unsubstituted.
6. The compound according to any one of the preceding statements, wherein R¹ represents —H, —C₁₋₆-alkyl, —C₁₋₆-alkylene-O—C₁₋₆-alkyl, —CHF₂, —CF₃, or -cyclopentyl, unsubstituted.
7. The compound according to any one of the preceding statements, wherein R³ represents
   —H;
   —OH;
   —C₁-C₆-alkyl, saturated or unsaturated, unsubstituted, mono- or poly substituted;
   —C₁-C₆-heteroalkyl, saturated or unsaturated, unsubstituted, mono- or poly substituted;
   3-14-membered cycloalkyl, saturated or unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted, in particular 3-14-membered cycloalkyl, saturated or non-aromatic unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted;
   3-14-membered heterocycloalkyl, saturated or unsaturated, unsubstituted, mono- or polysubstituted;
   wherein said 3-14-membered heterocycloalkyl is optionally connected through —C₁-C₆-alkylene- or —C₁-C₆-heteroalkylene-, in each case saturated or unsaturated, unsubstituted, mono- or polysubstituted; 6-14-membered aryl, unsubstituted, mono- or polysubstituted; wherein said 6-14-membered aryl is optionally connected through —C₁-C₆-alkylene- or —C₁-C₆-heteroalkylene-, in each case saturated or unsaturated, unsubstituted, mono- or polysubstituted; or
   5-14-membered heteroaryl, unsubstituted, mono- or polysubstituted; wherein said 5-14-membered heteroaryl is optionally connected through —C₁-C₆-alkylene- or —C₁-C₆-heteroalkylene-, in each case saturated or unsaturated, unsubstituted, mono- or polysubstituted.
8. The compound according to any one of the preceding statements, wherein R³ represents
   —H;
   —OH;
   —C₁-C₆-alkyl, saturated or unsaturated, unsubstituted, mono- or polysubstituted; or
   —C₁-C₆-heteroalkyl, saturated or unsaturated, unsubstituted, mono- or poly substituted.
9. The compound according to any one of the preceding statements, wherein R³ represents —H, —OH, —C₁₋₆-alkyl, —C₁₋₆-alkylene-OH, —C₁₋₆-alkylene-O—C₁₋₆-alkyl, —C₁₋₆-alkylene-NH₂, —C₁₋₆-alkylene-NH(C₁₋₆-alkyl), —C₁₋₆-alkylene-N(C₁₋₆-alkyl)₂, —CF₃, —CF₂H, —CFH₂, —CF₂Cl, —CFCl₂, —C₁₋₆-alkylene-CF₃, —C₁₋₆-alkylene-CF₂H, —C₁₋₆-alkylene-CFH₂, —C₁₋₆-alkylene-NH—C₁₋₆-alkylene-CF₃, or —C₁₋₆-alkylene-N(C₁₋₆-alkyl)-C₁₋₆-alkylene-CF₃.
10. The compound according to any one of the preceding statements, wherein R³ represents —H, —OH, or —C₁₋₆-alkyl, saturated, unsubstituted or monosubstituted with —OH.
11. The compound according to any one of the preceding statements, wherein R³ represents —H.
12. The compound according to any one of the preceding statements, wherein R⁴ represents
   —H;
   —S(═O)C₁₋₆-alkyl, saturated or unsaturated, unsubstituted, mono- or polysubstituted;
   —S(═O)₂—C₁₋₆-alkyl, saturated or unsaturated, unsubstituted, mono- or polysubstituted;
   —C₁-C₆-alkyl, saturated or unsaturated, unsubstituted, mono- or poly substituted;
   —C₁-C₆-heteroalkyl, saturated or unsaturated, unsubstituted, mono- or poly substituted;
   3-14-membered cycloalkyl, saturated or unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted, in particular 3-14-membered cycloalkyl, saturated or non-aromatic unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted;
   3-14-membered heterocycloalkyl, saturated or unsaturated, unsubstituted, mono- or polysubstituted;
   wherein said 3-14-membered heterocycloalkyl is optionally connected through —C₁-C₆-alkylene- or —C₁-C₆-heteroalkylene-, in each case saturated or unsaturated, unsubstituted, mono- or polysubstituted; 6-14-membered aryl, unsubstituted, mono- or polysubstituted; wherein said 6-14-membered aryl is optionally connected through —C₁-C₆-alkylene- or —C₁-C₆-heteroalkylene-, in each case saturated or unsaturated, unsubstituted, mono- or polysubstituted; or
   5-14-membered heteroaryl, unsubstituted, mono- or polysubstituted; wherein said 5-14-membered heteroaryl is optionally connected through —C₁-C₆-alkylene- or —C₁-C₆-heteroalkylene-, in each case saturated or unsaturated, unsubstituted, mono- or polysubstituted.
13. The compound according to any one of the preceding statements, wherein R⁴ represents
   —H;
   —S(═O)C₁₋₆-alkyl, saturated or unsaturated, unsubstituted, mono- or polysubstituted;
   —S(═O)₂—C₁₋₆-alkyl, saturated or unsaturated, unsubstituted, mono- or polysubstituted;
   —C₁-C₆-alkyl, saturated or unsaturated, unsubstituted, mono- or poly substituted;
   —C₁-C₆-heteroalkyl, saturated or unsaturated, unsubstituted, mono- or poly substituted;
   3-14-membered cycloalkyl, saturated or unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted, in particular 3-14-membered cycloalkyl, saturated or non-aromatic unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted;
   3-14-membered heterocycloalkyl, saturated or unsaturated, unsubstituted, mono- or polysubstituted;
   wherein said 3-14-membered heterocycloalkyl is optionally connected through —C₁-C₆-alkylene- or —C₁-C₆-heteroalkylene-, in each case saturated or unsaturated, unsubstituted, mono- or polysubstituted;
   —C₁₋₆-alkylene-phenyl unsubstituted, mono- or polysubstituted;
   -phenyl unsubstituted, mono- or polysubstituted; or
   5-14-membered heteroaryl, unsubstituted, mono- or polysubstituted; wherein said 5-14-membered heteroaryl is optionally connected through —C₁-C₆-alkylene- or —C₁-C₆-heteroalkylene-, in each case saturated or unsaturated, unsubstituted, mono- or polysubstituted.
14. The compound according to any one of the preceding statements, wherein R⁴ represents —S(═O)₂C₁₋₆-alkyl, saturated or unsaturated, unsubstituted, mono- or polysubstituted with substituents independently of one another selected from the group consisting of —F, —Cl, —C₁₋₆-alkyl, —C₁₋₆-alkylene-CF₃, —OH, ═O, —OC₁₋₆-alkyl, —C₁₋₆-alkylene-OH, —C₁₋₆-alkylene-O—C₁₋₆-alkyl, —NH₂, —NHC₁₋₆-alkyl, —N(C₁₋₆-alkyl)₂, —NHC(═O)O—C₁₋₆-alkyl, —N(C₁₋₆-alkyl)C(═O)O—C₁₋₆-alkyl, —C₁₋₆-alkylene-NHC(═O)O—C₁₋₆-alkyl, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —C(═O)N(C₁₋₆-alkyl)₂, —S(═O)₂C₁₋₆-alkyl, -phenyl, —C₁₋₆-alkylene-phenyl, 3-14-membered heterocycloalkyl, saturated or unsaturated, unsubstituted; and 5-14-membered heteroaryl, unsubstituted;
   —S(═O)₂(3-14-membered cycloalkyl), wherein said 3-14-membered cycloalkyl is selected from the group consisting of cyclopropyl, cyclobutyl, cyclopentyl, cyclohexyl, and cycloheptyl, in each case saturated or unsaturated, unsubstituted, mono- or polysubstituted with substituents independently of one another selected from the group consisting of —F, —Cl, —C₁₋₆-alkyl, —C₁₋₆-alkylene-CF₃, —OH, ═O, —OC₁₋₆-alkyl, —C₁₋₆-alkylene-OH, —C₁₋₆-alkylene-O—C₁₋₆-alkyl, —NH₂, —NHC₁₋₆-alkyl, —N(C₁₋₆-alkyl)₂, —NHC(═O)O—C₁₋₆-alkyl, —N(C₁₋₆-alkyl)C(═O)O—C₁₋₆-alkyl, —C₁₋₆-alkylene-NHC(═O)O—C₁₋₆-alkyl, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —C(═O)N(C₁₋₆-alkyl)₂, —S(═O)₂C₁₋₆-alkyl, -phenyl, —C₁₋₆-alkylene-phenyl, 3-14-membered heterocycloalkyl, saturated or unsaturated, unsubstituted; and 5-14-membered heteroaryl, unsubstituted;
   —C₁₋₆-alkyl, saturated or unsaturated, unsubstituted, mono- or polysubstituted with substituents independently of one another selected from the group consisting of —F, —Cl, —C₁₋₆-alkyl, —C₁₋₆-alkylene-CF₃, —OH, ═O, —OC₁₋₆-alkyl, —C₁₋₆-alkylene-OH, —C₁₋₆-alkylene-O—C₁₋₆-alkyl, —NH₂, —NHC₁₋₆-alkyl, —N(C₁₋₆-alkyl)₂, —NHC(═O)O—C₁₋₆-alkyl, —N(C₁₋₆-alkyl)C(═O)O—C₁₋₆-alkyl, —C₁₋₆-alkylene-NHC(═O)O—C₁₋₆-alkyl, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —C(═O)N(C₁₋₆-alkyl)₂, —S(═O)₂C₁₋₆-alkyl, -phenyl, —C₁₋₆-alkylene-phenyl, 3-14-membered heterocycloalkyl, saturated or unsaturated, unsubstituted; and 5-14-membered heteroaryl, unsubstituted;
   —C₁-C₆-heteroalkyl, saturated or unsaturated, unsubstituted, mono- or polysubstituted with substituents independently of one another selected from the group consisting of —F, —Cl, —C₁₋₆-alkyl, —C₁₋₆-alkylene-CF₃, —OH, ═O, —OC₁₋₆-alkyl, —C₁₋₆-alkylene-OH, —C₁₋₆-alkylene-O—C₁₋₆-alkyl, —NH₂, —NHC₁₋₆-alkyl, —N(C₁₋₆-alkyl)₂, —NHC(═O)O—C₁₋₆-alkyl, —N(C₁₋₆-alkyl)C(═O)O—C₁₋₆-alkyl, —C₁₋₆-alkylene-NHC(═O)O—C₁₋₆-alkyl, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —C(═O)N(C₁₋₆-alkyl)₂, —S(═O)₂C₁₋₆-alkyl, -phenyl, —C₁₋₆-alkylene-phenyl, 3-14-membered heterocycloalkyl, saturated or unsaturated, unsubstituted; and 5-14-membered heteroaryl, unsubstituted;
   3-14-membered cycloalkyl or —C₁₋₆-alkylene-(3-14-membered cycloalkyl), wherein —C₁₋₆-alkylene- is unsubstituted or monosubstituted with —OH, wherein said 3-14-membered cycloalkyl is selected from the group consisting of cyclopropyl, cyclobutyl, cyclopentyl, cyclohexyl, and cycloheptyl, in each case saturated or unsaturated, in each case unsubstituted, mono- or polysubstituted with substituents independently of one another selected from the group consisting of —F, —Cl, —C₁₋₆-alkyl, —C₁₋₆-alkylene-CF₃, —OH, ═O, —OC₁₋₆-alkyl, —C₁₋₆-alkylene-OH, —C₁₋₆-alkylene-O—C₁₋₆-alkyl, —NH₂, —NHC₁₋₆-alkyl, —N(C₁₋₆-alkyl)₂, —NHC(═O)O—C₁₋₆-alkyl, —N(C₁₋₆-alkyl)C(═O)O—C₁₋₆-alkyl, —C₁₋₆-alkylene-NHC(═O)O—C₁₋₆-alkyl, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —C(═O)N(C₁₋₆-alkyl)₂, —S(═O)₂C₁₋₆-alkyl, -phenyl, —C₁₋₆-alkylene-phenyl, 3-14-membered heterocycloalkyl, saturated or unsaturated, unsubstituted; and 5-14-membered heteroaryl, unsubstituted;
   3-14-membered heterocycloalkyl or —C₁₋₆-alkylene-(3-14-membered heterocycloalkyl), wherein —C₁₋₆-alkylene- is unsubstituted or monosubstituted with —OH, wherein said 3-14-membered heterocycloalkyl in each case is selected from the group consisting of azepane, 1,4-oxazepane, azetane, azetidine, aziridine, azocane, diazepane, dioxane, dioxolane, dithiane, dithiolane, imidazolidine, isothiazolidine, isoxalidine, morpholine, oxazolidine, oxepane, oxetane, oxirane, piperazine, piperidine, pyrazolidine, pyrrolidine, quinuclidine, tetrahydrofuran, tetrahydropyran, tetrahydrothiopyran, thiazolidine, thietane, thiirane, thiolane, thiomorpholine, indoline, dihydrobenzofuran, dihydrobenzothiophene, 1,1-dioxothiacyclohexane, 2-azaspiro[3.3]heptane, 2-oxaspiro[3.3]heptane, 7-azaspiro[3.5]nonane, 8-azabicyclo[3.2.1]octane, 9-azabicyclo[3.3.1]nonane, hexahydro-1H-pyrrolizine, hexahydrocyclopenta[c]pyrrole, octahydrocyclopenta[c]pyrrole, and octahydropyrrolo[1,2-a]pyrazin; in each case unsubstituted, mono- or polysubstituted with substituents independently of one another selected from the group consisting of —F, —Cl, —C₁₋₆-alkyl, —C₁₋₆-alkylene-CF₃, —OH, ═O, —OC₁₋₆-alkyl, —C₁₋₆-alkylene-OH, —C₁₋₆-alkylene-O—C₁₋₆-alkyl, —NH₂, —NHC₁₋₆-alkyl, —N(C₁₋₆-alkyl)₂, —NHC(═O)O—C₁₋₆-alkyl, —N(C₁₋₆-alkyl)C(═O)O—C₁₋₆-alkyl, —C₁₋₆-alkylene-NHC(═O)O—C₁₋₆-alkyl, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —C(═O)N(C₁₋₆-alkyl)₂, —S(═O)₂C₁₋₆-alkyl, -phenyl, —C₁₋₆-alkylene-phenyl, 3-14-membered heterocycloalkyl, saturated or unsaturated, unsubstituted; and 5-14-membered heteroaryl, unsubstituted;
   —C₁₋₆-alkylene-phenyl unsubstituted, mono- or polysubstituted with substituents independently of one another selected from the group consisting of —F, —Cl, —CN, —C₁₋₆-alkyl, —C₁₋₆-alkylene-CF₃, —OH, ═O, —OC₁₋₆-alkyl, —C₁₋₆-alkylene-OH, —C₁₋₆-alkylene-O—C₁₋₆-alkyl, —NH₂, —NHC₁₋₆-alkyl, —N(C₁₋₆-alkyl)₂, —NHC(═O)O—C₁₋₆-alkyl, —N(C₁₋₆-alkyl)C(═O)O—C₁₋₆-alkyl, —C₁₋₆-alkylene-NHC(═O)O—C₁₋₆-alkyl, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —C(═O)N(C₁₋₆-alkyl)₂, —S(═O)₂C₁₋₆-alkyl, -phenyl, —C₁₋₆-alkylene-phenyl, 3-14-membered heterocycloalkyl, saturated or unsaturated, unsubstituted; and 5-14-membered heteroaryl, unsubstituted;
   -phenyl unsubstituted, mono- or polysubstituted with substituents independently of one another selected from the group consisting of —F, —Cl, —CN, —C₁₋₆-alkyl, —C₁₋₆-alkylene-CF₃, —OH, ═O, —OC₁₋₆-alkyl, —C₁₋₆-alkylene-OH, —C₁₋₆-alkylene-O—C₁₋₆-alkyl, —NH₂, —NHC₁₋₆-alkyl, —N(C₁₋₆-alkyl)₂, —NHC(═O)O—C₁₋₆-alkyl, —N(C₁₋₆-alkyl)C(═O)O—C₁₋₆-alkyl, —C₁₋₆-alkylene-NHC(═O)O—C₁₋₆-alkyl, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —C(═O)N(C₁₋₆-alkyl)₂, —S(═O)₂C₁₋₆-alkyl, -phenyl, —C₁₋₆-alkylene-phenyl, 3-14-membered heterocycloalkyl, saturated or unsaturated, unsubstituted; and 5-14-membered heteroaryl, unsubstituted;
   5-14-membered heteroaryl or —C₁₋₆-alkylene-(5-14-membered heteroaryl), wherein —C₁₋₆-alkylene- is unsubstituted or monosubstituted with —OH, wherein said 5-14-membered heteroaryl in each case is selected from the group consisting of benzimidazole, benzisoxazole, benzoxazole, benzodioxole, benzofuran, benzothiadiazole, benzothiazole, benzothiophene, carbazole, cinnoline, dibenzofuran, furan, furazan, imidazole, imidazopyridine, indazole, indole, indolizine, isobenzofuran, isoindole, isoquinoline, isothiazole, isoxazole, naphthyridine, oxadiazole, oxazole, oxindole, phthalazine, purine, pyrazine, pyrazole, pyridazine, pyridine, pyrimidine, pyrrole, quinazoline, quinoline, quinoxaline, tetrazole, thiadiazole, thiazole, thiophene, triazine, triazole, and [1,2,4]triazolo[4,3-a]pyrimidine; in each case unsubstituted, mono- or polysubstituted with substituents independently of one another selected from the group consisting of —F, —Cl, —CN, —C₁₋₆-alkyl, —C₁₋₆-alkylene-CF₃, —OH, ═O, —OC₁₋₆-alkyl, —C₁₋₆-alkylene-OH, —C₁₋₆-alkylene-O—C₁₋₆-alkyl, —NH₂, —NHC₁₋₆-alkyl, —N(C₁₋₆-alkyl)₂, —NHC(═O)O—C₁₋₆-alkyl, —N(C₁₋₆-alkyl)C(═O)O—C₁₋₆-alkyl, —C₁₋₆-alkylene-NHC(═O)O—C₁₋₆-alkyl, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —C(═O)N(C₁₋₆-alkyl)₂, —S(═O)₂C₁₋₆-alkyl, -phenyl, —C₁₋₆-alkylene-phenyl, 3-14-membered heterocycloalkyl, saturated or unsaturated, unsubstituted; and 5-14-membered heteroaryl, unsubstituted.
15. The compound according to any one of the preceding statements, wherein R⁴ represents
   —H;
   —S(═O)₂C₁₋₆-alkyl, saturated, unsubstituted, monosubstituted or polysubstituted with —F;
   —S(═O)₂(3-14-membered cycloalkyl), saturated, unsubstituted;
   —C₁₋₆-alkyl, saturated, unsubstituted, mono- or polysubstituted with substituents independently of one another selected from the group consisting of —F, —Cl, —Br, —I, —CF₃, —CF₂H, —CFH₂, —CF₂Cl, —CFCl₂, —C₁₋₆-alkylene-CF₃, —C(═O)—C₁₋₆-alkyl, —C(═O)OH, —C(═O)—OC₁₋₆-alkyl, —C(═O)O—C₁₋₆-alkylene-CF₃, —OH, ═O, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C₁₋₆-alkylene-OH, —C₁₋₆-alkylene-O—C₁₋₆-alkyl, —C₁₋₆-alkylene-NHC(═O)O—C₁₋₆-alkyl, —NHC(═O)O—C₁₋₆-alkyl; —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂, 3-14-membered cycloalkyl, -phenyl unsubstituted, oxetanyl, or pyrimidinyl;
   3-14-membered cycloalkyl or —C₁₋₆-alkylene-(3-14-membered cycloalkyl), wherein —C₁₋₆-alkylene- is unsubstituted or mono- or polysubstituted with —F, —Cl, —Br, —I, —OH, wherein said 3-14-membered cycloalkyl is saturated, unsubstituted, monosubstituted or disubstituted with substituents independently of one another selected from the group consisting of —F, —Cl, —Br, —I, —CF₃, —CF₂H, —CFH₂, —CF₂Cl, —CFCl₂, —C₁₋₆-alkylene-CF₃, —C(═O)—C₁₋₆-alkyl, —C(═O)OH, —C(═O)—OC₁₋₆-alkyl, —C(═O)O—C₁₋₆-alkylene-CF₃, —OH, ═O, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C₁₋₆-alkylene-OH, —C₁₋₆-alkylene-O—C₁₋₆-alkyl, —C₁₋₆-alkylene-NHC(═O)O—C₁₋₆-alkyl, —NHC(═O)O—C₁₋₆-alkyl, —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂, 3-14-membered cycloalkyl, -phenyl unsubstituted, oxetanyl, or pyrimidinyl;
   3-14-membered heterocycloalkyl or —C₁₋₆-alkylene-(3-14-membered heterocycloalkyl), wherein —C₁₋₆-alkylene- is unsubstituted or mono- or polysubstituted with —F, —Cl, —Br, —I, —OH, wherein said 3-14-membered heterocycloalkyl in each case is selected from azetane, 1,4-oxazepane, pyrrolidine, piperidine, azepane, diazepane, tetrahydrofuran, tetrahydropyran, oxetane, morpholine, piperazine, hexahydrocyclopenta[c]pyrrole, octahydrocyclopenta[c]pyrrole, octahydropyrrolo[1,2-a]pyrazin, 8-azabicyclo[3.2.1]octane, 9-azabicyclo[3.3.1]nonane, quinuclidine, hexahydro-1H-pyrrolizine, 2-oxaspiro[3.3]heptane, 2-azaspiro[3.3]heptane, 7-azaspiro[3.5]nonane, 1,1-dioxothiacyclohexane, in each case unsubstituted, mono- or polysubstituted with substituents independently of one another selected from the group consisting of —F, —Cl, —Br, —I, —C₁₋₆-alkyl, —CF₃, —CF₂H, —CFH₂, —CF₂Cl, —CFCl₂, —C₁₋₆-alkylene-CF₃, —C(═O)—C₁₋₆-alkyl, —C(═O)OH, —C(═O)—OC₁₋₆-alkyl, —C(═O)O—C₁₋₆-alkylene-CF₃, —OH, ═O, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C₁₋₆-alkylene-OH, —C₁₋₆-alkylene-O—C₁₋₆-alkyl, —C₁₋₆-alkylene-NHC(═O)O—C₁₋₆-alkyl, —NHC(═O)O—C₁₋₆-alkyl, —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂, 3-14-membered heterocycloalkyl, -phenyl unsubstituted, 5 to 14-membered heteroaryl;
   5-14-membered heteroaryl or —C₁₋₆-alkylene-(5-14-membered heteroaryl), wherein —C₁₋₆-alkylene- is unsubstituted or monosubstituted with —F, —Cl, —Br, —I, —OH, wherein said 5-14-membered heteroaryl in each case is selected from the group consisting of pyridine, pyridazine, pyrazine, pyrazole, isoxazole, triazole, and [1,2,4]triazolo[4,3-a]pyrimidine, in each case unsubstituted, monosubstituted or disubstituted with substituents independently of one another selected from the group consisting of —F, —Cl, —Br, —I, —C₁₋₆-alkyl, —CF₃, —CF₂H, —CFH₂, —CF₂Cl, —CFCl₂, —C₁₋₆-alkylene-CF₃, —C(═O)—C₁₋₆-alkyl, —C(═O)OH, —C(═O)—OC₁₋₆-alkyl, —C(═O)O—C₁₋₆-alkylene-CF₃, —OH, ═O, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C₁₋₆-alkylene-OH, —C₁₋₆-alkylene-O—C₁₋₆-alkyl, —C₁₋₆-alkylene-NHC(═O)O—C₁₋₆-alkyl, —NHC(═O)O—C₁₋₆-alkyl, —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂, 3-14-membered cycloalkyl, 3-14-membered heterocycloalkyl, -phenyl unsubstituted, 5 to 14-membered heteroaryl.
16. The compound according to any one of the preceding statements, wherein R⁴ represents
   —H;
   —S(═O)₂C₁₋₆-alkyl, saturated, unsubstituted, monosubstituted or polysubstituted with —F;
   —S(═O)₂(3-14-membered cycloalkyl), saturated, unsubstituted;
   —C₁₋₆-alkyl, saturated, unsubstituted, monosubstituted or disubstituted with substituents independently of one another selected from the group consisting of —OH, —OC₁₋₆-alkyl, —N(C₁₋₆-alkyl)₂, —C₁₋₆-alkylene-NH₂, —C₁₋₆-alkylene-NH—C₁₋₆-alkyl, -phenyl unsubstituted;
   3-14-membered cycloalkyl or —C₁₋₆-alkylene-(3-14-membered cycloalkyl), wherein —C₁₋₆-alkylene- is unsubstituted or monosubstituted with —OH, wherein said 3-14-membered cycloalkyl is saturated, unsubstituted, monosubstituted or disubstituted with substituents independently of one another selected from the group consisting of —C₁₋₆-alkyl, —C₁₋₆-alkylene-NH₂, —C₁₋₆-alkylene-NH—C₁₋₆-alkylene-CF₃, —C₁₋₆-alkylene-OH, —C₁₋₆-alkylene-NHC(═O)O—C₁₋₆-alkyl, —OH, —OC₁₋₆-alkyl, —NH₂, —N(C₁₋₆-alkyl)₂, —NHC(═O)O—C₁₋₆-alkyl;
   3-14-membered heterocycloalkyl or —C₁₋₆-alkylene-(3-14-membered heterocycloalkyl), wherein —C₁₋₆-alkylene- is unsubstituted or monosubstituted with —OH, wherein said 3-14-membered heterocycloalkyl in each case is selected from azetane, 1,4-oxazepane, pyrrolidine, piperidine, azepane, diazepane, tetrahydrofuran, tetrahydropyran, oxetane, morpholine, piperazine, hexahydrocyclopenta[c]pyrrole, octahydrocyclopenta[c]pyrrole, octahydropyrrolo[1,2-a]pyrazin, 8-azabicyclo[3.2.1]octane, 9-azabicyclo-[3.3.1]nonane, quinuclidine, hexahydro-1H-pyrrolizine, 2-oxaspiro[3.3]heptane, 2-azaspiro[3.3]heptane, 7-azaspiro[3.5]nonane, 1,1-dioxothiacyclohexane, in each case unsubstituted, mono- or polysubstituted with substituents independently of one another selected from the group consisting of —F, —OH, ═O, —C₁₋₆-alkyl, —C₁₋₆-alkylene-CF₃, —C₁₋₆-alkylene-OH, —C₁₋₆-alkylene-O—C₁₋₆-alkyl, —NH₂, —N(C₁₋₆-alkyl)₂, —C₁₋₆-alkylene-NH₂, —C₁₋₆-alkylene-N(C₁₋₆-alkyl)₂, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —S(═O)₂C₁₋₆-alkyl, oxetanyl, pyrimidinyl, —C₁₋₆-alkylene-phenyl;
   phenyl unsubstituted;
   5-14-membered heteroaryl or —C₁₋₆-alkylene-(5-14-membered heteroaryl), wherein —C₁₋₆-alkylene- is unsubstituted or monosubstituted with —OH, wherein said 5-14-membered heteroaryl in each case is selected from the group consisting of pyridine, pyridazine, pyrazine, pyrazole, isoxazole, triazole, and [1,2,4]triazolo[4,3-a]pyrimidine, in each case unsubstituted, monosubstituted or disubstituted with substituents independently of one another selected from the group consisting of —C₁₋₆-alkyl, —OH.
17. The compound according to any one of the preceding statements, wherein
   R³ represent —H; and
   R⁴ represents
   —H;
   —S(═O)₂C₁₋₆-alkyl, saturated, unsubstituted, monosubstituted or polysubstituted with —F;
   —S(═O)₂(3-14-membered cycloalkyl), saturated, unsubstituted;
   —C₁₋₆-alkyl, saturated, unsubstituted, monosubstituted or disubstituted with substituents independently of one another selected from the group consisting of —OH, ═O, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-NH—C₁₋₆-alkyl, —C(═O)NH₂, —C(═O)—NH—C₁₋₃-alkyl, —C(═O)—N(C₁₋₃-alkyl)₂, or -phenyl unsubstituted;
   3-14-membered cycloalkyl or —C₁₋₆-alkylene-(3-14-membered cycloalkyl), wherein —C₁₋₆-alkylene- is unsubstituted or monosubstituted with —OH, wherein said 3-14-membered cycloalkyl is saturated, unsubstituted, monosubstituted or disubstituted with substituents independently of one another selected from the group consisting of —C₁₋₆-alkyl, —C₁₋₆-alkylene-NH₂, —C₁₋₆-alkylene-NH—C₁₋₆-alkylene-CF₃, —C₁₋₆-alkylene-OH, —C₁₋₆-alkylene-NHC(═O)O—C₁₋₆-alkyl, —OH, —OC₁₋₆-alkyl, —NH₂, —N(C₁₋₆-alkyl)₂, —NHC(═O)O—C₁₋₆-alkyl;
   3-14-membered heterocycloalkyl or —C₁₋₆-alkylene-(3-14-membered heterocycloalkyl), wherein —C₁₋₆-alkylene- is unsubstituted or monosubstituted with —OH, wherein said 3-14-membered heterocycloalkyl in each case is selected from azetane, 1,4-oxazepane, pyrrolidine, piperidine, azepane, diazepane, tetrahydrofuran, tetrahydropyran, oxetane, morpholine, piperazine, hexahydrocyclopenta[c]pyrrole, octahydrocyclopenta[c]pyrrole, octahydropyrrolo[1,2-a]pyrazin, 8-azabicyclo[3.2.1]octane, 9-azabicyclo-[3.3.1]nonane, quinuclidine, hexahydro-1H-pyrrolizine, 2-oxaspiro[3.3]heptane, 2-azaspiro[3.3]heptane, 7-azaspiro[3.5]nonane, 1,1-dioxothiacyclohexane, in each case unsubstituted, mono- or polysubstituted with substituents independently of one another selected from the group consisting of —F, —OH, ═O, —C₁₋₆-alkyl, —C₁₋₆-alkylene-CF₃, —C₁₋₆-alkylene-OH, —C₁₋₆-alkylene-O—C₁₋₆-alkyl, —NH₂, —N(C₁₋₆-alkyl)₂, —C₁₋₆-alkylene-NH₂, —C₁₋₆-alkylene-N(C₁₋₆-alkyl)₂, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —S(═O)₂C₁₋₆-alkyl, oxetanyl, pyrimidinyl, —C₁₋₆-alkylene-phenyl;
   -phenyl unsubstituted;
   5-14-membered heteroaryl or —C₁₋₆-alkylene-(5-14-membered heteroaryl), wherein —C₁₋₆-alkylene- is unsubstituted or monosubstituted with —OH, wherein said 5-14-membered heteroaryl in each case is selected from the group consisting of pyridine, pyridazine, pyrazine, pyrazole, isoxazole, triazole, and [1,2,4]triazolo[4,3-a]pyrimidine, in each case unsubstituted, monosubstituted or disubstituted with substituents independently of one another selected from the group consisting of —C₁₋₆-alkyl, —OH.
18. The compound according to any one of the preceding statements, wherein R³ and R⁴ together form a 5- or 6-membered heterocycle containing 1 or 2 heteroatoms selected from N, O and S, saturated or unsaturated, unsubstituted or mono- or poly substituted.
19. The compound according to any one of the preceding statements, wherein R³ and R⁴ together form a heterocycle selected from the group consisting of pyrrolidine, piperidine, morpholine, and piperazine, in each case unsubstituted, mono- or polysubstituted with substituents independently of one another selected from the group consisting of —C₁₋₆-alkyl, —NH₂, —NHCH₃, —N(CH₃)₂, —C(═O)NH—C₁₋₆-alkyl, —C(═O)N(C₁₋₆-alkyl)₂, —C(═O)O—C₁₋₆-alkyl, —NHC(═O)O—C₁₋₆-alkyl, -pyridyl unsubstituted, and 1,2,4-oxadiazole unsubstituted or monosubstituted with —C₁₋₆-alkyl.
20. The compound according to any one of the preceding statements, wherein R³ and R⁴ together form a pyrrolidine ring, unsubstituted or monosubstituted with —N(CH₃)₂;
   piperidine ring, unsubstituted or monosubstituted with a substituent selected from the group consisting of —C₁₋₆-alkyl, —NH₂, —N(CH₃)₂, —C(═O)NH—C₁₋₆-alkyl, —C(═O)O—C₁₋₆-alkyl, —NHC(═O)O—C₁₋₆-alkyl, and 1,2,4-oxadiazole unsubstituted or monosubstituted with —C₁₋₆-alkyl;
   morpholine ring, unsubstituted; or
   piperazine ring, unsubstituted or N-substituted with a substituent selected from the group consisting of —C₁₋₆-alkyl and -pyridyl unsubstituted.
21. The compound according to any one of the preceding statements, wherein R³ and R⁴ together with the nitrogen atom to which they are attached form a residue selected from the group consisting of:

| | | | |
|---|---|---|---|
| 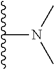 [SMILES: *N(C)C] | 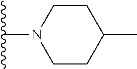 [SMILES: *N1CCC(C)CC1] | 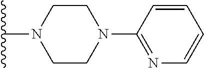 [SMILES: *N1CCN(c2ccccn2)CC1] | 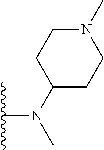 [SMILES: *N(C)C1CCN(C)CC1] |
| 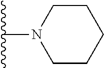 [SMILES: *N1CCCCC1] | 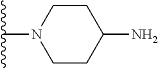 [SMILES: *N1CCC(N)CC1] | 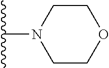 [SMILES: *N1CCOCC1] | 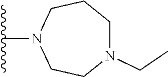 [SMILES: *N1CCCN(CC)CC1] |
| 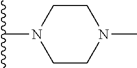 [SMILES: *N1CCN(C)CC1] | 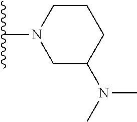 [SMILES: *N1CCCC(N(C)C)C1] | 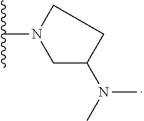 [SMILES: *N1CCC(N(C)C)C1] | |

22. The compound according to any one of the preceding statements, wherein W represents
   3-14-membered cycloalkyl, saturated or unsaturated, unsubstituted, mono- or disubstituted;
   6-14-membered aryl, unsubstituted, mono- or polysubstituted;
   3-14-membered heterocycloalkyl, saturated or unsaturated, mono- or polysubstituted;
   5-14-membered heteroaryl, mono- or polysubstituted; or
   —C₁-C₆-alkyl, —C₂-C₆-alkenyl, —C₂-C₆-alkynyl; in each case unsubstituted, mono- or disubstituted; in particular W represents
   3-14-membered cycloalkyl, saturated or non-aromatic unsaturated, unsubstituted, mono- or disubstituted;
   6-14-membered aryl, unsubstituted, mono- or polysubstituted;
   3-14-membered heterocycloalkyl, saturated or unsaturated, mono- or polysubstituted;
   5-14-membered heteroaryl, mono- or polysubstituted; or
   —C₁-C₆-alkyl, —C₂-C₆-alkenyl, —C₂-C₆-alkynyl; in each case unsubstituted, mono- or disubstituted.
23. The compound according to any one of the preceding statements, wherein W represents
   3-14-membered saturated cycloalkyl, 5-14-membered cycloalkenyl, 8-14-membered cycloalkynyl, unsubstituted, mono- or disubstituted;
   6-14-membered aryl, unsubstituted, mono- or polysubstituted;
   3-14-membered heterocycloalkyl, saturated or unsaturated, mono- or polysubstituted;
   5-14-membered heteroaryl, mono- or polysubstituted; or
   —C₁-C₆-alkyl, —C₂-C₆-alkenyl, —C₂-C₆-alkynyl; in each case unsubstituted, mono- or disubstituted.
24. The compound according to any one of the preceding statements, wherein W represents
   3-14-membered cycloalkyl, saturated or unsaturated, unsubstituted, mono- or polysubstituted with substituents independently selected from —F, —Cl, —Br, —I, —C₁₋₆-alkyl, —CF₃, —CF₂H, —CFH₂, —CF₂Cl, —CFCl₂, —C₁₋₆-alkylene-CF₃, —C(═O)—C₁₋₆-alkyl, —C(═O)OH, —C(═O)—OC₁₋₆-alkyl, —C(═O)O—C₁₋₆-alkylene-CF₃, —OH, ═O, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C₁₋₆-alkylene-OH, —C₁₋₆-alkylene-O—C₁₋₆-alkyl, —C₁₋₆-alkylene-NHC(═O)O—C₁₋₆-alkyl, —NHC(═O)O—C₁₋₆-alkyl, —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂, 3-14-membered cycloalkyl, 3-14-membered heterocycloalkyl, -phenyl unsubstituted, 5 to 14-membered heteroaryl, in particular 3-14-membered cycloalkyl, saturated or non-aromatic unsaturated, unsubstituted, mono- or polysubstituted with substituents independently selected from —F, —Cl, —Br, —I, —C₁₋₆-alkyl, —CF₃, —CF₂H, —CFH₂, —CF₂Cl, —CFCl₂, —C₁₋₆-alkylene-CF₃, —C(═O)—C₁₋₆-alkyl, —C(═O)OH, —C(═O)—OC₁₋₆-alkyl, —C(═O)O—C₁₋₆-alkylene-CF₃, —OH, ═O, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C₁₋₆-alkylene-OH, —C₁₋₆-alkylene-O—C₁₋₆-alkyl, —C₁₋₆-alkylene-NHC(═O)O—C₁₋₆-alkyl, —NHC(═O)O—C₁₋₆-alkyl, —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂, 3-14-membered cycloalkyl, 3-14-membered heterocycloalkyl, -phenyl unsubstituted, 5 to 14-membered heteroaryl;
   6-14-membered aryl, unsubstituted, mono- or polysubstituted independently selected from —F, —Cl, —Br, —I, —C₁₋₆-alkyl, —CF₃, —CF₂H, —CFH₂, —CF₂Cl, —CFCl₂, —C₁₋₆-alkylene-CF₃, —C(═O)—C₁₋₆-alkyl, —C(═O)OH, —C(═O)—OC₁₋₆-alkyl, —C(═O)O—C₁₋₆-alkylene-CF₃, —OH, ═O, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C₁₋₆-alkylene-OH, —C₁₋₆-alkylene-O—C₁₋₆-alkyl, —C₁₋₆-alkylene-NHC(═O)O—C₁₋₆-alkyl, —NHC(═O)O—C₁₋₆-alkyl, —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂, 3-14-membered cycloalkyl, 3-14-membered heterocycloalkyl, -phenyl unsubstituted, 5 to 14-membered heteroaryl;
   3-14-membered heterocycloalkyl, saturated or unsaturated, unsubstituted, mono- or polysubstituted independently selected from —F, —Cl, —Br, —I, —C₁₋₆-alkyl, —CF₃, —CF₂H, —CFH₂, —CF₂Cl, —CFCl₂, —C₁₋₆-alkylene-CF₃, —C(═O)—C₁₋₆-alkyl, —C(═O)OH, —C(═O)—OC₁₋₆-alkyl, —C(═O)O—C₁₋₆-alkylene-CF₃, —OH, ═O, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C₁₋₆-alkylene-OH, —C₁₋₆-alkylene-O—C₁₋₆-alkyl, —C₁₋₆-alkylene-NHC(═O)O—C₁₋₆-alkyl, —NHC(═O)O—C₁₋₆-alkyl, —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂, 3-14-membered cycloalkyl, 3-14-membered heterocycloalkyl, -phenyl unsubstituted, 5 to 14-membered heteroaryl;
   5-14-membered heteroaryl, unsubstituted, mono- or polysubstituted independently selected from —F, —Cl, —Br, —I, —C₁₋₆-alkyl, —CF₃, —CF₂H, —CFH₂, —CF₂Cl, —CFCl₂, —C₁₋₆-alkylene-CF₃, —C(═O)—C₁₋₆-alkyl, —C(═O)OH, —C(═O)—OC₁₋₆-alkyl, —C(═O)O—C₁₋₆-alkylene-CF₃, —OH, ═O, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C₁₋₆-alkylene-OH, —C₁₋₆-alkylene-O—C₁₋₆-alkyl, —C₁₋₆-alkylene-NHC(═O)O—C₁₋₆-alkyl, —NHC(═O)O—C₁₋₆-alkyl, —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂, 3-14-membered cycloalkyl, 3-14-membered heterocycloalkyl, -phenyl unsubstituted, 5 to 14-membered heteroaryl; or
   —C₁-C₆-alkyl, —C₂-C₆-alkenyl, —C₂-C₆-alkynyl; in each case unsubstituted, mono- or polysubstituted with substituents independently selected from —F, —Cl, —Br, —I, —C₁₋₆-alkyl, —CF₃, —CF₂H, —CFH₂, —CF₂Cl, —CFCl₂, —C₁₋₆-alkylene-CF₃, —C(═O)—C₁₋₆-alkyl, —C(═O)OH, —C(═O)—OC₁₋₆-alkyl, —C(═O)O—C₁₋₆-alkylene-CF₃, —OH, ═O, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C₁₋₆-alkylene-OH, —C₁₋₆-alkylene-O—C₁₋₆-alkyl, —C₁₋₆-alkylene-NHC(═O)O—C₁₋₆-alkyl, —NHC(═O)O—C₁₋₆-alkyl, —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂, 3-14-membered cycloalkyl, 3-14-membered heterocycloalkyl, -phenyl unsubstituted, 5 to 14-membered heteroaryl.
25. The compound according to any one of the preceding statements, wherein W represents
   3-10-membered cycloalkyl, saturated or unsaturated, unsubstituted, mono- or polysubstituted with substituents independently selected from —F, —Cl, —Br, —I, —C₁₋₆-alkyl, —CF₃, —CF₂H, —CFH₂, —CF₂Cl, —CFCl₂, —C₁₋₆-alkylene-CF₃, —C(═O)—C₁₋₆-alkyl, —C(═O)OH, —C(═O)—OC₁₋₆-alkyl, —C(═O)O—C₁₋₆-alkylene-CF₃, —OH, ═O, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C₁₋₆-alkylene-OH, —C₁₋₆-alkylene-O—C₁₋₆-alkyl, —C₁₋₆-alkylene-NHC(═O)O—C₁₋₆-alkyl, —NHC(═O)O—C₁₋₆-alkyl, —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂, 3-14-membered heterocycloalkyl, -phenyl unsubstituted, 5 to 14-membered heteroaryl, in particular 3-10-membered cycloalkyl, saturated or non-aromatic unsaturated, unsubstituted, mono- or polysubstituted with substituents independently selected from —F, —Cl, —Br, —I, —C₁₋₆-alkyl, —CF₃, —CF₂H, —CFH₂, —CF₂Cl, —CFCl₂, —C₁₋₆-alkylene-CF₃, —C(═O)—C₁₋₆-alkyl, —C(═O)OH, —C(═O)—OC₁₋₆-alkyl, —C(═O)O—C₁₋₆-alkylene-CF₃, —OH, ═O, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C₁₋₆-alkylene-OH, —C₁₋₆-alkylene-O—C₁₋₆-alkyl, —C₁₋₆-alkylene-NHC(═O)O—C₁₋₆-alkyl, —NHC(═O)O—C₁₋₆-alkyl, —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂, 3-14-membered heterocycloalkyl, -phenyl unsubstituted, 5 to 14-membered heteroaryl; or
   6-12-membered aryl, unsubstituted, mono- or polysubstituted independently selected from —F, —Cl, —Br, —I, —C₁₋₆-alkyl, —CF₃, —CF₂H, —CFH₂, —CF₂Cl, —CFCl₂, —C₁₋₆-alkylene-CF₃, —C(═O)—C₁₋₆-alkyl, —C(═O)OH, —C(═O)—OC₁₋₆-alkyl, —C(═O)O—C₁₋₆-alkylene-CF₃, —OH, ═O, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C₁₋₆-alkylene-OH, —C₁₋₆-alkylene-O—C₁₋₆-alkyl, —C₁₋₆-alkylene-NHC(═O)O—C₁₋₆-alkyl, —NHC(═O)O—C₁₋₆-alkyl, —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂, 3-14-membered cycloalkyl, 3-14-membered heterocycloalkyl, -phenyl unsubstituted, 5 to 14-membered heteroaryl;
   3-10-membered heterocycloalkyl, saturated or unsaturated, unsubstituted, mono- or polysubstituted independently selected from —F, —Cl, —Br, —I, —C₁₋₆-alkyl, —CF₃, —CF₂H, —CFH₂, —CF₂Cl, —CFCl₂, —C₁₋₆-alkylene-CF₃, —C(═O)—C₁₋₆-alkyl, —C(═O)OH, —C(═O)—OC₁₋₆-alkyl, —C(═O)O—C₁₋₆-alkylene-CF₃, —OH, ═O, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C₁₋₆-alkylene-OH, —C₁₋₆-alkylene-O—C₁₋₆-alkyl, —C₁₋₆-alkylene-NHC(═O)O—C₁₋₆-alkyl, —NHC(═O)O—C₁₋₆-alkyl, —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂, 3-14-membered cycloalkyl, 3-14-membered heterocycloalkyl, -phenyl unsubstituted, 5 to 14-membered heteroaryl;
   5-10-membered heteroaryl, unsubstituted, mono- or polysubstituted independently selected from —F, —Cl, —Br, —I, —C₁₋₆-alkyl, —CF₃, —CF₂H, —CFH₂, —CF₂Cl, —CFCl₂, —C₁₋₆-alkylene-CF₃, —C(═O)—C₁₋₆-alkyl, —C(═O)OH, —C(═O)—OC₁₋₆-alkyl, —C(═O)O—C₁₋₆-alkylene-CF₃, —OH, ═O, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C₁₋₆-alkylene-OH, —C₁₋₆-alkylene-O—C₁₋₆-alkyl, —C₁₋₆-alkylene-NHC(═O)O—C₁₋₆-alkyl, —NHC(═O)O—C₁₋₆-alkyl, —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂, 3-14-membered cycloalkyl, 3-14-membered heterocycloalkyl, -phenyl unsubstituted, 5 to 14-membered heteroaryl; or
   —C₁-C₆-alkyl, unsubstituted, mono- or polysubstituted with substituents independently selected from —F, —Cl, —Br, —I, —C₁₋₆-alkyl, —CF₃, —CF₂H, —CFH₂, —CF₂Cl, —CFCl₂, —C₁₋₆-alkylene-CF₃, —C(═O)—C₁₋₆-alkyl, —C(═O)OH, —C(═O)—OC₁₋₆-alkyl, —C(═O)O—C₁₋₆-alkylene-CF₃, —OH, ═O, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C₁₋₆-alkylene-OH, —C₁₋₆-alkylene-O—C₁₋₆-alkyl, —C₁₋₆-alkylene-NHC(═O)O—C₁₋₆-alkyl, —NHC(═O)O—C₁₋₆-alkyl, —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂, 3-14-membered heterocycloalkyl, -phenyl unsubstituted, 5 to 14-membered heteroaryl.
26. The compound according to any one of the preceding statements, wherein W represents
   3-14-membered cycloalkyl, saturated or unsaturated, unsubstituted, mono- or polysubstituted with substituents independently selected from —F, —Cl, —Br, —I, —C₁₋₆-alkyl, —CF₃, —CF₂H, —CFH₂, —CF₂Cl, —CFCl₂, —C₁₋₆-alkylene-CF₃, —C(═O)OH, —C(═O)—OC₁₋₆-alkyl, —OH, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-N(C₁₋₆-alkyl)₂, —C₁₋₆-alkylene-OH, —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂, in particular 3-14-membered cycloalkyl, saturated or non-aromatic unsaturated, unsubstituted, mono- or polysubstituted with substituents independently selected from —F, —Cl, —Br, —I, —C₁₋₆-alkyl, —CF₃, —CF₂H, —CFH₂, —CF₂Cl, —CFCl₂, —C₁₋₆-alkylene-CF₃, —C(═O)OH, —C(═O)—OC₁₋₆-alkyl, —OH, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-N(C₁₋₆-alkyl)₂, —C₁₋₆-alkylene-OH, —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂;
   6-14-membered aryl, unsubstituted, mono- or polysubstituted independently selected from —F, —Cl, —Br, —I, —C₁₋₆-alkyl, —CF₃, —CF₂H, —CFH₂, —CF₂Cl, —CFCl₂, —C₁₋₆-alkylene-CF₃, —C(═O)OH, —C(═O)—OC₁₋₆-alkyl, —OH, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-N(C₁₋₆-alkyl)₂, —C₁₋₆-alkylene-OH, —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂;
   3-14-membered heterocycloalkyl, saturated or unsaturated, unsubstituted, mono- or polysubstituted independently selected from —F, —Cl, —Br, —I, —C₁₋₆-alkyl, —CF₃, —CF₂H, —CFH₂, —CF₂Cl, —CFCl₂, —C₁₋₆-alkylene-CF₃, —C(═O)OH, —C(═O)—OC₁₋₆-alkyl, —OH, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-N(C₁₋₆-alkyl)₂, —C₁₋₆-alkylene-OH, —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂;
   5-14-membered heteroaryl, unsubstituted, mono- or polysubstituted independently selected from —F, —Cl, —Br, —I, —C₁₋₆-alkyl, —CF₃, —CF₂H, —CFH₂, —CF₂Cl, —CFCl₂, —C₁₋₆-alkylene-CF₃, —C(═O)OH, —C(═O)—OC₁₋₆-alkyl, —OH, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-N(C₁₋₆-alkyl)₂, —C₁₋₆-alkylene-OH, —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂; or
   —C₁-C₆-alkyl, —C₂-C₆-alkenyl, —C₂-C₆-alkynyl; in each case unsubstituted, mono- or polysubstituted with substituents independently selected from —F, —Cl, —Br, —I, —C₁₋₆-alkyl, —CF₃, —CF₂H, —CFH₂, —CF₂Cl, —CFCl₂, —C₁₋₆-alkylene-CF₃, —C(═O)OH, —C(═O)—OC₁₋₆-alkyl, —OH, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-N(C₁₋₆-alkyl)₂, —C₁₋₆-alkylene-OH, —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂.
27. The compound according to any one of the preceding statements, wherein W represents
   3-10-membered cycloalkyl, saturated, unsubstituted, mono- or disubstituted with substituents independently selected from —F, —Cl, —Br, —I, —C₁₋₆-alkyl, —CF₃, —CF₂H, —CFH₂, —CF₂Cl, —CFCl₂, —C₁₋₆-alkylene-CF₃, —C(═O)OH, —C(═O)—OC₁₋₆-alkyl, —OH, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-N(C₁₋₆-alkyl)₂, —C₁₋₆-alkylene-OH, —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂, 6-14-membered aryl, unsubstituted, mono- or polysubstituted independently selected from —F, —Cl, —Br, —I, —C₁₋₆-alkyl, —CF₃, —CF₂H, —CFH₂, —CF₂Cl, —CFCl₂, —C₁₋₆-alkylene-CF₃, —C(═O)OH, —C(═O)—OC₁₋₆-alkyl, —OH, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-N(C₁₋₆-alkyl)₂, —C₁₋₆-alkylene-OH, —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂;
   3-10-membered heterocycloalkyl, saturated unsubstituted, mono- or disubstituted independently selected from —F, —Cl, —Br, —I, —C₁₋₆-alkyl, —CF₃, —CF₂H, —CFH₂, —CF₂Cl, —CFCl₂, —C₁₋₆-alkylene-CF₃, —C(═O)OH, —C(═O)—OC₁₋₆-alkyl, —OH, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-N(C₁₋₆-alkyl)₂, —C₁₋₆-alkylene-OH, —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂; 5-10-membered heteroaryl, unsubstituted, mono- or disubstituted independently selected from —F, —Cl, —Br, —I, —C₁₋₆-alkyl, —CF₃, —CF₂H, —CFH₂, —CF₂Cl, —CFCl₂, —C₁₋₆-alkylene-CF₃, —C(═O)OH, —C(═O)—OC₁₋₆-alkyl, —OH, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-N(C₁₋₆-alkyl)₂, —C₁₋₆-alkylene-OH, —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂; or
   —C₁-C₆-alkyl, —C₂-C₆-alkenyl, —C₂-C₆-alkynyl; in each case unsubstituted, mono- or disubstituted with substituents independently selected from —F, —Cl, —Br, —I, —C₁₋₆-alkyl, —CF₃, —CF₂H, —CFH₂, —CF₂Cl, —CFCl₂, —C₁₋₆-alkylene-CF₃, —C(═O)OH, —C(═O)—OC₁₋₆-alkyl, —OH, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-N(C₁₋₆-alkyl)₂, —C₁₋₆-alkylene-OH, —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂.
28. The compound according to any one of the preceding statements, wherein R⁵ and R^{5′} independently of one another represent
   —H;
   —C₁-C₆-alkyl, saturated or unsaturated, unsubstituted, mono- or poly substituted;
   —C₁-C₆-heteroalkyl, saturated or unsaturated, unsubstituted, mono- or polysubstituted;
   3-14-membered cycloalkyl, saturated or unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted, in particular 3-14-membered cycloalkyl, saturated or non-aromatic unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted.
29. The compound according to any one of the preceding statements, wherein n is an integer selected from 1, 2 or 3; and
   R⁵ and R^{5′} independently of one another represent
   —H;
   —C₁-C₆-alkyl, saturated or unsaturated, unsubstituted, mono- or poly substituted;
   —C₁-C₆-heteroalkyl, saturated or unsaturated, unsubstituted, mono- or poly substituted;
   3-14-membered cycloalkyl, saturated or unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted, in particular 3-14-membered cycloalkyl, saturated or non-aromatic unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted.
30. The compound according to any one of the preceding statements, wherein R⁵ and R^{5′} independently of one another represent —H, —C₁-C₆-alkyl, or —C₁-C₆-alkylene-N(C₁-C₆-alkyl)₂.
31. The compound according to any one of the preceding statements, wherein R⁶ and R⁸ independently of one another represent
   —H;
   —F, —Cl, —Br, —I, —OH, —SH, —SF₅, —CN, —NO₂, —C(═O)OH, —NH₂;
   —C₁₋₆-alkyl, saturated or unsaturated, unsubstituted, mono- or polysubstituted;
   —O—C₁₋₆-alkyl, saturated or unsaturated, unsubstituted, mono- or polysubstituted;
   —NHC₁₋₆-alkyl, saturated or unsaturated, unsubstituted, mono- or polysubstituted;
   —N(C₁₋₆-alkyl)₂, saturated or unsaturated, unsubstituted, mono- or polysubstituted;
   —C(═O)OC₁₋₆-alkyl, saturated or unsaturated, unsubstituted, mono- or polysubstituted;
   —OC(═O)C₁₋₆-alkyl, saturated or unsaturated, unsubstituted, mono- or polysubstituted;
   —C₁₋₆-heteroalkyl, saturated or unsaturated, unsubstituted, mono- or polysubstituted.
32. The compound according to any one of the preceding statements, wherein R⁶ and R⁸ independently of one another represent
   —H, —F, —Cl, —Br, —I, —OH, —SH, —SF₅, —CN, —NO₂, —C(═O)OH, —NH₂,
   —C₁₋₆-alkyl, —CF₃, —CHF₂, —CH₂F,
   —O—C₁₋₆-alkyl, —OCF₃, —OCHF₂, —OCH₂F,
   —NHC₁₋₆-alkyl unsubstituted or substituted with one or more substituents independently of one another selected from —OH, ═O, —F, —Cl, —Br, —I, —SH, ═S, —CN, —CF₃, —CHF₂, —CH₂F, —OCF₃, —OCHF₂, —OCH₂F, SF₅, —NO₂, —C(═O)OH, —NH₂, and —C(═O)NH₂;
   —N(C₁₋₆-alkyl)₂ unsubstituted or substituted with one or more substituents independently of one another selected from —OH, ═O, —F, —Cl, —Br, —I, —SH, ═S, —CN, —CF₃, —CHF₂, —CH₂F, —OCF₃, —OCHF₂, —OCH₂F, SF₅, —NO₂, —C(═O)OH, —NH₂, and —C(═O)NH₂;
   —C(═O)OC₁₋₆-alkyl unsubstituted or substituted with one or more substituents independently of one another selected from —OH, ═O, —F, —Cl, —Br, —I, —SH, ═S, —CN, —CF₃, —CHF₂, —CH₂F, —OCF₃, —OCHF₂, —OCH₂F, SF₅, —NO₂, —C(═O)OH, —NH₂, and —C(═O)NH₂;
   —OC(═O)C₁₋₆-alkyl unsubstituted or substituted with one or more substituents independently of one another selected from —OH, ═O, —F, —Cl, —Br, —I, —SH, ═S, —CN, —CF₃, —CHF₂, —CH₂F, —OCF₃, —OCHF₂, —OCH₂F, SF₅, —NO₂, —C(═O)OH, —NH₂, and —C(═O)NH₂; or
   —C₁₋₆-heteroalkyl unsubstituted or substituted with one or more substituents independently of one another selected from —OH, ═O, —F, —Cl, —Br, —I, —SH, ═S, —CN, —CF₃, —CHF₂, —CH₂F, —OCF₃, —OCHF₂, —OCH₂F, SF₅, —NO₂, —C(═O)OH, —NH₂, and —C(═O)NH₂.
33. The compound according to any one of the preceding statements, wherein R⁶ represents —H, —F, —Cl, —CN, or —C₁-C₆-alkyl.
34. The compound according to any one of the preceding statements, wherein R⁶ does not represent —H.
35. The compound according to any one of the preceding statements, wherein R⁸ represents —H, —F, —Cl, —CN, or —C₁-C₆-alkyl.
36. The compound according to any one of the preceding statements, wherein R⁸ does not represent —H.
37. The compound according to any one of the preceding statements, wherein
   (i) R⁶ and R⁸ each represent —H; or
   (ii) R⁶ and R⁸ represent independently from each other —F, —Cl, —CN, or —CH₃; or
   (iii) one of R⁶ and R⁸ represents —H and the other of R⁶ and R⁸ represents —F, —Cl, —CN, or —CH₃.
38. The compound according to any one of the preceding statements, which is selected from the group consisting of Cpd 001 to Cpd 036 as mentioned herein or the physiologically acceptable salt thereof.
39. The compound according to any one of the preceding statements, wherein
   R¹ represents H, —CH₂F, —CHF₂, or —CF₃;
R³ represent —H;
R⁴ represents
   —C₁-C₆-alkyl, saturated or unsaturated, unsubstituted, mono- or poly substituted;
   3-14-membered cycloalkyl, saturated or unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted, in particular 3-14-membered cycloalkyl, saturated or non-aromatic unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted;
   3-14-membered heterocycloalkyl, saturated or unsaturated, unsubstituted, mono- or polysubstituted; wherein said 3-14-membered heterocycloalkyl is optionally connected through —C₁-C₆-alkylene- or —C₁-C₆-heteroalkylene-, in each case saturated or unsaturated, unsubstituted, mono- or polysubstituted; 6-14-membered aryl, unsubstituted, mono- or polysubstituted; wherein said 6-14-membered aryl is optionally connected through —C₁-C₆-alkylene- or —C₁-C₆-heteroalkylene-, in each case saturated or unsaturated, unsubstituted, mono- or polysubstituted; or
   5-14-membered heteroaryl, unsubstituted, mono- or polysubstituted; wherein said 5-14-membered heteroaryl is optionally connected through —C₁-C₆-alkylene- or —C₁-C₆-heteroalkylene-, in each case saturated or unsaturated, unsubstituted, mono- or polysubstituted;
   or R³ and R⁴ together form a 5- or 6-membered heterocycle containing 1 or 2 heteroatoms selected from N, O and S, saturated or unsaturated, unsubstituted or mono- or polysubstituted;
   R⁵ and R^{5′} independently of one another represent —H, —C₁-C₆-alkyl, or —C₁-C₆-alkylene-N(C₁-C₆-alkyl)₂;
   R⁶ and R⁸ independently of one another represents —H, —F, —Cl, —CN, or —C₁-C₆-alkyl;
   W represents
   3-14-membered cycloalkyl, saturated or unsaturated; unsubstituted, mono- or polysubstituted, in particular 3-14-membered cycloalkyl, saturated or non-aromatic unsaturated; unsubstituted, mono- or polysubstituted;
   6-14-membered aryl, unsubstituted, mono- or polysubstituted;
   3-14-membered heterocycloalkyl, saturated or unsaturated, mono- or polysubstituted;
   5-14-membered heteroaryl, mono- or polysubstituted; or
   —C₁-C₆-alkyl; unsubstituted, mono- or polysubstituted.
40. The compound according to any one of the preceding statements, wherein
   R¹ represents H, —CH₂F, —CHF₂, or —CF₃;
   R³ represent —H;
   R⁴ represents
   —C₁-C₆-alkyl, saturated or unsaturated, unsubstituted, mono- or poly substituted;
   3-14-membered saturated cycloalkyl, 5-14-membered cycloalkenyl, 8-14-membered cycloalkynyl, unsubstituted, mono- or polysubstituted; wherein each of said 3-14-membered cycloalkyl, 5-14-membered cycloalkenyl, 8-14-membered cycloalkynyl, is optionally connected through —C₁-C₆-alkylene- or —C₁-C₆-heteroalkylene-, in each case saturated or unsaturated, unsubstituted, mono- or polysubstituted;
   3-14-membered heterocycloalkyl, saturated or unsaturated, unsubstituted, mono- or polysubstituted; wherein said 3-14-membered heterocycloalkyl is optionally connected through —C₁-C₆-alkylene- or —C₁-C₆-heteroalkylene-, in each case saturated or unsaturated, unsubstituted, mono- or polysubstituted; 6-14-membered aryl, unsubstituted, mono- or polysubstituted; wherein said 6-14-membered aryl is optionally connected through —C₁-C₆-alkylene- or —C₁-C₆-heteroalkylene-, in each case saturated or unsaturated, unsubstituted, mono- or polysubstituted; or
   5-14-membered heteroaryl, unsubstituted, mono- or polysubstituted; wherein said 5-14-membered heteroaryl is optionally connected through —C₁-C₆-alkylene- or —C₁-C₆-heteroalkylene-, in each case saturated or unsaturated, unsubstituted, mono- or polysubstituted;
   or R³ and R⁴ together form a 5- or 6-membered heterocycle containing 1 or 2 heteroatoms selected from N, O and S, saturated or unsaturated, unsubstituted or mono- or polysubstituted;
   R⁵ and R^{5′} independently of one another represent —H, —C₁-C₆-alkyl, or —C₁-C₆-alkylene-N(C₁-C₆-alkyl)₂;
   R⁶ and R⁸ independently of one another represents —H, —F, —Cl, —CN, or —C₁-C₆-alkyl;
   W represents
   3-14-membered saturated cycloalkyl, 5-14-membered cycloalkenyl, 8-14-membered cycloalkynyl, unsubstituted, mono- or polysubstituted;
   6-14-membered aryl, unsubstituted, mono- or polysubstituted;
   3-14-membered heterocycloalkyl, saturated or unsaturated, mono- or polysubstituted;
   5-14-membered heteroaryl, mono- or polysubstituted; or
   —C₁-C₆-alkyl; unsubstituted, mono- or polysubstituted.
41. A pharmaceutical composition or a medicament comprising a compound according to any one of the preceding statements.
42. A compound according to any of the preceding statements or the pharmaceutical composition according to statement 41, for use as a medicament.
43. A compound according to any one of the preceding statements or a pharmaceutical composition according to statement 41 for use in the treatment of pain or epilepsy; preferably pain and epileptic encephalopathies.
44. The compound for use or the pharmaceutical composition for use according to statement 43, wherein the pain is selected from nociceptive pain, inflammatory pain, and neuropathic pain.
45. The compound for use or the pharmaceutical composition for use according to statement 43, wherein the pain is post-operative pain.
46. A method of prevention and/or treatment of TRPM3 mediated disorders, comprising administering to a subject an effective amount of a compound according to any one of statements 1 to 40, or a pharmaceutical composition according to claim 41.
47. A method of prevention and/or treatment of pain or epilepsy, such as epileptic encephalopathies, comprising administering to a subject an effective amount of a compound according to any one of statements 1 to 40, or a pharmaceutical composition according to claim 41.
   48. The method according to statement 47, wherein the pain is selected from nociceptive pain, inflammatory pain and neuropathic pain.

The first aspect of the invention is the provision of a compound of formula (I), a stereo-isomeric form, a physiologically acceptable salt, solvate and/or polymorph thereof

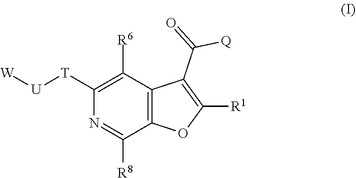

wherein
R¹ represents —F, —Cl, —Br, —I, —CN, —R^{w}, —OR^{w}, —OC(═O)R^{w}, —NR^{w}R^{x}, —NR^{w}C(═O)R^{X}, —SR^{w}, —S(═O)R^{w}, —S(═O)₂R^{w}, —C(═O)R^{w}, —C(═O)OR^{w}, or —C(═O)NR^{w}R^{x};
Q represents —NR³R⁴;
R³ represents —OH or —R^{Y1};
R⁴ represents —R^{Y2} or —S(═O)₂R^{Y3};
   or R³ and R⁴ together form a 4, 5, 6, 7 or 8 membered heterocycle containing 1 to 3 heteroatoms selected from N, O and S, saturated or unsaturated, unsubstituted or mono- or polysubstituted;
T represents —O— and U represents —(CR⁵R^{5′})ₙ—; or T represents —(CRR^{5′})ₙ— and U represents —O—; n is an integer selected from 1, 2, 3, 4, or 5; R⁵ and R^{5′} independently of one another represent —R^{Y4}; R⁶, and R⁸ independently of one another represent —F, —Cl, —Br, —I, —CN, —NO₂, —SF₅, —R^{w}, —OR^{w}, —OC(═O)R^{w}, —NR^{w}R^{X}, —NR^{w}C(═O)R^{X}, —SR^{w}, —S(═O)R^{w}, —S(═O)₂R^{w}, —C(═O)R^{w}, —C(═O)OR^{w}, or —C(═O)NR^{w}R^{x};
W represents
   3-14-membered cycloalkyl, saturated or unsaturated; unsubstituted, mono- or polysubstituted, in particular 3-14-membered cycloalkyl, saturated or non-aromatic unsaturated; unsubstituted, mono- or polysubstituted;
   6-14-membered aryl, unsubstituted, mono- or polysubstituted;
   3-14-membered heterocycloalkyl, saturated or unsaturated, mono- or polysubstituted;
   5-14-membered heteroaryl, mono- or polysubstituted; or
   —C₁-C₆-alkyl, —C₂-C₆-alkenyl, —C₂-C₆-alkynyl; in each case unsubstituted, mono- or polysubstituted;
wherein
R^{w} and R^{x} independently of one another in each case independently represent
   —H;
   —C₁-C₆-alkyl, saturated or unsaturated, unsubstituted, mono- or poly substituted;
   —C₁-C₆-heteroalkyl, saturated or unsaturated, unsubstituted, mono- or poly substituted;
   3-14-membered cycloalkyl, saturated or unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or poly substituted, in particular 3-14-membered cycloalkyl, saturated or non-aromatic unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted; or
   3-14-membered heterocycloalkyl, saturated or unsaturated, unsubstituted, mono- or polysubstituted; wherein said 3-14-membered heterocycloalkyl is optionally connected through —C₁-C₆-alkylene- or —C₁-C₆-heteroalkylene-, in each case saturated or unsaturated, unsubstituted, mono- or polysubstituted;
R^{Y1}, R^{Y2}, R^{Y3}, and R^{Y4} independently of one another in each case independently represent
   —H;
   —C₁-C₆-alkyl, saturated or unsaturated, unsubstituted, mono- or poly substituted;
   —C₁-C₆-heteroalkyl, saturated or unsaturated, unsubstituted, mono- or poly substituted;
   3-14-membered cycloalkyl, saturated or unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or poly substituted, in particular 3-14-membered cycloalkyl, saturated or non-aromatic unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted;
   3-14-membered heterocycloalkyl, saturated or unsaturated, unsubstituted, mono- or polysubstituted; wherein said 3-14-membered heterocycloalkyl is optionally connected through —C₁-C₆-alkylene- or —C₁-C₆-heteroalkylene-, in each case saturated or unsaturated, unsubstituted, mono- or polysubstituted;
   6-14-membered aryl, unsubstituted, mono- or polysubstituted; wherein said 6-14-membered aryl is optionally connected through —C₁-C₆-alkylene- or —C₁-C₆-heteroalkylene-, in each case saturated or unsaturated, unsubstituted, mono- or polysubstituted; or
   5-14-membered heteroaryl, unsubstituted, mono- or polysubstituted; wherein said 5-14-membered heteroaryl is optionally connected through —C₁-C₆-alkylene- or —C₁-C₆-heteroalkylene-, in each case saturated or unsaturated, unsubstituted, mono- or polysubstituted;
and wherein “mono- or polysubstituted” in each case independently means substituted with one or more substituents independently of one another selected from —F, —Cl, —Br, —I, —CN, —C₁₋₆-alkyl, —CF₃, —CF₂H, —CFH₂, —CF₂Cl, —CFCl₂, —C₁₋₆-alkylene-CF₃, —C₁₋₆-alkylene-CF₂H, —C₁₋₆-alkylene-CFH₂, —C₁₋₆-alkylene-NH—C₁₋₆-alkylene-CF₃, —C₁₋₆-alkylene-N(C₁₋₆-alkyl)-C₁₋₆-alkylene-CF₃, —C(═O)—C₁₋₆-alkyl, —C₁₋₆-alkylene-C(═O)—C₁₋₆-alkyl, —C(═O)OH, —C₁₋₆-alkylene-C(═O)—OH, —C(═O)—OC₁₋₆-alkyl, —C₁₋₆-alkylene-C(═O)—OC₁₋₆-alkyl, —C(═O)O—C₁₋₆-alkylene-CF₃, —C(═O)—NH₂, —C₁₋₆-alkylene-C(═O)—NH₂, —C(═O)—NH(C₁₋₆-alkyl), —C₁₋₆-alkylene-C(═O)—NH(C₁₋₆-alkyl), —C(═O)—N(C₁₋₆-alkyl)₂, —C₁₋₆-alkylene-C(═O)—N(C₁₋₆-alkyl)₂, —C(═O)—NH(OH), —C₁₋₆-alkylene-C(═O)—NH(OH), —OH, —C₁₋₆-alkylene-OH, ═O, —OCF₃, —OCF₂H, —OCFH₂, —OCF₂Cl, —OCFCl₂, —O—C₁₋₆-alkyl, —C₁₋₆-alkylene-O—C₁₋₆-alkyl, —O—C₁₋₆-alkylene-O—C₁₋₆-alkyl, —O—C₁₋₆-alkylene-NH₂, —O—C₁₋₆-alkylene-NH—C₁₋₆-alkyl, —O—C₁₋₆-alkylene-N(C₁₋₆-alkyl)₂, —O—C(═O)—C₁₋₆-alkyl, —C₁₋₆-alkylene-O—C(═O)—C₁₋₆-alkyl, —O—C(═O)—O—C₁₋₆-alkyl, —C₁₋₆-alkylene-O—C(═O)—O—C₁₋₆-alkyl, —O—C(═O)—NH(C₁₋₆-alkyl), —C₁₋₆-alkylene-O—C(═O)—NH(C₁₋₆-alkyl), —O—C(═O)—N(C₁₋₆-alkyl)₂, —C₁₋₆-alkylene-O—C(═O)—N(C₁₋₆-alkyl)₂, —O—S(═O)₂—NH₂, —C₁₋₆-alkylene-O—S(═O)₂—NH₂, —O—S(═O)₂—NH(C₁₋₆-alkyl), —C₁₋₆-alkylene-O—S(═O)₂—NH(C₁₋₆-alkyl), —O—S(═O)₂—N(C₁₋₆-alkyl)₂, —C₁₋₆-alkylene-O—S(═O)₂—N(C₁₋₆-alkyl)₂, —NH₂, —NO, —NO₂, —C₁₋₆-alkylene-NH₂, —NH(C₁₋₆-alkyl), —C₁₋₆-alkylene-NH(C₁₋₆-alkyl), —N(C₁₋₆-alkyl)₂, —C₁₋₆-alkylene-N(C₁₋₆-alkyl)₂, —NH—C(═O)—C₁₋₆-alkyl, —C₁₋₆-alkylene-NH—C(═O)—C₁₋₆-alkyl, —NH—C(═O)—O—C₁₋₆-alkyl, —C₁₋₆-alkylene-NH—C(═O)—O—C₁₋₆-alkyl, —NH—C(═O)—NH₂, —C₁₋₆-alkylene-NH—C(═O)—NH₂, —NH—C(═O)—NH(C₁₋₆-alkyl), —C₁₋₆-alkylene-NH—C(═O)—NH(C₁₋₆-alkyl), —NH—C(═O)—N(C₁₋₆-alkyl)₂, —C₁₋₆-alkylene-NH—C(═O)—N(C₁₋₆-alkyl)₂, —N(C₁₋₆-alkyl)-C(═O)—C₁₋₆-alkyl, —C₁₋₆-alkylene-N(C₁₋₆-alkyl)-C(═O)—C₁₋₆-alkyl, —N(C₁₋₆-alkyl)-C(═O)—O—C₁₋₆-alkyl, —C₁₋₆-alkylene-N(C₁₋₆-alkyl)-C(═O)—O—C₁₋₆-alkyl, —N(C₁₋₆-alkyl)-C(═O)—NH₂, —C₁₋₆-alkylene-N(C₁₋₆-alkyl)-C(═O)—NH₂, —N(C₁₋₆-alkyl)-C(═O)—NH(C₁₋₆-alkyl), —C₁₋₆-alkylene-N(C₁₋₆-alkyl)-C(═O)—NH(C₁₋₆-alkyl), —N(C₁₋₆-alkyl)-C(═O)—N(C₁₋₆-alkyl)₂, —C₁₋₆-alkylene-N(C₁₋₆-alkyl)-C(═O)—N(C₁₋₆-alkyl)₂, —NH—S(═O)₂OH, —C₁₋₆-alkylene-NH—S(═O)₂OH, —NH—S(═O)₂—C₁₋₆-alkyl, —C₁₋₆-alkylene-NH—S(═O)₂—C₁₋₆-alkyl, —NH—S(═O)₂—O—C₁₋₆-alkyl, —C₁₋₆-alkylene-NH—S(═O)₂—O—C₁₋₆-alkyl, —NH—S(═O)₂—NH₂, —C₁₋₆-alkylene-NH—S(═O)₂—NH₂, —NH—S(═O)₂—NH(C₁₋₆-alkyl), —C₁₋₆-alkylene-NH—S(═O)₂—NH(C₁₋₆-alkyl), —NH—S(═O)₂N(C₁₋₆-alkyl)₂, —C₁₋₆-alkylene-NH—S(═O)₂N(C₁₋₆-alkyl)₂, —N(C₁₋₆-alkyl)-S(═O)₂—OH, —C₁₋₆-alkylene-N(C₁₋₆-alkyl)-S(═O)₂—OH, —N(C₁₋₆-alkyl)-S(═O)₂—C₁₋₆-alkyl, —C₁₋₆-alkylene-N(C₁₋₆-alkyl)-S(═O)₂—C₁₋₆-alkyl, —N(C₁₋₆-alkyl)-S(═O)₂—O—C₁₋₆-alkyl, —C₁₋₆-alkylene-N(C₁₋₆-alkyl)-S(═O)₂—O—C₁₋₆-alkyl, —N(C₁₋₆-alkyl)-S(═O)₂—NH₂, —C₁₋₆-alkylene-N(C₁₋₆-alkyl)-S(═O)₂—NH₂, —N(C₁₋₆-alkyl)-S(═O)₂—NH(C₁₋₆-alkyl), —C₁₋₆-alkylene-N(C₁₋₆-alkyl)-S(═O)₂—NH(C₁₋₆-alkyl), —N(C₁₋₆-alkyl)-S(═O)₂—N(C₁₋₆-alkyl)₂, —C₁₋₆-alkylene-N(C₁₋₆-alkyl)-S(═O)₂—N(C₁₋₆-alkyl)₂, —SH, ═S, —SF₅, —SCF₃, —SCF₂H, —SCFH₂, —S—C₁₋₆-alkyl, —C₁₋₆-alkylene-S—C₁₋₆-alkyl, —S(═O)—C₁₋₆-alkyl, —C₁₋₆-alkylene-S(═O)—C₁₋₆-alkyl, —S(═O)₂—C₁₋₆-alkyl, —C₁₋₆-alkylene-S(═O)₂—C₁₋₆-alkyl, —S(═O)₂—OH, —C₁₋₆-alkylene-S(═O)₂—OH, —S(═O)₂—O—C₁₋₆-alkyl, —C₁₋₆-alkylene-S(═O)₂—O—C₁₋₆-alkyl, —S(═O)₂—NH₂, —C₁₋₆-alkylene-S(═O)₂—NH₂, —S(═O)₂—NH(C₁₋₆-alkyl), —C₁₋₆-alkylene-S(═O)₂—NH(C₁₋₆-alkyl), —S(═O)₂—N(C₁₋₆-alkyl)₂, —C₁₋₆-alkylene-S(═O)₂—N(C₁₋₆-alkyl)₂, 3-14-membered cycloalkyl, —C₁₋₆-alkylene-(3-14-membered cycloalkyl), 3 to 14-membered heterocycloalkyl, —C₁₋₆-alkylene-(3 to 14-membered heterocycloalkyl), -phenyl, —C₁₋₆-alkylene-phenyl, 5 to 14-membered heteroaryl, —C₁₋₆-alkylene-(5 to 14-membered heteroaryl), —O-(3-14-membered cycloalkyl), —O-(3 to 14-membered heterocycloalkyl), —O-phenyl, —O-(5 to 14-membered heteroaryl), —C(═O)-(3-14-membered cycloalkyl), —C(═O)-(3 to 14-membered heterocycloalkyl), —C(═O)-phenyl, —C(═O)-(5 to 14-membered heteroaryl), —S(═O)₂-(3-14-membered cycloalkyl), —S(═O)₂-(3 to 14-membered heterocycloalkyl), —S(═O)₂-phenyl, or —S(═O)₂-(5 to 14-membered heteroaryl); or the compound is 5-(benzyloxy)-2-methylfuro[2,3-c]pyridine-3-carboxylate.

In some embodiments T represents —O—, U represents —(CR⁵R^{5′})ₙ—; and n is an integer selected from 1, 2 or 3.

In some embodiments R¹ represents
—H, —F, —Cl, —Br, —I, CN;
C₁₋₆-alkyl, saturated or unsaturated, unsubstituted, mono- or poly substituted;
—C₁-C₆-heteroalkyl, saturated or unsaturated, unsubstituted, mono- or polysubstituted;
—OH;
—O—C₁₋₆-alkyl, saturated or unsaturated, unsubstituted, mono- or poly substituted;
—NH₂;
—NHC₁₋₆-alkyl saturated or unsaturated, unsubstituted, mono- or polysubstituted;
—N(C₁₋₆-alkyl)₂ saturated or unsaturated, unsubstituted, mono- or polysubstituted;
—NHC(═O)C₁₋₆-alkyl saturated or unsaturated, unsubstituted, mono- or polysubstituted;
—NC₁₋₆-alkylC(═O)C₁₋₆-alkyl in each case saturated or unsaturated, unsubstituted, mono- or polysubstituted;
—SH;
—S—C₁₋₆-alkyl, saturated or unsaturated, unsubstituted, mono- or polysubstituted;
—C(═O)C₁₋₆-alkyl, saturated or unsaturated, unsubstituted, mono- or polysubstituted;
—C(═O)OC₁₋₆-alkyl, saturated or unsaturated, unsubstituted, mono- or polysubstituted;
—C(═O)NH₂;
—C(═O)NHC₁₋₆-alkyl, saturated or unsaturated, unsubstituted, mono- or polysubstituted;
—C(═O)N(C₁₋₆-alkyl)₂, saturated or unsaturated, unsubstituted, mono- or polysubstituted;
—S(═O)C₁₋₆-alkyl, saturated or unsaturated, unsubstituted, mono- or polysubstituted;
—S(═O)₂—C₁₋₆-alkyl, saturated or unsaturated, unsubstituted, mono- or poly substituted;
—C₁-C₆-heteroalkyl, saturated or unsaturated, unsubstituted, mono- or polysubstituted; or
3-14-membered cycloalkyl, saturated or unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted, in particular 3-14-membered cycloalkyl, saturated or non-aromatic unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted;
3-14-membered heterocycloalkyl, saturated or unsaturated, unsubstituted, mono- or polysubstituted; wherein said 3-14-membered heterocycloalkyl is optionally connected through —C₁-C₆-alkylene- or —C₁-C₆-heteroalkylene-, in each case saturated or unsaturated, unsubstituted, mono- or polysubstituted.

In some embodiments R¹ represents
—H, —F, —Cl, —Br, —I, CN;
—C₁₋₆-alkyl, —C₂-C₆-alkenyl, —C₂-C₆-alkynyl, in each case unsubstituted, mono- or polysubstituted with substituents independently of one another selected from the group consisting of —F, —Cl, —Br, —I, —CF₃, —CF₂H, —CFH₂, —CF₂Cl, —CFCl₂, —C₁₋₆-alkylene-CF₃, —C(═O)—C₁₋₆-alkyl, —C(═O)OH, —C(═O)—OC₁₋₆-alkyl, —C(═O)O—C₁₋₆-alkylene-CF₃, —OH, ═O, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C₁₋₆-alkylene-OH, —C₁₋₆-alkylene-O—C₁₋₆-alkyl, —C₁₋₆-alkylene-NHC(═O)O—C₁₋₆-alkyl, —NHC(═O)O—C₁₋₆-alkyl; —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂;
—C₁-C₆-heteroalkyl unsubstituted, mono- or polysubstituted with substituents independently of one another selected from the group consisting of —F, —Cl, —Br, —I, —CF₃, —CF₂H, —CFH₂, —CF₂Cl, —CFCl₂, —C₁₋₆-alkylene-CF₃, —C(═O)—C₁₋₆-alkyl, —C(═O)OH, —C(═O)—OC₁₋₆-alkyl, —C(═O)O—C₁₋₆-alkylene-CF₃, —OH, ═O, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C₁₋₆-alkylene-OH, —C₁₋₆-alkylene-O—C₁₋₆-alkyl, —C₁₋₆-alkylene-NHC(═O)O—C₁₋₆-alkyl, —NHC(═O)O—C₁₋₆-alkyl; —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂;
—OH;
—O—C₁₋₆-alkyl, —O—C₂-C₆-alkenyl, —O—C₂-C₆-alkynyl, in each case unsubstituted, mono- or polysubstituted with substituents independently of one another selected from the group consisting of —F, —Cl, —Br, —I, —CF₃, —CF₂H, —CFH₂, —CF₂Cl, —CFCl₂, —C₁₋₆-alkylene-CF₃, —C(═O)—C₁₋₆-alkyl, —C(═O)OH, —C(═O)—OC₁₋₆-alkyl, —C(═O)O—C₁₋₆-alkylene-CF₃, —OH, ═O, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C₁₋₆-alkylene-OH, —C₁₋₆-alkylene-O—C₁₋₆-alkyl, —C₁₋₆-alkylene-NHC(═O)O—C₁₋₆-alkyl, —NHC(═O)O—C₁₋₆-alkyl; —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂;
—NH₂;
—NHC₁₋₆-alkyl unsubstituted, mono- or polysubstituted with substituents independently of one another selected from the group consisting of —F, —Cl, —Br, —I, —CF₃, —CF₂H, —CFH₂, —CF₂Cl, —CFCl₂, —C₁₋₆-alkylene-CF₃, —C(═O)—C₁₋₆-alkyl, —C(═O)OH, —C(═O)—OC₁₋₆-alkyl, —C(═O)O—C₁₋₆-alkylene-CF₃, —OH, ═O, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C₁₋₆-alkylene-OH, —C₁₋₆-alkylene-O—C₁₋₆-alkyl, —C₁₋₆-alkylene-NHC(═O)O—C₁₋₆-alkyl, —NHC(═O)O—C₁₋₆-alkyl; —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂;
—N(C₁₋₆-alkyl)₂ in each case unsubstituted, mono- or polysubstituted with substituents independently of one another selected from the group consisting of —F, —Cl, —Br, —I, —CF₃, —CF₂H, —CFH₂, —CF₂Cl, —CFCl₂, —C₁₋₆-alkylene-CF₃, —C(═O)—C₁₋₆-alkyl, —C(═O)OH, —C(═O)—OC₁₋₆-alkyl, —C(═O)O—C₁₋₆-alkylene-CF₃, —OH, ═O, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C₁₋₆-alkylene-OH, —C₁₋₆-alkylene-O—C₁₋₆-alkyl, —C₁₋₆-alkylene-NHC(═O)O—C₁₋₆-alkyl, —NHC(═O)O—C₁₋₆-alkyl; —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂;
—NHC(═O)C₁₋₆-alkyl unsubstituted, mono- or polysubstituted with substituents independently of one another selected from the group consisting of —F, —Cl, —Br, —I, —CF₃, —CF₂H, —CFH₂, —CF₂Cl, —CFCl₂, —C₁₋₆-alkylene-CF₃, —C(═O)—C₁₋₆-alkyl, —C(═O)OH, —C(═O)—OC₁₋₆-alkyl, —C(═O)O—C₁₋₆-alkylene-CF₃, —OH, ═O, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C₁₋₆-alkylene-OH, —C₁₋₆-alkylene-O—C₁₋₆-alkyl, —C₁₋₆-alkylene-NHC(═O)O—C₁₋₆-alkyl, —NHC(═O)O—C₁₋₆-alkyl; —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂;
—NC₁₋₆-alkylC(═O)C₁₋₆-alkyl in each case unsubstituted, mono- or polysubstituted with substituents independently of one another selected from the group consisting of —F, —Cl, —Br, —I, —CF₃, —CF₂H, —CFH₂, —CF₂Cl, —CFCl₂, —C₁₋₆-alkylene-CF₃, —C(═O)—C₁₋₆-alkyl, —C(═O)OH, —C(═O)—OC₁₋₆-alkyl, —C(═O)O—C₁₋₆-alkylene-CF₃, —OH, ═O, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C₁₋₆-alkylene-OH, —C₁₋₆-alkylene-O—C₁₋₆-alkyl, —C₁₋₆-alkylene-NHC(═O)O—C₁₋₆-alkyl, —NHC(═O)O—C₁₋₆-alkyl; —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂;
—SH;
—S—C₁₋₆-alkyl, —S—C₂-C₆-alkenyl, —S—C₂-C₆-alkynyl, in each case unsubstituted, mono- or polysubstituted with substituents independently of one another selected from the group consisting of —F, —Cl, —Br, —I, —CF₃, —CF₂H, —CFH₂, —CF₂Cl, —CFCl₂, —C₁₋₆-alkylene-CF₃, —C(═O)—C₁₋₆-alkyl, —C(═O)OH, —C(═O)—OC₁₋₆-alkyl, —C(═O)O—C₁₋₆-alkylene-CF₃, —OH, ═O, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C₁₋₆-alkylene-OH, —C₁₋₆-alkylene-O—C₁₋₆-alkyl, —C₁₋₆-alkylene-NHC(═O)O—C₁₋₆-alkyl, —NHC(═O)O—C₁₋₆-alkyl; —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂;
—C(═O)C₁₋₆-alkyl unsubstituted, mono- or polysubstituted with substituents independently of one another selected from the group consisting of —F, —Cl, —Br, —I, —CF₃, —CF₂H, —CFH₂, —CF₂Cl, —CFCl₂, —C₁₋₆-alkylene-CF₃, —C(═O)—C₁₋₆-alkyl, —C(═O)OH, —C(═O)—OC₁₋₆-alkyl, —C(═O)O—C₁₋₆-alkylene-CF₃, —OH, ═O, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C₁₋₆-alkylene-OH, —C₁₋₆-alkylene-O—C₁₋₆-alkyl, —C₁₋₆-alkylene-NHC(═O)O—C₁₋₆-alkyl, —NHC(═O)O—C₁₋₆-alkyl; —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂;
—C(═O)OC₁₋₆-alkyl unsubstituted, mono- or polysubstituted with substituents independently of one another selected from the group consisting of —F, —Cl, —Br, —I, —CF₃, —CF₂H, —CFH₂, —CF₂Cl, —CFCl₂, —C₁₋₆-alkylene-CF₃, —C(═O)—C₁₋₆-alkyl, —C(═O)OH, —C(═O)—OC₁₋₆-alkyl, —C(═O)O—C₁₋₆-alkylene-CF₃, —OH, ═O, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C₁₋₆-alkylene-OH, —C₁₋₆-alkylene-O—C₁₋₆-alkyl, —C₁₋₆-alkylene-NHC(═O)O—C₁₋₆-alkyl, —NHC(═O)O—C₁₋₆-alkyl; —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂;
—C(═O)NH₂;
—C(═O)NHC₁₋₆-alkyl unsubstituted, mono- or polysubstituted with substituents independently of one another selected from the group consisting of —F, —Cl, —Br, —I, —CF₃, —CF₂H, —CFH₂, —CF₂Cl, —CFCl₂, —C₁₋₆-alkylene-CF₃, —C(═O)—C₁₋₆-alkyl, —C(═O)OH, —C(═O)—OC₁₋₆-alkyl, —C(═O)O—C₁₋₆-alkylene-CF₃, —OH, ═O, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C₁₋₆-alkylene-OH, —C₁₋₆-alkylene-O—C₁₋₆-alkyl, —C₁₋₆-alkylene-NHC(═O)O—C₁₋₆-alkyl, —NHC(═O)O—C₁₋₆-alkyl; —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂;
—C(═O)N(C₁₋₆-alkyl)₂, in each case unsubstituted, mono- or polysubstituted with substituents independently of one another selected from the group consisting of —F, —Cl, —Br, —I, —CF₃, —CF₂H, —CFH₂, —CF₂Cl, —CFCl₂, —C₁₋₆-alkylene-CF₃, —C(═O)—C₁₋₆-alkyl, —C(═O)OH, —C(═O)—OC₁₋₆-alkyl, —C(═O)O—C₁₋₆-alkylene-CF₃, —OH, ═O, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C₁₋₆-alkylene-OH, —C₁₋₆-alkylene-O—C₁₋₆-alkyl, —C₁₋₆-alkylene-NHC(═O)O—C₁₋₆-alkyl, —NHC(═O)O—C₁₋₆-alkyl; —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂;
—S(═O)C₁₋₆-alkyl unsubstituted, mono- or polysubstituted with substituents independently of one another selected from the group consisting of —F, —Cl, —Br, —I, —CF₃, —CF₂H, —CFH₂, —CF₂Cl, —CFCl₂, —C₁₋₆-alkylene-CF₃, —C(═O)—C₁₋₆-alkyl, —C(═O)OH, —C(═O)—OC₁₋₆-alkyl, —C(═O)O—C₁₋₆-alkylene-CF₃, —OH, ═O, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C₁₋₆-alkylene-OH, —C₁₋₆-alkylene-O—C₁₋₆-alkyl, —C₁₋₆-alkylene-NHC(═O)O—C₁₋₆-alkyl, —NHC(═O)O—C₁₋₆-alkyl; —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂;
—S(═O)₂—C₁₋₆-alkyl unsubstituted, mono- or polysubstituted with substituents independently of one another selected from the group consisting of —F, —Cl, —Br, —I, —CF₃, —CF₂H, —CFH₂, —CF₂Cl, —CFCl₂, —C₁₋₆-alkylene-CF₃, —C(═O)—C₁₋₆-alkyl, —C(═O)OH, —C(═O)—OC₁₋₆-alkyl, —C(═O)O—C₁₋₆-alkylene-CF₃, —OH, ═O, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C₁₋₆-alkylene-OH, —C₁₋₆-alkylene-O—C₁₋₆-alkyl, —C₁₋₆-alkylene-NHC(═O)O—C₁₋₆-alkyl, —NHC(═O)O—C₁₋₆-alkyl; —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂;
—C₁-C₆-heteroalkyl unsubstituted, mono- or polysubstituted with substituents independently of one another selected from the group consisting of —F, —Cl, —Br, —I, —CF₃, —CF₂H, —CFH₂, —CF₂Cl, —CFCl₂, —C₁₋₆-alkylene-CF₃, —C(═O)—C₁₋₆-alkyl, —C(═O)OH, —C(═O)—OC₁₋₆-alkyl, —C(═O)O—C₁₋₆-alkylene-CF₃, —OH, ═O, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C₁₋₆-alkylene-OH, —C₁₋₆-alkylene-O—C₁₋₆-alkyl, —C₁₋₆-alkylene-NHC(═O)O—C₁₋₆-alkyl, —NHC(═O)O—C₁₋₆-alkyl; —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂; or
3-14-membered cycloalkyl unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted with substituents independently of one another selected from the group consisting of —F, —Cl, —Br, —I, —CF₃, —CF₂H, —CFH₂, —CF₂Cl, —CFCl₂, —C₁₋₆-alkylene-CF₃, —C(═O)—C₁₋₆-alkyl, —C(═O)OH, —C(═O)—OC₁₋₆-alkyl, —C(═O)O—C₁₋₆-alkylene-CF₃, —OH, ═O, —OC₁₋₆-alkyl, —NH₂, —NHC₁₋₆-alkyl, —N(C₁₋₆-allkyl)₂, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C₁₋₆-alkylene-OH, —C₁₋₆-alkylene-O—C₁₋₆-alkyl, —C₁₋₆-alkylene-NHC(═O)O—C₁₋₆-alkyl, —NHC(═O)O—C₁₋₆-alkyl; —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂;
3-14-membered heterocycloalkyl, saturated or unsaturated, unsubstituted, mono- or polysubstituted; wherein said 3-14-membered heterocycloalkyl is optionally connected through —C₁-C₆-alkylene- or —C₁-C₆-heteroalkylene-, in each case saturated or unsaturated, unsubstituted, mono- or polysubstituted unsubstituted, mono- or polysubstituted with substituents independently of one another selected from the group consisting of —F, —Cl, —Br, —I, —CF₃, —CF₂H, —CFH₂, —CF₂Cl, —CFCl₂, —C₁₋₆-alkylene-CF₃, —C(═O)—C₁₋₆-alkyl, —C(═O)OH, —C(═O)—OC₁₋₆-alkyl, —C(═O)O—C₁₋₆-alkylene-CF₃, —OH, ═O, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C₁₋₆-alkylene-OH, —C₁₋₆-alkylene-O—C₁₋₆-alkyl, —C₁₋₆-alkylene-NHC(═O)O—C₁₋₆-alkyl, —NHC(═O)O—C₁₋₆-alkyl; —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂.

In some embodiments R¹ represents —H, —F, —Cl, —Br, —I, —CN, —C₁₋₆-alkyl, —C₂-C₆-alkenyl, —C₂-C₆-alkynyl, —OH, —O—C₁₋₆-alkyl, —SH, —NH₂, —C₁₋₆-alkylene-O—C₁₋₆-alkyl, —C₁₋₆-alkylene-NH(C₁₋₆-alkyl), —C₁₋₆-alkylene-N(C₁₋₆-alkyl)₂, —CF₃, —CF₂H, —CFH₂, —CF₂Cl, —CFCl₂, —C₁₋₆-alkylene-CF₃, —C₁₋₆-alkylene-CF₂H, —C₁₋₆-alkylene-CFH₂, —C₁-6-alkylene-NH—C₁₋₆-alkylene-CF₃, —C₁₋₆-alkylene-N(C₁₋₆-alkyl)-C₁₋₆-alkylene-CF₃, —C(═O)C₁₋₆-alkyl, —C(═O)OC₁₋₆-alkyl, —C(═O)NH₂, —C(═O)NHC₁₋₆-alkyl, —C(═O)N(C₁₋₆-alkyl)₂, —S(═O)—C₁₋₆-alkyl, —S(═O)₂—C₁₋₆-alkyl, —O—C₁₋₆-alkyl, 3-14-membered cycloalkyl unsubstituted, or 3-14-membered heterocycloalkyl unsubstituted. Preferably, R¹ represents —H, —F, —Cl, —Br, —I, CN, —C₁₋₆-alkyl, —C₂-C₆-alkenyl, —C₂-C₆-alkynyl, —OH, —O—C₁₋₆-alkyl, —SH, —NH₂, —C₁₋₆-alkylene-O—C₁₋₆-alkyl, —C₁₋₆-alkylene-NH(C₁₋₆-alkyl), —C₁₋₆-alkylene-N(C₁₋₆-alkyl)₂, —CF₃, —CF₂H, —CFH₂, —CF₂Cl, —CFCl₂, —C₁₋₆-alkylene-CF₃, —C₁₋₆-alkylene-CF₂H, —C₁₋₆-alkylene-CFH₂, —C₁₋₆-alkylene-NH—C₁₋₆-alkylene-CF₃, —C₁₋₆-alkylene-N(C₁₋₆-alkyl)-C₁₋₆-alkylene-CF₃, —C(═O)C₁₋₆-alkyl, —C(═O)OC₁₋₆-alkyl, —C(═O)NH₂, —C(═O)NHC₁₋₆-alkyl, —C(═O)N(C₁₋₆-alkyl)₂, —S(═O)—C₁₋₆-alkyl, —S(═O)₂—C₁₋₆-alkyl, —O—C₁₋₆-alkyl, -cyclopropyl unsubstituted, cyclobutyl unsubstituted, cyclopentyl unsubstituted or cyclohexyl unsubstituted.

In some embodiments R¹ represents —H, CN, —C₁₋₆-alkyl, —C₁₋₆-alkylene-O—C₁₋₆-alkyl, —CH₂F, —CHF₂, —CF₃, —C(═O)NH₂, -cyclopropyl unsubstituted, cyclobutyl unsubstituted, cyclopentyl unsubstituted or cyclohexyl unsubstituted. Preferably, R¹ represents —H, —CH₂F, —CHF₂, —CF₃, —C(═O)NH₂, —CN, -cyclopropyl unsubstituted, cyclobutyl unsubstituted, cyclopentyl unsubstituted or cyclohexyl unsubstituted. Preferably, R¹ represents H, —C₁₋₆-alkyl, —C(═O)NH₂, —CHF₂, -cyclopropyl unsubstituted, cyclobutyl unsubstituted, cyclopentyl unsubstituted or cyclohexyl unsubstituted.

Preferably, R¹ represents —H, —CN, —C₁₋₃-alkyl, —CF₃, —CF₂H, —CFH₂, —CF₂Cl, —CFCl₂, —C₁₋₃-alkylene-CF₃, —C₁₋₃-alkylene-CF₂H, —C(═O)NH₂, or —C₁₋₃-alkylene-CFH₂; more preferably —H, —CH₃, or —CF₂H.

In some embodiments R³ represents
—H;
—OH;
—C₁-C₆-alkyl, saturated or unsaturated, unsubstituted, mono- or polysubstituted;
—C₁-C₆-heteroalkyl, saturated or unsaturated, unsubstituted, mono- or polysubstituted;
3-14-membered cycloalkyl, saturated or unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted, in particular 3-14-membered cycloalkyl, saturated or non-aromatic unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted;
3-14-membered heterocycloalkyl, saturated or unsaturated, unsubstituted, mono- or polysubstituted; wherein said 3-14-membered heterocycloalkyl is optionally connected through —C₁-C₆-alkylene- or —C₁-C₆-heteroalkylene-, in each case saturated or unsaturated, unsubstituted, mono- or polysubstituted;
6-14-membered aryl, unsubstituted, mono- or polysubstituted; wherein said 6-14-membered aryl is optionally connected through —C₁-C₆-alkylene- or —C₁-C₆-heteroalkylene-, in each case saturated or unsaturated, unsubstituted, mono- or polysubstituted; or
5-14-membered heteroaryl, unsubstituted, mono- or polysubstituted; wherein said 5-14-membered heteroaryl is optionally connected through —C₁-C₆-alkylene- or —C₁-C₆-heteroalkylene-, in each case saturated or unsaturated, unsubstituted, mono- or polysubstituted.

In some embodiments R³ represents
—H;
—OH;
—C₁-C₆-alkyl, —C₂-C₆-alkenyl, —C₂-C₆-alkynyl, in each case unsubstituted, mono- or polysubstituted with substituents independently of one another selected from the group consisting of —F, —Cl, —Br, —I, —CF₃, —CF₂H, —CFH₂, —CF₂Cl, —CFCl₂, —C₁₋₆-alkylene-CF₃, —C(═O)—C₁₋₆-alkyl, —C(═O)OH, —C(═O)—OC₁₋₆-alkyl, —C(═O)O—C₁₋₆-alkylene-CF₃, —OH, ═O, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C₁₋₆-alkylene-OH, —C₁₋₆-alkylene-O—C₁₋₆-alkyl, —C₁₋₆-alkylene-NHC(═O)O—C₁₋₆-alkyl, —NHC(═O)O—C₁₋₆-alkyl; —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂;
—C₁-C₆-heteroalkyl unsubstituted, mono- or polysubstituted with substituents independently of one another selected from the group consisting of —F, —Cl, —Br, —I, —CF₃, —CF₂H, —CFH₂, —CF₂Cl, —CFCl₂, —C₁₋₆-alkylene-CF₃, —C(═O)—C₁₋₆-alkyl, —C(═O)OH, —C(═O)—OC₁₋₆-alkyl, —C(═O)O—C₁₋₆-alkylene-CF₃, —OH, ═O, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C₁₋₆-alkylene-OH, —C₁₋₆-alkylene-O—C₁₋₆-alkyl, —C₁₋₆-alkylene-NHC(═O)O—C₁₋₆-alkyl, —NHC(═O)O—C₁₋₆-alkyl; —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂;
3-14-membered cycloalkyl, saturated or unsaturated, unsubstituted, mono- or polysubstituted; wherein said 3-14-membered cycloalkyl is optionally connected through —C₁-C₆-alkylene- or —C₁-C₆-heteroalkylene-, in each case unsubstituted, mono- or polysubstituted with substituents independently of one another selected from the group consisting of —F, —Cl, —Br, —I, —CF₃, —CF₂H, —CFH₂, —CF₂Cl, —CFCl₂, —C₁₋₆-alkylene-CF₃, —C(═O)—C₁₋₆-alkyl, —C(═O)OH, —C(═O)—OC₁₋₆-alkyl, —C(═O)O—C₁₋₆-alkylene-CF₃, —OH, ═O, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C₁₋₆-alkylene-OH, —C₁₋₆-alkylene-O—C₁₋₆-alkyl, —C₁₋₆-alkylene-NHC(═O)O—C₁₋₆-alkyl, —NHC(═O)O—C₁₋₆-alkyl; —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂, in particular 3-14-membered cycloalkyl, saturated or non-aromatic unsaturated, unsubstituted, mono- or polysubstituted; wherein said 3-14-membered cycloalkyl is optionally connected through —C₁-C₆-alkylene- or —C₁-C₆-heteroalkylene-, in each case unsubstituted, mono- or polysubstituted with substituents independently of one another selected from the group consisting of —F, —Cl, —Br, —I, —CF₃, —CF₂H, —CFH₂, —CF₂Cl, —CFCl₂, —C₁₋₆-alkylene-CF₃, —C(═O)—C₁₋₆-alkyl, —C(═O)OH, —C(═O)—OC₁₋₆-alkyl, —C(═O)O—C₁₋₆-alkylene-CF₃, —OH, ═O, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C₁₋₆-alkylene-OH, —C₁₋₆-alkylene-O—C₁₋₆-alkyl, —C₁₋₆-alkylene-NHC(═O)O—C₁₋₆-alkyl, —NHC(═O)O—C₁₋₆-alkyl; —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂;
3-14-membered heterocycloalkyl unsubstituted, mono- or polysubstituted; wherein said 3-14-membered heterocycloalkyl is optionally connected through —C₁-C₆-alkylene- or —C₁-C₆-heteroalkylene-, in each case saturated or unsaturated, unsubstituted, mono- or polysubstituted with substituents independently of one another selected from the group consisting of —F, —Cl, —Br, —I, —CF₃, —CF₂H, —CFH₂, —CF₂Cl, —CFCl₂, —C₁₋₆-alkylene-CF₃, —C(═O)—C₁₋₆-alkyl, —C(═O)OH, —C(═O)—OC₁₋₆-alkyl, —C(═O)O—C₁₋₆-alkylene-CF₃, —OH, ═O, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C₁₋₆-alkylene-OH, —C₁₋₆-alkylene-O—C₁₋₆-alkyl, —C₁₋₆-alkylene-NHC(═O)O-C₁₋₆-alkyl, —NHC(═O)O—C₁₋₆-alkyl; —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂;
6-14-membered aryl, unsubstituted, mono- or polysubstituted; wherein said 6-14-membered aryl is optionally connected through —C₁-C₆-alkylene- or —C₁-C₆-heteroalkylene-, in each case unsubstituted, mono- or polysubstituted with substituents independently of one another selected from the group consisting of —F, —Cl, —Br, —I, —CF₃, —CF₂H, —CFH₂, —CF₂Cl, —CFCl₂, —C₁₋₆-alkylene-CF₃, —C(═O)—C₁₋₆-alkyl, —C(═O)OH, —C(═O)—OC₁₋₆-alkyl, —C(═O)O—C₁₋₆-alkylene-CF₃, —OH, ═O, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C₁₋₆-alkylene-OH, —C₁₋₆-alkylene-O—C₁₋₆-alkyl, —C₁₋₆-alkylene-NHC(═O)O—C₁₋₆-alkyl, —NHC(═O)O—C₁₋₆-alkyl; —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂; or
5-14-membered heteroaryl, unsubstituted, mono- or polysubstituted; wherein said 5-14-membered heteroaryl is optionally connected through —C₁-C₆-alkylene- or —C₁-C₆-heteroalkylene-, in each case saturated or unsaturated, unsubstituted, mono- or polysubstituted with substituents independently of one another selected from the group consisting of —F, —Cl, —Br, —I, —CF₃, —CF₂H, —CFH₂, —CF₂Cl, —CFCl₂, —C₁₋₆-alkylene-CF₃, —C(═O)—C₁₋₆-alkyl, —C(═O)OH, —C(═O)—OC₁₋₆-alkyl, —C(═O)O—C₁₋₆-alkylene-CF₃, —OH, ═O, —OC₁₋₆-alkyl, —NH₂, —NHC₁₋₆-alkyl, —N(C₁₋₆-allkyl)₂, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C₁₋₆-alkylene-OH, —C₁₋₆-alkylene-O—C₁₋₆-alkyl, —C₁₋₆-alkylene-NHC(═O)O—C₁₋₆-alkyl, —NHC(═O)O—C₁₋₆-alkyl; —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂.

In some embodiments R³ represents
—H;
—OH;
—C₁-C₆-alkyl, saturated or unsaturated, unsubstituted, mono- or polysubstituted; or
—C₁-C₆-heteroalkyl, saturated or unsaturated, unsubstituted, mono- or polysubstituted.

Preferably, R³ represents —H, —OH, —C₁₋₆-alkyl, —C₁₋₆-alkylene-OH, —C₁₋₆-alkylene-C(═O)—NH₂, —C₁₋₆-alkylene-O—C₁₋₆-alkyl, —C₁₋₆-alkylene-NH₂, —C₁₋₆-alkylene-NH(C₁₋₆-alkyl), —C₁₋₆-alkylene-N(C₁₋₆-alkyl)₂, —CF₃, —CF₂H, —CFH₂, —CF₂Cl, —CFCl₂, —C₁₋₆-alkylene-CF₃, —C₁₋₆-alkylene-CF₂H, —C₁₋₆-alkylene-CFH₂, —C₁₋₆-alkylene-NH—C₁₋₆-alkylene-CF₃, or —C₁₋₆-alkylene-N(C₁₋₆-alkyl)-C₁₋₆-alkylene-CF₃. More preferably, R³ represents —H, —OH, —C₁₋₆-alkyl, —C₁₋₆-alkylene-OH, —C₁₋₆-alkylene-O—C₁₋₆-alkyl, —C₁₋₆-alkylene-NH₂, —C₁₋₆-alkylene-NH(C₁₋₆-alkyl), —C₁₋₆-alkylene-N(C₁₋₆-alkyl)₂, —CF₃, —CF₂H, —CFH₂, —CF₂Cl, —CFCl₂, —C₁₋₆-alkylene-CF₃, —C₁₋₆-alkylene-CF₂H, —C₁₋₆-alkylene-CFH₂, —C₁₋₆-alkylene-NH—C₁₋₆-alkylene-CF₃, or —C₁₋₆-alkylene-N(C₁₋₆-alkyl)-C₁₋₆-alkylene-CF₃. Preferably, R³ represents —H, —OH, or —C₁₋₆-alkyl, saturated, unsubstituted or monosubstituted with —OH. Preferably, R³ represents —H.

In some embodiment, R³ and R⁴ both do not represent —H. In some embodiments, R³ represents —H and R⁴ does not represent —H.

In some embodiments R⁴ represents
—H;
—S(═O)C₁₋₆-alkyl, saturated or unsaturated, unsubstituted, mono- or polysubstituted;
—S(═O)₂—C₁₋₆-alkyl, saturated or unsaturated, unsubstituted, mono- or poly substituted;
—C₁-C₆-alkyl, saturated or unsaturated, unsubstituted, mono- or polysubstituted;
—C₁-C₆-heteroalkyl, saturated or unsaturated, unsubstituted, mono- or polysubstituted;
3-14-membered cycloalkyl, saturated or unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted, in particular 3-14-membered cycloalkyl, saturated or non-aromatic unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted;
3-14-membered heterocycloalkyl, saturated or unsaturated, unsubstituted, mono- or polysubstituted; wherein said 3-14-membered heterocycloalkyl is optionally connected through —C₁-C₆-alkylene- or —C₁-C₆-heteroalkylene-, in each case saturated or unsaturated, unsubstituted, mono- or polysubstituted;
6-14-membered aryl, unsubstituted, mono- or polysubstituted; wherein said 6-14-membered aryl is optionally connected through —C₁-C₆-alkylene- or —C₁-C₆-heteroalkylene-, in each case saturated or unsaturated, unsubstituted, mono- or polysubstituted; or
5-14-membered heteroaryl, unsubstituted, mono- or polysubstituted; wherein said 5-14-membered heteroaryl is optionally connected through —C₁-C₆-alkylene- or —C₁-C₆-heteroalkylene-, in each case saturated or unsaturated, unsubstituted, mono- or polysubstituted.

Preferably, R⁴ represents
—S(═O)₂C₁₋₆-alkyl unsubstituted, mono- or polysubstituted with substituents independently of one another selected from the group consisting of —F, —Cl, —C₁₋₆-alkyl, —C₁₋₆-alkylene-CF₃, —OH, ═O, —OC₁₋₆-alkyl, —C₁₋₆-alkylene-OH, —C₁₋₆-alkylene-O—C₁₋₆-alkyl, —NH₂, —NHC₁₋₆-alkyl, —N(C₁₋₆-alkyl)₂, —NHC(═O)O—C₁₋₆-alkyl, —N(C₁₋₆-alkyl)C(═O)O—C₁₋₆-alkyl, —C₁₋₆-alkylene-NHC(═O)O—C₁₋₆-alkyl, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —C(═O)N(C₁₋₆-alkyl)₂, —S(═O)₂C₁₋₆-alkyl, -phenyl, —C₁₋₆-alkylene-phenyl, 3-14-membered heterocycloalkyl, saturated or unsaturated, unsubstituted; and 5-14-membered heteroaryl, unsubstituted;
—S(═O)₂(3-14-membered cycloalkyl), wherein said 3-14-membered cycloalkyl is selected from the group consisting of cyclopropyl, cyclobutyl, cyclopentyl, cyclohexyl, and cycloheptyl, in each case saturated or unsaturated, unsubstituted, mono- or polysubstituted with substituents independently of one another selected from the group consisting of —F, —Cl, —C₁₋₆-alkyl, —C₁₋₆-alkylene-CF₃, —OH, ═O, —OC₁₋₆-alkyl, —C₁₋₆-alkylene-OH, —C₁₋₆-alkylene-O—C₁₋₆-alkyl, —NH₂, —NHC₁₋₆-alkyl, —N(C₁₋₆-alkyl)₂, —NHC(═O)O—C₁₋₆-alkyl, —N(C₁₋₆-alkyl)C(═O)O—C₁₋₆-alkyl, —C₁₋₆-alkylene-NHC(═O)O—C₁₋₆-alkyl, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —C(═O)N(C₁₋₆-alkyl)₂, —S(═O)₂C₁₋₆-alkyl, -phenyl, —C₁₋₆-alkylene-phenyl, 3-14-membered heterocycloalkyl, saturated or unsaturated, unsubstituted; and 5-14-membered heteroaryl, unsubstituted;
—C₁₋₆-alkyl, —C₂-C₆-alkenyl, —C₂-C₆-alkynyl, unsubstituted, mono- or polysubstituted with substituents independently of one another selected from the group consisting of —F, —Cl, —C₁₋₆-alkyl, —C₁₋₆-alkylene-CF₃, —OH, ═O, —OC₁₋₆-alkyl, —C₁₋₆-alkylene-OH, —C₁₋₆-alkylene-O—C₁₋₆-alkyl, —NH₂, —NHC₁₋₆-alkyl, —N(C₁₋₆-alkyl)₂, —NHC(═O)O—C₁₋₆-alkyl, —N(C₁₋₆-alkyl)C(═O)O—C₁₋₆-alkyl, —C₁₋₆-alkylene-NHC(═O)O—C₁₋₆-alkyl, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —C(═O)N(C₁₋₆-alkyl)₂, —S(═O)₂C₁₋₆-alkyl, -phenyl, —C₁₋₆-alkylene-phenyl, 3-14-membered heterocycloalkyl, saturated or unsaturated, unsubstituted; and 5-14-membered heteroaryl, unsubstituted;
3-14-membered cycloalkyl or —C₁₋₆-alkylene-(3-14-membered cycloalkyl), wherein —C₁₋₆-alkylene- is unsubstituted or monosubstituted with —OH, wherein said 3-14-membered cycloalkyl is selected from the group consisting of cyclopropyl, cyclobutyl, cyclopentyl, cyclohexyl, and cycloheptyl, in each case saturated or unsaturated, in each case unsubstituted, mono- or polysubstituted with substituents independently of one another selected from the group consisting of —F, —Cl, —C₁₋₆-alkyl, —C₁₋₆-alkylene-CF₃, —OH, ═O, —OC₁₋₆-alkyl, —C₁₋₆-alkylene-OH, —C₁₋₆-alkylene-O—C₁₋₆-alkyl, —NH₂, —NHC₁₋₆-alkyl, —N(C₁₋₆-alkyl)₂, —NHC(═O)O—C₁₋₆-alkyl, —N(C₁₋₆-alkyl)C(═O)O—C₁₋₆-alkyl, —C₁₋₆-alkylene-NHC(═O)O—C₁₋₆-alkyl, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —C(═O)N(C₁₋₆-alkyl)₂, —S(═O)₂C₁₋₆-alkyl, -phenyl, —C₁₋₆-alkylene-phenyl, 3-14-membered heterocycloalkyl, saturated or unsaturated, unsubstituted; and 5-14-membered heteroaryl, unsubstituted;
3-14-membered heterocycloalkyl or —C₁₋₆-alkylene-(3-14-membered heterocycloalkyl), wherein —C₁₋₆-alkylene- is unsubstituted or monosubstituted with —OH, wherein said 3-14-membered heterocycloalkyl in each case is selected from the group consisting of azepane, 1,4-oxazepane, azetidine, aziridine, azocane, diazepane, dioxane, dioxolane, dithiane, dithiolane, imidazolidine, isothiazolidine, isoxalidine, morpholine, oxazolidine, oxepane, oxetane, oxirane, piperazine, piperidine, pyrazolidine, pyrrolidine, quinuclidine, tetrahydrofuran, tetrahydropyran, tetrahydrothiopyran, thiazolidine, thietane, thiirane, thiolane, thiomorpholine, indoline, dihydrobenzofuran, dihydrobenzothiophene, 1,1-dioxothiacyclohexane, 2-azaspiro[3.3]heptane, 2-oxaspiro[3.3]heptane, 7-azaspiro[3.5]nonane, 8-azabicyclo[3.2.1]octane, 9-azabicyclo[3.3.1]nonane, hexahydro-1H-pyrrolizine, hexahydrocyclopenta[c]pyrrole, octahydrocyclopenta[c]pyrrole, and octahydropyrrolo[1,2-a]pyrazin; in each case unsubstituted, mono- or polysubstituted with substituents independently of one another selected from the group consisting of —F, —Cl, —C₁₋₆-alkyl, —C₁₋₆-alkylene-CF₃, —OH, ═O, —OC₁₋₆-alkyl, —C₁₋₆-alkylene-OH, —C₁₋₆-alkylene-O—C₁₋₆-alkyl, —NH₂, —NHC₁₋₆-alkyl, —N(C₁₋₆-alkyl)₂, —NHC(═O)O—C₁₋₆-alkyl, —N(C₁₋₆-alkyl)C(═O)O—C₁₋₆-alkyl, —C₁₋₆-alkylene-NHC(═O)O—C₁₋₆-alkyl, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —C(═O)N(C₁₋₆-alkyl)₂, —S(═O)₂C₁₋₆-alkyl, -phenyl, —C₁₋₆-alkylene-phenyl, 3-14-membered heterocycloalkyl, saturated or unsaturated, unsubstituted; and 5-14-membered heteroaryl, unsubstituted;
phenyl unsubstituted, mono- or polysubstituted with substituents independently of one another selected from the group consisting of —F, —Cl, —CN, —C₁₋₆-alkyl, —C₁₋₆-alkylene-CF₃, —OH, ═O, —OC₁₋₆-alkyl, —C₁₋₆-alkylene-OH, —C₁₋₆-alkylene-O—C₁₋₆-alkyl, —NH₂, —NHC₁₋₆-alkyl, —N(C₁₋₆-alkyl)₂, —NHC(═O)O—C₁₋₆-alkyl, —N(C₁₋₆-alkyl)C(═O)O—C₁₋₆-alkyl, —C₁₋₆-alkylene-NHC(═O)O—C₁₋₆-alkyl, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —C(═O)N(C₁₋₆-alkyl)₂, —S(═O)₂C₁₋₆-alkyl, -phenyl, —C₁₋₆-alkylene-phenyl, 3-14-membered heterocycloalkyl, saturated or unsaturated, unsubstituted; and 5-14-membered heteroaryl, unsubstituted;
5-14-membered heteroaryl or —C₁₋₆-alkylene-(5-14-membered heteroaryl), wherein —C₁₋₆-alkylene- is unsubstituted or monosubstituted with —OH, wherein said 5-14-membered heteroaryl in each case is selected from the group consisting of benzimidazole, benzisoxazole, benzoxazole, benzofuran, benzothiadiazole, benzothiazole, benzothiophene, carbazole, cinnoline, dibenzofuran, furan, furazan, imidazole, imidazopyridine, indazole, indole, indolizine, isobenzofuran, isoindole, isoquinoline, isothiazole, isoxazole, naphthyridine, oxadiazole, oxazole, oxindole, phthalazine, purine, pyrazine, pyrazole, pyridazine, pyridine, pyrimidine, pyrrole, quinazoline, quinoline, quinoxaline, tetrazole, thiadiazole, thiazole, thiophene, triazine, triazole, and [1,2,4]triazolo[4,3-a]pyrimidine; in each case unsubstituted, mono- or polysubstituted with substituents independently of one another selected from the group consisting of —F, —Cl, —CN, —C₁₋₆-alkyl, —C₁₋₆-alkylene-CF₃, —OH, ═O, —OC₁₋₆-alkyl, —C₁₋₆-alkylene-OH, —C₁₋₆-alkylene-O—C₁₋₆-alkyl, —NH₂, —NHC₁₋₆-alkyl, —N(C₁₋₆-alkyl)₂, —NHC(═O)O—C₁₋₆-alkyl, —N(C₁₋₆-alkyl)C(═O)O—C₁₋₆-alkyl, —C₁₋₆-alkylene-NHC(═O)O—C₁₋₆-alkyl, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —C(═O)N(C₁₋₆-alkyl)₂, —S(═O)₂C₁₋₆-alkyl, -phenyl, —C₁₋₆-alkylene-phenyl, 3-14-membered heterocycloalkyl, saturated or unsaturated, unsubstituted; and 5-14-membered heteroaryl, unsubstituted.

Preferably, R⁴ represents
—H;
—S(═O)₂C₁₋₆-alkyl monosubstituted or polysubstituted with —F;
—S(═O)₂(3-14-membered cycloalkyl) unsubstituted;
—C₁₋₆-alkyl, —C₂-C₆-alkenyl, —C₂-C₆-alkynyl, monosubstituted or disubstituted with substituents independently of one another selected from the group consisting of —OH, ═O, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-NH—C₁₋₆-alkyl, —C(═O)NH₂, —C(═O)—NH—C₁₋₃-alkyl, —C(═O)—N(C₁₋₃-alkyl)₂, or -phenyl unsubstituted;
3-14-membered cycloalkyl or —C₁₋₆-alkylene-(3-14-membered cycloalkyl), wherein —C₁₋₆-alkylene- is unsubstituted or monosubstituted with —OH, wherein said 3-14-membered cycloalkyl is saturated, unsubstituted, monosubstituted or disubstituted with substituents independently of one another selected from the group consisting of —C₁₋₆-alkyl, —C₁₋₆-alkylene-NH₂, —C₁₋₆-alkylene-NH—C₁₋₆-alkylene-CF₃, —C₁₋₆-alkylene-OH, —C₁₋₆-alkylene-NHC(═O)O—C₁₋₆-alkyl, —OH, —OC₁₋₆-alkyl, —NH₂, —N(C₁₋₆-alkyl)₂, —NHC(═O)O—C₁₋₆-alkyl;
3-14-membered heterocycloalkyl or —C₁₋₆-alkylene-(3-14-membered heterocycloalkyl), wherein —C₁₋₆-alkylene- is unsubstituted or monosubstituted with —OH, wherein said 3-14-membered heterocycloalkyl in each case is selected from azetane, 1,4-oxazepane, pyrrolidine, piperidine, azepane, diazepane, tetrahydrofuran, tetrahydropyran, oxetane, morpholine, piperazine, hexahydrocyclopenta[c]pyrrole, octahydrocyclopenta[c]pyrrole, octahydropyrrolo[1,2-a]pyrazin, 8-azabicyclo[3.2.1]octane, 9-azabicyclo[3.3.1]nonane, quinuclidine, hexahydro-1H-pyrrolizine, 2-oxaspiro[3.3]heptane, 2-azaspiro[3.3]heptane, 7-azaspiro[3.5]nonane, 1,1-dioxothiacyclohexane, in each case unsubstituted, mono- or polysubstituted with substituents independently of one another selected from the group consisting of —F, —OH, ═O, —C₁₋₆-alkyl, —C₁₋₆-alkylene-CF₃, —C₁₋₆-alkylene-OH, —C₁₋₆-alkylene-O—C₁₋₆-alkyl, —NH₂, —N(C₁₋₆-alkyl)₂, —C₁₋₆-alkylene-NH₂, —C₁₋₆-alkylene-N(C₁₋₆-alkyl)₂, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —S(═O)₂C₁₋₆-alkyl, oxetanyl, pyrimidinyl, —C₁₋₆-alkylene-phenyl;
-phenyl unsubstituted;
5-14-membered heteroaryl or —C₁₋₆-alkylene-(5-14-membered heteroaryl), wherein —C₁₋₆-alkylene- is unsubstituted or monosubstituted with —OH, wherein said 5-14-membered heteroaryl in each case is selected from the group consisting of pyridine, pyridazine, pyrazine, pyrazole, isoxazole, triazole, and [1,2,4]triazolo[4,3-a]pyrimidine, in each case unsubstituted, monosubstituted or disubstituted with substituents independently of one another selected from the group consisting of —C₁₋₆-alkyl, —OH.

In some embodiments, R⁴ represents
a residue selected from the group consisting of:

| | | | | |
|---|---|---|---|---|
| 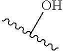 [SMILES: *O] | 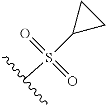 [SMILES: *S(=O)(=O)C1CC1] | 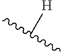 [SMILES: *[H]] | 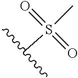 [SMILES: *S(C)(=O)=O] | 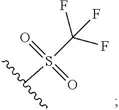 [SMILES: *S(=O)(=O)C(F)(F)F] |

or
C₁-C₆-alkyl unsubstituted, mono- or polysubstituted; preferably represents a residue —CR′R″—(CH₂)ₘ—OH, wherein m is an integer selected from 1, 2, 3, 4, 5, or 6, preferably selected from 1, 2 or 3; and wherein R′ and R″ independently of one another represent —H, —C₁₋₃-alkyl, —CF₃, —CF₂H, —CFH₂, —C₁₋₃-alkylene-CF₃, —C₁₋₃-alkylene-CF₂H, —C₁₋₃-alkylene-CFH₂, —CG-3-alkylene-O—C₁₋₃-alkyl, —C₁₋₃-alkylene-OH, —C(═O)—NH₂, —C(═O)—NH—C₁₋₃-alkyl, or —C(═O)—N(C₁₋₃-alkyl)₂; preferably —H, —CH₃, —C₁₋₃-alkylene-OH, C(═O)—NH₂, —C(═O)—NH—C₁₋₃-alkyl, or —C(═O)—N(C₁₋₃-alkyl)₂, preferably, at least R′ or R″ does not represent —H; preferably neither R′ nor R″ represents —H; preferably represents a residue selected from the group consisting of:

| | | | |
|---|---|---|---|
| 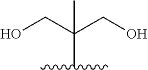 [SMILES: *C(C)(CO)CO] | 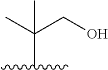 [SMILES: *C(C)(C)CO] | 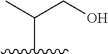 [SMILES: *C(C)CO] | 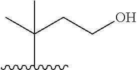 [SMILES: *C(C)(C)CCO] |
| 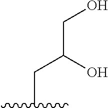 [SMILES: *CC(O)CO] | 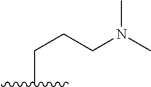 [SMILES: *CCCN(C)C] | 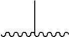 | 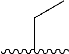 [SMILES: *CC] |
| 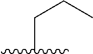 [SMILES: *CCC] | 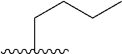 [SMILES: *CCCC] | 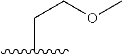 [SMILES: *CCOC] | 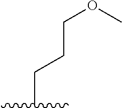 [SMILES: *CCCOC] |
| 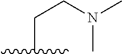 [SMILES: *CCN(C)C] | 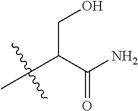 [SMILES: *C(CO)C(N)=O] | 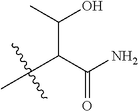 [SMILES: *C(C(N)=O)C(C)O] | 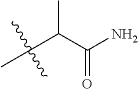 [SMILES: *C(C)C(N)=O] |
| 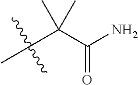 [SMILES: *C(C)(C)C(N)=O] | 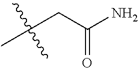 [SMILES: *CC(N)=O] | 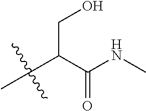 [SMILES: *C(CO)C(=O)NC] | 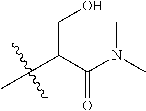 [SMILES: *C(CO)C(=O)N(C)C] |
| 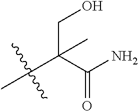 [SMILES: *C(C)(CO)C(N)=O] | 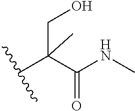 [SMILES: *C(C)(CO)C(=O)NC] | 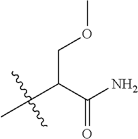 [SMILES: *C(COC)C(N)=O] | 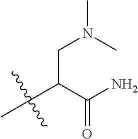 [SMILES: *C(CN(C)C)C(N)=O] |
| 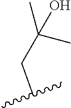 [SMILES: *CC(C)(C)O] | 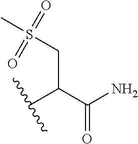 [SMILES: *C(CS(C)(=O)=O)C(N)=O] | | |

or;
a 3-14-membered cycloalkyl, saturated, unsubstituted, mono- or polysubstituted; preferably a 3-13-membered cycloalkyl, saturated, unsubstituted, mono or polysubstituted; preferably a 3-12-membered cycloalkyl, saturated, unsubstituted, mono or polysubstituted; preferably a 3-11-membered cycloalkyl, saturated, unsubstituted, mono or polysubstituted; preferably a 3-10-membered cycloalkyl, saturated, unsubstituted, mono or polysubstituted; preferably represents a cycloalkyl residue selected from the group consisting of

| | | | | |
|---|---|---|---|---|
| 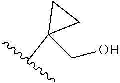 [SMILES: *C1(CO)CC1] | 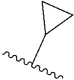 [SMILES: *C1CC1] | 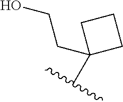 [SMILES: *C1(CCO)CCC1] | 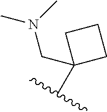 [SMILES: *C1(CN(C)C)CCC1] | 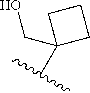 [SMILES: *C1(CO)CCC1] |
| 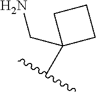 [SMILES: *C1(CN)CCC1] | 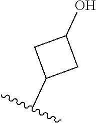 [SMILES: *C1CC(O)C1] | 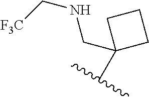 [SMILES: *C1(CNCC(F)(F)F)CCC1] | 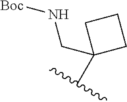 [SMILES: *C1(CNC(=O)OC(C)(C)C)CCC1] | 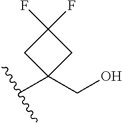 [SMILES: *C1(CO)CC(F)(F)C1] |
| 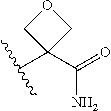 [SMILES: *C1(C(N)=O)COC1] | 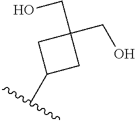 [SMILES: *C1CC(CO)(CO)C1] | 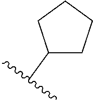 [SMILES: *C1CCCC1] | 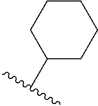 [SMILES: *C1CCCCC1] | 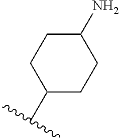 [SMILES: *C1CCC(N)CC1] |
| 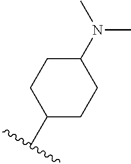 [SMILES: *C1CCC(N(C)C)CC1] | 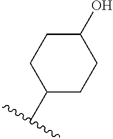 [SMILES: *C1CCC(O)CC1] | 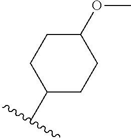 [SMILES: *C1CCC(OC)CC1] | 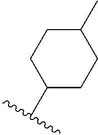 [SMILES: *C1CCC(C)CC1] | 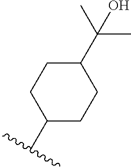 [SMILES: *C1CCC(C(C)(C)O)CC1] |
| 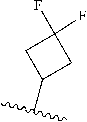 [SMILES: *C1CC(F)(F)C1] | 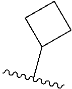 [SMILES: *C1CCC1] | 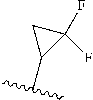 [SMILES: *C1CC1(F)F] | 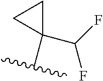 [SMILES: *C1(C(F)F)CC1] | 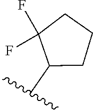 [SMILES: *C1CCCC1(F)F] |
| 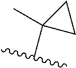 [SMILES: *C1(C)CC1] | 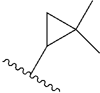 [SMILES: *C1CC1(C)C] | 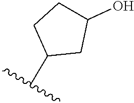 [SMILES: *C1CCC(O)C1] | 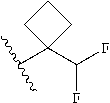 [SMILES: *C1(C(F)F)CCC1] | |

or;
3-14-membered cycloalkyl, saturated or unsaturated, unsubstituted, mono- or polysubstituted; wherein said 3-14-membered cycloalkyl is connected through —C₁-C₆-alkylene-, saturated or unsaturated, unsubstituted, mono- or polysubstituted; preferably a 3-12-membered cycloalkyl, saturated or unsaturated, unsubstituted, mono- or polysubstituted; wherein said 3-12-membered cycloalkyl is connected through —C₁-C₆-alkylene-, saturated or unsaturated, unsubstituted, mono- or polysubstituted; preferably a 3-10-membered cycloalkyl, saturated or unsaturated, unsubstituted, mono- or polysubstituted; wherein said 3-10-membered cycloalkyl is connected through —C₁-C₆-alkylene-, saturated or unsaturated, unsubstituted, mono- or polysubstituted; preferably a 3-8-membered cycloalkyl, saturated or unsaturated, unsubstituted, mono- or polysubstituted; wherein said 3-8-membered cycloalkyl is connected through —C₁-C₆-alkylene-, saturated or unsaturated, unsubstituted, mono- or polysubstituted, in particular 3-14-membered cycloalkyl, saturated or non-aromatic unsaturated, unsubstituted, mono- or polysubstituted; wherein said 3-14-membered cycloalkyl is connected through —C₁-C₆-alkylene-, saturated or unsaturated, unsubstituted, mono- or polysubstituted; preferably a 3-12-membered cycloalkyl, saturated or non-aromatic unsaturated, unsubstituted, mono- or polysubstituted; wherein said 3-12-membered cycloalkyl is connected through —C₁-C₆-alkylene-, saturated or unsaturated, unsubstituted, mono- or polysubstituted; preferably a 3-10-membered cycloalkyl, saturated or non-aromatic unsaturated, unsubstituted, mono- or polysubstituted; wherein said 3-10-membered cycloalkyl is connected through —C₁-C₆-alkylene-, saturated or unsaturated, unsubstituted, mono- or polysubstituted; preferably a 3-8-membered cycloalkyl, saturated or non-aromatic unsaturated, unsubstituted, mono- or polysubstituted; wherein said 3-8-membered cycloalkyl is connected through —C₁-C₆-alkylene-, saturated or unsaturated, unsubstituted, mono- or polysubstituted; preferably represents a residue selected from the group consisting of:

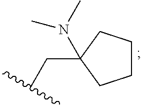 [SMILES: *CC1(N(C)C)CCCC1]

or
3-14-membered heterocycloalkyl, saturated, unsaturated, unsubstituted, mono- or polysubstituted; preferably 3-13-membered heterocycloalkyl, saturated, unsaturated, unsubstituted, mono- or polysubstituted; preferably 3-12-membered heterocycloalkyl, saturated, unsaturated, unsubstituted, mono- or polysubstituted; preferably 3-11-membered heterocycloalkyl, saturated, unsaturated, unsubstituted, mono- or polysubstituted; preferably 3-10-membered heterocycloalkyl, saturated, unsaturated, unsubstituted, mono- or poly substituted; preferably represents a heterocycloalkyl residue selected from the group consisting of:

| | | |
|---|---|---|
| 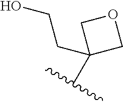 [SMILES: *C1(CCO)COC1] | 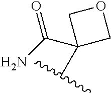 [SMILES: *C1(C(N)=O)COC1] | 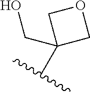 [SMILES: *C1(CO)COC1] |
| 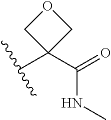 [SMILES: *C1(C(=O)NC)COC1] | 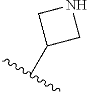 [SMILES: *C1CNC1] | 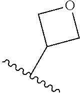 [SMILES: *C1COC1] |
| 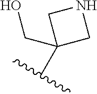 [SMILES: *C1(CO)CNC1] | 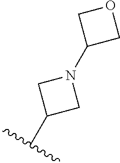 [SMILES: *C1CN(C2COC2)C1] | 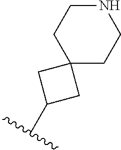 [SMILES: *C1CC2(CCNCC2)C1] |
| 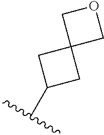 [SMILES: *C1CC2(COC2)C1] | 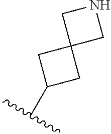 [SMILES: *C1CC2(CNC2)C1] | 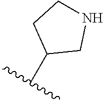 [SMILES: *C1CCNC1] |
| 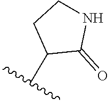 [SMILES: *C1CCNC1=O] | 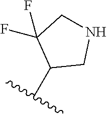 [SMILES: *C1CNCC1(F)F] | 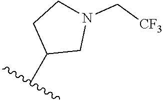 [SMILES: *C1CCN(CC(F)(F)F)C1] |
| 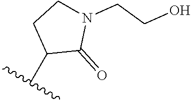 [SMILES: *C1CCN(CCO)C1=O] | 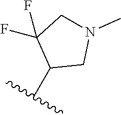 [SMILES: *C1CN(C)CC1(F)F] | 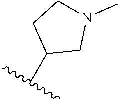 [SMILES: *C1CCN(C)C1] |
| 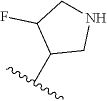 [SMILES: *C1CNCC1F] | 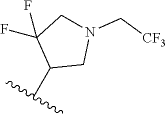 [SMILES: *C1CN(CC(F)(F)F)CC1(F)F] | 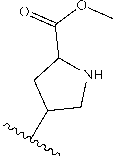 [SMILES: *C1CNC(C(=O)OC)C1] |
| 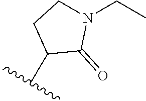 [SMILES: *C1CCN(CC)C1=O] | 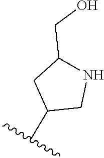 [SMILES: *C1CNC(CO)C1] | 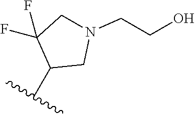 [SMILES: *C1CN(CCO)CC1(F)F] |
| 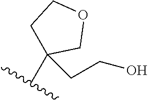 [SMILES: *C1(CCO)CCOC1] | 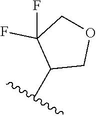 [SMILES: *C1COCC1(F)F] | 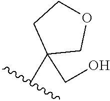 [SMILES: *C1(CO)CCOC1] |
| 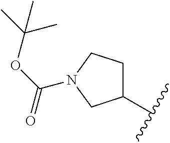 [SMILES: *C1CCN(C(=O)OC(C)(C)C)C1] | 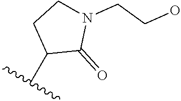 [SMILES: *C1CCN(CCO)C1=O] | 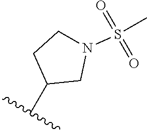 [SMILES: *C1CCN(S(C)(=O)=O)C1] |
| 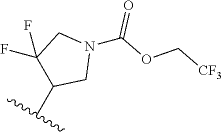 [SMILES: *C1CN(C(=O)OCC(F)(F)F)CC1(F)F] | 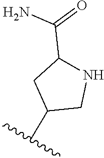 [SMILES: *C1CNC(C(N)=O)C1] | 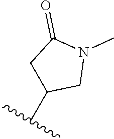 [SMILES: *C1CC(=O)N(C)C1] |
| 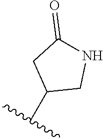 [SMILES: *C1CNC(=O)C1] | 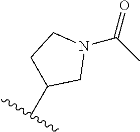 [SMILES: *C1CCN(C(C)=O)C1] | 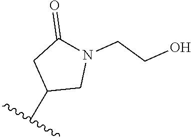 [SMILES: *C1CC(=O)N(CCO)C1] |
| 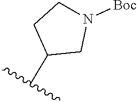 [SMILES: *C1CCN(C(=O)OC(C)(C)C)C1] | 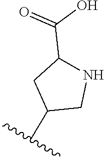 [SMILES: *C1CNC(C(=O)O)C1] | 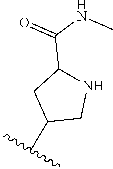 [SMILES: *C1CNC(C(=O)NC)C1] |
| 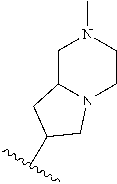 [SMILES: *C1CC2CN(C)CCN2C1] | 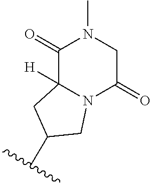 [SMILES: *C1CC2C(=O)N(C)CC(=O)N2C1] | 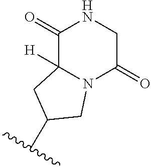 [SMILES: *C1CC2C(=O)NCC(=O)N2C1] |
| 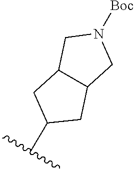 [SMILES: *C1CC2CN(C(=O)OC(C)(C)C)CC2C1] | 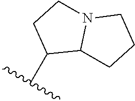 [SMILES: *C1CCN2CCCC12] | 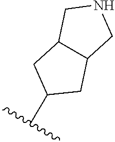 [SMILES: *C1CC2CNCC2C1] |
| 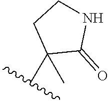 [SMILES: *C1(C)CCNC1=O] | 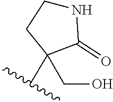 [SMILES: *C1(CO)CCNC1=O] | 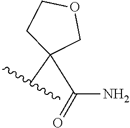 [SMILES: *C1(C(N)=O)CCOC1] |
| 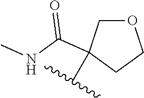 [SMILES: *C1(C(=O)NC)CCOC1] | 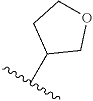 [SMILES: *C1CCOC1] | 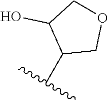 [SMILES: *C1COCC1O] |
| 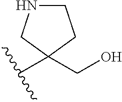 [SMILES: *C1(CO)CCNC1] | 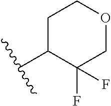 [SMILES: *C1CCOCC1(F)F] | 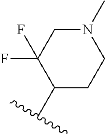 [SMILES: *C1CCN(C)CC1(F)F] |
| 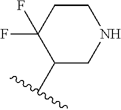 [SMILES: *C1CNCCC1(F)F] | 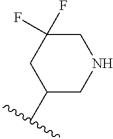 [SMILES: *C1CNCC(F)(F)C1] | 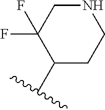 [SMILES: *C1CCNCC1(F)F] |
| 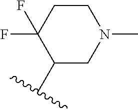 [SMILES: *C1CN(C)CCC1(F)F] | 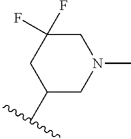 [SMILES: *C1CN(C)CC(F)(F)C1] | 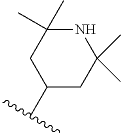 |
| 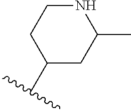 [SMILES: *C1CCNC(C)C1] | 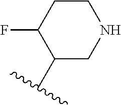 [SMILES: *C1CNCCC1F] | 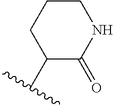 [SMILES: *C1CCCNC1=O] |
| 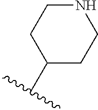 [SMILES: *C1CCNCC1] | 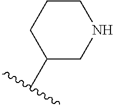 [SMILES: *C1CCCNC1] | 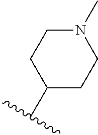 [SMILES: *C1CCN(C)CC1] |
| 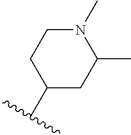 [SMILES: *C1CCN(C)C(C)C1] | 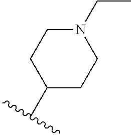 [SMILES: *C1CCN(CC)CC1] | 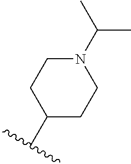 [SMILES: *C1CCN(C(C)C)CC1] |
| 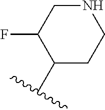 [SMILES: *C1CCNCC1F] | 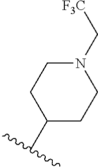 [SMILES: *C1CCN(CC(F)(F)F)CC1] | 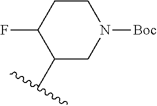 [SMILES: *C1CN(C(=O)OC(C)(C)C)CCC1F] |
| 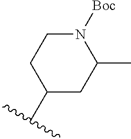 [SMILES: *C1CCN(C(=O)OC(C)(C)C)C(C)C1] | 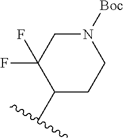 [SMILES: *C1CCN(C(=O)OC(C)(C)C)CC1(F)F] | 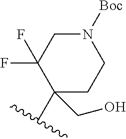 [SMILES: *C1(CO)CCN(C(=O)OC(C)(C)C)CC1(F)F] |
| 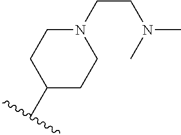 [SMILES: *C1CCN(CCN(C)C)CC1] | 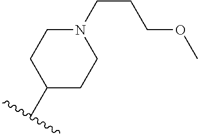 [SMILES: *C1CCN(CCCOC)CC1] | 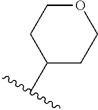 [SMILES: *C1CCOCC1] |
| 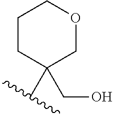 [SMILES: *C1(CO)CCCOC1] | 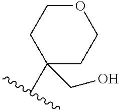 [SMILES: *C1(CO)CCOCC1] | 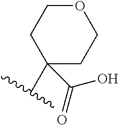 [SMILES: *C1(C(=O)O)CCOCC1] |
| 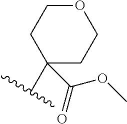 [SMILES: *C1(C(=O)OC)CCOCC1] | 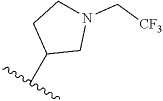 [SMILES: *C1CCN(CC(F)(F)F)C1] | 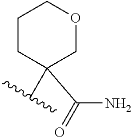 [SMILES: *C1(C(N)=O)CCCOC1] |
| 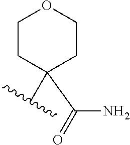 [SMILES: *C1(C(N)=O)CCOCC1] | 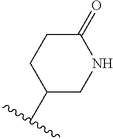 [SMILES: *C1CCC(=O)NC1] | 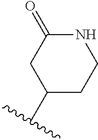 [SMILES: *C1CCNC(=O)C1] |
| 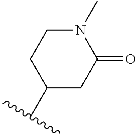 [SMILES: *C1CCN(C)C(=O)C1] | 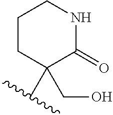 [SMILES: *C1(CO)CCCNC1=O] | 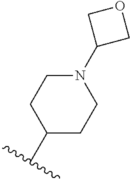 [SMILES: *C1CCN(C2COC2)CC1] |
| 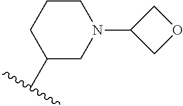 [SMILES: *C1CCCN(C2COC2)C1] | 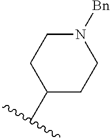 [SMILES: *C1CCN(Cc2ccccc2)CC1] | 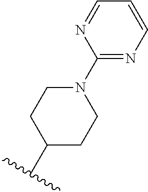 [SMILES: *C1CCN(c2ncccn2)CC1] |
| 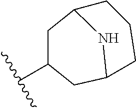 [SMILES: *C1CC2CCCC(C1)N2] | 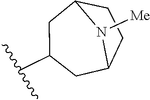 [SMILES: *C1CC2CCC(C1)N2C] | 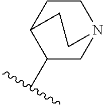 [SMILES: *C1CN2CCC1CC2] |
| 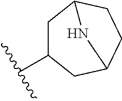 [SMILES: *C1CC2CCC(C1)N2] | 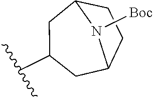 [SMILES: *C1CC2CCC(C1)N2C(=O)OC(C)(C)C] | 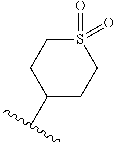 [SMILES: *C1CCS(=O)(=O)CC1] |
| 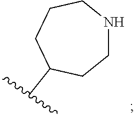 [SMILES: *C1CCCNCC1] | | |

more preferably the group consisting of:

| | | |
|---|---|---|
| 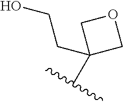 [SMILES: *C1(CCO)COC1] | 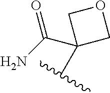 [SMILES: *C1(C(N)=O)COC1] | 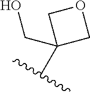 [SMILES: *C1(CO)COC1] |
| 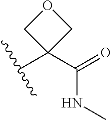 [SMILES: *C1(C(=O)NC)COC1] | 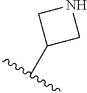 [SMILES: *C1CNC1] | 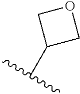 [SMILES: *C1COC1] |
| 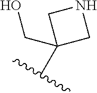 [SMILES: *C1(CO)CNC1] | 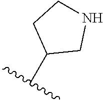 [SMILES: *C1CCNC1] | 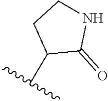 [SMILES: *C1CCNC1=O] |
| 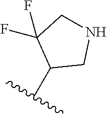 [SMILES: *C1CNCC1(F)F] | 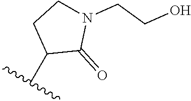 [SMILES: *C1CCN(CCO)C1=O] | 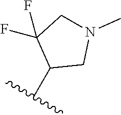 [SMILES: *C1CN(C)CC1(F)F] |
| 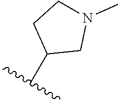 [SMILES: *C1CCN(C)C1] | 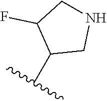 [SMILES: *C1CNCC1F] | 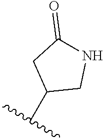 [SMILES: *C1CNC(=O)C1] |
| 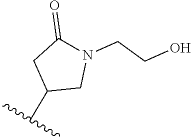 [SMILES: *C1CC(=O)N(CCO)C1] | 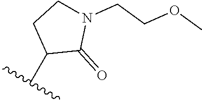 [SMILES: *C1CCN(CCOC)C1=O] | 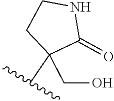 [SMILES: *C1(CO)CCNC1=O] |
| 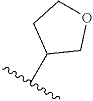 [SMILES: *C1CCOC1] | 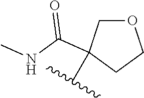 [SMILES: *C1(C(=O)NC)CCOC1] | 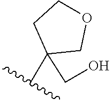 [SMILES: *C1(CO)CCOC1] |
| 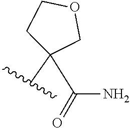 [SMILES: *C1(C(N)=O)CCOC1] | 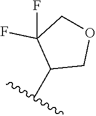 [SMILES: *C1COCC1(F)F] | 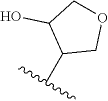 [SMILES: *C1COCC1O] |
| 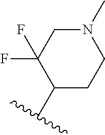 [SMILES: *C1CCN(C)CC1(F)F] | 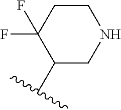 [SMILES: *C1CNCCC1(F)F] | 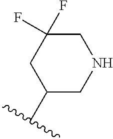 [SMILES: *C1CNCC(F)(F)C1] |
| 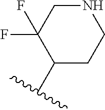 [SMILES: *C1CCNCC1(F)F] | 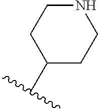 [SMILES: *C1CCNCC1] | 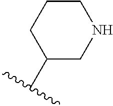 [SMILES: *C1CCCNC1] |
| 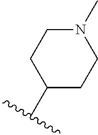 [SMILES: *C1CCN(C)CC1] | 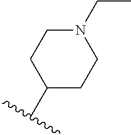 [SMILES: *C1CCN(CC)CC1] | 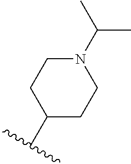 [SMILES: *C1CCN(C(C)C)CC1] |
| 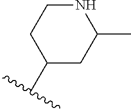 [SMILES: *C1CCNC(C)C1] | 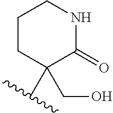 [SMILES: *C1(CO)CCCNC1=O] | 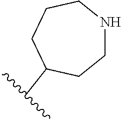 [SMILES: *C1CCCNCC1] |
| 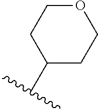 [SMILES: *C1CCOCC1] | 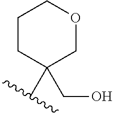 [SMILES: *C1(CO)CCCOC1] | 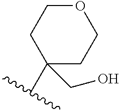 [SMILES: *C1(CO)CCOCC1] |
| 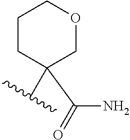 [SMILES: *C1(C(N)=O)CCCOC1] | 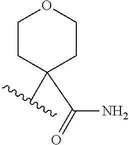 [SMILES: *C1(C(N)=O)CCOCC1] | |

3-14-membered heterocycloalkyl, saturated or unsaturated, unsubstituted, mono- or polysubstituted; wherein said 3-14-membered heterocycloalkyl is connected through-C₁-C₆-alkylene-, saturated or unsaturated, unsubstituted, mono- or polysubstituted; preferably a 3-12-membered heterocycloalkyl, saturated or unsaturated, unsubstituted, mono- or polysubstituted; wherein said 3-12-membered heterocycloalkyl is connected through —C₁-C₆-alkylene-, saturated or unsaturated, unsubstituted, mono- or polysubstituted; preferably a 3-10-membered heterocycloalkyl, saturated or unsaturated, unsubstituted, mono- or polysubstituted; wherein said 3-10-membered heterocycloalkyl is connected through —C₁-C₆-alkylene-, saturated or unsaturated, unsubstituted, mono- or polysubstituted; preferably a 3-8-membered heterocycloalkyl, saturated or unsaturated, unsubstituted, mono- or polysubstituted; wherein said 3-8-membered heterocycloalkyl is connected through —C₁-C₆-alkylene-, saturated or unsaturated, unsubstituted, mono- or polysubstituted; preferably represents a residue selected from the group consisting of:

| | | |
|---|---|---|
| 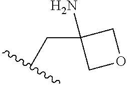 [SMILES: *CC1(N)COC1] | 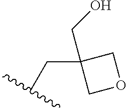 [SMILES: *CC1(CO)COC1] | 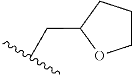 [SMILES: *CC1CCCO1] |
| 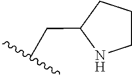 [SMILES: *CC1CCCN1] | 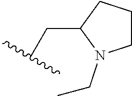 [SMILES: *CC1CCCN1CC] | 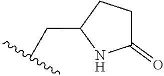 [SMILES: *CC1CCC(=O)N1] |
| 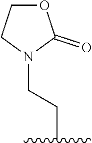 [SMILES: *CCN1CCOC1=O] | 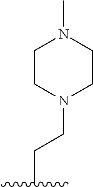 [SMILES: *CCN1CCN(C)CC1] | 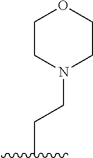 [SMILES: *CCN1CCOCC1] |
| 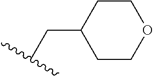 [SMILES: *CC1CCOCC1] | 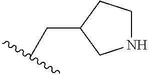 [SMILES: *CC1CCNC1] | 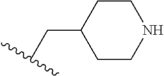 [SMILES: *CC1CCNCC1] |
| 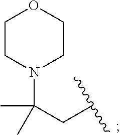 [SMILES: *CC(C)(C)N1CCOCC1] | | |

or
6-14-membered aryl, unsubstituted, mono- or polysubstituted; preferably a 6-12-membered aryl, unsubstituted, mono- or polysubstituted; preferably a 6-10-membered aryl, unsubstituted, mono- or polysubstituted; preferably a 6-8-membered aryl, unsubstituted, mono- or polysubstituted; or
6-14-membered aryl, unsubstituted, mono- or polysubstituted, wherein said 6-14-membered aryl is connected through —C₁-C₆-alkylene-, saturated or unsaturated, unsubstituted, mono- or polysubstituted; preferably a 6-12-membered aryl, unsubstituted, mono- or polysubstituted, wherein said 6-12-membered aryl is connected through —C₁-C₆-alkylene-, saturated or unsaturated, unsubstituted, mono- or polysubstituted; preferably a 6-10-membered aryl, unsubstituted, mono- or polysubstituted, wherein said 6-10-membered aryl is connected through —C₁-C₆-alkylene-, saturated or unsaturated, unsubstituted, mono- or polysubstituted; preferably represents a residue selected from the group consisting of:

| | | |
|---|---|---|
| 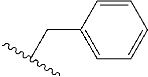 [SMILES: *Cc1ccccc1] | 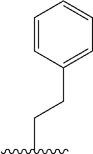 [SMILES: *CCc1ccccc1] | 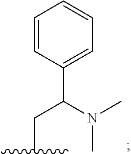 [SMILES: *CC(c1ccccc1)N(C)C] |

or
5-14-membered heteroaryl unsubstituted, mono- or polysubstituted; preferably a 5-12-membered heteroaryl unsubstituted, mono- or polysubstituted; preferably a 5-10-membered heteroaryl unsubstituted, mono- or polysubstituted; preferably a 5-9-membered heteroaryl unsubstituted, mono- or polysubstituted; preferably represents a residue selected from the group consisting of:

| | | |
|---|---|---|
| 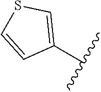 [SMILES: *c1ccsc1] | 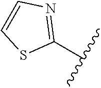 [SMILES: *c1nccs1] | 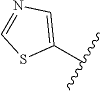 [SMILES: *c1cncs1] |
| 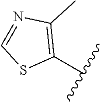 [SMILES: *c1scnc1C] | 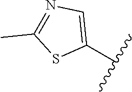 [SMILES: *c1cnc(C)s1] | 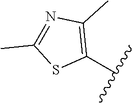 [SMILES: *c1sc(C)nc1C] |
| 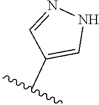 [SMILES: *c1cn[nH]c1] | 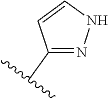 [SMILES: *c1cc[nH]n1] | 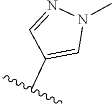 [SMILES: *c1cnn(C)c1] |
| 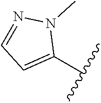 [SMILES: *c1ccnn1C] | 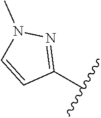 [SMILES: *c1ccn(C)n1] | 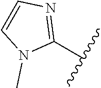 [SMILES: *c1nccn1C] |
| 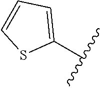 [SMILES: *c1cccs1] | 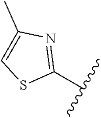 [SMILES: *c1nc(C)cs1] | 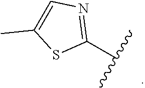 [SMILES: *c1ncc(C)s1] |
| 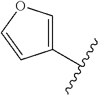 [SMILES: *c1ccoc1] | 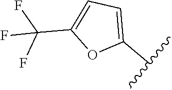 [SMILES: *c1ccc(C(F)(F)F)o1] | 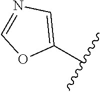 [SMILES: *c1cnco1] |
| 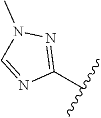 [SMILES: *c1ncn(C)n1] | 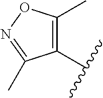 [SMILES: *c1c(C)noc1C] | 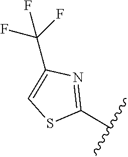 [SMILES: *c1nc(C(F)(F)F)cs1] |
| 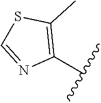 [SMILES: *c1ncsc1C] | 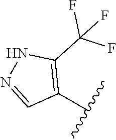 [SMILES: *c1cn[nH]c1C(F)(F)F] | 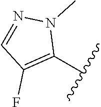 [SMILES: *c1c(F)cnn1C] |
| 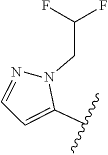 [SMILES: *c1ccnn1CC(F)F] | 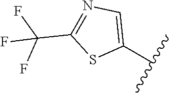 [SMILES: *c1cnc(C(F)(F)F)s1] | 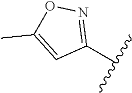 [SMILES: *c1cc(C)on1] |
| 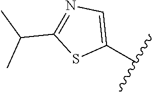 [SMILES: *c1cnc(C(C)C)s1] | 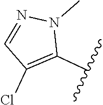 [SMILES: *c1c(Cl)cnn1C] | 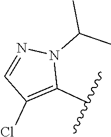 [SMILES: *c1c(Cl)cnn1C(C)C] |
| 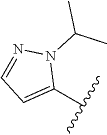 [SMILES: *c1ccnn1C(C)C] | 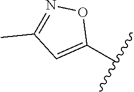 [SMILES: *c1cc(C)no1] | 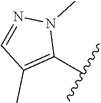 [SMILES: *c1c(C)cnn1C] |
| 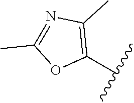 [SMILES: *c1oc(C)nc1C] | 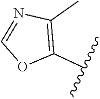 [SMILES: *c1ocnc1C] | 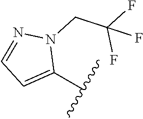 [SMILES: *c1ccnn1CC(F)(F)F] |
| 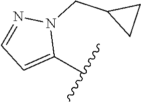 [SMILES: *c1ccnn1CC1CC1] | 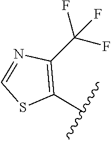 [SMILES: *c1scnc1C(F)(F)F] | 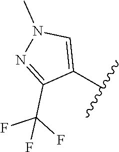 [SMILES: *c1cn(C)nc1C(F)(F)F] |
| 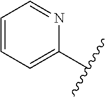 [SMILES: *c1ccccn1] | 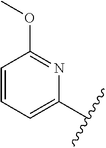 [SMILES: *c1cccc(OC)n1] | 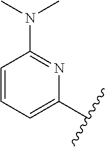 [SMILES: *c1cccc(N(C)C)n1] |
| 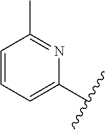 [SMILES: *c1cccc(C)n1] | 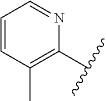 [SMILES: *c1ncccc1C] | 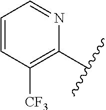 [SMILES: *c1ncccc1C(F)(F)F] |
| 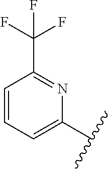 [SMILES: *c1cccc(C(F)(F)F)n1] | 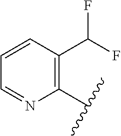 [SMILES: *c1ncccc1C(F)F] | 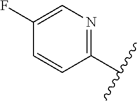 [SMILES: *c1ccc(F)cn1] |
| 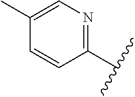 [SMILES: *c1ccc(C)cn1] | 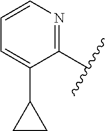 [SMILES: *c1ncccc1C1CC1] | 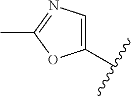 [SMILES: *c1cnc(C)o1] |
| 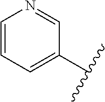 [SMILES: *c1cccnc1] | 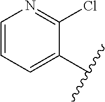 [SMILES: *c1cccnc1Cl] | 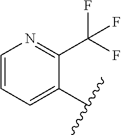 [SMILES: *c1cccnc1C(F)(F)F] |
| 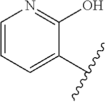 [SMILES: *c1cccnc1O] | 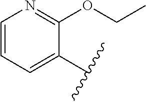 [SMILES: *c1cccnc1OCC] | 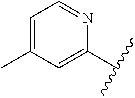 [SMILES: *c1cc(C)ccn1] |
| 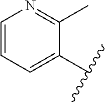 [SMILES: *c1cccnc1C] | 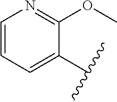 [SMILES: *c1cccnc1OC] | 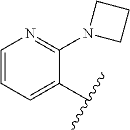 [SMILES: *c1cccnc1N1CCC1] |
| 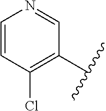 [SMILES: *c1cnccc1Cl] | 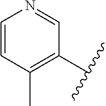 [SMILES: *c1cnccc1C] | 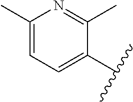 [SMILES: *c1ccc(C)nc1C] |
| 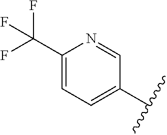 [SMILES: *c1ccc(C(F)(F)F)nc1] | 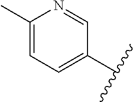 [SMILES: *c1ccc(C)nc1] | 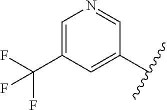 [SMILES: *c1cncc(C(F)(F)F)c1] |
| 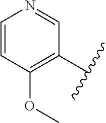 [SMILES: *c1cnccc1OC] | 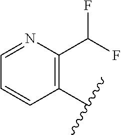 [SMILES: *c1cccnc1C(F)F] | 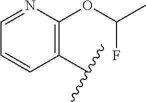 [SMILES: *c1cccnc1OC(C)F] |
| 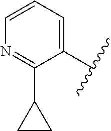 [SMILES: *c1cccnc1C1CC1] | 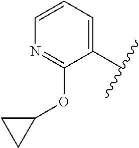 [SMILES: *c1cccnc1OC1CC1] | 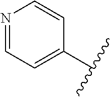 [SMILES: *c1ccncc1] |
| 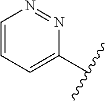 [SMILES: *c1cccnn1] | 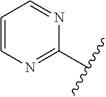 [SMILES: *c1ncccn1] | 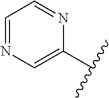 [SMILES: *c1cnccn1] |
| 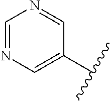 [SMILES: *c1cncnc1] | 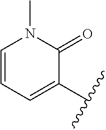 [SMILES: *c1cccn(C)c1=O] | 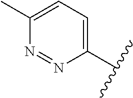 [SMILES: *c1ccc(C)nn1] |
| 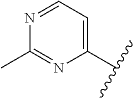 [SMILES: *c1ccnc(C)n1] | [symbol] ; | |

[symbol]

indicates data missing or illegible when filed

more preferably represents a residue selected from the group consisting of:

| | | |
|---|---|---|
| 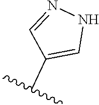 [SMILES: *c1cn[nH]c1] | 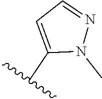 [SMILES: *c1ccnn1C] | 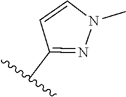 [SMILES: *c1ccn(C)n1] |
| 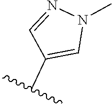 [SMILES: *c1cnn(C)c1] | 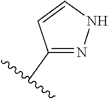 [SMILES: *c1cc[nH]n1] | 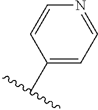 [SMILES: *c1ccncc1] |
| 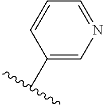 [SMILES: *c1cccnc1] | 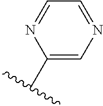 [SMILES: *c1cnccn1] | 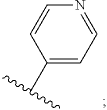 [SMILES: *c1ccncc1] |

or
5-14-membered heteroaryl, unsubstituted, mono- or polysubstituted; wherein said 5-14-membered heteroaryl is connected through —C₁-C₆-alkylene-, saturated or unsaturated, unsubstituted, mono- or polysubstituted; preferably 5-12-membered heteroaryl, unsubstituted, mono- or polysubstituted; wherein said 5-12-membered heteroaryl is connected through —C₁-C₆-alkylene-, saturated or unsaturated, unsubstituted, mono- or polysubstituted; 5-10-membered heteroaryl, unsubstituted, mono- or polysubstituted; wherein said 5-10-membered heteroaryl is connected through —C₁-C₆-alkylene-, saturated or unsaturated, unsubstituted, mono- or polysubstituted; 5-8-membered heteroaryl, unsubstituted, mono- or polysubstituted; wherein said 5-8-membered heteroaryl is connected through —C₁-C₆-alkylene-, saturated or unsaturated, unsubstituted, mono- or polysubstituted; preferably represents a residue selected from the group consisting of:

| | | |
|---|---|---|
| 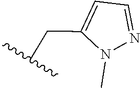 [SMILES: *Cc1ccnn1C] | 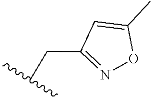 [SMILES: *Cc1cc(C)on1] | 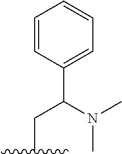 [SMILES: *CC(c1ccccc1)N(C)C] |
| 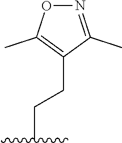 | 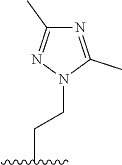 | 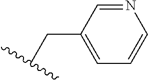 [SMILES: *Cc1cccnc1] |
| 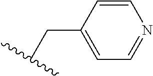 [SMILES: *Cc1ccncc1] | 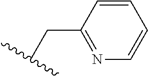 [SMILES: *Cc1ccccn1] | 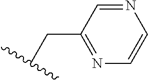 [SMILES: *Cc1cnccn1] |
| 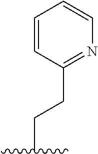 [SMILES: *CCc1ccccn1] | 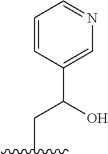 [SMILES: *CC(O)c1cccnc1] | 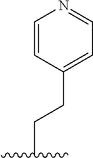 [SMILES: *CCc1ccncc1] |
| 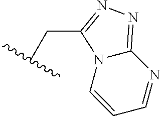 [SMILES: *Cc1nnc2ncccn12] | 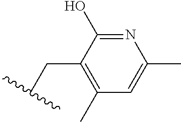 [SMILES: *Cc1c(C)cc(C)nc1O] | 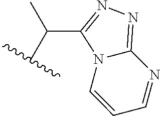 [SMILES: *C(C)c1nnc2ncccn12] |
| 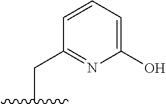 [SMILES: *Cc1cccc(O)n1] | 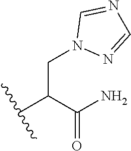 [SMILES: *C(Cn1cncn1)C(N)=O] | |

In some embodiments, R⁴ represents a residue according to general formula (A),

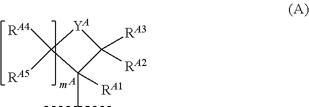

wherein
m^{A} is 0 or 1;
Y^{A} is selected from —O—, —NR^{A6}— and —CR^{A7}R^{A8}—; and
R^{A1}, R^{A2}, R^{A3}, R^{A4}, R^{A5}, R^{A6}, R^{A7}, and R^{A8} independently of one another represent —H, —F, —C₁₋₃-alkyl, —C₁₋₃-alkylene-OH, —C₁₋₃-alkylene-NH₂, —C₁₋₃-alkylene-NH(C₁₋₃-alkyl), —C₁₋₃-alkylene-N(C₁₋₃-alkyl)₂, —C₁₋₃-alkylene-NH(C₁₋₃-alkylene-CF₃), —C₁₋₃-alkylene-C(═O)NH₂, —C₁₋₃-alkylene-NH—C(═O)OC₁₋₄-alkyl, —C(═O)NH₂, —C(═O)—NH—C₁₋₃-alkyl, —C(═O)—N(C₁₋₃-alkyl)₂, or -3-oxetanyl; or R^{A7} and RAS together with the carbon atom to which they are attached form a ring and represent —CH₂OCH₂—, —CH₂OCH₂CH₂— or —CH₂CH₂OCH₂CH₂—, —CH₂NHCH₂—, —CH₂NHCH₂CH₂— or —CH₂CH₂NHCH₂CH₂—.

In some embodiments, R⁴ represents a residue according to general formula (A) as defined above, wherein
m^{A} is 0 or 1;
Y^{A} is selected from —O— and —CR^{A7}R^{A8}—; and
R^{A1}, R^{A2}, R^{A3}, R^{A4}, R^{A5}, R^{A7}, and R^{A8} independently of one another represent —H, —C₁₋₃-alkylene-OH, —C₁₋₃-alkylene-N(C₁₋₃-alkyl)₂, or —C(═O)NH₂; preferably with the proviso that only one of R^{A1}, R^{A2}, R^{A3}, R^{A4}, R^{A5}, R^{A7}, and R^{A8} represents a residue that is not —H.

In some embodiments, R⁴ represents a residue according to general formula (A) as defined above, wherein
m^{A} is 0 or 1;
Y^{A} is selected from —O— and —CR^{A7}R^{A8}—; and
R^{A1} represents —C₁₋₃-alkylene-OH, —C₁₋₃-alkylene-N(C₁₋₃-alkyl)₂, or —C(═O)NH₂; and
R^{A2}, R^{A3}, R^{A4}, R^{A5}, R^{A7}, and R^{A8} represent —H.

In some embodiments, R⁴ represents a residue according to general formula (B),

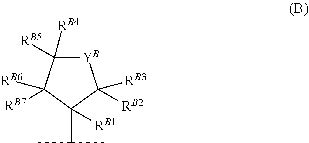

wherein
Y^{B} is selected from —O—, —NR^{B8}— and —CR^{B9}R^{B10}—; and
R^{B1}, R^{B2}, R^{B3}, R^{B4}, R^{B5}, R^{B6}, R^{B7}, R^{B8}, R^{B9} and R^{B10} independently of one another represent —H, —F, —OH, —C(═O)—NH₂, —C(═O)—NH—C₁₋₃-alkyl, —C(═O)—N(C₁₋₃-alkyl)₂, —C₁₋₃-alkyl, —C₁₋₃-alkylene-OH, —C₁₋₃-alkylene-O—C₁₋₃-alkyl, —C₁₋₃-alkylene-CF₃, —C₁₋₃-alkylene-CO₂H, or —C₁₋₃-alkylene-C(═O)O—C₁₋₃-alkyl; or R^{B2} and R^{B3} together represent ═0; or R^{B4} and R^{B5} together represent ═O.

In some embodiments, R⁴ represents a residue according to general formula (B) as defined above, wherein
Y^{B} is selected from —O— and —NR^{B8}—; and
R^{B1}, R^{B2}, R^{B3}, R^{B4}, R^{B5}, R^{B6}, R^{B7}, R^{B8} independently of one another represent —H, —F, —C₁₋₃-alkyl, —C₁₋₃-alkylene-OH, —C₁₋₃-alkylene-CF₃, or —C(═O)—NH₂; or R^{B2} and R^{B3} together represent ═O; or R^{B4} and R^{B5} together represent ═O; preferably with the proviso that only 1, 2 or 3 of R^{A1}, R^{A2}, R^{A3}, R^{A4}, R^{A5}, R^{A7}, and R^{A8} represent a residue that is not —H; preferably with the proviso that at least one of R^{A1}, R^{A2}, R^{A3}, R^{A4}, R^{A5}, R^{A7}, and R^{A8} represent a residue that is not —H.

In some embodiments, R⁴ represents a residue according to general formula (C),

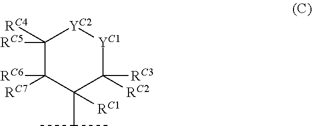

wherein
Y^{C1} is selected from —O—, —S(═O)₂—, —NR^{C8}— and —CR^{C9}R^{C10}— and Y^{C2} represents —CR^{C11}R^{C12}—; or Y^{C1} represents —CR^{C9}R^{C10}— and Y^{C2} is selected from —O—, —S(═O)₂—, and —NR^{C8}—;
R^{C1}, R^{C2}, R^{C3}, R^{C4}, R^{C5}, R^{C6}, R^{C7}, R^{C8}, R^{C9}, R^{C10}, R^{C11} and R^{C12} independently of one another represent —H, —F, —OH, —C(═O)OC₁₋₃-alkyl, —NH₂, —NH(C₁₋₃-alkyl), —N(C₁₋₃-alkyl)₂, —C₁₋₃-alkyl, —C₁₋₃-alkylene-OH, —C₁₋₃-alkylene-CF₃, —C(═O)—NH₂, —C(═O)—NH—C₁₋₃-alkyl, or —C(═O)N—(C₁₋₃-alkyl)₂; or R^{C2} and R^{C3} together represent ═O; or R^{C4} and R^{C5} together represent ═O; or R^{C9} and R^{C10} together represent ═O; or R^{C11} and R^{C12} together represent ═O.

In some embodiments, R⁴ represents a residue according to general formula (C) as defined above, wherein
Y^{C1} is selected from —O— or —NR^{C8}— and Y^{C2} represents —CR^{C11}R^{C12}— or Y^{C1} represents —CR^{C9}R^{C10}— and Y^{C2} is selected from —O—, and —NR^{C8}—;
R^{C1}, R^{C2}, R^{C3}, R^{C4}, R^{C5}, R^{C6}, R^{C7}, R^{C8}, R^{C9}, R^{C10}, R^{C11} and R^{C12} independently of one another represent —H, —F, —C₁₋₃-alkyl, —C₁₋₃-alkylene-OH or —C(═O)—NH₂; preferably with the proviso that only 1, 2 or 3 of R^{C1}, R^{C2}, R^{C3}, R^{C4}, R^{C5}, R^{C6}, R^{C7}, R^{C8}, R^{C9}, R^{C10}, R^{C11} and R^{C12} represent a residue that is not —H; preferably with the proviso that at least one of R^{C1}, R^{C2}, R^{C3}, R^{C4}, R^{C5}, R^{C6}, R^{C7}, R^{C8}, R^{C9}, R^{C10}, R^{C11} and R^{C12} represent a residue that is not —H.

In some embodiments, R⁴ represents
(i) a residue —CR′R″—(CH₂)ₘ—OH, wherein m is an integer of from 1 to 6, preferably from 1 to 3; and wherein R′ and R″ independently of one another represent —H, —C₁₋₃-alkyl, —CF₃, —CF₂H, —CFH₂, —C₁₋₃-alkylene-CF₃, —C₁₋₃-alkylene-CF₂H, —C₁₋₃-alkylene-CFH₂, —C₁₋₃-alkylene-O—C₁₋₃-alkyl, or —C₁₋₃-alkylene-OH; preferably —H, —CH₃, or —C₁₋₃-alkylene-OH. In a preferred embodiment, at least R′ or R″ does not represent —H. In a preferred embodiment, neither R′ nor R″ represents —H; or
(ii) a residue according to general formula (D),

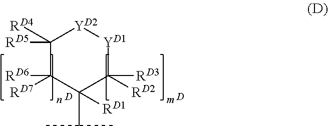

wherein
m^{D} and n^{D} independently of one another are 0, 1, 2, or 3; preferably with the proviso that m^{D}+n^{D}≤3;
Y^{D1} is selected from —O—, —S(═O)₂—, —NR^{D8}— and —CR^{D9}R^{D10}— and Y^{D2} represents —CR^{D11}R^{D12}—; or Y^{D1} represents —CR^{D9}R^{D10}— and Y^{D2} is selected from —O—, —S(═O)₂—, and —NR^{D8}—;
R^{D1}, R^{D2}, R^{D3}, R^{D4}, R^{D5}, R^{D6}, R^{D7}, R^{D8}, R^{D9}, R^{D10}, R^{D11} and R^{D12} independently of one another represent —H, —F, —OH, —C₁₋₃-alkylene-OH, —C(═O)NH₂, —C₁-alkylene-C(O)NH₂, —C(═O)O—CH₂-alkyl, —NH₂, alkylene-NH₂, —NH(C₁₋₃-alkyl), —N(C₁₋₃-alkyl)₂, —NH(C₁₋₃-alkylene-CF₃), —C₁₋₃-alkylene-OCH₃, —C₁₋₃-alkyl, —C₁₋₃-alkylene-CF₃; or R^{D2} and R^{D3} together represent ═O; or R^{D4} and R^{D5} together represent ═O; or R^{D9} and R^{D10} together represent ═O; or R^{D11} and R^{D12} together represent ═O;
preferably wherein
m^{D} and n^{D} independently of one another are 0, 1, 2 or 3; preferably with the proviso that m^{D}+n^{D}≤3;
Y^{D1} is selected from —O—, —NR^{D8}— and —CR^{D9}R^{D10}— and Y^{D2} represents —CR^{D11}R^{D12}—; or Y^{D1} represents —CR^{D9}R^{D10}— and Y^{D2} is selected from —O— and —NR^{D8}—;
R^{D1}, R^{D2}, R^{D3}, R^{D4}, R^{D5}, R^{D6}, R^{D7}, R^{D8}, R^{D9}, R^{D10}, R^{D11} and RD¹² independently of one another represent —H, —F, —OH, —C₁₋₃-alkylene-OH, —C(═O)NH₂, —CH₂NH₂, —CH₂N(CH₃)₂, —NHCH₂CF₃, —CH₃, or —CH₂CF₃; or R^{D2} and R^{D3} together represent ═O; or R^{D4} and R^{D5} together represent ═O; or R^{D9} and R^{D10} together represent ═O; or R^{D11} and R^{D12} together represent ═O; preferably with the proviso that only 1, 2 or 3 of R^{D1}, R^{D2}, R^{D3}, R^{D4}, R^{D5}, R^{D6}, R^{D7}, R^{D8}, R^{D9}, R^{D10}, R^{D11} and R^{D1} represent a residue that is not —H; preferably with the proviso that at least one of R^{D1}, R^{D2}, R^{D3}, R^{D4}, R^{D5}, R^{D6}, R^{D7}, R^{D8}, R^{D9}, R^{D10}, R^{D11} and R^{D12} represent a residue that is not —H.

In some embodiments, R⁴ represents a residue according to general formulae (A), (B), (C), (D) or a residue of formula —CR′R″—(CH₂)ₘ—OH as described herein.

In some embodiments, R⁴ represents a residue selected from the group consisting of: —CH₃,

| | | |
|---|---|---|
| 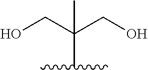 [SMILES: *C(C)(CO)CO] | 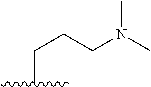 [SMILES: *CCCN(C)C] | 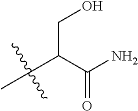 [SMILES: *C(CO)C(N)=O] |
| 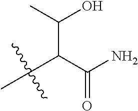 [SMILES: *C(C(N)=O)C(C)O] | 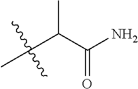 [SMILES: *C(C)C(N)=O] | 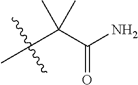 [SMILES: *C(C)(C)C(N)=O] |
| 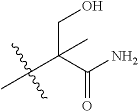 [SMILES: *C(C)(CO)C(N)=O] | 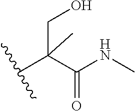 [SMILES: *C(C)(CO)C(=O)NC] | 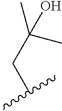 [SMILES: *CC(C)(C)O] |
| 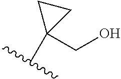 [SMILES: *C1(CO)CC1] | 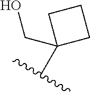 [SMILES: *C1(CO)CCC1] | 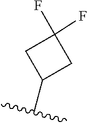 [SMILES: *C1CC(F)(F)C1] |
| 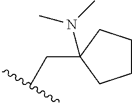 [SMILES: *CC1(N(C)C)CCCC1] | 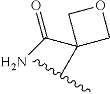 [SMILES: *C1(C(N)=O)COC1] | 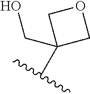 [SMILES: *C1(CO)COC1] |
| 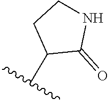 [SMILES: *C1CCNC1=O] | 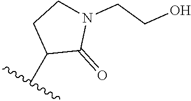 [SMILES: *C1CCN(CCO)C1=O] | 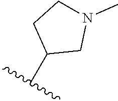 [SMILES: *C1CCN(C)C1] |
| 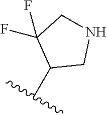 [SMILES: *C1CNCC1(F)F] | 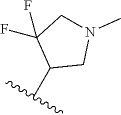 [SMILES: *C1CN(C)CC1(F)F] | 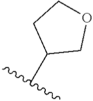 [SMILES: *C1CCOC1] |
| 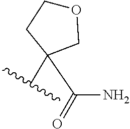 [SMILES: *C1(C(N)=O)CCOC1] | 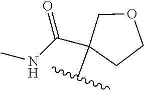 [SMILES: *C1(C(=O)NC)CCOC1] | 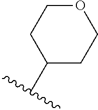 [SMILES: *C1CCOCC1] |
| 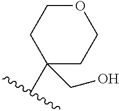 [SMILES: *C1(CO)CCOCC1] | 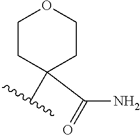 [SMILES: *C1(C(N)=O)CCOCC1] | 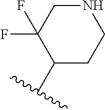 [SMILES: *C1CCNCC1(F)F] |
| 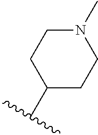 [SMILES: *C1CCN(C)CC1] | 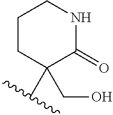 [SMILES: *C1(CO)CCCNC1=O] | 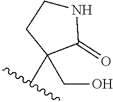 [SMILES: *C1(CO)CCNC1=O] |
| 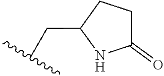 [SMILES: *CC1CCC(=O)N1] | 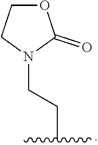 [SMILES: *CCN1CCOC1=O] | 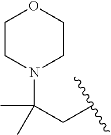 [SMILES: *CC(C)(C)N1CCOCC1] |
| 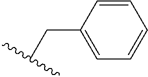 [SMILES: *Cc1ccccc1] | 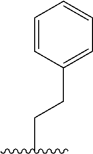 [SMILES: *CCc1ccccc1] | 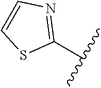 [SMILES: *c1nccs1] |
| 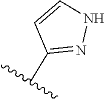 [SMILES: *c1cc[nH]n1] | 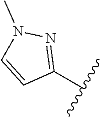 [SMILES: *c1ccn(C)n1] | 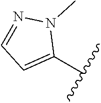 [SMILES: *c1ccnn1C] |
| 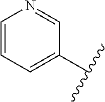 [SMILES: *c1cccnc1] | 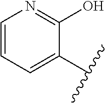 [SMILES: *c1cccnc1O] | 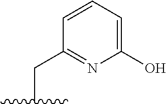 [SMILES: *Cc1cccc(O)n1] |
| 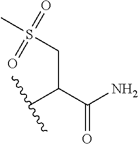 [SMILES: *C(CS(C)(=O)=O)C(N)=O] | | |

In some embodiments R³ is H and R⁴ is any of the embodiments described hereinabove.

In some embodiments R⁵ and R^{5′} independently of one another represent
—H;
—C₁-C₆-alkyl, saturated or unsaturated, unsubstituted, mono- or polysubstituted;
—C₁-C₆-heteroalkyl, saturated or unsaturated, unsubstituted, mono- or polysubstituted;
3-14-membered cycloalkyl, saturated or unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted, in particular 3-14-membered cycloalkyl, saturated or non-aromatic unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted.

Preferably, R⁵ and R^{5′} independently of one another represent —H, —C₁-C₆-alkyl, or —C₁-C₆-alkylene-N(C₁-C₆-alkyl)₂.

In some embodiments at least one of R⁵ and R^{5′} is not —H.

In some embodiments, R⁵ and R^{5′} are both —H.

In preferred embodiments, T represents —O— and U represents —(CR⁵R^{5′})ₙ and the resultant moiety —O—(CR⁵R^{5′})ₙ— represents a residue selected from the group consisting of:

| | | |
|---|---|---|
| 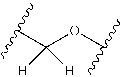 [SMILES: *CO*] | 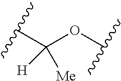 [SMILES: *OC(*)C] | 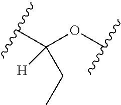 [SMILES: *OC(*)CC] |
| 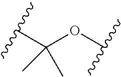 [SMILES: *OC(*)(C)C] | 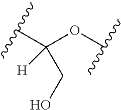 [SMILES: *OC(*)CO] | 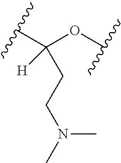 [SMILES: *OC(*)CCN(C)C] |
| 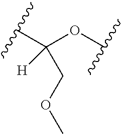 [SMILES: *OC(*)COC] | 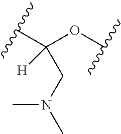 [SMILES: *OC(*)CN(C)C] | 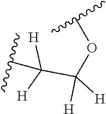 [SMILES: *CCO*] |

In some embodiments, R⁵ represents —H and R^{5′} represents a residue selected from the group consisting of —H, —C₁₋₃-alkyl, —CF₃, —CF₂H, —CFH₂, —C₁₋₃-alkylene-CF₃, —C₁₋₃-alkylene-CF₂H, —C₁₋₃-alkylene-CFH₂, and —C₁₋₃-alkylene-OH; preferably —H or C₁₋₃-alkyl.

In some embodiments R⁶ and R⁸ independently of one another represent
—H;
—F, —Cl, —Br, —I, —OH, —SH, —SF₅, —CN, —NO₂, —C(═O)OH, —NH₂;
—C₁₋₆-alkyl, saturated or unsaturated, unsubstituted, mono- or polysubstituted;
—O—C₁₋₆-alkyl, saturated or unsaturated, unsubstituted, mono- or polysubstituted;
—NHC₁₋₆-alkyl, saturated or unsaturated, unsubstituted, mono- or polysubstituted;
—N(C₁₋₆-alkyl)₂, saturated or unsaturated, unsubstituted, mono- or polysubstituted;
—C(═O)OC₁₋₆-alkyl, saturated or unsaturated, unsubstituted, mono- or polysubstituted;
—OC(═O)C₁₋₆-alkyl, saturated or unsaturated, unsubstituted, mono- or polysubstituted;
—C₁₋₆-heteroalkyl, saturated or unsaturated, unsubstituted, mono- or polysubstituted.

In some embodiments R⁶ and R⁸ independently of one another represent
—H;
—F, —Cl, —Br, —I, —OH, —SH, —SF₅, —CN, —NO₂, —C(═O)OH, —NH₂;
—C₁₋₆-alkyl, —C₂-C₆-alkenyl, —C₂-C₆-alkynyl, in each case saturated or unsaturated, unsubstituted, mono- or polysubstituted with substituents independently of one another selected from the group consisting of —F, —Cl, —C₁₋₆-alkyl, —C₁₋₆-alkylene-CF₃, —OH, ═O, —OC₁₋₆-alkyl, —C₁₋₆-alkylene-OH, —C₁₋₆-alkylene-O—C₁₋₆-alkyl, —NH₂, —NHC₁₋₆-alkyl, —N(C₁₋₆-alkyl)₂, —NHC(═O)O—C₁₋₆-alkyl, —N(C₁₋₆-alkyl)C(═O)O—C₁₋₆-alkyl, —C₁₋₆-alkylene-NHC(═O)O—C₁₋₆-alkyl, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —C(═O)N(C₁₋₆-alkyl)₂, —S(═O)₂C₁₋₆-alkyl, -phenyl, —C₁₋₆-alkylene-phenyl, 3-14-membered heterocycloalkyl, saturated or unsaturated, unsubstituted; and 5-14-membered heteroaryl, unsubstituted; —O—C₁₋₆-alkyl, saturated or unsaturated, unsubstituted, mono- or polysubstituted with substituents independently of one another selected from the group consisting of —F, —Cl, —C₁₋₆-alkyl, —C₁₋₆-alkylene-CF₃, —OH, ═O, —OC₁₋₆-alkyl, —C₁₋₆-alkylene-OH, —C₁₋₆-alkylene-O—C₁₋₆-alkyl, —NH₂, —NHC₁₋₆-alkyl, —N(C₁₋₆-alkyl)₂, —NHC(═O)O—C₁₋₆-alkyl, —N(C₁₋₆-alkyl)C(═O)O—C₁₋₆-alkyl, —C₁₋₆-alkylene-NHC(═O)O—C₁₋₆-alkyl, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —C(═O)N(C₁₋₆-alkyl)₂, —S(═O)₂C₁₋₆-alkyl, -phenyl, —C₁₋₆-alkylene-phenyl, 3-14-membered heterocycloalkyl, saturated or unsaturated, unsubstituted; and 5-14-membered heteroaryl, unsubstituted;
—NHC₁₋₆-alkyl, saturated or unsaturated, unsubstituted, mono- or polysubstituted with substituents independently of one another selected from the group consisting of —F, —Cl, —C₁₋₆-alkyl, —C₁₋₆-alkylene-CF₃, —OH, ═O, —OC₁₋₆-alkyl, —C₁₋₆-alkylene-OH, —C₁₋₆-alkylene-O—C₁₋₆-alkyl, —NH₂, —NHC₁₋₆-alkyl, —N(C₁₋₆-alkyl)₂, —NHC(═O)O—C₁₋₆-alkyl, —N(C₁₋₆-alkyl)C(═O)O—C₁₋₆-alkyl, —C₁₋₆-alkylene-NHC(═O)O—C₁₋₆-alkyl, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —C(═O)N(C₁₋₆-alkyl)₂, —S(═O)₂C₁₋₆-alkyl, -phenyl, —C₁₋₆-alkylene-phenyl, 3-14-membered heterocycloalkyl, saturated or unsaturated, unsubstituted; and 5-14-membered heteroaryl, unsubstituted;
—N(C₁₋₆-alkyl)₂, saturated or unsaturated, unsubstituted, mono- or polysubstituted with substituents independently of one another selected from the group consisting of —F, —Cl, —C₁₋₆-alkyl, —C₁₋₆-alkylene-CF₃, —OH, ═O, —OC₁₋₆-alkyl, —C₁₋₆-alkylene-OH, —C₁₋₆-alkylene-O—C₁₋₆-alkyl, —NH₂, —NHC₁₋₆-alkyl, —N(C₁₋₆-alkyl)₂, —NHC(═O)O—C₁₋₆-alkyl, —N(C₁₋₆-alkyl)C(═O)O—C₁₋₆-alkyl, —C₁₋₆-alkylene-NHC(═O)O—C₁₋₆-alkyl, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —C(═O)N(C₁₋₆-alkyl)₂, —S(═O)₂C₁₋₆-alkyl, -phenyl, —C₁₋₆-alkylene-phenyl, 3-14-membered heterocycloalkyl, saturated or unsaturated, unsubstituted; and 5-14-membered heteroaryl, unsubstituted;
—C(═O)OC₁₋₆-alkyl, saturated or unsaturated, unsubstituted, mono- or polysubstituted with substituents independently of one another selected from the group consisting of —F, —Cl, —C₁₋₆-alkyl, —C₁₋₆-alkylene-CF₃, —OH, ═O, —OC₁₋₆-alkyl, —C₁₋₆-alkylene-OH, —C₁₋₆-alkylene-O—C₁₋₆-alkyl, —NH₂, —NHC₁₋₆-alkyl, —N(C₁₋₆-alkyl)₂, —NHC(═O)O—C₁₋₆-alkyl, —N(C₁₋₆-alkyl)C(═O)O—C₁₋₆-alkyl, —C₁₋₆-alkylene-NHC(═O)O—C₁₋₆-alkyl, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —C(═O)N(C₁₋₆-alkyl)₂, —S(═O)₂C₁₋₆-alkyl, -phenyl, —C₁₋₆-alkylene-phenyl, 3-14-membered heterocycloalkyl, saturated or unsaturated, unsubstituted; and 5-14-membered heteroaryl, unsubstituted;
—OC(═O)C₁₋₆-alkyl, saturated or unsaturated, unsubstituted, mono- or polysubstituted with substituents independently of one another selected from the group consisting of —F, —Cl, —C₁₋₆-alkyl, —C₁₋₆-alkylene-CF₃, —OH, ═O, —OC₁₋₆-alkyl, —C₁₋₆-alkylene-OH, —C₁₋₆-alkylene-O—C₁₋₆-alkyl, —NH₂, —NHC₁₋₆-alkyl, —N(C₁₋₆-alkyl)₂, —NHC(═O)O—C₁₋₆-alkyl, —N(C₁₋₆-alkyl)C(═O)O—C₁₋₆-alkyl, —C₁₋₆-alkylene-NHC(═O)O—C₁₋₆-alkyl, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —C(═O)N(C₁₋₆-alkyl)₂, —S(═O)₂C₁₋₆-alkyl, -phenyl, —C₁₋₆-alkylene-phenyl, 3-14-membered heterocycloalkyl, saturated or unsaturated, unsubstituted; and 5-14-membered heteroaryl, unsubstituted —C₁₋₆-heteroalkyl, saturated or unsaturated, unsubstituted, mono- or polysubstituted with substituents independently of one another selected from the group consisting of —F, —Cl, —C₁₋₆-alkyl, —C₁₋₆-alkylene-CF₃, —OH, ═O, —OC₁₋₆-alkyl, —C₁₋₆-alkylene-OH, —C₁₋₆-alkylene-O—C₁₋₆-alkyl, —NH₂, —NHC₁₋₆-alkyl, —N(C₁₋₆-alkyl)₂, —NHC(═O)O—C₁₋₆-alkyl, —N(C₁₋₆-alkyl)C(═O)O—C₁₋₆-alkyl, —C₁₋₆-alkylene-NHC(═O)O—C₁₋₆-alkyl, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —C(═O)N(C₁₋₆-alkyl)₂, —S(═O)₂C₁₋₆-alkyl, -phenyl, —C₁₋₆-alkylene-phenyl, 3-14-membered heterocycloalkyl, saturated or unsaturated, unsubstituted; and 5-14-membered heteroaryl, unsubstituted..

Preferably, Rand R⁸ independently of one another represent
—H, —F, —Cl, —Br, —I, —OH, —SH, —SF₅, —CN, —NO₂, —C(═O)OH, —NH₂,
—C₁₋₆-alkyl, —CF₃, —CHF₂, —CH₂F,
—O—C₁₋₆-alkyl, —OCF₃, —OCHF₂, —OCH₂F,
—NHC₁₋₆-alkyl unsubstituted or substituted with one or more substituents independently of one another selected from —OH, ═O, —F, —Cl, —Br, —I, —SH, ═S, —CN, —CF₃, —CHF₂, —CH₂F, —OCF₃, —OCHF₂, —OCH₂F, SF₅, —NO₂, —C(═O)OH, —NH₂, and —C(═O)NH₂;
—N(C₁₋₆-alkyl)₂ unsubstituted or substituted with one or more substituents independently of one another selected from —OH, ═O, —F, —Cl, —Br, —I, —SH, ═S, —CN, —CF₃, —CHF₂, —CH₂F, —OCF₃, —OCHF₂, —OCH₂F, SF₅, —NO₂, —C(═O)OH, —NH₂, and —C(═O)NH₂;
—C(═O)OC₁₋₆-alkyl unsubstituted or substituted with one or more substituents independently of one another selected from —OH, ═O, —F, —Cl, —Br, —I, —SH, ═S, —CN, —CF₃, —CHF₂, —CH₂F, —OCF₃, —OCHF₂, —OCH₂F, SF₅, —NO₂, —C(═O)OH, —NH₂, and —C(═O)NH₂;
—OC(═O)C₁₋₆-alkyl unsubstituted or substituted with one or more substituents independently of one another selected from —OH, ═O, —F, —Cl, —Br, —I, —SH, ═S, —CN, —CF₃, —CHF₂, —CH₂F, —OCF₃, —OCHF₂, —OCH₂F, SF₅, —NO₂, —C(═O)OH, —NH₂, and —C(═O)NH₂; or
   —C₁₋₆-heteroalkyl unsubstituted or substituted with one or more substituents independently of one another selected from —OH, ═O, —F, —Cl, —Br, —I, —SH, ═S, —CN, —CF₃, —CHF₂, —CH₂F, —OCF₃, —OCHF₂, —OCH₂F, SF₅, —NO₂, —C(═O)OH, —NH₂, and —C(═O)NH₂.

In some embodiments, R⁶ and R⁸ independently of one another represents a residue selected from the group consisting of —H, —F, —Cl, —Br, —I, —CN, C₁₋₃-alkyl, —CF₃, —CF₂H, and —CFH₂; preferably —H or —F.

In some embodiments R⁶ represents —H, —F, —Cl, —CN, or —C₁-C₆-alkyl.

In some embodiments, R⁶ represents a residue selected from the group consisting of —H, —F, —Cl, —CN or —CH₃; preferably —H, —F, —CN or —CH₃

In some embodiments R⁶ does not represent —H.

In some embodiments, R⁶ and R⁸ represent independently from each other a residue selected from the group consisting of —H, —F, —Cl, —Br, —I, —CN, C₁₋₆-alkyl, —CF₃, —CF₂H, —CFH₂; or

| | | | |
|---|---|---|---|
| 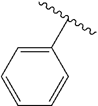 [SMILES: *c1ccccc1] | 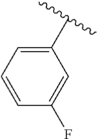 [SMILES: *c1cccc(F)c1] | 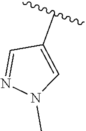 [SMILES: *c1cnn(C)c1] | 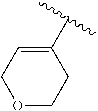 [SMILES: *C1=CCOCC1] |

In some embodiments R⁸ represents —H, —F, —Cl, —CN, or —C₁-C₆-alkyl.

In some embodiments R⁸ does not represent —H.

In some embodiments, R⁸ represents a residue selected from the group consisting of —H, —F, —Cl, —CN or CH₃; preferably —F

In some embodiments
(i) R⁶ and R⁸ each represent —H; or
(ii) R⁶ and R⁸ represent independently from each other —F, —Cl, —CN, or —CH₃; or
(iii) one of R⁶ and R⁸ represents —H and the other of R⁶ and R⁸ represents —F, —Cl, —CN, or —CH₃.

In some embodiments W represents
3-14-membered cycloalkyl, saturated or unsaturated, unsubstituted, mono- or polysubstituted; preferably a 3-12-membered cycloalkyl, saturated or unsaturated, unsubstituted, mono- or polysubstituted; preferably a 3-10-membered cycloalkyl, saturated or unsaturated, unsubstituted, mono- or polysubstituted; preferably a 3-8-membered cycloalkyl, saturated or unsaturated, unsubstituted, mono- or polysubstituted; preferably a 3-6-membered cycloalkyl, saturated or unsaturated, unsubstituted, mono- or polysubstituted; wherein said cycloalkyl is unsubstituted, monosubstituted or trisubstituted; preferably said cycloalkyl is unsubstituted, monosubstituted or disubstituted, in particular 3-14-membered cycloalkyl, saturated or non-aromatic unsaturated, unsubstituted, mono- or polysubstituted; preferably a 3-12-membered cycloalkyl, saturated or non-aromatic unsaturated, unsubstituted, mono- or polysubstituted; preferably a 3-10-membered cycloalkyl, saturated or non-aromatic unsaturated, unsubstituted, mono- or polysubstituted; preferably a 3-8-membered cycloalkyl, saturated or non-aromatic unsaturated, unsubstituted, mono- or polysubstituted; preferably a 3-6-membered cycloalkyl, saturated or non-aromatic unsaturated, unsubstituted, mono- or polysubstituted; wherein said cycloalkyl is unsubstituted, monosubstituted or trisubstituted; preferably said cycloalkyl is unsubstituted, monosubstituted or disubstituted; 6-14-membered aryl, unsubstituted, mono- or polysubstituted; preferably a 6-12-membered aryl, unsubstituted, mono- or polysubstituted; preferably a 6-10-membered aryl, unsubstituted, mono- or polysubstituted; wherein said aryl is unsubstituted, monosubstituted or trisubstituted; preferably said aryl is unsubstituted, monosubstituted or disubstituted; or
3-14-membered heterocycloalkyl, saturated or unsaturated, unsubstituted, mono- or polysubstituted; preferably a 3-12-membered heterocycloalkyl, saturated or unsaturated, unsubstituted, mono- or polysubstituted; preferably a 3-10-membered heterocycloalkyl, saturated or unsaturated, unsubstituted, mono- or polysubstituted; preferably a 3-8-membered heterocycloalkyl, saturated or unsaturated, unsubstituted, mono- or polysubstituted; preferably a 3-6-membered heterocycloalkyl, saturated or unsaturated, unsubstituted, mono- or polysubstituted; wherein said heterocycloalkyl is unsubstituted, monosubstituted or trisubstituted; preferably said heterocycloalkyl is unsubstituted, monosubstituted or disubstituted;
5-14-membered heteroaryl, unsubstituted, mono- or polysubstituted; preferably a 5-12-membered heteroaryl, unsubstituted, mono- or polysubstituted; preferably a 5-10-membered heteroaryl, unsubstituted, mono- or polysubstituted; preferably a 5-9-membered heteroaryl, unsubstituted, mono- or polysubstituted; preferably a 5-8-membered heteroaryl, unsubstituted, mono- or polysubstituted; wherein said heteroaryl is unsubstituted, monosubstituted or trisubstituted; preferably said heteroaryl is unsubstituted, monosubstituted or disubstituted;
—C₁-C₆-alkyl, —C₂-C₆-alkenyl, —C₂-C₆-alkynyl; wherein said alkyl, alkenyl or alkynyl is unsubstituted, monosubstituted or trisubstituted; preferably said alkyl, alkenyl or alkynyl is unsubstituted, monosubstituted or disubstituted.

In some embodiments W represents
3-14-membered cycloalkyl, 3-14-membered cycloalkenyl, 3-14-membered cycloalkynyl, each of said groups, unsubstituted, mono- or polysubstituted; preferably a 3-12-membered cycloalkyl, 3-12-membered cycloalkenyl, 3-12-membered cycloalkynyl, each of said groups, unsubstituted, mono- or polysubstituted; preferably a 3-10-membered cycloalkyl, 3-10-membered cycloalkenyl, 3-10-membered cycloalkynyl, each of said groups, unsubstituted, mono- or polysubstituted; preferably a 3-8-membered cycloalkyl, 3-8-membered cycloalkenyl, 3-8-membered cycloalkynyl, each of said groups, unsubstituted, mono- or polysubstituted; preferably a 3-6-membered cycloalkyl, 3-6-membered cycloalkenyl, 3-6-membered cycloalkynyl, each of said groups, unsubstituted, mono- or polysubstituted; wherein said cycloalkyl is unsubstituted, monosubstituted or trisubstituted; preferably said cycloalkyl is unsubstituted, monosubstituted or disubstituted; 6-14-membered aryl, unsubstituted, mono- or polysubstituted; preferably a 6-12-membered aryl, unsubstituted, mono- or polysubstituted; preferably a 6-10-membered aryl, unsubstituted, mono- or polysubstituted; wherein said aryl is unsubstituted, monosubstituted or trisubstituted; preferably said aryl is unsubstituted, monosubstituted or disubstituted; or
3-14-membered heterocycloalkyl, saturated or unsaturated, unsubstituted, mono- or polysubstituted; preferably a 3-12-membered heterocycloalkyl, saturated or unsaturated, unsubstituted, mono- or polysubstituted; preferably a 3-10-membered heterocycloalkyl, saturated or unsaturated, unsubstituted, mono- or polysubstituted; preferably a 3-8-membered heterocycloalkyl, saturated or unsaturated, unsubstituted, mono- or polysubstituted; preferably a 3-6-membered heterocycloalkyl, saturated or unsaturated, unsubstituted, mono- or polysubstituted; wherein said heterocycloalkyl is unsubstituted, monosubstituted or trisubstituted; preferably said heterocycloalkyl is unsubstituted, monosubstituted or disubstituted;
5-14-membered heteroaryl, unsubstituted, mono- or polysubstituted; preferably a 5-12-membered heteroaryl, unsubstituted, mono- or polysubstituted; preferably a 5-10-membered heteroaryl, unsubstituted, mono- or polysubstituted; preferably a 5-9-membered heteroaryl, unsubstituted, mono- or polysubstituted; preferably a 5-8-membered heteroaryl, unsubstituted, mono- or polysubstituted; wherein said heteroaryl is unsubstituted, monosubstituted or trisubstituted; preferably said heteroaryl is unsubstituted, monosubstituted or disubstituted;
—C₁-C₆-alkyl, —C₂-C₆-alkenyl, —C₂-C₆-alkynyl; wherein said alkyl, alkenyl or alkynyl is unsubstituted, monosubstituted or trisubstituted; preferably said alkyl, alkenyl or alkynyl is unsubstituted, monosubstituted or disubstituted.

In some embodiments W represents
3-14-membered cycloalkyl, saturated unsubstituted, mono- or polysubstituted; preferably a 3-12-membered cycloalkyl, saturated, unsubstituted, mono- or polysubstituted; preferably a 3-10-membered cycloalkyl, saturated, unsubstituted, mono- or polysubstituted; preferably a 3-8-membered cycloalkyl, saturated, unsubstituted, mono- or polysubstituted; preferably a 3-6-membered cycloalkyl, saturated, unsubstituted, mono- or polysubstituted; wherein said cycloalkyl is unsubstituted, monosubstituted or trisubstituted; preferably said cycloalkyl is unsubstituted, monosubstituted or disubstituted; 6-14-membered aryl, unsubstituted, mono- or polysubstituted; preferably a 6-12-membered aryl, unsubstituted, mono- or polysubstituted; preferably a 6-10-membered aryl, unsubstituted, mono- or polysubstituted; wherein said aryl is unsubstituted, monosubstituted or trisubstituted; preferably said aryl is unsubstituted, monosubstituted or disubstituted; or
3-14-membered heterocycloalkyl, saturated or unsaturated, unsubstituted, mono- or polysubstituted; preferably a 3-12-membered heterocycloalkyl, saturated or unsaturated, unsubstituted, mono- or polysubstituted; preferably a 3-10-membered heterocycloalkyl, saturated or unsaturated, unsubstituted, mono- or polysubstituted; preferably a 3-8-membered heterocycloalkyl, saturated or unsaturated, unsubstituted, mono- or polysubstituted; preferably a 3-6-membered heterocycloalkyl, saturated or unsaturated, unsubstituted, mono- or polysubstituted; wherein said heterocycloalkyl is unsubstituted, monosubstituted or trisubstituted; preferably said heterocycloalkyl is unsubstituted, monosubstituted or disubstituted;
5-14-membered heteroaryl, unsubstituted, mono- or polysubstituted; preferably a 5-12-membered heteroaryl, unsubstituted, mono- or polysubstituted; preferably a 5-10-membered heteroaryl, unsubstituted, mono- or polysubstituted; preferably a 5-9-membered heteroaryl, unsubstituted, mono- or polysubstituted; preferably a 5-8-membered heteroaryl, unsubstituted, mono- or polysubstituted; wherein said heteroaryl is unsubstituted, monosubstituted or trisubstituted; preferably said heteroaryl is unsubstituted, monosubstituted or disubstituted;
—C₁-C₆-alkyl, —C₂-C₆-alkenyl, —C₂-C₆-alkynyl; in each case unsubstituted, mono- or disubstituted wherein the substituents independently of one another are selected from the group consisting of —F, —Cl, —Br, —I, —C₁₋₆-alkyl, —CF₃, —CF₂H, —CFH₂, —CF₂Cl, —CFCl₂, —C₁₋₆-alkylene-CF₃, —C(═O)—C₁₋₆-alkyl, —C(═O)OH, —C(═O)—OC₁₋₆-alkyl, —C(═O)O—C₁₋₆-alkylene-CF₃, —OH, ═O, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C₁₋₆-alkylene-OH, —C₁₋₆-alkylene-O—C₁₋₆-alkyl, —C₁₋₆-alkylene-NHC(═O)O—C₁₋₆-alkyl, —NHC(═O)O—C₁₋₆-alkyl, —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂, 3-14-membered heterocycloalkyl, -phenyl unsubstituted, 5 to 14-membered heteroaryl.

In some embodiments W represents 3-10-membered cycloalkyl, unsubstituted, mono- or polysubstituted, 6-12-membered aryl, unsubstituted, mono- or polysubstituted, 3-10-membered heterocycloalkyl, saturated or unsaturated, mono- or polysubstituted, 5-12-membered heteroaryl, mono- or polysubstituted —C₁-C₆-alkyl, haloC₁₋₆alkyl, —C₂-C₆-alkenyl, —C₂-C₆-alkynyl; preferably W represents 3-10-membered cycloalkyl, unsubstituted, mono- or disubstituted, 6-12-membered aryl, unsubstituted, mono- or disubstituted, 3-10-membered heterocycloalkyl, saturated or unsaturated, mono- or disubstituted, 5-12-membered heteroaryl, mono- or substituted, methyl, ethyl, n-propyl, i-propyl, n-butyl, i-butyl and t-butyl; pentyl, hexyl —CF₃, —CF₂H, —CFH₂, —CF₂Cl, —CFCl₂, —CCl₃, —C₁₋₆-alkylene-CC or —C₁₋₆-alkylene-CF₃.

In some embodiments W represents
a 3-14-membered cycloalkyl, saturated, unsubstituted, mono- or polysubstituted; preferably a 3-13-membered cycloalkyl, saturated, unsubstituted, mono or polysubstituted; preferably a 3-12-membered cycloalkyl, saturated, unsubstituted, mono or polysubstituted; preferably a 3-11-membered cycloalkyl, saturated, unsubstituted, mono or polysubstituted; preferably a 3-10-membered cycloalkyl, saturated, unsubstituted, mono or polysubstituted; preferably represents a cycloalkyl residue selected from the group consisting of:

| | | |
|---|---|---|
| 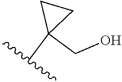 [SMILES: *C1(CO)CC1] | 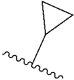 [SMILES: *C1CC1] | 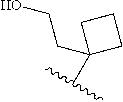 [SMILES: *C1(CCO)CCC1] |
| 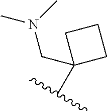 [SMILES: *C1(CN(C)C)CCC1] | 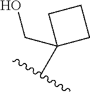 [SMILES: *C1(CO)CCC1] | 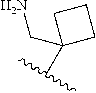 [SMILES: *C1(CN)CCC1] |
| 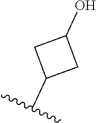 [SMILES: *C1CC(O)C1] | 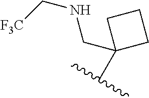 [SMILES: *C1(CNCC(F)(F)F)CCC1] | 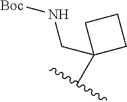 [SMILES: *C1(CNC(=O)OC(C)(C)C)CCC1] |
| 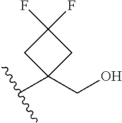 [SMILES: *C1(CO)CC(F)(F)C1] | 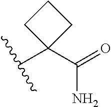 [SMILES: *C1(C(N)=O)CCC1] | 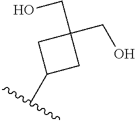 [SMILES: *C1CC(CO)(CO)C1] |
| 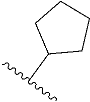 [SMILES: *C1CCCC1] | 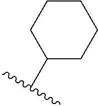 [SMILES: *C1CCCCC1] | 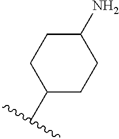 [SMILES: *C1CCC(N)CC1] |
| 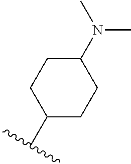 [SMILES: *C1CCC(N(C)C)CC1] | 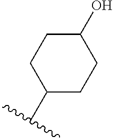 [SMILES: *C1CCC(O)CC1] | 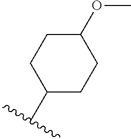 [SMILES: *C1CCC(OC)CC1] |
| 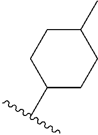 [SMILES: *C1CCC(C)CC1] | 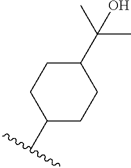 [SMILES: *C1CCC(C(C)(C)O)CC1] | 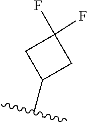 [SMILES: *C1CC(F)(F)C1] |
| 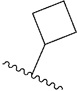 [SMILES: *C1CCC1] | 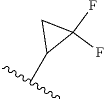 [SMILES: *C1CC1(F)F] | 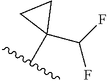 [SMILES: *C1(C(F)F)CC1] |
| 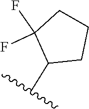 [SMILES: *C1CCCC1(F)F] | 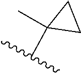 [SMILES: *C1(C)CC1] | 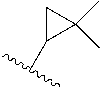 [SMILES: *C1CC1(C)C] |
| 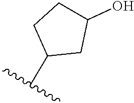 [SMILES: *C1CCC(O)C1] | 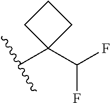 [SMILES: *C1(C(F)F)CCC1] | 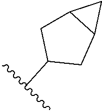 [SMILES: *C1CC2CC2C1] |
| 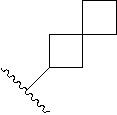 [SMILES: *C1CC2(CCC2)C1] | | |

6-14-membered aryl, unsubstituted, mono- or polysubstituted; preferably a 6-12-membered aryl, unsubstituted, mono- or polysubstituted; preferably a 6-10-membered aryl, unsubstituted, mono- or polysubstituted; preferably a 6-8-membered aryl, unsubstituted, mono- or polysubstituted; preferably represents a phenyl residue selected from the group consisting of:

| | | |
|---|---|---|
| 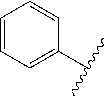 [SMILES: *c1ccccc1] | 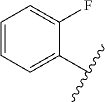 [SMILES: *c1ccccc1F] | 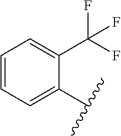 [SMILES: *c1ccccc1C(F)(F)F] |
| 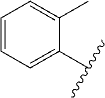 [SMILES: *c1ccccc1C] | 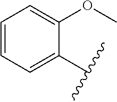 [SMILES: *c1ccccc1OC] | 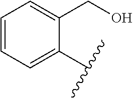 [SMILES: *c1ccccc1CO] |
| 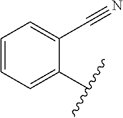 [SMILES: *c1ccccc1C#N] | 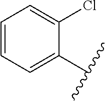 [SMILES: *c1ccccc1Cl] | 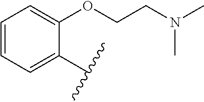 [SMILES: *c1ccccc1OCCN(C)C] |
| 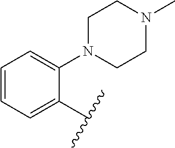 [SMILES: *c1ccccc1N1CCN(C)CC1] | 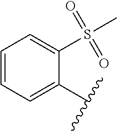 [SMILES: *c1ccccc1S(C)(=O)=O] | 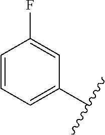 [SMILES: *c1cccc(F)c1] |
| 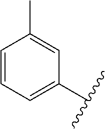 [SMILES: *c1cccc(C)c1] | 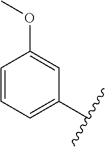 [SMILES: *c1cccc(OC)c1] | 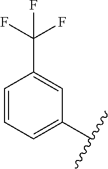 [SMILES: *c1cccc(C(F)(F)F)c1] |
| 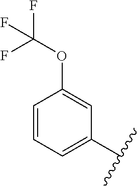 [SMILES: *c1cccc(OC(F)(F)F)c1] | 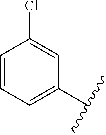 [SMILES: *c1cccc(Cl)c1] | 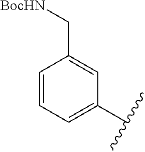 [SMILES: *c1cccc(CNC(=O)OC(C)(C)C)c1] |
| 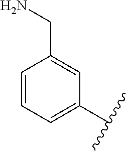 [SMILES: *c1cccc(CN)c1] | 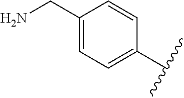 [SMILES: *c1ccc(CN)cc1] | 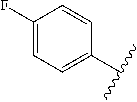 [SMILES: *c1ccc(F)cc1] |
| 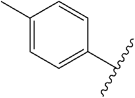 [SMILES: *c1ccc(C)cc1] | 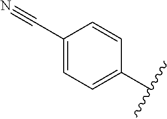 [SMILES: *c1ccc(C#N)cc1] | 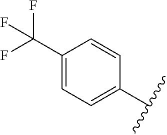 [SMILES: *c1ccc(C(F)(F)F)cc1] |
| 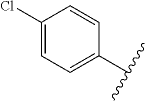 [SMILES: *c1ccc(Cl)cc1] | 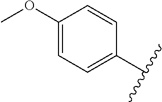 [SMILES: *c1ccc(OC)cc1] | 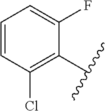 [SMILES: *c1c(F)cccc1Cl] |
| 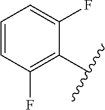 [SMILES: *c1c(F)cccc1F] | 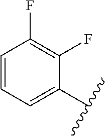 [SMILES: *c1cccc(F)c1F] | 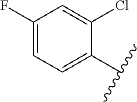 [SMILES: *c1ccc(F)cc1Cl] |
| 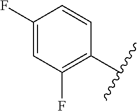 [SMILES: *c1ccc(F)cc1F] | 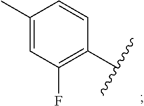 [SMILES: *c1ccc(C)cc1F] | |

3-14-membered heterocycloalkyl, saturated, unsaturated, unsubstituted, mono- or polysubstituted; preferably 3-13-membered heterocycloalkyl, saturated, unsaturated, unsubstituted, mono- or polysubstituted; preferably 3-12-membered heterocycloalkyl, saturated, unsaturated, unsubstituted, mono- or polysubstituted; preferably 3-11-membered heterocycloalkyl, saturated, unsaturated, unsubstituted, mono- or polysubstituted; preferably 3-10-membered heterocycloalkyl, saturated, unsaturated, unsubstituted, mono- or poly substituted; preferably represents a heterocycloalkyl residue selected from the group consisting of:

| | | |
|---|---|---|
| 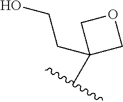 [SMILES: *C1(CCO)COC1] | 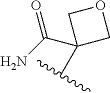 [SMILES: *C1(C(N)=O)COC1] | 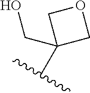 [SMILES: *C1(CO)COC1] |
| 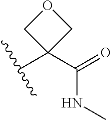 [SMILES: *C1(C(=O)NC)COC1] | 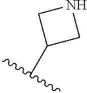 [SMILES: *C1CNC1] | 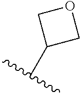 [SMILES: *C1COC1] |
| 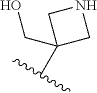 [SMILES: *C1(CO)CNC1] | 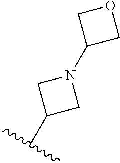 [SMILES: *C1CN(C2COC2)C1] | 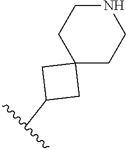 [SMILES: *C1CC2(CCNCC2)C1] |
| 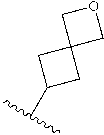 [SMILES: *C1CC2(COC2)C1] | 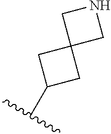 [SMILES: *C1CC2(CNC2)C1] | 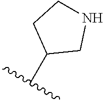 [SMILES: *C1CCNC1] |
| 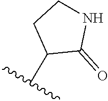 [SMILES: *C1CCNC1=O] | 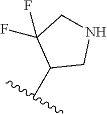 [SMILES: *C1CNCC1(F)F] | 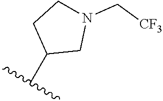 [SMILES: *C1CCN(CC(F)(F)F)C1] |
| 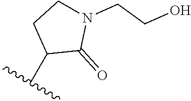 [SMILES: *C1CCN(CCO)C1=O] | 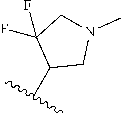 [SMILES: *C1CN(C)CC1(F)F] | 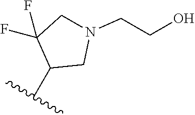 [SMILES: *C1CN(CCO)CC1(F)F] |
| 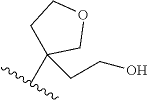 [SMILES: *C1(CCO)CCOC1] | 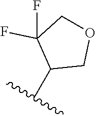 [SMILES: *C1COCC1(F)F] | 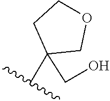 [SMILES: *C1(CO)CCOC1] |
| 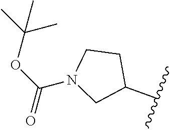 [SMILES: *C1CCN(C(=O)OC(C)(C)C)C1] | 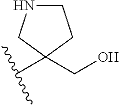 [SMILES: *C1(CO)CCNC1] | 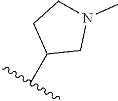 [SMILES: *C1CCN(C)C1] |
| 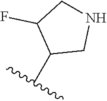 [SMILES: *C1CNCC1F] | 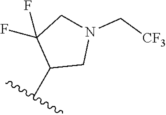 [SMILES: *C1CN(CC(F)(F)F)CC1(F)F] | 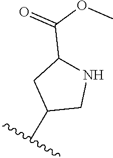 [SMILES: *C1CNC(C(=O)OC)C1] |
| 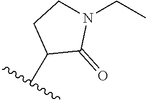 [SMILES: *C1CCN(CC)C1=O] | 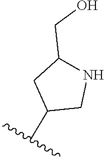 [SMILES: *C1CNC(CO)C1] | 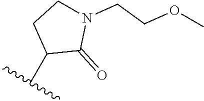 [SMILES: *C1CCN(CCOC)C1=O] |
| 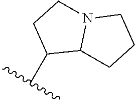 [SMILES: *C1CCN2CCCC12] | 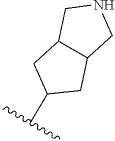 [SMILES: *C1CC2CNCC2C1] | 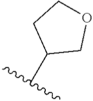 [SMILES: *C1CCOC1] |
| 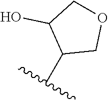 [SMILES: *C1COCC1O] | 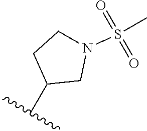 [SMILES: *C1CCN(S(C)(=O)=O)C1] | 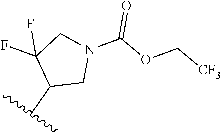 [SMILES: *C1CN(C(=O)OCC(F)(F)F)CC1(F)F] |
| 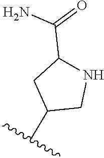 [SMILES: *C1CNC(C(N)=O)C1] | 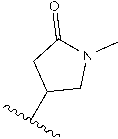 [SMILES: *C1CC(=O)N(C)C1] | 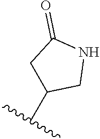 [SMILES: *C1CNC(=O)C1] |
| 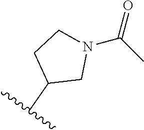 [SMILES: *C1CCN(C(C)=O)C1] | 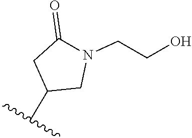 [SMILES: *C1CC(=O)N(CCO)C1] | 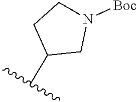 [SMILES: *C1CCN(C(=O)OC(C)(C)C)C1] |
| 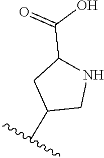 [SMILES: *C1CNC(C(=O)O)C1] | 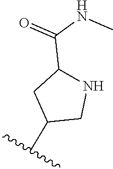 [SMILES: *C1CNC(C(=O)NC)C1] | 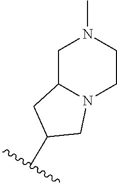 [SMILES: *C1CC2CN(C)CCN2C1] |
| 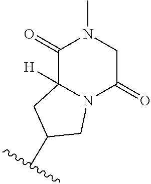 [SMILES: *C1CC2C(=O)N(C)CC(=O)N2C1] | 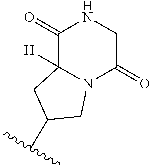 [SMILES: *C1CC2C(=O)NCC(=O)N2C1] | 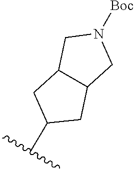 [SMILES: *C1CC2CN(C(=O)OC(C)(C)C)CC2C1] |
| 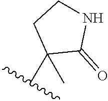 [SMILES: *C1(C)CCNC1=O] | 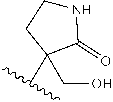 [SMILES: *C1(CO)CCNC1=O] | 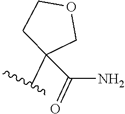 [SMILES: *C1(C(N)=O)CCOC1] |
| 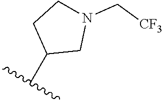 [SMILES: *C1CCN(CC(F)(F)F)C1] | 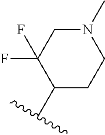 [SMILES: *C1CCN(C)CC1(F)F] | 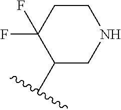 [SMILES: *C1CNCCC1(F)F] |
| 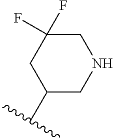 [SMILES: *C1CNCC(F)(F)C1] | 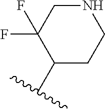 [SMILES: *C1CCNCC1(F)F] | 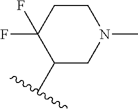 [SMILES: *C1CN(C)CCC1(F)F] |
| 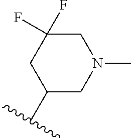 [SMILES: *C1CN(C)CC(F)(F)C1] | 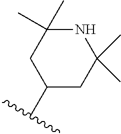 | 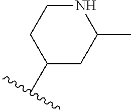 [SMILES: *C1CCNC(C)C1] |
| 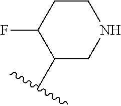 [SMILES: *C1CNCCC1F] | 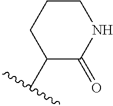 [SMILES: *C1CCCNC1=O] | 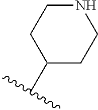 [SMILES: *C1CCNCC1] |
| 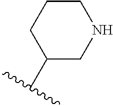 [SMILES: *C1CCCNC1] | 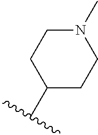 [SMILES: *C1CCN(C)CC1] | 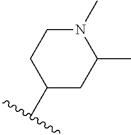 [SMILES: *C1CCN(C)C(C)C1] |
| 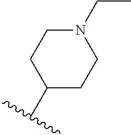 [SMILES: *C1CCN(CC)CC1] | 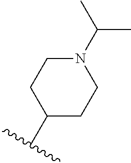 [SMILES: *C1CCN(C(C)C)CC1] | 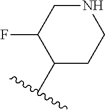 [SMILES: *C1CCNCC1F] |
| 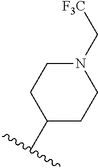 [SMILES: *C1CCN(CC(F)(F)F)CC1] | 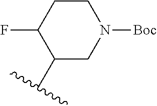 [SMILES: *C1CN(C(=O)OC(C)(C)C)CCC1F] | 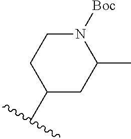 [SMILES: *C1CCN(C(=O)OC(C)(C)C)C(C)C1] |
| 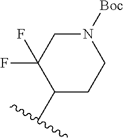 [SMILES: *C1CCN(C(=O)OC(C)(C)C)CC1(F)F] | 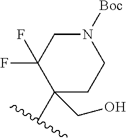 [SMILES: *C1(CO)CCN(C(=O)OC(C)(C)C)CC1(F)F] | 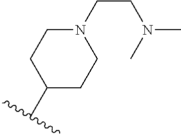 [SMILES: *C1CCN(CCN(C)C)CC1] |
| 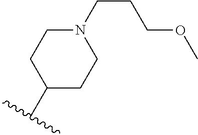 [SMILES: *C1CCN(CCCOC)CC1] | 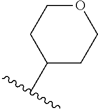 [SMILES: *C1CCOCC1] | 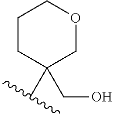 [SMILES: *C1(CO)CCCOC1] |
| 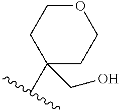 [SMILES: *C1(CO)CCOCC1] | 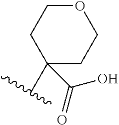 [SMILES: *C1(C(=O)O)CCOCC1] | 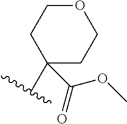 [SMILES: *C1(C(=O)OC)CCOCC1] |
| 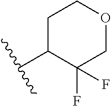 [SMILES: *C1CCOCC1(F)F] | 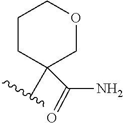 [SMILES: *C1(C(N)=O)CCCOC1] | 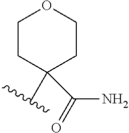 [SMILES: *C1(C(N)=O)CCOCC1] |
| 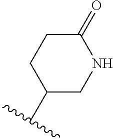 [SMILES: *C1CCC(=O)NC1] | 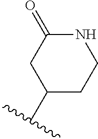 [SMILES: *C1CCNC(=O)C1] | 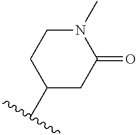 [SMILES: *C1CCN(C)C(=O)C1] |
| 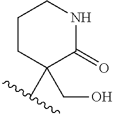 [SMILES: *C1(CO)CCCNC1=O] | 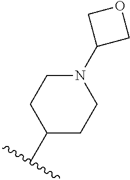 [SMILES: *C1CCN(C2COC2)CC1] | 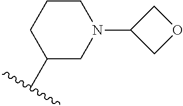 [SMILES: *C1CCCN(C2COC2)C1] |
| 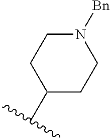 [SMILES: *C1CCN(Cc2ccccc2)CC1] | 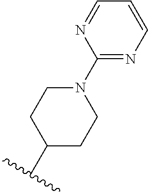 [SMILES: *C1CCN(c2ncccn2)CC1] | 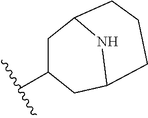 [SMILES: *C1CC2CCCC(C1)N2] |
| 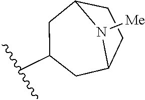 [SMILES: *C1CC2CCC(C1)N2C] | 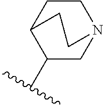 [SMILES: *C1CN2CCC1CC2] | 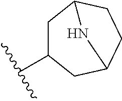 [SMILES: *C1CC2CCC(C1)N2] |
| 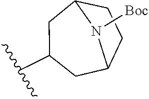 [SMILES: *C1CC2CCC(C1)N2C(=O)OC(C)(C)C] | 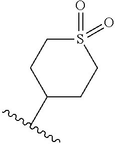 [SMILES: *C1CCS(=O)(=O)CC1] | 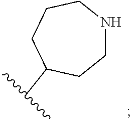 [SMILES: *C1CCCNCC1] |

more preferably selected from the group consisting of:

| | | |
|---|---|---|
| 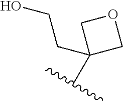 [SMILES: *C1(CCO)COC1] | 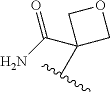 [SMILES: *C1(C(N)=O)COC1] | 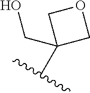 [SMILES: *C1(CO)COC1] |
| 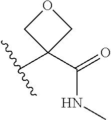 [SMILES: *C1(C(=O)NC)COC1] | 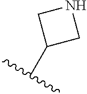 [SMILES: *C1CNC1] | 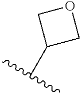 [SMILES: *C1COC1] |
| 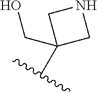 [SMILES: *C1(CO)CNC1] | 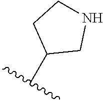 [SMILES: *C1CCNC1] | 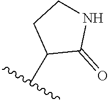 [SMILES: *C1CCNC1=O] |
| 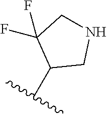 [SMILES: *C1CNCC1(F)F] | 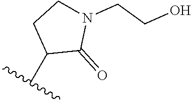 [SMILES: *C1CCN(CCO)C1=O] | 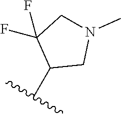 [SMILES: *C1CN(C)CC1(F)F] |
| 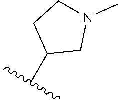 [SMILES: *C1CCN(C)C1] | 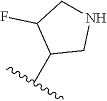 [SMILES: *C1CNCC1F] | 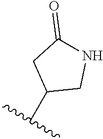 [SMILES: *C1CNC(=O)C1] |
| 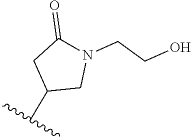 [SMILES: *C1CC(=O)N(CCO)C1] | 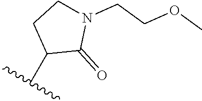 [SMILES: *C1CCN(CCOC)C1=O] | 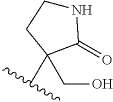 [SMILES: *C1(CO)CCNC1=O] |
| 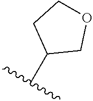 [SMILES: *C1CCOC1] | 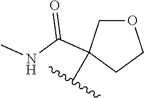 [SMILES: *C1(C(=O)NC)CCOC1] | 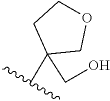 [SMILES: *C1(CO)CCOC1] |
| 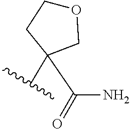 [SMILES: *C1(C(N)=O)CCOC1] | 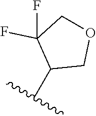 [SMILES: *C1COCC1(F)F] | 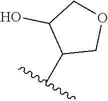 [SMILES: *C1COCC1O] |
| 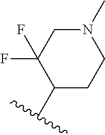 [SMILES: *C1CCN(C)CC1(F)F] | 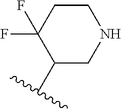 [SMILES: *C1CNCCC1(F)F] | 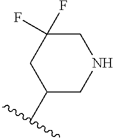 [SMILES: *C1CNCC(F)(F)C1] |
| 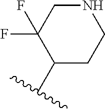 [SMILES: *C1CCNCC1(F)F] | 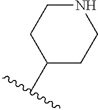 [SMILES: *C1CCNCC1] | 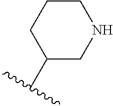 [SMILES: *C1CCCNC1] |
| 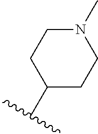 [SMILES: *C1CCN(C)CC1] | 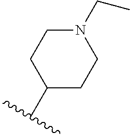 [SMILES: *C1CCN(CC)CC1] | 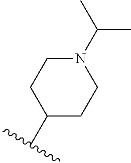 [SMILES: *C1CCN(C(C)C)CC1] |
| 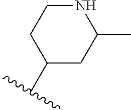 [SMILES: *C1CCNC(C)C1] | 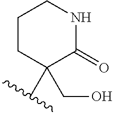 [SMILES: *C1(CO)CCCNC1=O] | 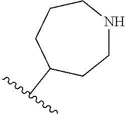 [SMILES: *C1CCCNCC1] |
| 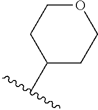 [SMILES: *C1CCOCC1] | 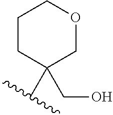 [SMILES: *C1(CO)CCCOC1] | 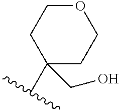 [SMILES: *C1(CO)CCOCC1] |
| 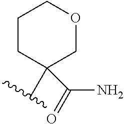 [SMILES: *C1(C(N)=O)CCCOC1] | 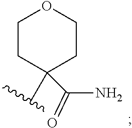 [SMILES: *C1(C(N)=O)CCOCC1] | |

5-14-membered heteroaryl unsubstituted, mono- or polysubstituted; preferably a 5-12-membered heteroaryl unsubstituted, mono- or polysubstituted; preferably a 5-10-membered heteroaryl unsubstituted, mono- or polysubstituted; preferably a 5-9-membered heteroaryl unsubstituted, mono- or polysubstituted; more preferably represents a residue selected from the group consisting of:

| | | |
|---|---|---|
| 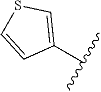 [SMILES: *c1ccsc1] | 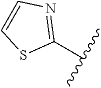 [SMILES: *c1nccs1] | 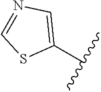 [SMILES: *c1cncs1] |
| 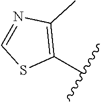 [SMILES: *c1scnc1C] | 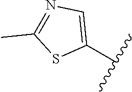 [SMILES: *c1cnc(C)s1] | 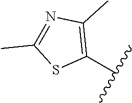 [SMILES: *c1sc(C)nc1C] |
| 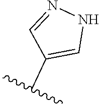 [SMILES: *c1cn[nH]c1] | 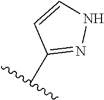 [SMILES: *c1cc[nH]n1] | 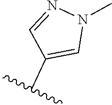 [SMILES: *c1cnn(C)c1] |
| 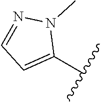 [SMILES: *c1ccnn1C] | 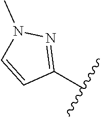 [SMILES: *c1ccn(C)n1] | 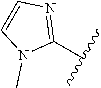 [SMILES: *c1nccn1C] |
| 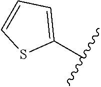 [SMILES: *c1cccs1] | 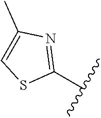 [SMILES: *c1nc(C)cs1] | 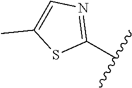 [SMILES: *c1ncc(C)s1] |
| 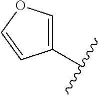 [SMILES: *c1ccoc1] | 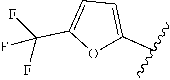 [SMILES: *c1ccc(C(F)(F)F)o1] | 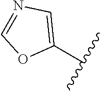 [SMILES: *c1cnco1] |
| 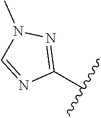 [SMILES: *c1ncn(C)n1] | 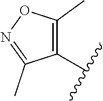 [SMILES: *c1c(C)noc1C] | 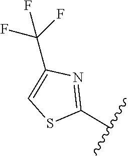 [SMILES: *c1nc(C(F)(F)F)cs1] |
| 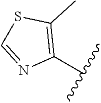 [SMILES: *c1ncsc1C] | 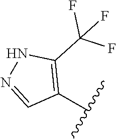 [SMILES: *c1cn[nH]c1C(F)(F)F] | 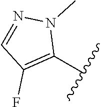 [SMILES: *c1c(F)cnn1C] |
| 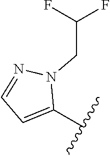 [SMILES: *c1ccnn1CC(F)F] | 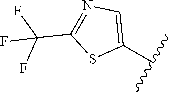 [SMILES: *c1cnc(C(F)(F)F)s1] | 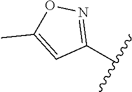 [SMILES: *c1cc(C)on1] |
| 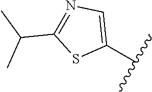 [SMILES: *c1cnc(C(C)C)s1] | 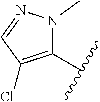 [SMILES: *c1c(Cl)cnn1C] | 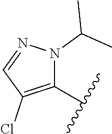 [SMILES: *c1c(Cl)cnn1C(C)C] |
| 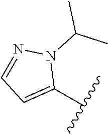 [SMILES: *c1ccnn1C(C)C] | 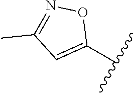 [SMILES: *c1cc(C)no1] | 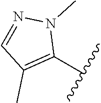 [SMILES: *c1c(C)cnn1C] |
| 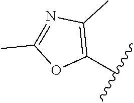 [SMILES: *c1oc(C)nc1C] | 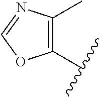 [SMILES: *c1ocnc1C] | 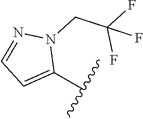 [SMILES: *c1ccnn1CC(F)(F)F] |
| 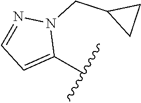 [SMILES: *c1ccnn1CC1CC1] | 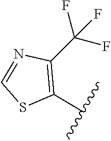 [SMILES: *c1scnc1C(F)(F)F] | 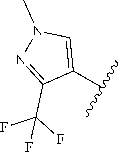 [SMILES: *c1cn(C)nc1C(F)(F)F] |
| 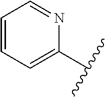 [SMILES: *c1ccccn1] | 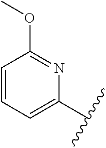 [SMILES: *c1cccc(OC)n1] | 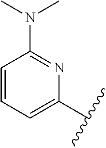 [SMILES: *c1cccc(N(C)C)n1] |
| 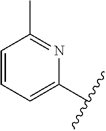 [SMILES: *c1cccc(C)n1] | 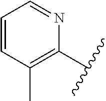 [SMILES: *c1ncccc1C] | 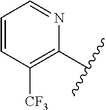 [SMILES: *c1ncccc1C(F)(F)F] |
| 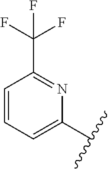 [SMILES: *c1cccc(C(F)(F)F)n1] | 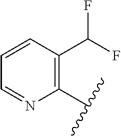 [SMILES: *c1ncccc1C(F)F] | 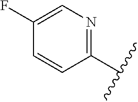 [SMILES: *c1ccc(F)cn1] |
| 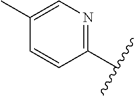 [SMILES: *c1ccc(C)cn1] | 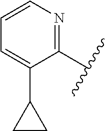 [SMILES: *c1ncccc1C1CC1] | 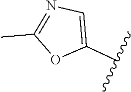 [SMILES: *c1cnc(C)o1] |
| 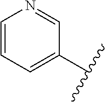 [SMILES: *c1cccnc1] | 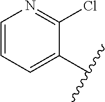 [SMILES: *c1cccnc1Cl] | 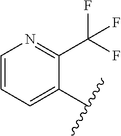 [SMILES: *c1cccnc1C(F)(F)F] |
| 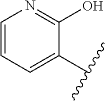 [SMILES: *c1cccnc1O] | 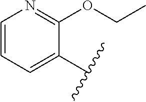 [SMILES: *c1cccnc1OCC] | 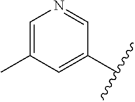 [SMILES: *c1cncc(C)c1] |
| 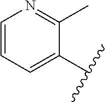 [SMILES: *c1cccnc1C] | 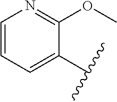 [SMILES: *c1cccnc1OC] | 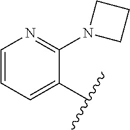 [SMILES: *c1cccnc1N1CCC1] |
| 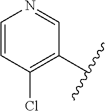 [SMILES: *c1cnccc1Cl] | 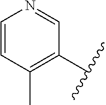 [SMILES: *c1cnccc1C] | 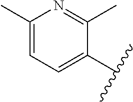 [SMILES: *c1ccc(C)nc1C] |
| 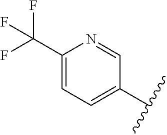 [SMILES: *c1ccc(C(F)(F)F)nc1] | 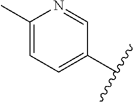 [SMILES: *c1ccc(C)nc1] | 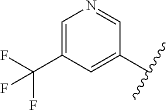 [SMILES: *c1cncc(C(F)(F)F)c1] |
| 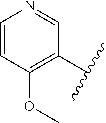 [SMILES: *c1cnccc1OC] | 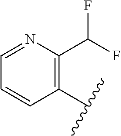 [SMILES: *c1cccnc1C(F)F] | 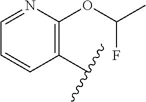 [SMILES: *c1cccnc1OC(C)F] |
| 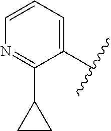 [SMILES: *c1cccnc1C1CC1] | 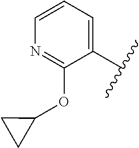 [SMILES: *c1cccnc1OC1CC1] | 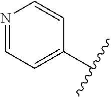 [SMILES: *c1ccncc1] |
| 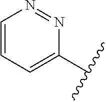 [SMILES: *c1cccnn1] | 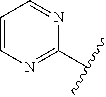 [SMILES: *c1ncccn1] | 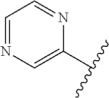 [SMILES: *c1cnccn1] |
| 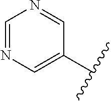 [SMILES: *c1cncnc1] | 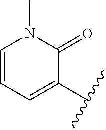 [SMILES: *c1cccn(C)c1=O] | 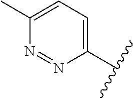 [SMILES: *c1ccc(C)nn1] |
| 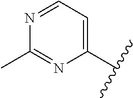 [SMILES: *c1ccnc(C)n1] | [symbol] ; | |

[symbol]

indicates data missing or illegible when filed

more preferably represents a residue selected from the group consisting of:

| | | |
|---|---|---|
| 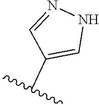 [SMILES: *c1cn[nH]c1] | 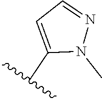 [SMILES: *c1ccnn1C] | 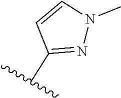 [SMILES: *c1ccn(C)n1] |
| 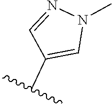 [SMILES: *c1cnn(C)c1] | 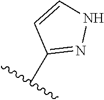 [SMILES: *c1cc[nH]n1] | 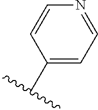 [SMILES: *c1ccncc1] |
| 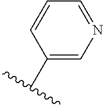 [SMILES: *c1cccnc1] | 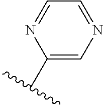 [SMILES: *c1cnccn1] | 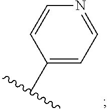 [SMILES: *c1ccncc1] |

—C₁-C₆-alkyl, —C₂-C₆-alkenyl, —C₂-C₆-alkynyl; in each case unsubstituted, mono- or polysubstituted with substituents independently of one another selected from the group consisting of —F, —Cl, —Br, —I, —C₁₋₆-alkyl, —CF₃, —CF₂H, —CFH₂, —CF₂Cl, —CFCl₂, —C₁₋₆-alkylene-CF₃, —C(═O)—C₁₋₆-alkyl, —C(═O)OH, —C(═O)—OC₁₋₆-alkyl, —C(═O)O—C₁₋₆-alkylene-CF₃, —OH, ═O, —OC₁₋₆-alkyl, —NH₂, —NH—C₁₋₆-alkyl, —N(C₁₋₆-alkyl)₂, —C₁₋₆-alkylene-NH₂, —Cis-6 alkylene-NHI—C₁₋₆-alkyl, —C₁₋₆-alkylene-N(C₁₋₆-alkyl)₂, —C₁₋₆-alkylene-NH—C₁₋₆-alkylene-CF₃, —C₁₋₆-alk(ylene-OH, —C₁₋₆-alkylene-O—C₁₋₆-alkyl, —C₁₋₆-alkylene-NH—C(═O)O—C₁₋₆-alkyl, —NH—C(═O)O—C₁₋₆-alkyl, —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂, 3-14-membered heterocycloalkyl, -phenyl unsubstituted, 5 to 14-membered heteroaryl.

In some embodiments the 5-14-membered heteroaryl within the definition of W is selected from benzimidazole, benzisoxazole, benzoxazole, benzodioxole, benzofuran, benzothiadiazole, benzothiazole, benzothiophene, carbazole, cinnoline, dibenzofuran, furan, furazan, imidazole, imidazopyridine, indazole, indole, indolizine, isobenzofuran, isoindole, isoquinoline, isothiazole, isoxazole, naphthyridine, oxadiazole, oxazole, oxindole, phthalazine, purine, pyrazine, pyrazole, pyridazine, pyridine, pyrimidine, pyrrole, quinazoline, quinoline, quinoxaline, tetrazole, thiadiazole, thiazole, thiophene, triazine, triazole, and [1,2,4]triazolo[4,3-a]pyrimidine; in each case unsubstituted, mono- or polysubstituted; preferably the definition is selected from benzimidazole, benzisoxazole, benzodioxole, benzofuran, benzothiazole, benzothiophene, cinnoline, dibenzofuran, furan, thiophene, furazan, imidazole, imidazopyridine, indole, isobenzofuran, isoindole, isoquinoline, isoxazole, oxadiazole, oxazole, oxindole, pyrazine, pyrazole, pyridazine, pyridine, pyrimidine, pyrrole, quinoline, tetrazole, thiadiazole, thiazole, thiophene, triazine, triazole, and [1,2,4]triazolo[4,3-a]pyrimidine; in each case unsubstituted, mono- or polysubstituted; preferably the definition is selected from furan, thiophene, imidazole, pyrazole, oxazole, isoxazole, thiazole, triazole, pyridine, isoquinoline, benzothiazole, pyridazine, pyrimidine, imidazopyridine, in each case unsubstituted, mono- or polysubstituted; preferably the definition is selected from furan-2-yl, furan-3-yl, thiophen-2-yl, thiophen-3-yl, pyrazol-5-yl, oxazol-5-yl, isoxazol-4-yl, thiazol-2-yl, thiazol-5-yl, 1,2,4-triazol-3-yl, 1,2,3-triazol-4-yl, pyridin-2-yl, pyridin-3-yl, pyridin-4-yl, isoquinolin-1-yl, isoquinolin-5-yl, benzo[d]thiazol-2-yl, pyridazin-3-yl, pyrimidin-5-yl, and imidazo[1,2-a]pyridin-6-yl, in each case unsubstituted, mono- or polysubstituted.

In some embodiments the 3-14-membered heterocycloalkyl within the definition of W is selected from azepane, 1,4-oxazepane, azetidine, aziridine, azocane, diazepane, dioxane, dioxolane, dithiane, dithiolane, imidazolidine, isothiazolidine, isoxalidine, morpholine, oxazolidine, oxepane, oxetane, oxirane, piperazine, piperidine, pyrazolidine, pyrrolidine, quinuclidine, tetrahydrofuran, tetrahydropyran, tetrahydrothiopyran, thiazolidine, thietane, thiirane, thiolane, thiomorpholine, indoline, dihydrobenzofuran, dihydrobenzothiophene, 1,1-dioxothiacyclohexane, 2-azaspiro[3.3]heptane, 2-oxaspiro[3.3]heptane, 7-azaspiro[3.5]nonane, 8-azabicyclo[3.2.1]octane, 9-azabicyclo[3.3.1]nonane, hexahydro-1H-pyrrolizine, hexahydrocyclopenta[c]pyrrole, octahydrocyclopenta[c]pyrrole, and octahydropyrrolo[1,2-a]pyrazin, in each case unsubstituted, mono- or polysubstituted; preferably the definition is selected from azepane, azetidine, aziridine, diazepane, dioxane, dioxolane, dithiane, dithiolane, imidazolidine, isoxalidine, morpholine, oxazolidine, oxepane, oxetane, oxirane, piperazine, piperidine, pyrazolidine, pyrrolidine, quinuclidine, tetrahydrofuran, tetrahydropyran, thiazolidine, thiolane, indoline, dihydrobenzofuran, dihydrobenzothiophene, 2-azaspiro[3.3]heptane, 2-oxaspiro[3.3]heptane, 7-azaspiro[3.5]nonane, hexahydro-1H-pyrrolizine, hexahydrocyclopenta[c]pyrrole, octahydrocyclopenta[c]pyrrole, and octahydropyrrolo[1,2-a]pyrazin, in each case unsubstituted, mono- or polysubstituted; preferably the definition is selected from azepane, azetidine, aziridine, diazepane, dioxane, dioxolane, dithiolane, imidazolidine, isoxalidine, morpholine, oxazolidine, oxepane, oxetane, oxirane, piperazine, piperidine, pyrazolidine, pyrrolidine, tetrahydrofuran, tetrahydropyran, thiazolidine, thiolane, indoline, dihydrobenzofuran, dihydrobenzothiophene, in each case unsubstituted, mono- or polysubstituted; preferably the definition is selected from azetidine, dioxane, imidazolidine, isoxalidine, morpholine, oxazolidine, oxetane, oxirane, piperazine, piperidine, pyrazolidine, pyrrolidin-1-yl, pyrrolidin-2-yl, tetrahydrofuran-1-yl, tetrahydrofuran-2-yl, tetrahydropyran-1-yl, tetrahydropyran-2-yl, tetrahydropyran-3-yl, tetrahydropyran-4-yl, thiazolidine, thiolane, and indoline, in each case unsubstituted, mono- or polysubstituted.

In some embodiments, the 5-14-membered heteroaryl within the definition of W represents a residue according to general formula (E)

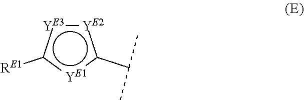

wherein
Y^{E1} represents —N═, —NR^{E2}—, S, O, or —CR^{E3}═; Y^{E2} represents —N═, —NR^{E3}—, S, O, or —CR^{E4}═; and Y^{E3} represent —N═, —NR^{E4}—, S, or —CR^{E5}═; with the proviso that at least one of Y^{E1} Y^{E2} and Y^{E3} is not —CR^{E3}═, —CR^{E4}═, and —CR^{E5}═, respectively. In another preferred embodiment, W represents a residue according to general formula (E) wherein YE1 represents —N═, —NRE2-, S, or —CRE3=; YE2 represents —N═, —NRE3-, S, or —CRE4=; and YE3 represent —N═, —NRE4-, S, or —CRE5=; with the proviso that at least one of YE1, YE2, and YE3 is not —CRE3=, —CR^{E4}═, and —CR^{E5}═, respectively.
R^{E1}, R^{E2}, R^{E3} and R^{E4} independently of one another represent —H, —CH₃, —CH₂-cyclopropyl, —CH₂CF₃, —CH₂CHF₂ or —CF₃; more in particular R^{E1}, R^{E2}, R^{E3} and R^{E4} independently of one another represent —H, —CH₃, or —CF₃; preferably with the proviso that only one of R^{E1}, R^{E2}, R^{E3} and R^{E4} represents a residue that is not —H.

In some embodiments, the 5-14-membered heteroaryl within the definition of W represents a residue according to general formula (F′)

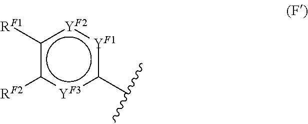

wherein
Y^{F1} represents —N═ or —CR^{F4}═; and Y^{F2} represents —N═ or —CR^{F5}═; and Y^{F3} represents —N═ or —CR^{F3}═; with the proviso that at least one of Y^{F1} and Y^{F2} is not —CR^{F4}═ and —CR^{F5}═, respectively;
R^{F1}, R^{F2}, R^{F3}, R^{F4}, and R^{F5} independently of one another represent —H, —CH₃, —CF₃, —OH, —OCH₃, —OCH₂CH₃, —Cl, or -azetidinyl; preferably with the proviso that only one of R^{F1}, R^{F2}, R^{F3}, R^{F4} and R^{F5} represents a residue that is not —H.

In some embodiments, the 5-14-membered heteroaryl within the definition of W represents a residue according to general formula (F)

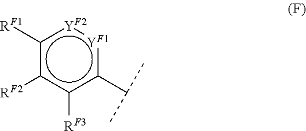

wherein
Y^{F1} represents —N═ or —CR^{F4}═; and Y^{F2} represents —N═ or —CR^{F3}═; with the proviso that at least one of Y^{F1} and Y^{F2} is not —CR^{F4}═ and FC^{R5}═, respectively;
R^{F1}, R^{F2}, R^{F3}, R^{F4} and R^{F5} independently of one another represent —H, —CH₃, —CF₃, —OH, —OCH₃, —OCH₂CH₃, —Cl, or -azetidinyl; preferably with the proviso that only one of R^{F1}, R^{F2}, R^{F3}, R^{F4} and R^{F5} represents a residue that is not —H.

In some embodiments, the 5-14-membered heteroaryl within the definition of W represents a residue according to general formula (G) or (H)

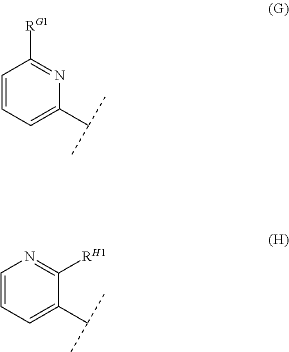

wherein R^{G1} and R^{H1} are selected from the group consisting of —H, —CH₃, —CF₃, —OH, —OCH₃, —OCH₂CH₃, —Cl, azetidinyl, -cyclopropyl, —O-cyclopropyl, and —CHF₂, or wherein R^{G1} and R^{H1} are selected from the group consisting of —H, —CH₃, —CF₃, —OH, —OCH₃, —OCH₂CH₃, —Cl, and azetidinyl.

In some embodiments, the 5-14-membered heteroaryl within the definition of W represents a residue according to general formula (G′) or (H′)

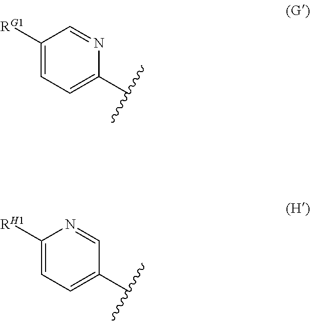

wherein R^{G1} and R^{H1} are selected from the group consisting of —H, —CH₃, —CF₃, —OH, —OCH₃, —OCH₂CH₃, —Cl, azetidinyl, -cyclopropyl, —O-cyclopropyl, and —CHF₂, or wherein R^{G1} and R^{H1} are selected from the group consisting of —H, —CH₃, —CF₃, —OH, —OCH₃, —OCH₂CH₃, —Cl, and azetidinyl,

In some embodiments, the 5-14-membered heteroaryl within the definition of W represents a residue according to general formula (E), (F), (F′), (G), (H), (G′) or (H′), as defined hereinabove.

In some embodiments the terminology “mono- or polysubstituted” in each case independently means substituted with one or more substituents independently of one another selected from —F, —Cl, —Br, —I, —CN, —C₁₋₆-alkyl, —CF₃, —CF₂H, —CFH₂, —CF₂Cl, —CFCl₂, —C₁₋₆-alkylene-CF₃, —C₁₋₆-alkylene-CF₂H, —C₁₋₆-alkylene-CFH₂, —C₁₋₆-alkylene-NH—C₁₋₆-alkylene-CF₃, —C₁₋₆-alkylene-N(C₁₋₆-alkyl)-C₁₋₆-alkylene-CF₃, —C(═O)—C₁₋₆-alkyl, —C₁₋₆-alkylene-C(═O)—C₁₋₆-alkyl, —C(═O)OH, —C₁₋₆-alkylene-C(═O)—OH, —C(═O)—OC₁₋₆-alkyl, —C₁₋₆-alkylene-C(═O)—OC₁₋₆-alkyl, —C(═O)O—C₁₋₆-alkylene-CF₃, —C(═O)—NH₂, —C₁₋₆-alkylene-C(═O)—NH₂, —C(═O)—NH(C₁₋₆-alkyl), —C₁₋₆-alkylene-C(═O)—NH(C₁₋₆-alkyl), —C(═O)—N(C₁₋₆-alkyl)₂, —C₁₋₆-alkylene-C(═O)—N(C₁₋₆-alkyl)₂, —C(═O)—NH(OH), —C₁₋₆-alkylene-C(═O)—NH(OH), —OH, —C₁₋₆-alkylene-OH, ═O, —OCF₃, —OCF₂H, —OCFH₂, —OCF₂Cl, —OCFCl₂, —O—C₁₋₆-alkyl, —C₁₋₆-alkylene-O—C₁₋₆-alkyl, —O—C₁₋₆-alkylene-O—C₁₋₆-alkyl, —O—C₁₋₆-alkylene-NH₂, —O—C₁₋₆-alkylene-NH—C₁₋₆-alkyl, —O—C₁₋₆-alkylene-N(C₁₋₆-alkyl)₂, —O—C(═O)—C₁₋₆-alkyl, —C₁₋₆-alkylene-O—C(═O)—C₁₋₆-alkyl, —O—C(═O)—O—C₁₋₆-alkyl, —C₁₋₆-alkylene-O—C(═O)—O—C₁₋₆-alkyl, —O—C(═O)—NH(C₁₋₆-alkyl), —C₁₋₆-alkylene-O—C(═O)—NH(C₁₋₆-alkyl), —O—C(═O)—N(C₁₋₆-alkyl)₂, —C₁₋₆-alkylene-O—C(═O)—N(C₁₋₆-alkyl)₂, —O—S(═O)₂—NH₂, —C₁₋₆-alkylene-O—S(═O)₂—NH₂, —O—S(═O)₂—NH(C₁₋₆-alkyl), —C₁₋₆-alkylene-O—S(═O)₂—NH(C₁₋₆-alkyl), —O—S(═O)₂—N(C₁₋₆-alkyl)₂, —C₁₋₆-alkylene-O—S(═O)₂—N(C₁₋₆-alkyl)₂, —NH₂, —NO, —NO₂, —C₁₋₆-alkylene-NH₂, —NH(C₁₋₆-alkyl), —C₁₋₆-alkylene-NH(C₁₋₆-alkyl), —N(C₁₋₆-alkyl)₂, —C₁₋₆-alkylene-N(C₁₋₆-alkyl)₂, —NH—C(═O)—C₁₋₆-alkyl, —C₁₋₆-alkylene-NH—C(═O)—C₁₋₆-alkyl, —NH—C(═O)—O—C₁₋₆-alkyl, —C₁₋₆-alkylene-NH—C(═O)—O—C₁₋₆-alkyl, —NH—C(═O)—NH₂, —C₁₋₆-alkylene-NH—C(═O)—NH₂, —NH—C(═O)—NH(C₁₋₆-alkyl), —C₁₋₆-alkylene-NH—C(═O)—NH(C₁₋₆-alkyl), —NH—C(═O)—N(C₁₋₆-alkyl)₂, —C₁₋₆-alkylene-NH—C(═O)—N(C₁₋₆-alkyl)₂, —N(C₁₋₆-alkyl)-C(═O)—C₁₋₆-alkyl, —C₁₋₆-alkylene-N(C₁₋₆-alkyl)-C(═O)—C₁₋₆-alkyl, —N(C₁₋₆-alkyl)-C(═O)—O—C₁₋₆-alkyl, —C₁₋₆-alkylene-N(C₁₋₆-alkyl)-C(═O)—O—C₁₋₆-alkyl, —N(C₁₋₆-alkyl)-C(═O)—NH₂, —C₁₋₆-alkylene-N(C₁₋₆-alkyl)-C(═O)—NH₂, —N(C₁₋₆-alkyl)-C(═O)—NH(C₁₋₆-alkyl), —C₁₋₆-alkylene-N(C₁₋₆-alkyl)-C(═O)—NH(C₁₋₆-alkyl), —N(C₁₋₆-alkyl)-C(═O)—N(C₁₋₆-alkyl)₂, —C₁₋₆-alkylene-N(C₁₋₆-alkyl)-C(═O)—N(C₁₋₆-alkyl)₂, —NH—S(═O)₂OH, —C₁₋₆-alkylene-NH—S(═O)₂OH, —NH—S(═O)₂—C₁₋₆-alkyl, —C₁₋₆-alkylene-NH—S(═O)₂—C₁₋₆-alkyl, —NH—S(═O)₂—O—C₁₋₆-alkyl, —C₁₋₆-alkylene-NH—S(═O)₂—O—C₁₋₆-alkyl, —NH—S(═O)₂—NH₂, —C₁₋₆-alkylene-NH—S(═O)₂—NH₂, —NH—S(═O)₂—NH(C₁₋₆-alkyl), —C₁₋₆-alkylene-NH—S(═O)₂—NH(C₁₋₆-alkyl), —NH—S(═O)₂N(C₁₋₆-alkyl)₂, —C₁₋₆-alkylene-NH—S(═O)₂N(C₁₋₆-alkyl)₂, —N(C₁₋₆-alkyl)-S(═O)₂—OH, —C₁₋₆-alkylene-N(C₁₋₆-alkyl)-S(═O)₂—OH, —N(C₁₋₆-alkyl)-S(═O)₂—C₁₋₆-alkyl, —C₁₋₆-alkylene-N(C₁₋₆-alkyl)-S(═O)₂—C₁₋₆-alkyl, —N(C₁₋₆-alkyl)-S(═O)₂—O—C₁₋₆-alkyl, —C₁₋₆-alkylene-N(C₁₋₆-alkyl)-S(═O)₂—O—C₁₋₆-alkyl, —N(C₁₋₆-alkyl)-S(═O)₂—NH₂, —C₁₋₆-alkylene-N(C₁₋₆-alkyl)-S(═O)₂—NH₂, —N(C₁₋₆-alkyl)-S(═O)₂—NH(C₁₋₆-alkyl), —C₁₋₆-alkylene-N(C₁₋₆-alkyl)-S(═O)₂—NH(C₁₋₆-alkyl), —N(C₁₋₆-alkyl)-S(═O)₂—N(C₁₋₆-alkyl)₂, —C₁₋₆-alkylene-N(C₁₋₆-alkyl)-S(═O)₂—N(C₁₋₆-alkyl)₂, —SH, ═S, —SF₅, —SCF₃, —SCF₂H, —SCFH₂, —S—C₁₋₆-alkyl, —C₁₋₆-alkylene-S—C₁₋₆-alkyl, —S(═O)—C₁₋₆-alkyl, —C₁₋₆-alkylene-S(═O)—C₁₋₆-alkyl, —S(═O)₂—C₁₋₆-alkyl, —C₁₋₆-alkylene-S(═O)₂—C₁₋₆-alkyl, —S(═O)₂—OH, —C₁₋₆-alkylene-S(═O)₂—OH, —S(═O)₂—O—C₁₋₆-alkyl, —C₁₋₆-alkylene-S(═O)₂—O—C₁₋₆-alkyl, —S(═O)₂—NH₂, —C₁₋₆-alkylene-S(═O)₂—NH₂, —S(═O)₂—NH(C₁₋₆-alkyl), —C₁₋₆-alkylene-S(═O)₂—NH(C₁₋₆-alkyl), —S(═O)₂—N(C₁₋₆-alkyl)₂, —C₁₋₆-alkylene-S(═O)₂—N(C₁₋₆-alkyl)₂, 3-14-membered cycloalkyl, —C₁₋₆-alkylene-(3-14-membered cycloalkyl), 3 to 14-membered heterocycloalkyl, —C₁₋₆-alkylene-(3 to 14-membered heterocycloalkyl), -phenyl, —C₁₋₆-alkylene-phenyl, 5 to 14-membered heteroaryl, —C₁₋₆-alkylene-(5 to 14-membered heteroaryl), —O-(3-14-membered cycloalkyl), —O-(3 to 14-membered heterocycloalkyl), —O-phenyl, —O-(5 to 14-membered heteroaryl), —C(═O)-(3-14-membered cycloalkyl), —C(═O)-(3 to 14-membered heterocycloalkyl), —C(═O)-phenyl, —C(═O)-(5 to 14-membered heteroaryl), —S(═O)₂-(3-14-membered cycloalkyl), —S(═O)₂-(3 to 14-membered heterocycloalkyl), —S(═O)₂-phenyl, or —S(═O)₂-(5 to 14-membered heteroaryl); preferably the terminology in each case independently means substituted with one or more substituents independently of one another selected from —F, —Cl, —Br, —I, —CN, —C₁₋₆-alkyl, —CF₃, —CF₂H, —CFH₂, —CF₂Cl, —CFCl₂, —C₁₋₆-alkylene-CF₃, —C₁₋₆-alkylene-CF₂H, —C₁₋₆-alkylene-CFH₂, —C₁₋₆-alkylene-NH—C₁₋₆-alkylene-CF₃, —C₁₋₆-alkylene-N(C₁₋₆-alkyl)-C₁₋₆-alkylene-CF₃, —C(═O)OH, —C₁₋₆-alkylene-C(═O)—OH, —C(═O)—OC₁₋₆-alkyl, —C₁₋₆-alkylene-C(═O)—OC₁₋₆-alkyl, —C(═O)O—C₁₋₆-alkylene-CF₃, —C(═O)—NH₂, —C₁₋₆-alkylene-C(═O)—NH₂, —C(═O)—NH(C₁₋₆-alkyl), —C₁₋₆-alkylene-C(═O)—NH(C₁₋₆-alkyl), —C(═O)—N(C₁₋₆-alkyl)₂, —C₁₋₆-alkylene-C(═O)—N(C₁₋₆-alkyl)₂, —OH, —C₁₋₆-alkylene-OH, ═O, —OCF₃, —OCF₂H, —OCFH₂, —OCF₂Cl, —OCFCl₂, —O—C₁₋₆-alkyl, —C₁₋₆-alkylene-O—C₁₋₆-alkyl, —O—C(═O)—C₁₋₆-alkyl, —C₁₋₆-alkylene-O—C(═O)—C₁₋₆-alkyl, —O—C(═O)—NH(C₁₋₆-alkyl), —NH₂, —NO, —NO₂, —C₁₋₆-alkylene-NH₂, —NH(C₁₋₆-alkyl), —C₁₋₆-alkylene-NH(C₁₋₆-alkyl), —N(C₁₋₆-alkyl)₂, —C₁₋₆-alkylene-N(C₁₋₆-alkyl)₂, —NH—C(═O)—C₁₋₆-alkyl, —C₁₋₆-alkylene-NH—C(═O)—C₁₋₆-alkyl, —NH—C(═O)—O—C₁₋₆-alkyl, —C₁₋₆-alkylene-NH—C(═O)—O—C₁₋₆-alkyl, —N(C₁₋₆-alkyl)-C(═O)—C₁₋₆-alkyl, —C₁₋₆-alkylene-N(C₁₋₆-alkyl)-C(═O)—C₁₋₆-alkyl, —N(C₁₋₆-alkyl)-C(═O)—O—C₁₋₆-alkyl, —C₁₋₆-alkylene-N(C₁₋₆-alkyl)-C(═O)—O—C₁₋₆-alkyl, —N(C₁₋₆-alkyl)-C(═O)—NH₂, —NH—S(═O)₂OH, —C₁₋₆-alkylene-NH—S(═O)₂OH, —NH—S(═O)₂—C₁₋₆-alkyl, —C₁₋₆-alkylene-NH—S(═O)₂—C₁₋₆-alkyl, —NH—S(═O)₂—O—C₁₋₆-alkyl, —C₁₋₆-alkylene-NH—S(═O)₂—O—C₁₋₆-alkyl, —N(C₁₋₆-alkyl)-S(═O)₂—OH, —C₁₋₆-alkylene-N(C₁₋₆-alkyl)-S(═O)₂—OH, —N(C₁₋₆-alkyl)-S(═O)₂—C₁₋₆-alkyl, —C₁₋₆-alkylene-N(C₁₋₆-alkyl)-S(═O)₂—C₁₋₆-alkyl, —N(C₁₋₆-alkyl)-S(═O)₂—O—C₁₋₆-alkyl, —C₁₋₆-alkylene-N(C₁₋₆-alkyl)-S(═O)₂—O—C₁₋₆-alkyl, —N(C₁₋₆-alkyl)-S(═O)₂—NH₂, —C₁₋₆-alkylene-N(C₁₋₆-alkyl)-S(═O)₂—NH₂, —N(C₁₋₆-alkyl)-S(═O)₂—NH(C₁₋₆-alkyl), —C₁₋₆-alkylene-N(C₁₋₆-alkyl)-S(═O)₂—NH(C₁₋₆-alkyl), —N(C₁₋₆-alkyl)-S(═O)₂—N(C₁₋₆-alkyl)₂, —C₁₋₆-alkylene-N(C₁₋₆-alkyl)-S(═O)₂—N(C₁₋₆-alkyl)₂, —SH, ═S, —SF₅, —SCF₃, —SCF₂H, —SCFH₂, —S—C₁₋₆-alkyl, —C₁₋₆-alkylene-S—C₁₋₆-alkyl, —S(═O)₂—C₁₋₆-alkyl, —C₁₋₆-alkylene-S(═O)₂—C₁₋₆-alkyl, —S(═O)₂—OH, —C₁₋₆-alkylene-S(═O)₂—OH, —S(═O)₂—O—C₁₋₆-alkyl, —C₁₋₆-alkylene-S(═O)₂—O—C₁₋₆-alkyl, —S(═O)₂—NH₂, —C₁₋₆-alkylene-S(═O)₂—NH₂, —S(═O)₂—NH(C₁₋₆-alkyl), —C₁₋₆-alkylene-S(═O)₂—NH(C₁₋₆-alkyl), —S(═O)₂—N(C₁₋₆-alkyl)₂, —C₁₋₆-alkylene-S(═O)₂—N(C₁₋₆-alkyl)₂, 3-14-membered cycloalkyl, —C₁₋₆-alkylene-(3-14-membered cycloalkyl), 3 to 14-membered heterocycloalkyl, —C₁₋₆-alkylene-(3 to 14-membered heterocycloalkyl), -phenyl, —C₁₋₆-alkylene-phenyl, 5 to 14-membered heteroaryl, —C₁₋₆-alkylene-(5 to 14-membered heteroaryl), —O-(3-14-membered cycloalkyl), —O-(3 to 14-membered heterocycloalkyl), —O-phenyl, —O-(5 to 14-membered heteroaryl), —S(═O)₂-(3-14-membered cycloalkyl), —S(═O)₂-(3 to 14-membered heterocycloalkyl), —S(═O)₂-phenyl, or —S(═O)₂-(5 to 14-membered heteroaryl); preferably the terminology in each case independently means substituted with one or more substituents independently of one another selected from —F, —Cl, —Br, —I, —CN, —C₁₋₆-alkyl, —CF₃, —CF₂H, —CFH₂, —CF₂Cl, —CFCl₂, —C₁₋₆-alkylene-CF₃, —C₁₋₆-alkylene-CF₂H, —C₁₋₆-alkylene-CFH₂, —C₁₋₆-alkylene-NH—C₁₋₆-alkylene-CF₃, —C₁₋₆-alkylene-N(C₁₋₆-alkyl)-C₁₋₆-alkylene-CF₃, —C(═O)OH, —C₁₋₆-alkylene-C(═O)—OH, —C(═O)—OC₁₋₆-alkyl, —C₁₋₆-alkylene-C(═O)—OC₁₋₆-alkyl, —C(═O)O—C₁₋₆-alkylene-CF₃, —C(═O)—NH₂, —C₁₋₆-alkylene-C(═O)—NH₂, —C(═O)—NH(C₁₋₆-alkyl), —C₁₋₆-alkylene-C(═O)—NH(C₁₋₆-alkyl), —C(═O)—N(C₁₋₆-alkyl)₂, —C₁₋₆-alkylene-C(═O)—N(C₁₋₆-alkyl)₂, —OH, —C₁₋₆-alkylene-OH, ═O, —OCF₃, —OCF₂H, —OCFH₂, —OCF₂Cl, —OCFCl₂, —O—C₁₋₆-alkyl, —C₁₋₆-alkylene-O—C₁₋₆-alkyl, —O—C(═O)—C₁₋₆-alkyl, —C₁₋₆-alkylene-O—C(═O)—C₁₋₆-alkyl, —O—C(═O)—NH(C₁₋₆-alkyl), —NH₂, —NO, —NO₂, —C₁₋₆-alkylene-NH₂, —NH(C₁₋₆-alkyl), —C₁₋₆-alkylene-NH(C₁₋₆-alkyl), —N(C₁₋₆-alkyl)₂, —C₁₋₆-alkylene-N(C₁₋₆-allyl)₂, —NH—C(═O)—C₁₋₆-alkyl, —C₁₋₆-alkylene-NH—C(═O)—C₁₋₆-alkyl, —N(C₁₋₆-alkyl)-C(═O)—C₁₋₆-alkyl, —C₁₋₆-alkylene-N(C₁₋₆-alkyl)-C(═O)—C₁₋₆-alkyl, —C₁₋₆-alkylene-N(C₁₋₆-alkyl)-S(═O)₂—OH, —N(C₁₋₆-alkyl)-S(═O)₂—C₁₋₆-alkyl, —C₁₋₆-alkylene-N(C₁₋₆-alkyl)-S(═O)₂—C₁₋₆-alkyl, —N(C₁₋₆-alkyl)-S(═O)₂—O—C₁₋₆-alkyl, —C₁₋₆-alkylene-N(C₁₋₆-alkyl)-S(═O)₂—O—C₁₋₆-alkyl, —N(C₁₋₆-alkyl)-S(═O)₂—NH₂, —C₁₋₆-alkylene-N(C₁₋₆-alkyl)-S(═O)₂—NH₂, —N(C₁₋₆-alkyl)-S(═O)₂—NH(C₁₋₆-alkyl), —C₁₋₆-alkylene-N(C₁₋₆-alkyl)-S(═O)₂—NH(C₁₋₆-alkyl), —N(C₁₋₆-alkyl)-S(═O)₂—N(C₁₋₆-alkyl)₂, —C₁₋₆-alkylene-N(C₁₋₆-alkyl)-S(═O)₂—N(C₁₋₆-alkyl)₂, —SH, —S—C₁₋₆-alkyl, —C₁₋₆-alkylene-S—C₁₋₆-alkyl, —S(═O)₂—C₁₋₆-alkyl, —C₁₋₆-alkylene-S(═O)₂—C₁₋₆-alkyl, —S(═O)₂—OH, —C₁₋₆-alkylene-S(═O)₂—OH, —S(═O)₂—O—C₁₋₆-alkyl, —C₁₋₆-alkylene-S(═O)₂—O—C₁₋₆-alkyl, —S(═O)₂—NH₂, —C₁₋₆-alkylene-S(═O)₂—NH₂, —S(═O)₂—NH(C₁₋₆-alkyl), —C₁₋₆-alkylene-S(═O)₂—NH(C₁₋₆-alkyl), —S(═O)₂—N(C₁₋₆-alkyl)₂, —C₁₋₆-alkylene-S(═O)₂—N(C₁₋₆-alkyl)₂, 3-14-membered cycloalkyl, —C₁₋₆-alkylene-(3-14-membered cycloalkyl), 3 to 14-membered heterocycloalkyl, —C₁₋₆-alkylene-(3 to 14-membered heterocycloalkyl), -phenyl, —C₁₋₆-alkylene-phenyl, 5 to 14-membered heteroaryl, —C₁₋₆-alkylene-(5 to 14-membered heteroaryl), —O-(3-14-membered cycloalkyl), —O-(3 to 14-membered heterocycloalkyl), —O-phenyl, —S(═O)₂-(3-14-membered cycloalkyl), —S(═O)₂-(3 to 14-membered heterocycloalkyl), —S(═O)₂-phenyl, or —S(═O)₂-(5 to 14-membered heteroaryl); preferably the terminology in each case independently means substituted with one or more substituents independently of one another selected from —F, —Cl, —Br, —I, —CN, —C₁₋₆-alkyl, —CF₃, —CF₂H, —CFH₂, —CF₂Cl, —CFCl₂, —C₁₋₆-alkylene-CF₃, —C₁₋₆-alkylene-CF₂H, —C₁₋₆-alkylene-CFH₂, —C₁₋₆-alkylene-NH—C₁₋₆-alkylene-CF₃, —C₁₋₆-alkylene-N(C₁₋₆-alkyl)-C₁₋₆-alkylene-CF₃, —C(═O)OH, —C₁₋₆-alkylene-C(═O)—OH, —C(═O)—OC₁₋₆-alkyl, —C₁₋₆-alkylene-C(═O)—OC₁₋₆-alkyl, —C(═O)O—C₁₋₆-alkylene-CF₃, —C(═O)—NH₂, —C₁₋₆-alkylene-C(═O)—NH₂, —C(═O)—NH(C₁₋₆-alkyl), —C₁₋₆-alkylene-C(═O)—NH(C₁₋₆-alkyl), —C(═O)—N(C₁₋₆-alkyl)₂, —C₁₋₆-alkylene-C(═O)—N(C₁₋₆-alkyl)₂, —OH, —C₁₋₆-alkylene-OH, ═O, —OCF₃, —OCF₂H, —OCFH₂, —OCF₂Cl, —OCFCl₂, —O—C₁₋₆-alkyl, —C₁₋₆-alkylene-O—C₁₋₆-alkyl, —O—C(═O)—C₁₋₆-alkyl, —C₁₋₆-alkylene-O—C(═O)—C₁₋₆-alkyl, —O—C(═O)—NH(C₁₋₆-alkyl), —NH₂, —NO₂, —C₁₋₆-alkylene-NH₂, —NH(C₁₋₆-alkyl), —C₁₋₆-alkylene-NH(C₁₋₆-alkyl), —N(C₁₋₆-alkyl)₂, —C₁₋₆-alkylene-N(C₁₋₆-alkyl)₂, —NH—C(═O)—C₁₋₆-alkyl, —C₁₋₆-alkylene-NH—C(═O)—C₁₋₆-alkyl, —N(C₁₋₆-alkyl)-C(═O)—C₁₋₆-alkyl, —C₁₋₆-alkylene-N(C₁₋₆-alkyl)-C(═O)—C₁₋₆-alkyl, —N(C₁₋₆-alkyl)-S(═O)₂—C₁₋₆-alkyl, —N(C₁₋₆-alkyl)-S(═O)₂—O—C₁₋₆-alkyl, —SH, —S—C₁₋₆-alkyl, —C₁₋₆-alkylene-S—Cl-6-alkyl, —S(═O)₂—C₁₋₆-alkyl, —C₁₋₆-alkylene-S(═O)₂—C₁₋₆-alkyl, —S(═O)₂—OH, —C₁₋₆-alkylene-S(═O)₂—OH, —S(═O)₂—O—C₁₋₆-alkyl, —S(═O)₂—NH₂, —S(═O)₂—NH(C₁₋₆-alkyl), —S(═O)₂—N(C₁₋₆-alkyl)₂, 3-12-membered cycloalkyl, —C₁₋₆-alkylene-(3-12-membered cycloalkyl), 3 to 12-membered heterocycloalkyl, —C₁₋₆-alkylene-(3 to 12-membered heterocycloalkyl), -phenyl, —C₁₋₆-alkylene-phenyl, 5 to 12-membered heteroaryl, —C₁₋₆-alkylene-(5 to 12-membered heteroaryl); preferably the terminology in each case independently means substituted with one or more substituents independently of one another selected from —F, —Cl, —Br, —I, —CN, —C₁₋₆-alkyl, —CF₃, —CF₂H, —CFH₂, —CF₂Cl, —CFCl₂, —C₁₋₆-alkylene-CF₃, —C₁₋₆-alkylene-CF₂H, —C₁₋₆-alkylene-CFH₂, —C₁₋₆-alkylene-NH—C₁₋₆-alkylene-CF₃, —C(═O)OH, —C₁₋₆-alkylene-C(═O)—OH, —C(═O)—OC₁₋₆-alkyl, —C(═O)O—C₁₋₆-alkylene-CF₃, —C(═O)—NH₂, —C(═O)—NH(C₁₋₆-alkyl), C(═O)—N(C₁₋₆-alkyl)₂, —C₁₋₆-alkylene-C(═O)—N(C₁₋₆-alkyl)₂, —OH, —C₁₋₆-alkylene-OH, ═O, —OCF₃, —OCF₂H, —OCFH₂, —OCF₂Cl, —OCFCl₂, —O—C₁₋₆-alkyl, —O—C(═O)—C₁₋₆-alkyl, —O—C(═O)—NH(C₁₋₆-alkyl), —NH₂, —NO₂, —C₁₋₆-alkylene-NH₂, —NH(C₁₋₆-alkyl), —C₁₋₆-alkylene-NH(C₁₋₆-alkyl), —N(C₁₋₆-allyl)₂, —C₁₋₆-alkylene-N(C₁₋₆-alkyl)₂, —N(C₁₋₆-alkyl)-S(═O)₂—C₁₋₆-alkyl, —N(C₁₋₆-alkyl)-S(═O)₂—O—C₁₋₆-alkyl, —SH, —S—C₁₋₆-alkyl, —S(═O)₂—C₁₋₆-alkyl, —S(═O)₂—OH, —S(═O)₂—O—C₁₋₆-alkyl, —S(═O)₂—NH₂, —S(═O)₂—NH(C₁₋₆-alkyl), —S(═O)₂—N(C₁₋₆-alkyl)₂, 3-12-membered cycloalkyl, —C₁₋₆-alkylene-(3-12-membered cycloalkyl), 3 to 12-membered heterocycloalkyl, —C₁₋₆-alkylene-(3 to 12-membered heterocycloalkyl), -phenyl, —C₁₋₆-alkylene-phenyl, 5 to 12-membered heteroaryl, —C₁₋₆-alkylene-(5 to 12-membered heteroaryl); preferably the terminology in each case independently means substituted with one or more substituents independently of one another selected from —F, —Cl, —Br, —I, —CN, —C₁₋₅-alkyl, —CF₃, —CF₂H, —CFH₂, —CF₂Cl, —CFCl₂, —C₁—-alkylene-CF₃, —C₁₋₅-alkylene-NH—C₁₋₅-alkylene-CF₃, —C(═O)OH, —C₁-s-alkylene-C(═O)—OH, —C(═O)—OC₁₋₅-alkyl, —C(═O)—NH₂, —C(═O)—NH(C₁-5-alkyl), C(═O)—N(C₁₋₅-alkyl)₂, —OH, —C₁₋₅-alkylene-OH, ═O, —OCF₃, —OCF₂H, —OCFH₂, —OCF₂Cl, —OCFCl₂, —O—C₁₋₅-alkyl, —O—C(═O)—C₁₋₅-alkyl, —O—C(═O)—NH(C₁₋₆-alkyl), —NH₂, —NO₂, —C₁₋₅-alkylene-NH₂, —NH(C₁-5-alkyl), —C₁₋₅-alkylene-NH(C₁₋₅-alkyl), —N(C₁₋₅-alkyl)₂, —C₁₋₅-alkylene-N(C₁₋₅-alkyl)₂, —N(C₁₋₅-alkyl)-S(═O)₂—C₁₋₅-alkyl, —N(C₁₋₅-alkyl)-S(═O)₂—O—C₁₋₅-alkyl, —SH, —S—C₁₋₅alkyl, —S(═O)₂—C₁₋₅alkyl, —S(═O)₂—OH, —S(═O)₂—O—C₁₋₆-alkyl, —S(═O)₂—NH₂, —S(═O)₂—NH(C₁₋₆-alkyl), —S(═O)₂—N(C₁₋₆-alkyl)₂, 3-10-membered cycloalkyl, —C₁₋₅-alkylene-(3-10-membered cycloalkyl), 3 to 10-membered heterocycloalkyl, —C₁₋₅-alkylene-(3 to 10-membered heterocycloalkyl), -phenyl, —C₁₋₅-alkylene-phenyl, 5 to 10-membered heteroaryl, —C₁₋₅-alkylene-(5 to 10-membered heteroaryl).

In some embodiments the 3-14-membered cycloalkyl within the definition of W is unsubstituted, mono- or disubstituted with a substituent selected from the group consisting of —F, —Cl, —Br, —I, —CN, C₁₋₃-alkyl, —CF₃, —CF₂H, —CFH₂, —CF₂Cl, —CFCl₂, —C₁₋₃-alkylene-CF₃, —C₁₋₃-alkylene-CF₂H, —C₁₋₃-alkylene-CFH₂, —OCF₃, —OCF₂H, —OCFH₂, —OCF₂Cl, —OCFCl₂, —O—C₁₋₃-alkyl, —C₁₋₃-alkylene-O—C₁₋₃-alkyl, and —C₁₋₃-alkylene-OH; preferably —F, —Cl, —Br, —I, —CN, —CH₃, —CF₃, —CF₂H, —CFH₂, —OCF₃, and —OCH₃.

In some embodiments the 6-14-membered aryl within the definition of W is unsubstituted, mono- or disubstituted with a substituent selected from the group consisting of —F, —Cl, —Br, —I, —CN, C₁₋₃-alkyl, —CF₃, —CF₂H, —CFH₂, —CF₂Cl, —CFCl₂, —C₁₋₃-alkylene-CF₃, —C₁₋₃-alkylene-CF₂H, —C₁₋₃-alkylene-CFH₂, —OCF₃, —OCF₂H, —OCFH₂, —OCF₂Cl, —OCFCl₂, —O—C₁₋₃-alkyl, —C₁₋₃-alkylene-O—C₁₋₃-alkyl, and —C₁₋₃-alkylene-OH; preferably —F, —Cl, —Br, —I, —CN, —CH₃, —CF₃, —CF₂H, —CFH₂, —OCF₃, and —OCH₃.

In some embodiments the 5-14-membered heteroaryl within the definition of W is unsubstituted, mono- or disubstituted with a substituent selected from the group consisting of —F, —Cl, —Br, —I, —CN, C₁₋₃-alkyl, —CF₃, —CF₂H, —CFH₂, —CF₂Cl, —CFCl₂, —C₁₋₃-alkylene-CF₃, —C₁₋₃-alkylene-CF₂H, —C₁₋₃-alkylene-CFH₂, —OCF₃, —OCF₂H, —OCFH₂, —OCF₂Cl, —OCFCl₂, —O—C₁₋₃-alkyl, —C₁₋₃-alkylene-O—C₁₋₃-alkyl, and —C₁₋₃-alkylene-OH; preferably —F, —Cl, —Br, —I, —CN, —CH₃, —CF₃, —CF₂H, —CFH₂, —OCF₃, and —OCH₃.

In some embodiments the —C₁-C₆-alkyl within the definition of W is unsubstituted, mono- or disubstituted with a substituent selected from the group consisting of —F, —Cl, —Br, —I, —CN, C₁₋₃-alkyl, —CF₃, —CF₂H, —CFH₂, —CF₂Cl, —CFCl₂, —OCF₃, —OCF₂H, —OCFH₂, —OCF₂Cl, —OCFCl₂, —O—C₁₋₃-alkyl, and —OH; preferably —F, —Cl, —Br, —I, —CN, —CH₃, —CF₃, —CF₂H, —CFH₂, —OCF₃, and —OCH₃.

In some embodiments the 3-14-membered heterocycloalkyl within the definition of W is unsubstituted, mono- or disubstituted with a substituent selected from the group consisting of —F, —Cl, —Br, —I, —CN, C₁₋₃-alkyl, —CF₃, —CF₂H, —CFH₂, —CF₂Cl, —CFCl₂, —C₁₋₃-alkylene-CF₃, —C₁₋₃-alkylene-CF₂H, —C₁₋₃-alkylene-CFH₂, —OCF₃, —OCF₂H, —OCFH₂, —OCF₂Cl, —OCFCl₂, —O—C₁₋₃-alkyl, —C₁₋₃-alkylene-O—C₁₋₃-alkyl, and —C₁₋₃-alkylene-OH; preferably —F, —Cl, —Br, —I, —CN, —CH₃, —CF₃, —CF₂H, —CFH₂, —OCF₃, and —OCH₃.

In some embodiment of the invention, the compound is selected from the group consisting of
Cpd 001—(2S)-3-hydroxy-2-({2-methyl-5-[(2-methyl-1,3-thiazol-5-yl)methoxy]furo[2,3-c]pyridin-3-yl}formamido)propanamide;
Cpd 002—(2S,3R)-2-({5-[(2-fluorophenyl)methoxy]-2-methylfuro[2,3-c]pyridin-3-yl}formamido)-3-hydroxybutanamide;
Cpd 003—(2S,3R)-3-hydroxy-2-({2-methyl-5-[(2-methyl-1,3-thiazol-5-yl)methoxy]furo[2,3-c]pyridin-3-yl}formamido)butanamide;
Cpd 004—(2S,3R)-3-hydroxy-2-({2-methyl-5-[(4-methyl-1,3-thiazol-5-yl)methoxy]furo[2,3-c]pyridin-3-yl}formamido)butanamide;
Cpd 005—2-({2-methyl-5-[(4-methyl-1,3-thiazol-5-yl)methoxy]furo[2,3-c]pyridin-3-yl}formamido)propanamide;
Cpd 006—2-({2-methyl-5-[(pyridin-2-yl)methoxy]furo[2,3-c]pyridin-3-yl}formamido)propanamide;
Cpd 007—2-({5-[(2-fluorophenyl)methoxy]-2-methylfuro[2,3-c]pyridin-3-yl}formamido)-2-methylpropanamide;
Cpd 008—2-({5-[(2-fluorophenyl)methoxy]-2-methylfuro[2,3-c]pyridin-3-yl}formamido)-3-hydroxypropanamide;
Cpd 009—2-({5-[(2-fluorophenyl)methoxy]-2-methylfuro[2,3-c]pyridin-3-yl}formamido)propanamide;
Cpd 010—2-{[5-(benzyloxy)-2-methylfuro[2,3-c]pyridin-3-yl]formamido}propanamide;
Cpd 011—2-methyl-2-({2-methyl-5-[(4-methyl-1,3-thiazol-5-yl)methoxy]furo[2,3-c]pyridin-3-yl}formamido)propanamide;
Cpd 012—2-methyl-2-({2-methyl-5-[(pyridin-2-yl)methoxy]furo[2,3-c]pyridin-3-yl}formamido)propanamide;
Cpd 013—3-hydroxy-2-({2-methyl-5-[(4-methyl-1,3-thiazol-5-yl)methoxy]furo[2,3-c]pyridin-3-yl}formamido)propanamide;
Cpd 014—3-hydroxy-2-({2-methyl-5-[(pyridin-2-yl)methoxy]furo[2,3-c]pyridin-3-yl}formamido)propanamide;
Cpd 015—5-(benzyloxy)-N-(4,4-difluoropyrrolidin-3-yl)-2-methylfuro[2,3-c]pyridine-3-carboxamide;
Cpd 016—5-(benzyloxy)-N-[4-(hydroxymethyl)oxan-4-yl]-2-methylfuro[2,3-c]pyridine-3-carboxamide;
Cpd 017—5-[(2-fluorophenyl)methoxy]-N-[1-(2-hydroxyethyl)-2-oxopyrrolidin-3-yl]-2-methylfuro[2,3-c]pyridine-3-carboxamide;
Cpd 018—5-[(2-fluorophenyl)methoxy]-N-[1-(hydroxymethyl)cyclopropyl]-2-methylfuro[2,3-c]pyridine-3-carboxamide;
Cpd 019—N-(3-carbamoyloxetan-3-yl)-2-methyl-5-[(4-methyl-1,3-thiazol-5-yl)methoxy]furo[2,3-c]pyridine-3-carboxamide;
Cpd 020—N-(3-carbamoyloxetan-3-yl)-2-methyl-5-[(pyridin-2-yl)methoxy]furo[2,3-c]pyridine-3-carboxamide;
Cpd 021—N-(4,4-difluoropyrrolidin-3-yl)-2-methyl-5-[(4-methyl-1,3-thiazol-5-yl)methoxy]furo[2,3-c]pyridine-3-carboxamide;
Cpd 022—N-(4,4-difluoropyrrolidin-3-yl)-2-methyl-5-(2-phenylethoxy)furo[2,3-c]pyridine-3-carboxamide;
Cpd 023—N-(4,4-difluoropyrrolidin-3-yl)-2-methyl-5-[2-(4-methyl-1,3-thiazol-5-yl)ethoxy]furo[2,3-c]pyridine-3-carboxamide;
Cpd 024—N-(4-carbamoyloxan-4-yl)-2-methyl-5-[(pyridin-2-yl)methoxy]furo[2,3-c]pyridine-3-carboxamide;
Cpd 025—N-[(4R)-3,3-difluoropiperidin-4-yl]-2-methyl-5-{[2-(trifluoromethyl)pyridin-3-yl]methoxy}furo[2,3-c]pyridine-3-carboxamide;
Cpd 026—N-[(4S)-3,3-difluoropiperidin-4-yl]-2-methyl-5-{[2-(trifluoromethyl)pyridin-3-yl]methoxy}furo[2,3-c]pyridine-3-carboxamide;
Cpd 027—N-[1-(2-hydroxyethyl)-2-oxopyrrolidin-3-yl]-2-methyl-5-[(pyridin-2-yl)methoxy]furo[2,3-c]pyridine-3-carboxamide;
Cpd 028—N-[1-(hydroxymethyl)cyclobutyl]-2-methyl-5-[(4-methyl-1,3-thiazol-5-yl)methoxy]furo[2,3-c]pyridine-3-carboxamide;
Cpd 029—N-[1-(hydroxymethyl)cyclobutyl]-2-methyl-5-[(pyridin-2-yl)methoxy]furo[2,3-c]pyridine-3-carboxamide;
Cpd 030—N-[1-(hydroxymethyl)cyclopropyl]-2-methyl-5-[(4-methyl-1,3-thiazol-5-yl)methoxy]furo[2,3-c]pyridine-3-carboxamide;
Cpd 031—N-[1-(hydroxymethyl)cyclopropyl]-2-methyl-5-[(pyridin-2-yl)methoxy]furo[2,3-c]pyridine-3-carboxamide;
Cpd 032—N-[1-(hydroxymethyl)cyclopropyl]-5-[(2-methoxypyridin-3-yl)methoxy]-2-methylfuro[2,3-c]pyridine-3-carboxamide;
Cpd 033—N-[3-(hydroxymethyl)oxetan-3-yl]-2-methyl-5-[(4-methyl-1,3-thiazol-5-yl)methoxy]furo[2,3-c]pyridine-3-carboxamide;
Cpd 034—N-[3-(hydroxymethyl)oxetan-3-yl]-2-methyl-5-[(pyridin-2-yl)methoxy]furo[2,3-c]pyridine-3-carboxamide;
Cpd 035—N-[4-(hydroxymethyl)oxan-4-yl]-2-methyl-5-[(4-methyl-1,3-thiazol-5-yl)methoxy]furo[2,3-c]pyridine-3-carboxamide;
or a physiologically acceptable salt thereof.

The present invention also encompasses pharmaceutical composition comprising at least one compound of the present invention. The present invention also encompasses pharmaceutical composition comprising at least one compound of the invention and at least one carrier, excipient or diluent acceptable for pharmaceutical purposes.

In some embodiments, the present invention relates to the use of at least one compound of formula (I), or any subgroups thereof, in (the preparation of a composition for) the prevention and/or treatment of pain or epilepsy; preferably pain or epileptic encephalopathy.

In some embodiments, the present invention relates to a method of prevention and/or of treatment of metabolic pain, wherein the pain is selected from nociceptive pain, inflammatory pain, and neuropathic pain; preferably post-operative pain.

All definitions, preferred embodiments and preferred meanings of Q, T, U, W, R¹, R³, R⁴, R⁵, R^{5′}, n, R⁶, R⁸ including the preferred substituents also analogously apply the compounds according to the invention, which are not necessarily restricted for use in the treatment of pain or epilepsy. Thus, this aspect of the invention relates to the compounds of the invention as such, compositions comprising the compounds of the invention, medicaments comprising the compounds of the invention, and the compounds of the invention for use in the prevention and/or treatment of TRPM3 mediated disorders such as pain, epilepsy and/or inflammatory hypersensitivity; and/or for counteracting pain and/or inflammatory hypersensitivity. Preferably, the pain is selected from nociceptive pain, inflammatory pain, and neuropathic pain. More preferably, the pain is post-operative pain.

In some embodiments of the invention, the compound is selected from the group consisting of Cpd 001 to Cpd 036 as mentioned above and the physiologically acceptable salts thereof.

Another aspect of the invention relates to a pharmaceutical composition or a medicament comprising a compound according to the invention as described above.

The compounds of the invention have been shown to be antagonists of TRPM3 and the invention therefore provides the compounds as such, the compounds for use as a medicine, more specifically for use as a medicine in the prevention or treatment of TRPM3 mediated disorders in a subject with a therapeutically effective amount of a compound of the invention.

In some embodiments, the compound of the invention is the sole pharmacologically active compound to be administered for therapy. In another embodiment of the invention, the compound of the invention may be employed in combination with other therapeutic agents for the treatment or prophylaxis of TRPM3 mediated disorders. The invention therefore also relates to the use of a composition comprising:
one or more compounds of the formulae and embodiments herein, and
one or more further therapeutic or preventive agents that are used for the prevention or treatment of TRPM3 mediated disorders as biologically active agents in the form of a combined preparation for simultaneous, separate or sequential use.

The pharmaceutical composition or combined preparation according to this invention may contain compounds of the invention over a broad content range depending on the contemplated use and the expected effect of the preparation. Generally, the content of the compounds of the invention of the combined preparation is within the range of 0.1 to 99.9% by weight, preferably from 1 to 99% by weight, more preferably from 5 to 95% by weight.

In view of the fact that, when several active ingredients are used in combination, they do not necessarily bring out their joint therapeutic effect directly at the same time in the mammal to be treated, the corresponding composition may also be in the form of a medical kit or package containing the two ingredients in separate but adjacent repositories or compartments. In the latter context, each active ingredient may therefore be formulated in a way suitable for an administration route different from that of the other ingredient, e.g., one of them may be in the form of an oral or parenteral formulation whereas the other is in the form of an ampoule for intravenous injection or an aerosol.

Those of skill in the art will also recognize that the compounds of the invention may exist in many different protonation states, depending on, among other things, the pH of their environment. While the structural formulae provided herein depict the compounds in only one of several possible protonation states, it will be understood that these structures are illustrative only, and that the invention is not limited to any particular protonation state—any and all protonated forms of the compounds are intended to fall within the scope of the invention.

The term “pharmaceutically acceptable salts” as used herein means the therapeutically active non-toxic salt forms which the compounds of formulae herein are able to form. Therefore, the compounds of this invention optionally comprise salts of the compounds herein, especially pharmaceutically acceptable non-toxic salts containing, for example, Na⁺, Li⁺, K⁺, Ca²⁺ and Mg²⁺. Such salts may include those derived by combination of appropriate cations such as alkali and alkaline earth metal ions or ammonium and quaternary amino ions with an acid anion moiety, typically a carboxylic acid. The compounds of the invention may bear multiple positive or negative charges. The net charge of the compounds of the invention may be either positive or negative. Any associated counter ions are typically dictated by the synthesis and/or isolation methods by which the compounds are obtained. Typical counter ions include, but are not limited to ammonium, sodium, potassium, lithium, halides, acetate, trifluoroacetate, etc., and mixtures thereof. It will be understood that the identity of any associated counter ion is not a critical feature of the invention, and that the invention encompasses the compounds in association with any type of counter ion. Moreover, as the compounds can exist in a variety of different forms, the invention is intended to encompass not only forms of the compounds that are in association with counter ions (e.g., dry salts), but also forms that are not in association with counter ions (e.g., aqueous or organic solutions). Metal salts typically are prepared by reacting the metal hydroxide with a compound of this invention. Examples of metal salts which are prepared in this way are salts containing Li⁺, Na⁺, and K⁺. A less soluble metal salt can be precipitated from the solution of a more soluble salt by addition of the suitable metal compound. In addition, salts may be formed from acid addition of certain organic and inorganic acids to basic centers, typically amines, or to acidic groups. Examples of such appropriate acids include, for instance, inorganic acids such as hydrohalogen acids, e.g., hydrochloric or hydrobromic acid, sulfuric acid, nitric acid, phosphoric acid and the like; or organic acids such as, for example, acetic, propanoic, hydroxyacetic, 2-hydroxypropanoic, 2-oxopropanoic, lactic, pyruvic, oxalic (i.e. ethanedioic), malonic, succinic (i.e. butanedioic acid), maleic, fumaric, malic, tartaric, citric, methanesulfonic, ethanesulfonic, benzenesulfonic, p-toluenesulfonic, cyclohexanesulfamic, salicylic (i.e. 2-hydroxybenzoic), p-aminosalicylic and the like. Furthermore, this term also includes the solvates which the compounds of formulae herein as well as their salts are able to form, such as for example hydrates, alcoholates and the like. Finally, it is to be understood that the compositions herein comprise compounds of the invention in their unionized, as well as zwitterionic form, and combinations with stoichiometric amounts of water as in hydrates.

Also included within the scope of this invention are the salts of the parental compounds with one or more amino acids, especially the naturally-occurring amino acids found as protein components. The amino acid typically is one bearing a side chain with a basic or acidic group, e.g., lysine, arginine or glutamic acid, or a neutral group such as glycine, serine, threonine, alanine, isoleucine, or leucine.

The compounds of the invention also include physiologically acceptable salts thereof. Examples of physiologically acceptable salts of the compounds of the invention include salts derived from an appropriate base, such as an alkali metal (for example, sodium), an alkaline earth (for example, magnesium), ammonium and NX⁴⁺ (wherein X is —C₁₋₆-alkyl). Physiologically acceptable salts of a hydrogen atom or an amino group include salts of organic carboxylic acids such as acetic, benzoic, lactic, fumaric, tartaric, maleic, malonic, malic, isethionic, lactobionic and succinic acids; organic sulfonic acids, such as methanesulfonic, ethanesulfonic, benzenesulfonic and p-toluenesulfonic acids; and inorganic acids, such as hydrochloric, sulfuric, phosphoric and sulfamic acids. Physiologically acceptable salts of a compound containing a hydroxy group include the anion of said compound in combination with a suitable cation such as Na⁺ and NX4⁺ (wherein X typically is independently selected from —H or a —C₁₋₄-alkyl group). However, salts of acids or bases which are not physiologically acceptable may also find use, for example, in the preparation or purification of a physiologically acceptable compound. All salts, whether or not derived form a physiologically acceptable acid or base, are within the scope of the invention.

As used herein and unless otherwise stated, the term “enantiomer” means each individual optically active form of a compound of the invention, having an optical purity or enantiomeric excess (as determined by methods standard in the art) of at least 80% (i.e., at least 90% of one enantiomer and at most 10% of the other enantiomer), preferably at least 90% and more preferably at least 98%.

The term “isomers” as used herein means all possible isomeric forms, including tautomeric and stereochemical forms, which the compounds of formulae herein may possess, but not including position isomers. Typically, the structures shown herein exemplify only one tautomeric or resonance form of the compounds, but the corresponding alternative configurations are contemplated as well. Unless otherwise stated, the chemical designation of compounds denotes the mixture of all possible stereochemically isomeric forms, said mixtures containing all diastereomers and enantiomers (since the compounds of formulae herein may have at least one chiral center) of the basic molecular structure, as well as the stereochemically pure or enriched compounds. More particularly, stereogenic centers may have either the R- or S-configuration, and multiple bonds may have either cis- or trans-configuration.

Pure isomeric forms of the said compounds are defined as isomers substantially free of other enantiomeric or diastereomeric forms of the same basic molecular structure. In particular, the term “stereoisomerically pure” or “chirally pure” relates to compounds having a stereoisomeric excess of at least about 80% (i.e., at least 90% of one isomer and at most 10% of the other possible isomers), preferably at least 90%, more preferably at least 94% and most preferably at least 97%. The terms “enantiomerically pure” and “diastereomerically pure” should be understood in a similar way, having regard to the enantiomeric excess, respectively the diastereomeric excess, of the mixture in question.

Separation of stereoisomers is accomplished by standard methods known to those in the art. One enantiomer of a compound of the invention can be separated substantially free of its opposing enantiomer by a method such as formation of diastereomers using optically active resolving agents (“Stereochemistry of Carbon Compounds,” (1962) by E. L. Eliel, McGraw Hill; Lochmuller, C. H., (1975) J. Chromatogr., 113:(3) 283-302). Separation of isomers in a mixture can be accomplished by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure enantiomers, or (3) enantiomers can be separated directly under chiral conditions. Under method (1), diastereomeric salts can be formed by reaction of enantiomerically pure chiral bases such as brucine, quinine, ephedrine, strychnine, a-methyl-b-phenylethylamine (amphetamine), and the like with asymmetric compounds bearing acidic functionality, such as carboxylic acid and sulfonic acid. The diastereomeric salts may be induced to separate by fractional crystallization or ionic chromatography. For separation of the optical isomers of amino compounds, addition of chiral carboxylic or sulfonic acids, such as camphorsulfonic acid, tartaric acid, mandelic acid, or lactic acid can result in formation of the diastereomeric salts. Alternatively, by method (2), the substrate to be resolved may be reacted with one enantiomer of a chiral compound to form a diastereomeric pair (Eliel, E. and Wilen, S. (1994) Stereochemistry of Organic Compounds, John Wiley & Sons, Inc., p. 322). Diastereomeric compounds can be formed by reacting asymmetric compounds with enantiomerically pure chiral derivatizing reagents, such as menthyl derivatives, followed by separation of the diastereomers and hydrolysis to yield the free, enantiomerically enriched compound. A method of determining optical purity involves making chiral esters, such as a menthyl ester or Mosher ester, a-methoxy-a-(trifluoromethyl)phenyl acetate (Jacob III. (1982) J. Org. Chem. 47:4165), of the racemic mixture, and analyzing the NMR spectrum for the presence of the two atropisomeric diastereomers. Stable diastereomers can be separated and isolated by normal- and reverse-phase chromatography following methods for separation of atropisomeric naphthyl-isoquinolines (Hoye, T., WO 96/15111). Under method (3), a racemic mixture of two asymmetric enantiomers is separated by chromatography using a chiral stationary phase. Suitable chiral stationary phases are, for example, polysaccharides, in particular cellulose or amylose derivatives. Commercially available polysaccharide based chiral stationary phases are ChiralCel® CA, OA, OB5, OC5, OD, OF, OG, OJ and OK, and Chiralpak® AD, AS, OP(+) and OT(+). Appropriate eluents or mobile phases for use in combination with said polysaccharide chiral stationary phases are hexane and the like, modified with an alcohol such as ethanol, isopropanol and the like. (“Chiral Liquid Chromatography” (1989) W. J. Lough, Ed. Chapman and Hall, New York; Okamoto, (1990) “Optical resolution of dihydropyridine enantiomers by High-performance liquid chromatography using phenylcarbamates of polysaccharides as a chiral stationary phase”, J. of Chromatogr. 513:375-378).

The terms cis and trans are used herein in accordance with Chemical Abstracts nomenclature and include reference to the position of the substituents on a ring moiety. The absolute stereochemical configuration of the compounds of the formulae described herein may easily be determined by those skilled in the art while using well-known methods such as, for example, X-ray diffraction.

When a compound is crystallized from a solution or slurry, it can be crystallized in a different arrangement lattice of spaces (this property is called “polymorphism”) to form crystals with different crystalline forms, each of which is known as “polymorphs”. The term “Polymorph” as used herein therefore, refers to a crystal form of a compound of Formula (I), where the molecules are localized in the three-dimensional lattice sites. Different polymorphs of the compound of Formula (I) may be different from each other in one or more physical properties, such as solubility and dissolution rate, true specific gravity, crystal form, accumulation mode, flowability and/or solid-state stability. etc.

Compounds of the invention and their physiologically acceptable salts (hereafter collectively referred to as the active ingredients) may be administered by any route appropriate to the condition to be treated, suitable routes including oral, rectal, nasal, topical (including ocular, buccal and sublingual), vaginal and parenteral (including subcutaneous, intramuscular, intranasal, intravenous, intraarterial, intradermal, intrathecal and epidural). The preferred route of administration may vary with for example the condition of the recipient.

The therapeutically effective amount of the preparation of the compound(s), especially for the treatment of TRPM3 mediated disorders in humans and other mammals or in animals, preferably is a TRPM3 ion channel inhibiting amount of the compounds as defined herein and corresponds to an amount which ensures a plasma level of between 1 μg/ml and 100 mg/ml, optionally of 10 mg/ml.

Suitable dosages of the compounds or compositions of the invention should be used to treat or prevent the TRPM3 mediated disorders in a subject. Depending upon the pathologic condition to be treated and the patient's condition, the said effective amount may be divided into several sub-units per day or may be administered at more than one day intervals.

The invention further provides (pharmaceutical) compositions comprising one or more compounds of the invention, more in particular of all the Formula (I) and other formulas and embodiments described herein and the more particular aspects or embodiments thereof. Furthermore, the invention provides the compounds or (pharmaceutical) compositions of the invention, more in particular of all the Formula (I) and other formulas and embodiments described herein and the more particular aspects or embodiments thereof, for use as a medicine, more in particular for use in the treatment of pain or epilepsy. The TRPM3 mediated disorders are selected from pain, epilepsy and an inflammatory hypersensitivity condition.

The compounds of the invention may be formulated with conventional carriers and excipients, which will be selected in accord with ordinary practice. Tablets will contain excipients, glidants, fillers, binders and the like. Aqueous formulations are prepared in sterile form, and when intended for delivery by other than oral administration generally will be isotonic. Formulations optionally contain excipients such as those set forth in the “Handbook of Pharmaceutical Excipients” (1986).

Subsequently, the term “pharmaceutically acceptable carrier” as used herein means any material or substance with which the active ingredient is formulated in order to facilitate its application or dissemination to the locus to be treated, for instance by dissolving, dispersing or diffusing the said composition, and/or to facilitate its storage, transport or handling without impairing its effectiveness. The pharmaceutically acceptable carrier may be a solid or a liquid or a gas which has been compressed to form a liquid, i.e., the compositions of this invention can suitably be used as concentrates, emulsions, solutions, granulates, dusts, sprays, aerosols, suspensions, ointments, creams, tablets, pellets or powders.

Suitable pharmaceutical carriers for use in the said pharmaceutical compositions and their formulation are well known to those skilled in the art, and there is no particular restriction to their selection within the invention. They may also include additives such as wetting agents, dispersing agents, stickers, adhesives, emulsifying agents, surface-active agents, solvents, coatings, antibacterial and antifungal agents, isotonic agents and the like, provided the same are consistent with pharmaceutical practice, i.e., carriers and additives which do not create permanent damage to mammals. The pharmaceutical compositions of the invention may be prepared in any known manner, for instance by homogeneously mixing, coating and/or grinding the active ingredients, in a one-step or multi-steps procedure, with the selected carrier material and, where appropriate, the other additives such as surface-active agents. may also be prepared by micronisation, for instance in view to obtain them in the form of microspheres usually having a diameter of about 1 to 10 gm, namely for the manufacture of microcapsules for controlled or sustained release of the active ingredients.

While it is possible for the compounds of the invention to be administered alone it is preferable to present them as pharmaceutical formulations. The formulations, both for veterinary and for human use, of the invention comprise at least one active ingredient, as above described, together with one or more pharmaceutically acceptable carriers therefore and optionally other therapeutic ingredients. The carrier(s) optimally are “acceptable” in the sense of being compatible with the other ingredients of the formulation and not deleterious to the recipient thereof. The formulations include those suitable for oral, rectal, nasal, topical (including buccal and sublingual), vaginal or parenteral (including subcutaneous, intramuscular, intravenous, intradermal, intrathecal and epidural) administration. The formulations may conveniently be presented in unit dosage form and may be prepared by any of the methods well known in the art of pharmacy. Such methods include the step of bringing into association the active ingredient with the carrier which constitutes one or more accessory ingredients. In general, the formulations are prepared by uniformly and intimately bringing into association the active ingredient with liquid carriers or finely divided solid carriers or both, and then, if necessary, shaping the product.

Formulations of the invention suitable for oral administration may be presented as discrete units such as capsules, cachets or tablets each containing a predetermined amount of the active ingredient; as a powder or granules; as solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein. For infections of the eye or other external tissues e.g., mouth and skin, the formulations are optionally applied as a topical ointment or cream containing the active ingredient(s) in an amount of, for example, 0.075 to 20% w/w (including active ingredient(s) in a range between 0.1% and 20% in increments of 0.1% w/w such as 0.6% w/w, 0.7% w/w, etc.), preferably 0.2 to 15% w/w and most preferably 0.5 to 10% w/w. When formulated in an ointment, the active ingredients may be employed with either a paraffinic or a water-miscible ointment base. Alternatively, the active ingredients may be formulated in a cream with an oil-in-water cream base. If desired, the aqueous phase of the cream base may include, for example, at least 30% w/w of a polyhydric alcohol, i.e., an alcohol having two or more hydroxyl groups such as propylene glycol, butane 1,3-diol, mannitol, sorbitol, glycerol and polyethylene glycol (including PEG400) and mixtures thereof. The topical formulations may desirably include a compound which enhances absorption or penetration of the active ingredient through the skin or other affected areas. Examples of such dermal penetration enhancers include dimethylsulfoxide and related analogs.

The oily phase of the emulsions of this invention may be constituted from known ingredients in a known manner. While the phase may comprise merely an emulsifier (otherwise known as an emulgent), it desirably comprises a mixture of at least one emulsifier with a fat or an oil or with both a fat and an oil. Optionally, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations.

The choice of suitable oils or fats for the formulation is based on achieving the desired cosmetic properties, since the solubility of the active compound in most oils likely to be used in pharmaceutical emulsion formulations is very low. Thus, the cream should optionally be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredient is dissolved or suspended in a suitable carrier, especially an aqueous solvent for the active ingredient. The active ingredient is optionally present in such formulations in a concentration of 0.5 to 20%, advantageously 0.5 to 10% particularly about 1.5% w/w. Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate. Formulations suitable for nasal administration wherein the carrier is a solid include a coarse powder having a particle size for example in the range 20 to 500 microns (including particle sizes in a range between 20 and 500 microns in increments of 5 microns such as 30 microns, 35 microns, etc.), which is administered in the manner in which snuff is taken, i.e. by rapid inhalation through the nasal passage from a container of the powder held close up to the nose. Suitable formulations wherein the carrier is a liquid, for administration as for example a nasal spray or as nasal drops, include aqueous or oily solutions of the active ingredient. Formulations suitable for aerosol administration may be prepared according to conventional methods and may be delivered with other therapeutic agents.

Formulations suitable for vaginal administration may be presented as pessaries, tampons, creams, gels, pastes, foams or spray formulations containing in addition to the active ingredient such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage formulations are those containing a daily dose or unit daily sub-dose, as herein above recited, or an appropriate fraction thereof, of an active ingredient.

It should be understood that in addition to the ingredients particularly mentioned above the formulations of this invention may include other agents conventional in the art having regard to the type of formulation in question, for example those suitable for oral administration may include flavoring agents.

Compounds of the invention can be used to provide controlled release pharmaceutical formulations containing as active ingredient one or more compounds of the invention (“controlled release formulations”) in which the release of the active ingredient can be controlled and regulated to allow less frequency dosing or to improve the pharmacokinetic or toxicity profile of a given invention compound. Controlled release formulations adapted for oral administration in which discrete units comprising one or more compounds of the invention can be prepared according to conventional methods.

Another embodiment of this invention relates to various precursor or “prodrug” forms of the compounds of the invention. It may be desirable to formulate the compounds of the invention in the form of a chemical species which itself is not significantly biologically-active, but which when delivered to the animal, mammal or human will undergo a chemical reaction catalyzed by the normal function of the body, inter alia, enzymes present in the stomach or in blood serum, said chemical reaction having the effect of releasing a compound as defined herein. The term “prodrug” thus relates to these species which are converted in vivo into the active pharmaceutical ingredient.

The prodrugs of the compounds of the invention can have any form suitable to the formulator, for example, esters are non-limiting common pro-drug forms. In the present case, however, the pro-drug may necessarily exist in a form wherein a covalent bond is cleaved by the action of an enzyme present at the target locus. For example, a C—C covalent bond may be selectively cleaved by one or more enzymes at said target locus and, therefore, a pro-drug in a form other than an easily hydrolysable precursor, inter alia an ester, an amide, and the like, may be used. The counterpart of the active pharmaceutical ingredient in the pro-drug can have different structures such as an amino acid or peptide structure, alkyl chains, sugar moieties and others as known in the art.

For the purpose of the invention the term “therapeutically suitable pro-drug” is defined herein as “a compound modified in such a way as to be transformed in vivo to the therapeutically active form, whether by way of a single or by multiple biological transformations, when in contact with the tissues of the animal, mammal or human to which the pro-drug has been administered, and without undue toxicity, irritation, or allergic response, and achieving the intended therapeutic outcome”.

More specifically the term “prodrug” as used herein, relates to an inactive or significantly less active derivative of a compound such as represented by the structural formulae herein described, which undergoes spontaneous or enzymatic transformation within the body in order to release the pharmacologically active form of the compound. For a comprehensive review, reference is made to Rautio J. et al. (“Prodrugs: design and clinical applications” Nature Reviews Drug Discovery, 2008, doi: 10.1038/nrd2468).

Representative compounds of the invention can be synthesized in accordance with the general synthetic methods described below and illustrated in the schemes that follow. Since the schemes are an illustration, the invention should not be construed as being limited by the specific chemical reaction and specific conditions described in the schemes and examples. The various starting material used in the schemes are commercially available or may be prepared by methods well within the skill persons versed in the art. The variables are as defined herein and within the skill of persons verses in the art.

### EXAMPLES

The following examples are provided for the purpose of illustrating the invention and by no means should be interpreted to limit the scope of the invention.

Representative compounds of the present invention can be synthesized in accordance with the general synthetic methods described below and are illustrated in the schemes that follow. Since the schemes are an illustration, the invention should not be construed as being limited by the specific chemical reaction and specific conditions described in the schemes and examples. The various starting materials used in the schemes are commercially available or may be prepared by methods well within the skill of persons versed in the art. The variables are as defined herein and within the skill of persons versed in the art.

Abbreviations used in the instant specification, particularly in the schemes and examples, are as follows: ACN—acetonitrile, AcOH—Acetic acid, ADDP—1,1′-(Azodicarbonyl)dipiperidide, aq.—Aqueous, AIBN—Azobisisobutyronitrile, CAN—Ceric ammonium nitrate, COMU—(1-Cyano-2-ethoxy-2-oxoethylidenaminooxy)dimethylamino-morpholino-carbenium, hexafluorophosphate, DABCO—1,4-diazabicyclo[2.2.2]octane, DAST—Diethylaminosulfur trifluoride, DBU—1,8-Diazabicyclo[5.4.0]undec-7-ene, DCC—N,N′-dicyclohexylcarbodiimide, DCM—Dichloromethane, DEAD—Diethyl azodicarboxylate, DIA—Diastereomer, DIAD—Diisopropyl azodicarboxylate, DEA—Diethylamine, DIPEA—Diisopropyl-ethyl amine, DME—1,2-Dimethoxyethane, DMF—N,N-Dimethylformamide, DMSO—Dimethylsulfoxide, DTBAD—tert-Butylazodicarboxylate, EDCI or EDC—1-Ethyl-3-(3-dimethylaminopropyl), carbodiimide, En—Enantiomer, Et2O—Diethyl ether, EtOH—Ethanol, EtOAc—Ethyl acetate, Eq.—Equivalent, FA—Formic acid, FCC—Flash column chromatography, h—Hour, HATU—O-(7-Azabenzotriazol-1-yl)-N,N,N′,N′-tetramethyluronium hexafluorophosphate, HPLC—High performance liquid chromatography, IPA—isopropyl alcohol, LAH—Lithiumaluminiumhydrid, LG—Leaving group, MeOH—methanol, MgSO4—Magnesium sulfate, min.—Minute, Ms—Methanesulfonyl, Na2SO4—Sodium sulfate, NBS—N-Bromosuccinimide, NMP—1-Methyl-2-pyrrolidinone, Pd(PPh3)4—Tetrakis-(triphenylphosphine)-palladium(0), Pd2(dba)3—Tris(dibenzylideneacetone)dipalladium, Pet ether—Petroleum ether, PPh3—Triphenylphosphine, PS-DIEA—Diisoprpropyl-ethyl amine supported on PolyStyrene, PS—PPh3—Triphenylphosphine supported on PolyStyrene, PyBop—Benzotriazol-1-yl-oxytripyrrolidinophosphonium hexafluorophosphate, PTSA—p-Toluenesulfonic acid, RF: ratio of frontiers, RM—Reaction mixture, RP—Reverse phase, RT—Room temperature, sat.—Saturated, SEM—[2-(Trimethylsilyl)ethoxy]methyl acetal, SFC—Supercritical fluid chromatography, SPE—Solid Phase Extraction, TBDMS—Tert-Butyldimethylsilyl ether, TBAF—Tetrabutylammonium fluoride hydrate, TBAI—Tetrabutylammonium iodide, TEA—Triethylamine, THF—Tetrahydrofuran, TFA—Trifluoroacetic acid, TLC—thin layer chromatography, TPP—triphenyl phosphine, IPA—isopropyl alcohol, TMS—trimethyl silyl, T3P—Propylphosphonic anhydride.

The compounds of interest have a structure according to the general formula (I) and all other formulas described herein and embodiments thereof can be prepared as outlined in the general chemical scheme 1.

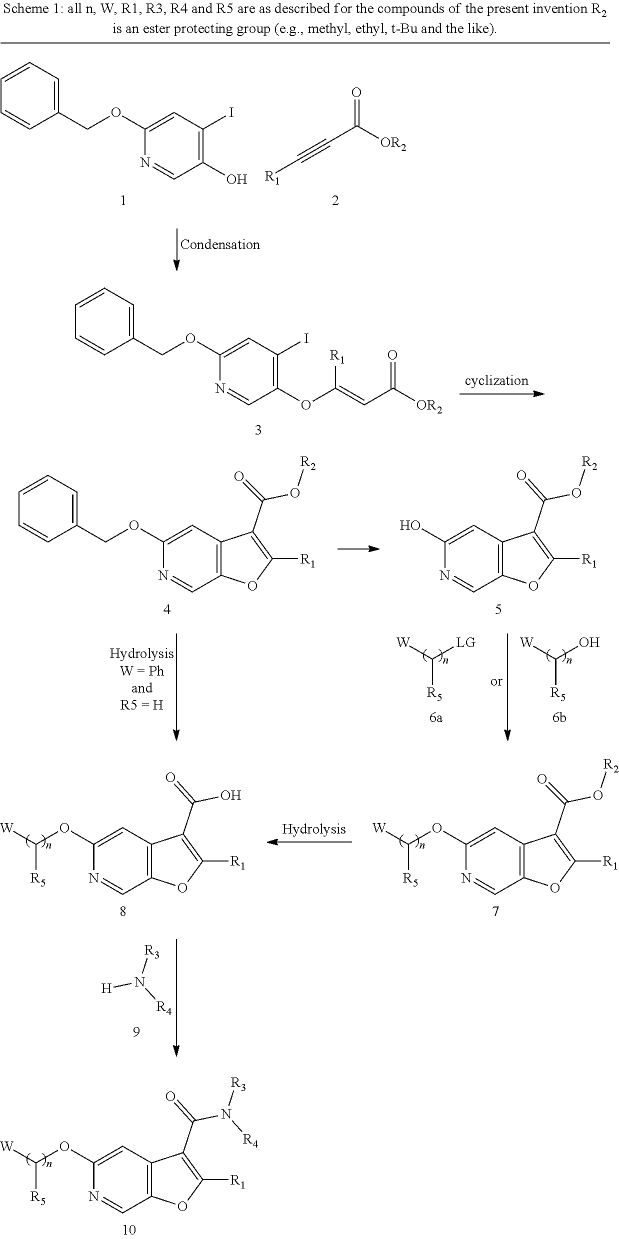

6-(Benzyloxy)-4-iodopyridin-3-ol of formula 1, may be condensed with an alkyne of formula 2 (commercially available or synthesized by procedures known to those skilled in the art), wherein R² is an ester protecting group (e.g., methyl, ethyl, t-Bu and the like), in the presence of a base (e.g., potassium carbonate, DBU and the like) in a polar solvent (e.g., DCM, MeCN, and the like) at a temperature ranging from 0 to 100° C. to provide intermediates of formula 3. Intermediates of formula 3 may then be cyclized into the desired compounds of formula 4 mediated by a catalyst (e.g., Bis(tri-tert-butylphosphine)palladium(0) and the likes) in presence of base (e.g., Et3N, pyridine, DBU and the likes) in a solvent (e.g., MeCN, DMF, NMP, and the like). Ester derivatives 4 may then be converted into the intermediate compounds of formula 5 via a hydrogenation reactions with a reducing agent (e.g., hydrogen gas, ammonium formate, cyclohexadiene and the like) using a catalyst (more preferably Pd or Pt) in a solvent (e.g., THF, EtOH, and the like). Intermediates of formula 5 may then be converted into the desired compounds of formula 7 via nucleophilic substitution using intermediates of formula 6a (commercially available or synthesized), wherein LG is a leaving group, in the presence of a base (e.g., DIPEA, DBU, triethylamine, Cs₂CO₃, and the like) in a polar solvent (e.g., acetonitrile, DMF, NMP, and the like), with or without a chelating agent (e.g., 18-crown-6, cis-anti-cis-dicyclohexano-18-crown-6, and the like) at a temperature ranging from 0 to 100° C. Alternatively, intermediates of formula 5 may also be reacted with intermediates of formula 6b (commercially available or synthesized) in the presence of an azodicarboxylate reagent (e.g., DEAD, DIAD, ADDP, and the like) and a phosphine (e.g., tributylphosphine, triphenylphosphine and the like) in a solvent (e.g., THF, toluene, and the like) at a temperature ranging from 0 to 100° C., to provide the desired compounds of formula 7. Ester derivatives 7 may then be converted into the desired compounds of formula 8 via standard saponification reactions. Alternatively, benzyl derivatives 4 may then be converted into the desired compounds of formula 8 via standard saponification reactions The desired compounds of formula 10 may be obtained from acid derivatives of formula 8 by reaction with amine derivatives of formula 9 (commercially available or synthesized by procedures known in the art or as set forth in the examples below) under standard peptide coupling conditions (e.g., DCC, EDCI, HATU, PyBop and the like) in a polar aprotic solvent (e.g., DCM, DMF and the like). Alternatively, carboxylic acid derivatives of formula 8, may be converted into acid chloride derivatives by procedures known to those skilled in the art or as set forth in the examples below, and then reacted with amines of formula 9 to obtain the desired compounds of formula 10 by procedures known to those skilled in the art or as set forth in the examples below.

### EXAMPLES

Structure and Compound CODE

| | |
|---|---|
| 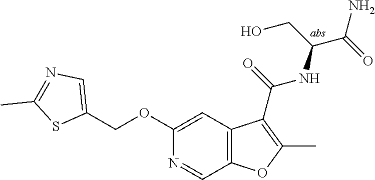 [SMILES: Cc1ncc(COc2cc3c(C(=O)N[C@@H](CO)C(N)=O)c(C)oc3cn2)s1] | Cpd 001 |
| 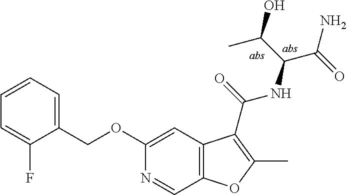 [SMILES: Cc1oc2cnc(OCc3ccccc3F)cc2c1C(=O)N[C@H](C(N)=O)[C@@H](C)O] | Cpd 002 |
| 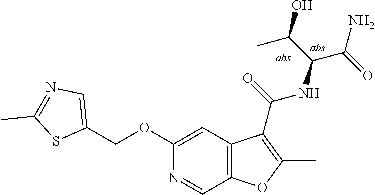 [SMILES: Cc1ncc(COc2cc3c(C(=O)N[C@H](C(N)=O)[C@@H](C)O)c(C)oc3cn2)s1] | Cpd 003 |
| 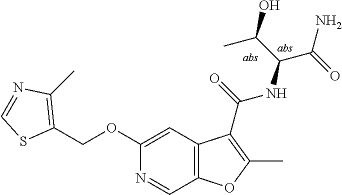 [SMILES: Cc1ncsc1COc1cc2c(C(=O)N[C@H](C(N)=O)[C@@H](C)O)c(C)oc2cn1] | Cpd 004 |
| 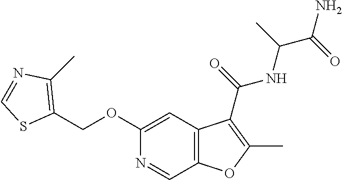 [SMILES: Cc1ncsc1COc1cc2c(C(=O)NC(C)C(N)=O)c(C)oc2cn1] | Cpd 005 |
| 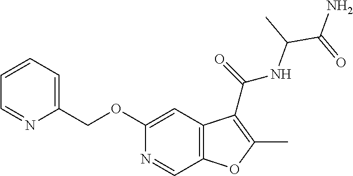 [SMILES: Cc1oc2cnc(OCc3ccccn3)cc2c1C(=O)NC(C)C(N)=O] | Cpd 006 |
| 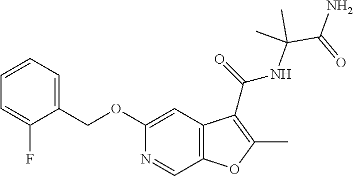 [SMILES: Cc1oc2cnc(OCc3ccccc3F)cc2c1C(=O)NC(C)(C)C(N)=O] | Cpd 007 |
| 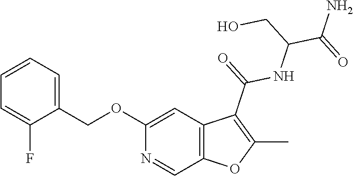 [SMILES: Cc1oc2cnc(OCc3ccccc3F)cc2c1C(=O)NC(CO)C(N)=O] | Cpd 008 |
| 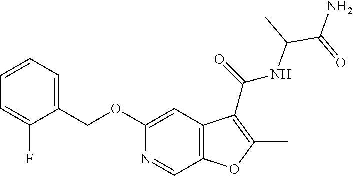 [SMILES: Cc1oc2cnc(OCc3ccccc3F)cc2c1C(=O)NC(C)C(N)=O] | Cpd 009 |
| 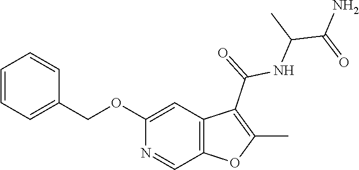 [SMILES: Cc1oc2cnc(OCc3ccccc3)cc2c1C(=O)NC(C)C(N)=O] | Cpd 010 |
| 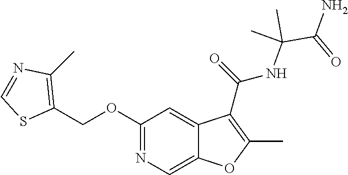 [SMILES: Cc1ncsc1COc1cc2c(C(=O)NC(C)(C)C(N)=O)c(C)oc2cn1] | Cpd 011 |
| 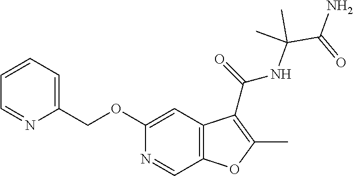 [SMILES: Cc1oc2cnc(OCc3ccccn3)cc2c1C(=O)NC(C)(C)C(N)=O] | Cpd 012 |
| 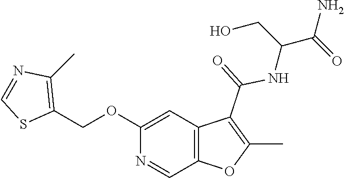 [SMILES: Cc1ncsc1COc1cc2c(C(=O)NC(CO)C(N)=O)c(C)oc2cn1] | Cpd 013 |
| 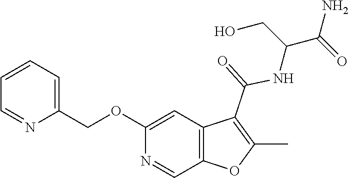 [SMILES: Cc1oc2cnc(OCc3ccccn3)cc2c1C(=O)NC(CO)C(N)=O] | Cpd 014 |
| 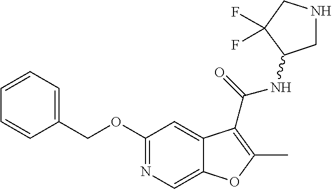 [SMILES: Cc1oc2cnc(OCc3ccccc3)cc2c1C(=O)NC1CNCC1(F)F] | Cpd 015 |
| 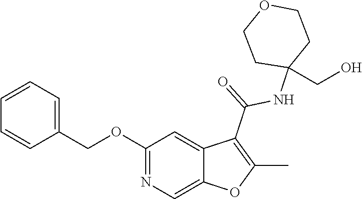 [SMILES: Cc1oc2cnc(OCc3ccccc3)cc2c1C(=O)NC1(CO)CCOCC1] | Cpd 016 |
| 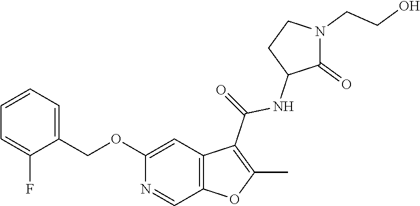 [SMILES: Cc1oc2cnc(OCc3ccccc3F)cc2c1C(=O)NC1CCN(CCO)C1=O] | Cpd 017 |
| 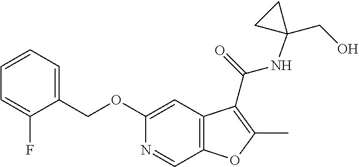 [SMILES: Cc1oc2cnc(OCc3ccccc3F)cc2c1C(=O)NC1(CO)CC1] | Cpd 018 |
| 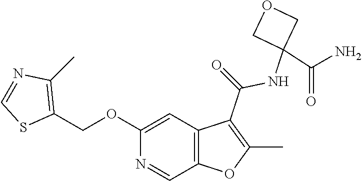 [SMILES: Cc1ncsc1COc1cc2c(C(=O)NC3(C(N)=O)COC3)c(C)oc2cn1] | Cpd 019 |
| 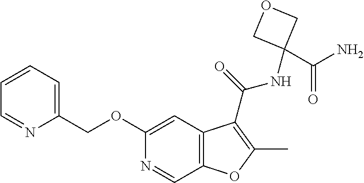 [SMILES: Cc1oc2cnc(OCc3ccccn3)cc2c1C(=O)NC1(C(N)=O)COC1] | Cpd 020 |
| 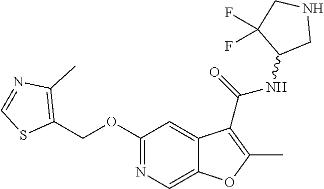 [SMILES: Cc1ncsc1COc1cc2c(C(=O)NC3CNCC3(F)F)c(C)oc2cn1] | Cpd 021 |
| 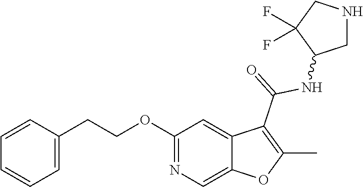 [SMILES: Cc1oc2cnc(OCCc3ccccc3)cc2c1C(=O)NC1CNCC1(F)F] | Cpd 022 |
| 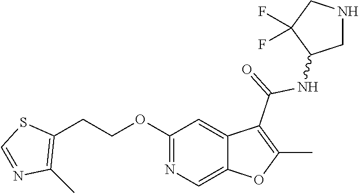 [SMILES: Cc1ncsc1CCOc1cc2c(C(=O)NC3CNCC3(F)F)c(C)oc2cn1] | Cpd 023 |
| 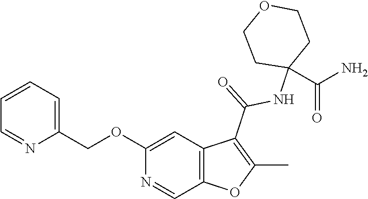 [SMILES: Cc1oc2cnc(OCc3ccccn3)cc2c1C(=O)NC1(C(N)=O)CCOCC1] | Cpd 024 |
| 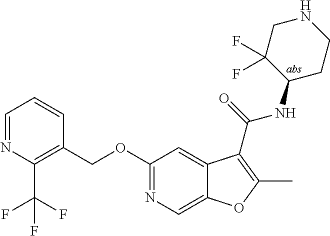 [SMILES: Cc1oc2cnc(OCc3cccnc3C(F)(F)F)cc2c1C(=O)N[C@@H]1CCNCC1(F)F] | Cpd 025 |
| 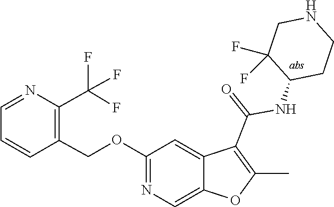 [SMILES: Cc1oc2cnc(OCc3cccnc3C(F)(F)F)cc2c1C(=O)N[C@H]1CCNCC1(F)F] | Cpd 026 |
| 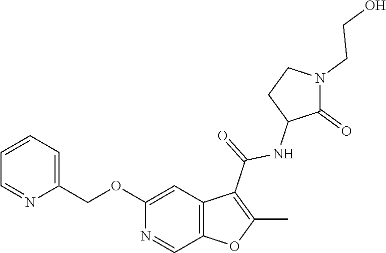 [SMILES: Cc1oc2cnc(OCc3ccccn3)cc2c1C(=O)NC1CCN(CCO)C1=O] | Cpd 027 |
| 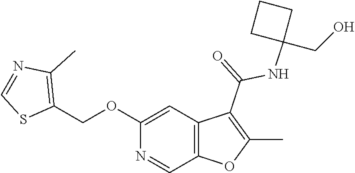 [SMILES: Cc1ncsc1COc1cc2c(C(=O)NC3(CO)CCC3)c(C)oc2cn1] | Cpd 028 |
| 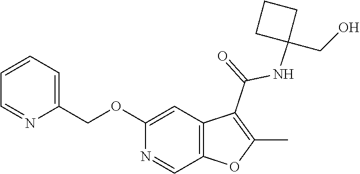 [SMILES: Cc1oc2cnc(OCc3ccccn3)cc2c1C(=O)NC1(CO)CCC1] | Cpd 029 |
| 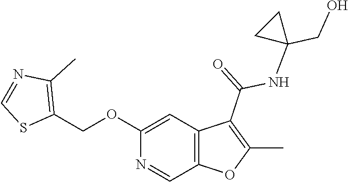 [SMILES: Cc1ncsc1COc1cc2c(C(=O)NC3(CO)CC3)c(C)oc2cn1] | Cpd 030 |
| 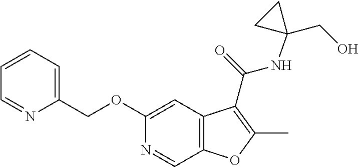 [SMILES: Cc1oc2cnc(OCc3ccccn3)cc2c1C(=O)NC1(CO)CC1] | Cpd 031 |
| 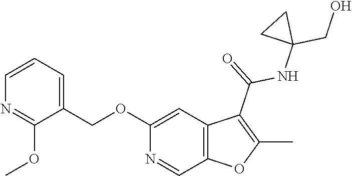 [SMILES: COc1ncccc1COc1cc2c(C(=O)NC3(CO)CC3)c(C)oc2cn1] | Cpd 032 |
| 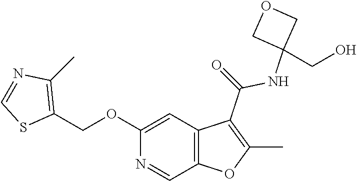 [SMILES: Cc1ncsc1COc1cc2c(C(=O)NC3(CO)COC3)c(C)oc2cn1] | Cpd 033 |
| 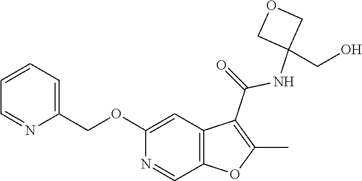 [SMILES: Cc1oc2cnc(OCc3ccccn3)cc2c1C(=O)NC1(CO)COC1] | Cpd 034 |
| 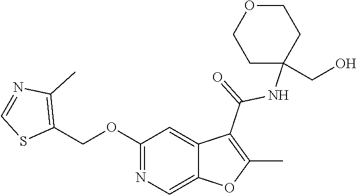 [SMILES: Cc1ncsc1COc1cc2c(C(=O)NC3(CO)CCOCC3)c(C)oc2cn1] | Cpd 035 |
| 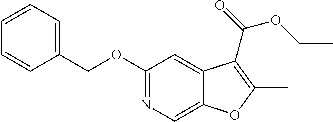 [SMILES: CCOC(=O)c1c(C)oc2cnc(OCc3ccccc3)cc12] | Cpd 036 |

The examples depicted above are provided for the purpose of illustrating the present invention and by no means should be interpreted to limit the scope of the present invention.

Part A represents the preparation of the compounds whereas Part B represents the pharmacological examples.

All starting materials which are not explicitly described were either commercially available (the details of suppliers such as for example ABCR, Apollo Scientific Combi-Blocks, Enamine, FluoroChem, MatrixScientific, Maybridge, Merck, TCI etc. can be found in the SciFinder® Database for example) or the synthesis thereof has already been described precisely in the specialist literature (experimental guidelines can be found in the Reaxys® Database or the SciFinder® Database respectively, for example) or can be prepared using the conventional methods known to the person skilled in the art.

The reactions were, if necessary, carried out under an inert atmosphere (mostly argon and N₂). The number of equivalents of reagents and the amounts of solvents employed as well as the reaction temperatures and times can vary slightly between different reactions carried out by analogous methods. The work-up and purification methods were adapted according to the characteristic properties of each compound and can vary slightly for analogous methods. The yields of the compounds prepared are not optimized.

The indication “equivalents” (“eq.” or “eq” or “equiv.”) means molar equivalents, “RT” or “rt” means room temperature T (23±7° C.), “M” are indications of concentration in mol/l, “sol.” means solution, “conc.” means concentrated. The mixing ratios of solvents are usually stated in the volume/volume ratio.

Key analytical characterization was carried out by means of ¹H-NMR spectroscopy and/or mass spectrometry (MS, m/z for [M+H]⁺ and/or for [M−H]⁻) for all the exemplary compounds and selected intermediate products. In certain cases, where e.g., regioisomers and/or diastereomers could be/were formed, additional analytics, such as, e.g., ¹³C NMR and NOE (nuclear overhauser effect) NMR experiments were in some cases performed.

Analytical instruments employed were e.g., for NMR analysis a BRUKER 400 MHz or a BRUKER 500 MHz machine (Software Topspin), alternatively a BRUKER AVANCE 300 MHz and 400 Mhz was employed. For LC/MS analysis e.g., an Agilent 1290 infinity, Mass:6150 SQD(ESI/APCI) or an Agilent 1200 SERIES, Mass:6130 SQD(ESI/APCI) (Software Chemistation) was employed. Analytical HPLCs were measured e.g., on Waters (Software Empower), an Agilent-1200-ELSD (Software Chemistation) or an Agilent-1260 (Software OpenLAB). Analytical SFC were performed e.g., on a PIC solution (Software: SFC PICLAB ONLINE), a WATERS-X5 (Software MASSLYNX) or a WATERS-UPC2 (Empower).

Preparative HPLC were performed e.g., on a Waters 2998 (Software Empower) or a YMC (Software K-Prep). Preparative SFC were performed e.g., on a Waters, SFC—200 (Software Chromscope or Super chrome), a Waters, SFC-80 (Super chrome) or a PIC, PIC-175 (Software 510-100).

Structures of example compounds that contain stereocentres are drawn and named with absolute stereochemistry, if known. In case of unknown absolute stereochemistry, the compounds can be either racemic, a mixture of diastereomers, a pure diastereomer of unknown stereochemistry, or a pure enantiomer of unknown stereochemistry. Dia 1 and Dia 2 means that diastereiosomers were separated but the stereochemistry is unknown. En 1 and En 2 means that both enantiomers were separated but the absolute configuration is unknown. No suffix given after the compound code means that a compound containing stereocentres was obtained as a racemic mixture or a mixture of diastereomers, respectively, unless the chemical name of the compound specifies the exact stereochemistry

The LC/MS analysis mentioned in the experimental part were performed on Alliance Waters HPLC (equipped with a PDA detector) connected to a mass spectrometer mass spectrometer Waters 3100 Mass Detector with ESI mode; or on Acquity UPLC Waters (equipped with a PDA detector) connected to a mass spectrometer mass SQD2 Detector with ESI mode; or on Acquity UPLC Waters (equipped with a PDA detector) connected to a mass spectrometer mass Xevo TQS Detector with ESI mode. Column Acquity BEH (50 mm×2.1 mm) 1.7 μm was used with a mobile phase A of 0.05% of formic acid in water or 0.05% of TFA in water and a mobile phase B of 0.05% of formic acid in MeCN or 0.05% of TFA in MeCN. Or a column Xbridge C18 (75 mm×4.6 mm) 3.5 μm was used with a mobile phase A of 5 mM of Ammonium Bicarbonate in water and a mobile phase B of MeCN.

### Synthesis of ethyl 5-(benzyloxy)-2-methylfuro[2,3-c]pyridine-3-carboxylate (Cpd 036)

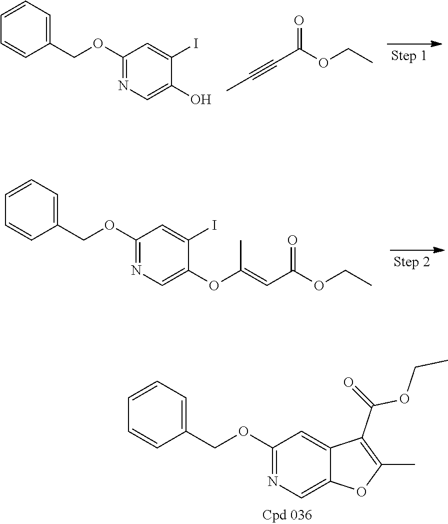

Step 1: Four 20 mL vials were charged with a mixture of 6-benzyloxy-4-iodo-pyridin-3-ol (1000 mg, 3.06 mmol), Ethyl 2-butynoate (0.58 mL, 4.89 mmol), and Potassium carbonate (2112 mg, 15.28 mmol), in MeCN (9.4 mL), was stirred at 120° C. for 3 h. The combined 4 vials were partitioned between water and DCM. The organic layer was concentrated under reduced pressure. The residue was purified by FCC on silica gel using a gradient of EtOAc (5-40%) in heptane to afford 3160.3 mg (59% yield) of ethyl (E)-3-[(6-benzyloxy-4-iodo-3-pyridyl)oxy]but-2-enoate. ESI/APCI(+): 440 (M+H); ESI/APCI(−): 438 (M−H); 1H NMR (DMSO-d6) d (ppm): 8.03 (s, 1H), 7.54 (s, 1H), 7.43-7.48 (m, 2H), 7.39 (t, J=7.3 Hz, 2H), 7.30-7.36 (m, 1H), 5.33 (s, 2H), 4.62 (s, 1H), 4.02 (q, J=7.0 Hz, 2H), 2.46 (s, 3H), 1.14 (t, J=7.0 Hz, 3H).

Step 2: Three 20 mL microwave vial was charged with a solution of ethyl (E)-3-[(6-benzyloxy-4-iodo-3-pyridyl)oxy]but-2-enoate (830 mg, 1.89 mmol) in MeCN (13 mL). The solution was evacuated and back filled with argon. This process was repeated three times. Et3N (1.33 mL, 9.45 mmol) followed by Bis(tri-tert-butylphosphine)palladium(0) (48.28 mg, 0.09 mmol) were added to the solution. The RM was stirred in a microwave oven at 125° C. for 30 minutes. The RM of the three vials were combined and then concentrated under reduced pressure. The residue was purified by FCC on silica gel using a gradient of EtOAc (0-30%) in heptane to afford 1626 mg (92% yield) of ethyl 5-(benzyloxy)-2-methylfuro[2,3-c]pyridine-3-carboxylate (Cpd 036) as a white solid.

### Synthesis of 5-(benzyloxy)-N-[4-(hydroxymethyl)oxan-4-yl]-2-methylfuro[2,3-c]pyridine-3-carboxamide (Cpd 016)

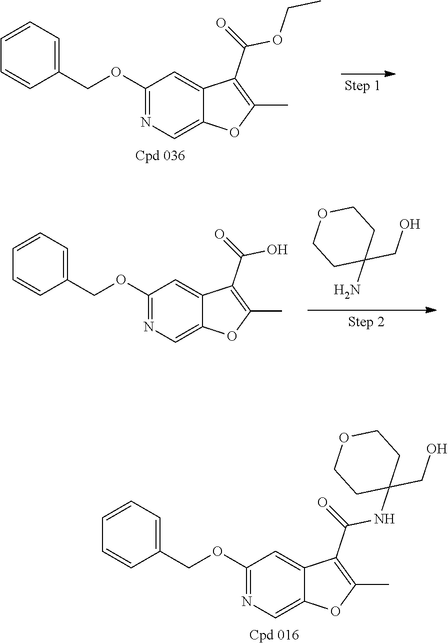

Step 1: An aqueous solution of NaOH (2N) (17.1 mL, 34.2 mmol) was added to a solution of ethyl 5-benzyloxy-2-methyl-furo[2,3-c]pyridine-3-carboxylate (1700 mg, 5.46 mmol) inMeOH (17 mL) and 1,4-Dioxane (10 mL). The mixture was stirred at RT for 18 h. A 4M solution of HCl in dioxane (9 mL) was added (mixture pH 1-2). The pH of the mixture was then adjusted to 4 by addition of an aqueous solution of sodium carbonate 1M. The mixture was concentrated under reduced pressure. Cold water was added to the residue. The solid material was collected by filtration and dried to afford 5-benzyloxy-2-methyl-furo[2,3-c]pyridine-3-carboxylic acid (1531.9 mg, 99% yield) as a beige solid.

Step 2: A mixture of 5-benzyloxy-2-methyl-furo[2,3-c]pyridine-3-carboxylic acid (250 mg, 0.88 mmol), HATU (335.56 mg, 0.88 mmol), (4-aminooxan-4-yl)methanol (115.76 mg, 0.8800 mmol), and DIPEA (0.46 mL, 2.65 mmol) in DMF (7.5 mL) was stirred overnight at RT. The RM was partitioned between a 1 M aqueous solution of sodium carbonate and dichloromethane. The organic layer was washed twice with a 1M aqueous solution of sodium carbonate. The organic layer was dried over magnesium sulphate, filtered, and concentrated under reduced pressure. The residue was purified by FCC on silica gel using a gradient of MeOH (0-6%) in DCM to afford 5-benzyloxy-N-[4-(hydroxymethyl)tetrahydropyran-4-yl]-2-methyl-furo[2,3-c]pyridine-3-carboxamide (249.5 mg, 69.9% yield) as a beige solid.

The following compounds were prepared in a similar manner (use of appropriate reagents (chiral or racemic) and purification methods (including chiral HPLC or chiral SFC) known to the person skilled in the art) as described for Cpd 016: Cpd 010-En1, Cpd 010-En2

### Synthesis of 5-(benzyloxy)-N-(4,4-difluoropyrrolidin-3-yl)-2-methylfuro[2,3-c]pyridine-3-carboxamide (Cpd 015-En1)

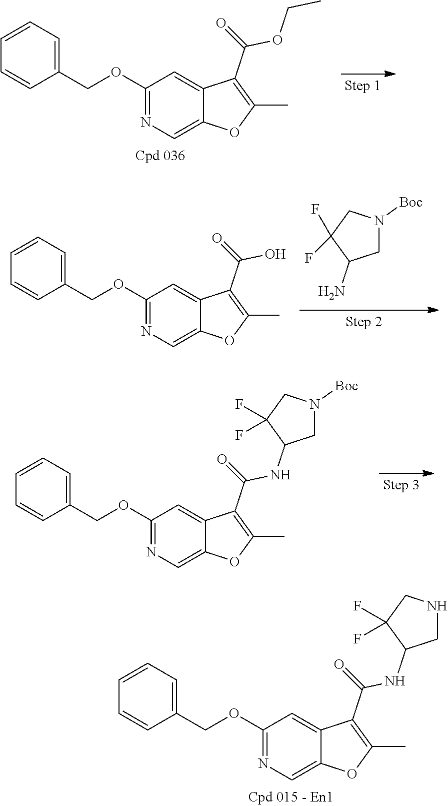

Step 1: An aqueous solution of NaOH (2N) (17.1 mL, 34.2 mmol) was added to a solution of ethyl 5-benzyloxy-2-methyl-furo[2,3-c]pyridine-3-carboxylate (1700 mg, 5.46 mmol) inMeOH (17 mL) and 1,4-Dioxane (10 mL). The mixture was stirred at RT for 18 h. A 4M solution of HCl in dioxane (9 mL) was added (mixture pH 1-2). The pH of the mixture was then adjusted to 4 by addition of an aqueous solution of sodium carbonate 1M. The mixture was concentrated under reduced pressure. Cold water was added to the residue. The solid material was collected by filtration and dried to afford 5-benzyloxy-2-methyl-furo[2,3-c]pyridine-3-carboxylic acid (1531.9 mg, 99% yield) as a beige solid.

Step 2: A mixture of 5-benzyloxy-2-methyl-furo[2,3-c]pyridine-3-carboxylic acid (95.mg, 0.34 mmol), HATU (127.51 mg, 0.34 mmol), tert-butyl (+)-4-amino-3,3-difluoropyrrolidine-1-carboxylate (74.53 mg, 0.34 mmol), and DIPEA (0.17 mL, 1.01 mmol) in DMF (2.85 mL) was stirred overnight at RT. The RM was partitioned between water and DCM. The aqueous layer was extracted twice with DCM. Combined organic extracts were dried over magnesium sulphate, filtered, and concentrated under reduced pressure. The residue was purified by FCC on silica gel using a gradient of EtOAc (5-60%) in heptane to afford tert-butyl (4R*)-4-[(5-benzyloxy-2-methyl-furo[2,3-c]pyridine-3-carbonyl)amino]-3,3-difluoro-pyrrolidine-1-carboxylate (112.2 mg, 0.2302 mmol, 69% yield) as a beige solid. ESJ/APCI(+): 488 (M+H); ESJ/APCI(−): 486 (M−H).

Step 3: HCl in dioxane (4N, 3.5 mL, 14 mmol) was added to a solution of tert-butyl (4R)-4-[(5-benzyloxy-2-methyl-furo[2,3-c]pyridine-3-carbonyl)amino]-3,3-difluoro-pyrrolidine-1-carboxylate (110.mg, 0.23 mmol) in DCM (3.5 mL). The mixture was stirred at RT for 1 h. The RM was concentrated under reduced pressure. The residue was purified by a column of supported SiliaBond®-PropylSulfonic Acid using a gradient of MeOH (0-100%) in DCM before elution with a 3N solution of ammonia in MeOH. The residue was re-purified by FCC on silica gel using a gradient of MeOH (0-8%) in DCM to afford 5-(benzyloxy)-N-(4,4-difluoropyrrolidin-3-yl)-2-methylfuro[2,3-c]pyridine-3-carboxamide (Cpd 015-En1) (67.3 mg, 77% yield) as a white solid.

The following compound was prepared in a similar manner (use of appropriate reagents (chiral or racemic) and purification methods (including chiral HPLC or chiral SFC) known to the person skilled in the art) as described for Cpd 015-En1: Cpd 015-En2

### Synthesis of N-[4-(hydroxymethyl)oxan-4-yl]-2-methyl-5-[(4-methyl-1,3-thiazol-5-yl)methoxy]furo[2,3-c]pyridine-3-carboxamide (Cud 035)

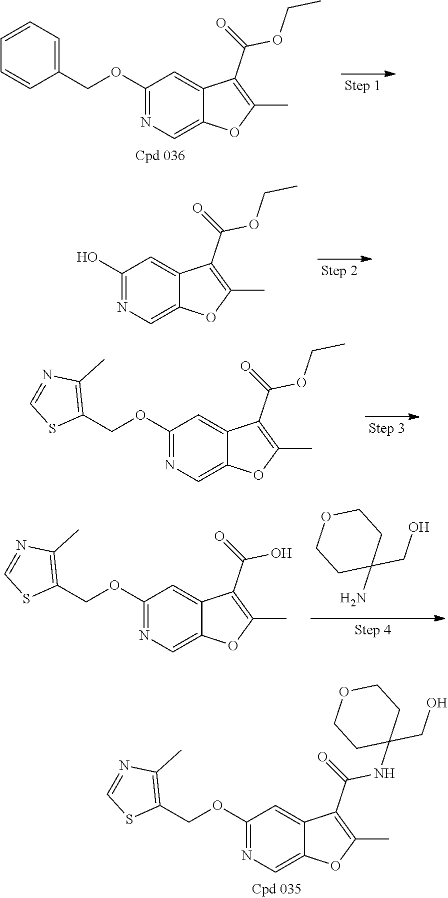

Step 1: Palladium on activated carbon, 10% Pd, unreduced (213.33 mg, 2 mmol) was added to a degassed solution of ethyl 5-benzyloxy-2-methyl-furo[2,3-c]pyridine-3-carboxylate (Cpd 036) (1600 mg, 5.14 mmol) in a mixture of Ethanol (42 mL) and THF (21 mL). The mixture was degassed and then fitted with a balloon of hydrogen. The RM was stirred for 8 h at RT. The RM was then filtered. The solid material was collected and suspended in 250 mL of a ¼ mixture of MeOH and DCM. The mixture was filtered. The solid material was again collected and treated as above with 250 mL of a ¼ mixture MeOH and DCM. Combined filtrates were concentrated under reduced pressure to afford ethyl 5-hydroxy-2-methyl-furo[2,3-c]pyridine-3-carboxylate (1109.3 mg, 5.0147 mmol, 98% yield) as a white solid. ESJ/APCI(+): 222 (M+H); ESJ/APCI(−): 220 (M−H); 1H NMR (DMSO-d6) d (ppm): 10.88 (br. s., 1H), 8.22 (s, 1H), 6.80 (s, 1H), 4.32 (q, J=7.1 Hz, 2H), 2.72 (s, 3H), 1.35 (t, J=7.0 Hz, 3H).

Step 2: Tributylphosphine (0.91 mL, 3.4 mmol) was added dropwise to a stirred mixture of ethyl 5-hydroxy-2-methyl-furo[2,3-c]pyridine-3-carboxylate (540 mg, 2.44 mmol), (4-methyl-1,3-thiazol-5-yl)methanol (0.45 mL, 3.66 mmol) and ADDP (862 mg, 3.4 2 mmol) in dry THF (47.25 mL) under argon. The mixture was stirred for 2 h and was concentrated under reduced pressure. The residue was purified by FCC on silica gel using a gradient of EtOAc (10-100%) in heptane followed by a gradient of MeOH (0-8%) in EtOAc to afford ethyl 2-methyl-5-[(4-methylthiazol-5-yl)methoxy]furo[2,3-c]pyridine-3-carboxylate (272 mg, 0.8167 mmol, 33% yield) as a white solid. ESJ/APCI(+): 333 (M+H); 1H NMR (DMSO-d6) d (ppm): 8.95 (s, 1H), 8.57 (s, 1H), 7.11 (s, 1H), 5.56 (s, 2H), 4.33 (q, J=7.1 Hz, 2H), 2.77 (s, 3H), 2.46 (s, 3H), 1.35 (t, J=7.0 Hz, 3H).

Step 3: An aqueous solution of NaOH (3 mL, 6 mmol) was added to a solution of ethyl 2-methyl-5-[(4-methylthiazol-5-yl)methoxy]furo[2,3-c]pyridine-3-carboxylate (272 mg, 0.82 mmol) in MeOH (3 mL) and 1,4-Dioxane (1.5 mL). The RM was stirred at RT for 3 h and a 4M solution of HCl in dioxane (1.5 mL) was added. The mixture was concentrated under reduced pressure. Cold water was added to the residue. The solid material was collected by filtration and dried to afford to afford 2-methyl-5-[(4-methylthiazol-5-yl)methoxy]furo[2,3-c]pyridine-3-carboxylic acid (182 mg, 73% yield) as a white solid. ESI/APCI(+): 305 (M+H); ESJ/APCI(−): 303 (M−H).

Step 4: A mixture of 2-methyl-5-[(4-methylthiazol-5-yl)methoxy]furo[2,3-c]pyridine-3-carboxylic acid (60 mg, 0.2 mmol), HATU (74.9 mg, 0.2 mmol), (4-aminooxan-4-yl)methanol (25.86 mg, 0.2 mmol), and DIPEA (0.1 mL, 0.59 mmol) in DMF (1.7 mL) was stirred overnight at RT and was partitioned between water and DCM. After separation, the aqueous layer was extracted twice with DCM. Combined organic extracts were dried over magnesium sulphate, filtered, and concentrated under reduced pressure. The residue was purified by FCC on silica gel using a gradient of methanol (0-8%) in DCM to afford N-[4-(hydroxymethyl)oxan-4-yl]-2-methyl-5-[(4-methyl-1,3-thiazol-5-yl)methoxy]furo[2,3-c]pyridine-3-carboxamide (Cpd 035) (66.8 mg, 81% yield) as a white solid.

The following compounds were prepared in a similar manner (use of appropriate reagents (chiral or racemic) and purification methods (including chiral HPLC or chiral SFC) known to the person skilled in the art) as described for Cpd 035: Cpd 001, Cpd 003, Cpd 004, Cpd 005-En1, Cpd 005-En2, Cpd 011, Cpd 013-En1, Cpd 013-En2, Cpd 019, Cpd 028, Cpd 030 and Cpd 033.

### Synthesis of N-[(4R)-3,3-difluoropiperidin-4-yl]-2-methyl-5-{[2-(trifluoromethyl)pyridin-3-yl]methoxy}furo[2,3-c]pyridine-3-carboxamide (Cpd 025)

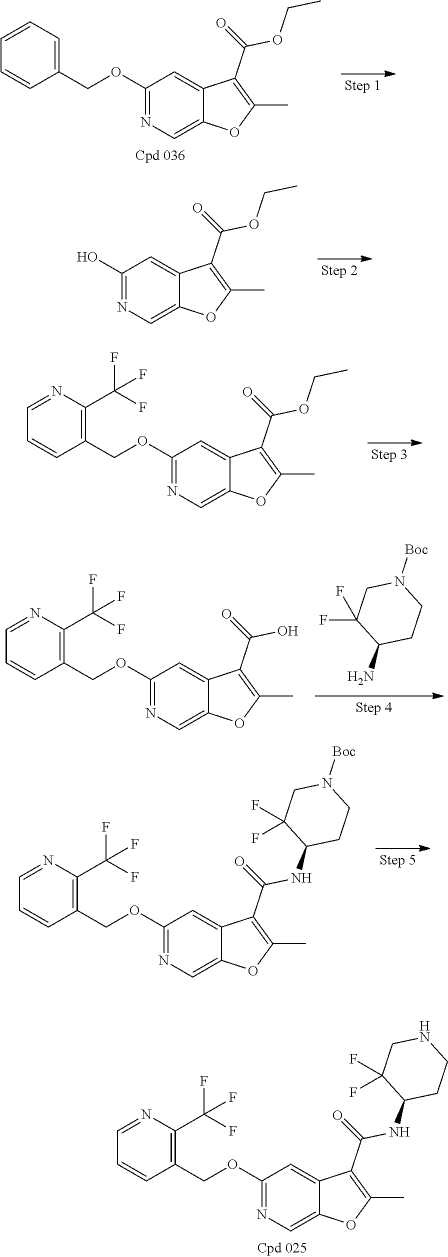

Step 1: Palladium on activated carbon, 10% Pd, unreduced (213.33 mg, 2 mmol) was added to a degassed solution of ethyl 5-benzyloxy-2-methyl-furo[2,3-c]pyridine-3-carboxylate (Cpd 036) (1600 mg, 5.14 mmol) in a mixture of Ethanol (42 mL) and THF (21 mL). The mixture was degassed and then fitted with a balloon of hydrogen. The RM was stirred for 8 h at RT. The RM was then filtered. The solid material was collected and suspended in 250 mL of a ¼ mixture of MeOH and DCM. The mixture was filtered. The solid material was again collected and treated as above with 250 mL of a ¼ mixture MeOH and DCM. Combined filtrates were concentrated under reduced pressure to afford ethyl 5-hydroxy-2-methyl-furo[2,3-c]pyridine-3-carboxylate (1109.3 mg, 5.0147 mmol, 98% yield) as a white solid. ESI/APCI(+): 222 (M+H); ESI/APCI(−): 220 (M−H); 1H NMR (DMSO-d6) d (ppm): 10.88 (br. s., 1H), 8.22 (s, 1H), 6.80 (s, 1H), 4.32 (q, J=7.1 Hz, 2H), 2.72 (s, 3H), 1.35 (t, J=7.0 Hz, 3H).

Step 2: Tributylphosphine (0.81 mL, 3.04 mmol) was added dropwise to a stirred mixture of ethyl 5-hydroxy-2-methyl-furo[2,3-c]pyridine-3-carboxylate (480 mg, 2.17 mmol), (2-(Trifluoromethyl)pyridin-3-yl)methanol (0.55 mL, 3.25 mmol) and ADDP (766.48 mg, 3.04 mmol) in dry THF (42 mL) under argon. The RM was stirred for 1 h. The RM was concentrated under reduced pressure. The residue was purified by FCC on silica gel using a gradient of EtOAc (10-100%) in heptane to afford ethyl 2-methyl-5-[[2-(trifluoromethyl)-3-pyridyl]methoxy]furo[2,3-c]pyridine-3-carboxylate (272.3 mg 33% yield) as a white solid. ESI/APCI(+): 381 (M+H); 1H NMR (DMSO-d6) d (ppm): 8.70 (d, J=4.3 Hz, 1H), 8.54 (s, 1H), 8.21 (d, J=7.9 Hz, 1H), 7.75 (dd, J=7.8, 4.7 Hz, 1H), 7.23 (s, 1H), 5.60 (s, 2H), 4.35 (q, J=7.0 Hz, 2H), 2.78 (s, 3H), 1.36 (t, J=7.0 Hz, 3H).

Step 3: An aqueous solution of NaOH (2N) (3 mL, 6 mmol) was added to a solution of ethyl 2-methyl-5-[[2-(trifluoromethyl)-3-pyridyl]methoxy]furo[2,3-c]pyridine-3-carboxylate (272 mg, 0.72 mmol) inMeOH (3 mL) and 1,4-Dioxane (1.5 mL). The mixture was stirred at RT for 23 h and a 4M solution of HCl in dioxane (1.5 mL) was added. The mixture was concentrated under reduced pressure. Cold water was added to the residue. The solid material was collected by filtration and dried to afford 2-methyl-5-[[2-(trifluoromethyl)-3-pyridyl]methoxy]furo[2,3-c]pyridine-3-carboxylic acid (185 mg, 73% yield) as a white solid. ESI/APCI(+): 353 (M+H); ESJ/APCI(−): 351 (M−H).

Step 4: A mixture of HATU (97.15 mg, 0.2600 mmol), tert-butyl (R)-4-amino-3,3-difluoropiperidine-1-carboxylate (60.36 mg, 0.26 mmol), DIPEA (0.13 mL, 0.77 mmol), and 2-methyl-5-[[2-(trifluoromethyl)-3-pyridyl]methoxy]furo[2,3-c]pyridine-3-carboxylic acid (90.mg, 0.26 mmol) in DMF (2.2 mL) was stirred overnight at RT and was partitioned between water and DCM. The aqueous layer was extracted twice with DCM. Combined organic extracts were dried over magnesium sulphate, filtered, and concentrated under reduced pressure. The residue was purified by FCC on silica gel using a gradient of EtOAc (5-60%) in heptane to afford tert-butyl (4R)-3,3-difluoro-4-[[2-methyl-5-[[2-(trifluoromethyl)-3-pyridyl]methoxy]furo[2,3-c]pyridine-3-carbonyl]amino]piperidine-1-carboxylate (115.7 mg, 79% yield) as a yellow solid. ESJ/APCI(+): 571 (M+H); ESI/APCI(−): 569 (M−H).

Step 5: A 4M dioxane solution of HCl (3 mL, 12 mmol) was added to a solution of tert-butyl (4R)-3,3-difluoro-4-[[2-methyl-5-[[2-(trifluoromethyl)-3-pyridyl]methoxy]furo[2,3-c]pyridine-3-carbonyl]amino]piperidine-1-carboxylate (114 mg, 0.20 mmol) in DCM (3 mL). The mixture was stirred at RT for 1 h and was concentrated under reduced pressure. The residue was purified by a column of supported SiliaBond-PropylSulfonic Acid using a gradient of MeOH (0-100%) in DCM before elution with a 3N solution of ammonia in MeOH to afford N-[(4R)-3,3-difluoropiperidin-4-yl]-2-methyl-5-{[2-(trifluoromethyl)pyridin-3-yl]methoxy}furo[2,3-c]pyridine-3-carboxamide (Cpd 025) (86.7 mg, 91% yield) as a white solid.

The following compounds were prepared in a similar manner (use of appropriate reagents (chiral or racemic) and purification methods (including chiral HPLC or chiral SFC) known to the person skilled in the art) as described for Cpd 025: Cpd 021-En1, Cpd 021-En2, Cpd 022-En1, Cpd 023-En1 and Cpd 026.

### Synthesis of 5-[(2-fluorophenyl)methoxy]-N-[1-(hydroxymethyl)cyclopropyl]-2-methylfuro[2,3-c]pyridine-3-carboxamide (Cud 018)

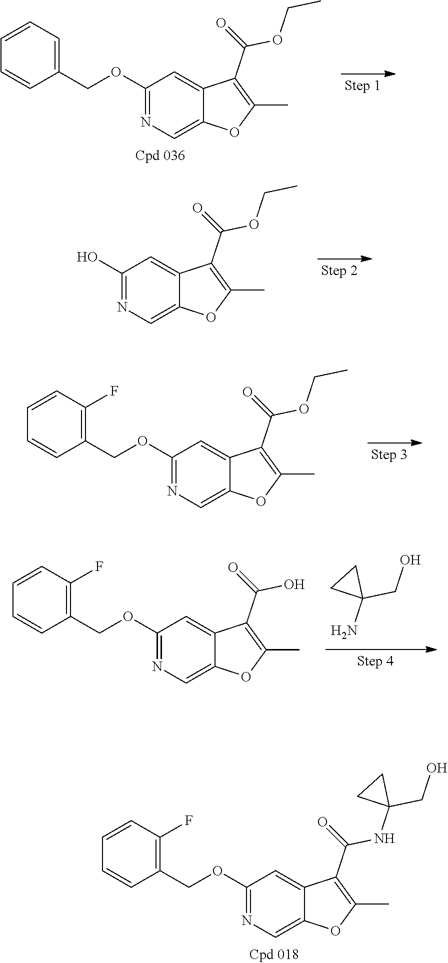

Step 1: Palladium on activated carbon, 10% Pd, unreduced (213.33 mg, 2 mmol) was added to a degassed solution of ethyl 5-benzyloxy-2-methyl-furo[2,3-c]pyridine-3-carboxylate (Cpd 036) (1600 mg, 5.14 mmol) in a mixture of Ethanol (42 mL) and THF (21 mL). The mixture was degassed and then fitted with a balloon of hydrogen. The RM was stirred for 8 h at RT. The RM was then filtered. The solid material was collected and suspended in 250 mL of a ¼ mixture of MeOH and DCM. The mixture was filtered. The solid material was again collected and treated as above with 250 mL of a ¼ mixture MeOH and DCM. Combined filtrates were concentrated under reduced pressure to afford ethyl 5-hydroxy-2-methyl-furo[2,3-c]pyridine-3-carboxylate (1109.3 mg, 5.0147 mmol, 98% yield) as a white solid. ESJ/APCI(+): 222 (M+H); ESJ/APCI(−): 220 (M−H); 1H NMR (DMSO-d6) d (ppm): 10.88 (br. s., 1H), 8.22 (s, 1H), 6.80 (s, 1H), 4.32 (q, J=7.1 Hz, 2H), 2.72 (s, 3H), 1.35 (t, J=7.0 Hz, 3H).

Step 2: To a stirred solution of ethyl 5-hydroxy-2-methylfuro[2,3-c]pyridine-3-carboxylate (490 mg, 2.217 mmol), Ag₂CO₃ (916.9 mg, 2.438 mmol) in THF (10 mL), was stirred for 30 min. After 30 min, 1-(Bromomethyl)-2-fluorobenzene was added to the RM. RM was stirred at RT for 12 hours and was concentrated under reduced pressure. The residue was partitioned between water and DCM. The aqueous layer was extracted with DCM. Combined organic extracts were concentrated under reduced pressure. The residue was purified by FCC on silica gel using a gradient of EtOAc (5-40%) in hexane to afford ethyl 5-((2-fluorobenzyl)oxy)-2-methylfuro[2,3-c]pyridine-3-carboxylate (38% yield) as an off-white solid. TLC system: 70% EtOAc in Pet ether; RF: 0.35.

Step 3: To a stirred solution of ethyl 5-((2-fluorobenzyl)oxy)-2-methylfuro[2,3-c]pyridine-3-carboxylate (250 mg, 0.759 mmol), in 1,4-Dioxane (3 mL), NaOH (221.8 mg, 5.547 mmol) in water (3 ml), and MeOH (3 ml) were added at 0° C. The RM was stirred at RT for 16 h. The RM was concentrated under reduced pressure. The residue was partitioned between water and DCM. After separation, the aqueous layer was extracted with DCM. Combined organic extracts were concentrated under reduced pressure. The residue was purified by FCC on silica gel using a gradient of EtOAc (5-40%) in hexane to afford 5-((2-fluorobenzyl)oxy)-2-methylfuro[2,3-c]pyridine-3-carboxylic acid (78.7% yield) as an off-white solid. TLC system: 5% MeOH in Pet DCM; RF: 0.15.

Step 4: To a solution of 5-((2-fluorobenzyl)oxy)-2-methylfuro[2,3-c]pyridine-3-carboxylic acid (90 mg, 0.299 mmol) in DMF (2.5 mL) was added DIPEA (115.7 mg, 0.897 mmol) followed by HATU (170 mg, 0.448 mmol). The RM was stirred at RT for 15 min and then 2-Amino-2-methylpropanamide (44.3 mg, 0.358 mmol) was added. The RM was stirred at RT for 18 h. RM was diluted with EtOAc (100 mL) and washed with 1N HCl (2×100 mL), brine (5×100 mL), dried over anhydrous sodium sulphate, and concentrated to under reduced pressure to give crude. Crude was purified by FCC on silica gel using a gradient of 0-25% EtOAc in heptane to provide 5-[(2-fluorophenyl)methoxy]-N-[1-(hydroxymethyl)cyclopropyl]-2-methylfuro[2,3-c]pyridine-3-carboxamide (Cpd 018) (36.3% yield) as an off-white solid. 50% EtOAc in Pet ether; RF: 0.35. The following compounds were prepared in a similar manner (use of appropriate reagents (chiral or racemic) and purification methods (including chiral HPLC or chiral SFC) known to the person skilled in the art) as described for Cpd 018: Cpd 002, Cpd 006-En1, Cpd 006-En2, Cpd 007, Cpd 008-En1, Cpd 008-En2, Cpd 009-En1, Cpd 009-En2, Cpd 012, Cpd 014-En1, Cpd 014-En2, Cpd 017-En1, Cpd 017-En2, Cpd 020, Cpd 024, Cpd 027, Cpd 029, Cpd 031, Cpd 032 and Cpd 034.

Analytical data

| | [M + H]+ | ¹H NMR |
|---|---|---|
| Cpd | (m/z): | (δ ppm) |
| Cpd 001 | 391.1 | (500 MHz, DMSO-d6) δ = 8.54 (s, 1H), 7.69 (s, 1H), 7.71 (s, 1H), 7.46 (s, 1H), 7.16 |
| | | (s, 1H), 7.10 (s, 1H), 5.55 (s, 2H), 4.99-5.01 (t, 1H), 4.42- 4.46 (m, 1H), 3.72 (t, 2H), |
| | | 2.68 (s, 3H), 2.63 (s, 3H). |
| Cpd 002 | 402.1 | (400 MHz, DMSO) δ = 8.52 (s, 1H), 7.54-7.57 (m, 1H), 7.41-7.53 (m, 3H), 7.38-7.39 |
| | | (m, 2H), 7.15-7.25 (m, 2H), 5.43 (s, 2H), 5.06 (d, 1H), 4.33-4.35 (m, 1H), 4.10-4.13 |
| | | (m, 1H), 2.69 (s, 3H), 1.12 (s, 3H). |
| Cpd 003 | 405.1 | (400 MHz, DMSO) δ = 8.56 (s, 1H), 7.71 (s, 1H), 7.44-7.45 (m, 2H), 7.09-7.14 (m, |
| | | 2H), 5.55 (s, 2H), 5.06 (d, 1H), 4.32-4.34 (m, 1H), 4.12 (bs, 1H), 2.69 (s, 3H), 2.59 (s, |
| | | 3H), 1.12 (d, 3H). |
| Cpd 004 | 405.1 | (400 MHz, DMSO) δ = 8.94 (s, 1H), 8.55 (s, 1H), 7.43-7.46 (m, 2H), 7.14 (s, 1H), |
| | | 7.10 (s, 1H), 5.57 (s, 2H), 5.05 (d, 1H), 4.32-4.34 (m, 2H), 4.10-4.13 (m, 1H), 2.69 |
| | | (s, 3H), 2.50 (s, 3H), 1.12 (s, 3H). |
| Cpd 005- | 375.1 | (400 MHz, DMSO-d6): δ 8.94 (s, 1H), 8.52 (d, 1H), 7.97 (d, 1H), 7.46 (s, 1H), 7.06- |
| En1 | | 7.08 (m, 2H), 5.56 (s, 2H), 4.42-4.46 (m, 1H), 2.66 (s, 3H), 2.45 (s, 3H), 1.33 (d, 3H). |
| Cpd 005- | 375.1 | (400 MHz, DMSO-d6): δ 8.94 (s, 1H), 8.53 (d, 1H), 7.97 (d, 1H), 7.46 (s, 3H), 7.06- |
| En2 | | 7.08 (m, 2H), 5.56 (s, 2H), 4.42-4.46 (m, 1H), 2.66 (s, 3H), 2.45 (s, 3H), 1.33 (d, 3H). |
| Cpd 006_ | 355.2 | (500 MHz, DMSO-d6) δ = 8.55 (d, 1H), 8.46 (s, 1H), 7.99 (d, 1H), 7.77-7.80 (m, 1H), |
| En1 | | 7.48 (s, 1H), 7.45 (d, 1H), 7.30-7.32 (m, 1H), 7.20 (s, 1H), 7.08 (s, 1H), 5.45 (s, 2H), |
| | | 4.43-4.49 (m, 1H), 2.67 (s, 3H), 1.35 (d, 3H). |
| Cpd 006_ | 355.2 | (500 MHz, DMSO-d6) δ = 8.55 (d, 1H), 8.46 (s, 1H), 7.99 (d, 1H), 7.77-7.80 (m, 1H), |
| En2 | | 7.45-7.48 (s, 1H), 7.45 (d, 1H), 7.30-7.32 (m, 1H), 7.20 (s, 1H ), 7.08 (s, 1H ), 5.45 (s, |
| | | 2H), 4.44-4.48 (m, 1H), 2.67 (s, 3H), 1.35 (d, 3H). |
| Cpd 007 | 386.1 | (400 MHz, DMSO-d6): δ 8.50 (s, 1H), 7.87 (s, 1H), 7.56 (t, 1H), 7.37-7.43 (m, 1H), |
| | | 7.32 (s, 1H), 7.17-7.26 (m, 3H), 7.07 (s, 1H), 5.42 (s, 2H), 2.67 (s, 3H), 1.52 (s, 6H) |
| Cpd 008- | 388.1 | (400 MHz, DMSO-d6): δ 8.51 (s, 1H), 7.70 (d, 1H), 7.53-7.57 (t, 1H), 7.46 (bs, 1H), |
| En1 | | 7.41-7.43 (m, 1H), 7.19-7.39 (m, 4H), 5.43 (s, 2H), 5.03 (bs, 1H), 4.42-4.47 (m, |
| | | 1H), 3.72 (d, 2H), 2.68 (s, 3H). |
| Cpd 008- | 388.1 | (400 MHz, DMSO-d6): δ 8.51 (s, 1H), 7.70 (d, 1H), 7.53-7.57 (t, 1H), 7.46 (s, 1H), |
| En2 | | 7.41-7.43 (m, 1H), 7.16-7.39 (m, 4H), 5.4 (s, 2H), 5.03 (bs, 1H), 4.42-4.47 (m, 1H), |
| | | 3.72 (d, 2H), 2.68 (s, 3H). |
| Cpd 009- | 372.1 | (400 MHz, DMSO) δ = 8.49 (d, 1H), 7.98 (d, 1H), 7.55 (t, 1H), 7.46 (bs, 1H), 7.37- |
| En1 | | 7.41 (m, 1H), 7.19 -7.26 (m, 2H), 7.16 (s, 1H), 7.06 (s, 1H), 5.42 (s, 2H), 4.45 (t, 1H), |
| | | 2.66 (s, 3H), 1.34 (d, 3H). |
| Cpd 009- | 372.1 | (400 MHz, DMSO) δ = 8.49 (d, 1H), 7.98 (d, 1H), 7.55 (t, 1H), 7.46 (bs, 1H), 7.37 - |
| En2 | | 7.41 (m, 1H), 7.26-7.19 (m, 2H), 7.13 (s, 1H), 7.06 (s, 1H), 5.42 (s, 2H), 4.45 (t, 1H), |
| | | 2.66 (s, 3H), 1.34 (d, 3H). |
| Cpd 010- | 354.1 | (400 MHz, DMSO) δ = 8.48 (d, 1H), 7.98 (d, 1H), 7.44-7.47 (m, 3H), 7.31-7.39 (m, |
| En1 | | 3H), 7.13 (s, 1H), 7.07 (s, 1H), 5.38 (s, 2H), 4.43-4.47 (m, 1H), 2.66 (s, 3H), 1.34 (d, |
| | | 3H). |
| Cpd 010- | 354.1 | (400 MHz, DMSO) δ = 8.48 (s, 1H), 7.98 (d, 1H), 7.44-7.46 (m, 3H), 7.31-7.39 (m, |
| En2 | | 3H), 7.13 (s, 1H), 7.07 (bs, 1H), 5.38 (s, 2H), 4.43-4.47 (m, 1H), 2.66 (s, 3H), 1.35 (d, |
| | | 3H). |
| Cpd 011 | 389 | (400 MHz, DMSO) δ = 8.94 (s, 1H), 8.53 (d, 1H), 7.87 (s, 1H), 7.33 (s, 1H), 7.07- |
| | | 7.12 (m, 2H), 5.56 (s, 2H), 2.67 (s, 3H), 2.49 (m, 3H), 1.51 (s, 6H). |
| Cpd 012 | 369.1 | (500 MHz, DMSO-d6) δ = 8.55 (d, 1H), 8.47 (s, 1H), 7.89 (s, 1H), 7.78-7.81 (t, 1H), |
| | | 7.46 (d, 1H), 7.35 (s, 1H), 7.30 -7.33 (m, 1H), 7.25 (s 1H), 7.11 (s, 1H), 5.45 (s, 2H), |
| | | 2.68 (s, 3H), 1.54 (s, 6H). |
| Cpd 013- | 391.1 | (400 MHz, DMSO) δ = 8.94 (s, 1H), 8.54 (s, 1H), 7.73 (d, 1H), 7.47 (s, 1H), 7.15 (s, |
| En1 | | 1H), 7.11 (s, 1H), 5.56 (s, 2H), 5.05-5.09 (bs, 1H), 4.42-4.45 (m, 1H), 3.71-3.73 |
| | | (m, 2H), 2.68 (s, 3H), 2.45 (s, 3H). |
| Cpd 013- | 391.1 | (400 MHz, DMSO) δ = 8.93 (s, 1H), 8.53 (s, 1H), 7.67-7.69 (d, 1H), 7.44 (s, 1H), 7.14 |
| En2 | | (s, 1H), 7.11 (s, 1H), 5.56 (s, 2H), 4.98 (t, 1H), 4.42-4.46 (m, 1H), 3.71-3.73 (m, 2H), |
| | | 2.68 (s, 3H), 2.45 (s, 3H). |
| Cpd 014- | 371.1 | (500 MHz, DMSO-d6) δ = 8.57 (d, 1H), 8.47 (s, 1H), 7.83 (t, 1H), 7.72 (d, 1H), 7.48- |
| En1 | | 7.50 (m, 2H), 7.34-7.36 (m, 1H), 7.24 (s, 1H), 7.18 (s, 1H), 5.47 (s, 2H), 4.45-4.47 (m, |
| | | 1H), 3.74-3.75 (m, 2H), 3.36-3.42 (m, 1H), 2.69 (s, 3H). |
| Cpd 014- | 371.1 | (500 MHz, DMSO-d6) δ = 8.55 (d, 1H), 8.47 (s, 1H), 7.76-7.81 (m, 1H), 7.74 (d, 1H), |
| En2 | | 7.44-7.49 (m, 2H), 7.30-7.32 (m, 1H), 7.23 (s, 1H), 7.17 (s, 1H), 5.46 (s, 2H), 5.06 |
| | | (brs, 1H), 4.44-4.48 (m, 1H), 3.74-3.75 (d, 2H), 2.71 (s, 3H). |
| Cpd 015- | 388 | (DMSO-d6) d: 8.50 (s, 1H), 8.23 (d, J = 8.5 Hz, 1H), 7.42-7.49 (m, 2H), 7.38 (t, J = |
| En1 | | 7.2 Hz, 2H), 7.28-7.34 (m, 1H), 7.04 (s, 1H), 5.30-5.44 (m, 2H), 4.61 (dt, J = 16.3, 7.9 |
| | | Hz, 1H), 3.19-3.29 (m, 2H), 2.94-3.08 (m, 1H), 2.78-2.92 (m, 1H), 2.65 (s, 3H) |
| Cpd 015- | 388 | (DMSO-d6) d: 8.50 (s, 1H), 8.23 (d, J = 8.5 Hz, 1H), 7.42-7.50 (m, 2H), 7.38 (t, J = |
| En2 | | 7.2 Hz, 2H), 7.28-7.34 (m, 1H), 7.04 (s, 1H), 5.38 (s, 2H), 4.61 (dt, J = 16.3, 7.9 Hz, |
| | | 1H), 3.18-3.29 (m, 1H), 2.93-3.08 (m, 1H), 2.80-2.92 (m, 1H), 2.65 (s, 3H) |
| Cpd 016 | 488 | (DMSO-d6) d: 8.47 (s, 1H), 7.51 (s, 1H), 7.45 (d, J = 7.32 Hz, 2H), 7.37 (t, J = 7.5 Hz, |
| | | 2H), 7.27-7.34 (m, 1H), 7.01 (s, 1H), 5.38 (s, 2H), 4.88 (t, J = 5.5 Hz, 1H), 3.54-3.74 |
| | | (m, 6H), 2.64 (s, 3H), 2.16 (d, J = 13.7 Hz, 2H), 1.53-1.67 (m, 2H) |
| Cpd 017- | 428.1 | (400 MHz, DMSO-d6): δ 8.50 (s, 1H), 8.21 (d, 1H), 7.55 (t, 1H), 7.37-7.42 (m, 1H), |
| En1 | | 7.17-7.25 (m, 3H), 5.42 (s, 2H), 4.64-4.72 (m, 2H), 3.27-3.53 (m, 5H), 3.14-3.19 |
| | | (m, 1H), 2.63 (s, 3H), 2.49-2.50 (m, 1H), 2.31-2.36 (m, 1H), 1.30 (s, 1H). |
| Cpd 017- | 428.1 | (400 MHz, DMSO-d6): δ 8.50 (s, 1H), 8.22 (d, 1H), 7.56 (t, 1H), 7.37-7.42 (m, 1H), |
| En2 | | 7.17-7.25 (m, 3H), 5.42 (s, 2H), 4.64-4.72 (m, 2H), 3.27-3.53 (m, 5H), 3.14-3.19 |
| | | (m, 1H), 2.63 (s, 3H), 2.49-2.50 (m, 1H), 2.31-2.36 (m, 1H), 1.30 (s, 1H). |
| Cpd 018 | 371.1 | (400 MHz, DMSO-d6): δ 8.47 (d, 1H), 8.21 (s, 1H), 7.56 (t, 1H), 7.37-7.53 (m, 1H), |
| | | 7.22-7.25 (m, 2H), 7.07 (s, 1H), 5.41 (s, 2H), 4.73 (t, 1H), 3.53 (d, 2H), 2.61 (s, 3H), |
| | | 0.75 (d, 4H). |
| Cpd 019 | | (600 MHz, DMSO) d 8.939 (s, 1H), 8.703 (s, 1H), 8.537 (s, 1H), 7.290 (s, 1H), 7.221 (s, |
| | | 1H), 7.134 (s, 1H), 5.578 (s, 2H), 4.843 (d, 2H), 4.663 (d, 2H), 2.692 (s, 3H), 2.459 (s, |
| | | 3H) |
| Cpd 020 | | (600 MHz, DMSO) d 8.731 (s, 1H), 8.555 (ddd, 1H), 8.478 (d, 1H), 7.797 (td, 1H), |
| | | 7.529-7.439 (m, 1H), 7.401 (d, 1H), 7.315 (ddd, 1H), 7.236 (s, 1H), 7.154 (s, 1H), |
| | | 5.468 (s, 2H), 4.863 (d, 2H), 4.686 (d, 2H), 2.700 (s, 3H). |
| Cpd 021- | 409 | (DMSO-d6) d: 8.95 (s, 1H), 8.54 (s, 1H), 8.23 (d, J = 8.5 Hz, 1H), 6.99 (s, 1H), 5.49- |
| En1 | | 5.66 (m, 2H), 4.61 (dquin, J = 16.4, 7.9 Hz, 1H), 3.20-3.29 (m, 2H), 3.02 (dt, J = 17.9, |
| | | 13.2 Hz, 1H), 2.88 (dd, J = 11.4, 8.4 Hz, 1H), 2.65 (s, 3H), 2.46 (s, 3H) |
| Cpd 021- | 409 | (DMSO-d6) d: 8.95 (s, 1H), 8.55 (s, 1H), 8.23 (d, J = 8.9 Hz, 1H), 6.98 (s, 1H), 5.51- |
| En2 | | 5.65 (m, 2H), 4.61 (dquin, J = 16.3, 8.0 Hz, 1H), 3.20-3.29 (m, 2H), 3.03 (dt, J = 17.9, |
| | | 13.2 Hz, 1H), 2.88 (dd, J = 11.4, 8.4 Hz, 1H), 2.65 (s, 3H), 2.46 (s, 3H) |
| Cpd 022- | 402 | (DMSO-d6) d: 8.48 (s, 1H), 8.23 (d, J = 8.5 Hz, 1H), 7.32 (d, J = 4.0 Hz, 4H), 7.17- |
| En1 | | 7.26 (m, 1H), 6.94 (s, 1H), 4.61 (dt, J = 16.3, 8.0 Hz, 1H), 4.49 (t, J = 6.7 Hz, 2H), 3.17- |
| | | 3.28 (m, 2H), 2.95-3.11 (m, 3H), 2.82-2.92 (m, 1H), 2.64 (s, 3H) |
| Cpd 023- | 471 | (DMSO-d6) d: 8.83 (s, 1H), 8.48 (s, 1H), 8.26 (d, J = 8.5 Hz, 1H), 6.95 (s, 1H), 4.62 |
| En1 | | (dt, J = 16.2, 8.1 Hz, 1H), 4.44 (t, J = 6.0 Hz, 2H), 3.15-3.28 (m, 4H), 3.02 (dt, J = |
| | | 17.6, 13.3 Hz, 1H), 2.87 (dd, J = 11.1, 8.7 Hz, 1H), 2.64 (s, 3H), 2.35 (s, 3H) |
| Cpd 024 | | (600 MHz, DMSO) d 8.548 (ddd, 1H), 8.462 (d, 1H), 7.928 (s, 1H), 7.791 (td, 1H), |
| | | 7.491-7.437 (m, 1H), 7.309 (ddd, 1H), 7.143 (d, 1H), 7.067 (s, 1H), 6.920 (s, 1H), |
| | | 5.458 (s, 2H), 3.751 (dt, 2H), 3.631 (td, 2H), 2.671 (s, 3H), 2.073 (d, 2H), 2.022 (ddd, |
| | | 2H). |
| Cpd 025 | 471 | (DMSO-d6) d: 8.70 (d, J = 4.0 Hz, 1H), 8.49 (s, 1H), 8.34 (d, J = 8.9 Hz, 1H), 8.19 (d, J |
| | | = 7.9 Hz, 1H), 7.75 (dd, J = 7.6, 4.9 Hz, 1H), 7.03 (s, 1H), 5.60 (s, 2H), 4.38-4.59 (m, |
| | | 1H), 3.08-3.19 (m, 1H), 2.78-2.97 (m, 2H), 2.58-2.70 (m, 4H), 1.62-1.86(m, 2H). |
| Cpd 026 | 471 | (DMSO-d6) d: 8.70 (d, J = 4.3 Hz, 1H), 8.49 (s, 1H), 8.34 (d, J = 9.2 Hz, 1H), 8.19 (d, |
| | | J = 7.9 Hz, 1H), 7.75 (dd, J = 7.6, 4.9 Hz, 1H), 7.03 (s, 1H), 5.60 (s, 2H), 4.38-4.57 (m, |
| | | 1H), 3.08-3.18 (m, 1H), 2.75-2.99 (m, 2H), 2.59-2.69 (m, 4H), 1.62-1.86 (m, 2H) |
| Cpd 027 | 411.2 | (500 MHz, DMSO-d6) δ = 8.56 (d, 1H), 8.46 (s, 1H), 8.25 (d, 1H), 7.79-7.82 (m, 1H), |
| | | 7.46 (d, 1H), 7.31-7.33 (m, 1H), 7.24 (s, 1H), 5.46 (s, 2H), 4.65-4.72 (m, 2H), 3.51- |
| | | 3.54 (m, 2H), 3.39-3.47 (m, 3H), 3.14-3.21 (m, 1H), 2.67 (s, 3H), 2.34-2.37 (m, 1H), |
| | | 1.95-2.00 (m, 1H). |
| Cpd 028 | | (600 MHz, DMSO) d 8.932 (s, 0H), 8.493 (s, 1H), 7.811 (s, 1H), 7.002 (s, 1H), 5.555 (s, |
| | | 2H), 4.846 (t, 1H), 3.644 (d, 2H), 2.627 (s, 3H), 2.453 (s, 3H), 2.293 (qd, 2H), 2.118 (dt, |
| | | 2H), 1.897-1.681 (m, 3H). |
| Cpd 029 | | (600 MHz, DMSO) d 8.547 (ddd, 1H), 8.428 (d, 1H), 7.826 (s, 1H), 7.789 (td, 1H), |
| | | 7.459 (dt, 1H), 7.309 (ddd, 1H), 7.121 (d, 1H), 5.445 (s, 2H), 3.661 (s, 2H), 2.630 (s, |
| | | 3H), 2.310 (tdd, 2H), 2.167-2.086 (m, 2H), 1.911-1.809 (m, 1H), 1.753 (dp, 1H). |
| Cpd 030 | 374.1 | (400 MHz, DMSO-d6): δ 8.94 (s, 1H), 8.49 (d, 1H), 8.21 (s, 1H), 7.02 (d, 1H), 5.55 (s, |
| | | 2H), 4.73 (t, 1H), 3.52 (d, 2H), 2.61 (s, 3H), 2.45 (s, 3H), 0.74 (d, 4H). |
| Cpd 031 | 354.1 | (500 MHz, DMSO-d6) δ = 8.55 (d, 1H), 8.43 (s, 1H), 8.24 (s, 1H), 7.76-7.80 (m, 1H), |
| | | 7.44-7.46 (d, 1H), 7.30-7.32 (m, 1H), 7.14 (s, 1H), 5.44 (s, 2H), 4.78 (brs, 1H), 3.54 |
| | | (s, 2H), 2.61 (s, 3H), 0.76-0.77 (d, 4H). |
| Cpd 032 | 384.1 | (400 MHz, DMSO-d6): δ 8.45 (s, 1H), 8.22 (s, 1H), 8.13 (dd, 1H), 7.77 (dd, 1H), 7.11 |
| | | (s, 1H), 6.98-7.01 (m, 1H), 5.32 (s, 2H), 4.76 (bs, 1H), 3.91 (s, 3H), 3.53 (s, 2H) 2.61 |
| | | (s, 3H), 0.75 (d, 4H), |
| Cpd 033 | | (600 MHz, DMSO) d 8.934 (s, 1H), 8.515 (s, 1H), 8.342 (s, 1H), 7.108 (s, 1H), 5.562 (s, |
| | | 2H), 5.145 (t, 1H), 4.655 (d, 2H), 4.518 (d, 2H), 3.756 (d, 2H), 2.651 (s, 3H), 2.453 (s, |
| | | 3H). |
| Cpd 034 | | (600 MHz, DMSO) d 8.575-8.526 (m, 1H), 8.456 (s, 1H), 8.369 (s, 1H), 7.792 (td, |
| | | 1H), 7.464 (d, 1H), 7.311 (dd, 1H), 7.228 (d, 1H), 5.454 (s, 2H), 5.162 (t, 1H), 4.676 (d, |
| | | 2H), 4.538 (d, 2H), 3.776 (d, 2H), 2.658 (s, 3H). |
| Cpd 035 | 418 | (DMSO-d6) d: 8.94 (s, 1H), 8.51 (s, 1H), 7.51 (s, 1H), 6.95 (s, 1H), 5.56 (s, 2H), 4.87 |
| | | (t, J = 5.5 Hz, 1H), 3.50-3.77 (m, 6H), 2.64 (s, 3H), 2.45 (s, 3H), 2.15 (d, J = 13.4 Hz, |
| | | 2H), 1.52-1.71 (m, 2H) |
| Cpd 036 | 312 | (DMSO-d6) d: 8.53 (s, 1H), 7.43-7.49 (m, 2H), 7.38 (t, J = 7.3 Hz, 2H), 7.28-7.34 (m, |
| | | 1H), 7.16 (s, 1H), 5.38 (s, 2H), 4.34 (q, J = 7.0 Hz, 2H), 2.77 (s, 3H), 1.35 (t, J = 7.2 Hz, |
| | | 3H) |

Chiral analytical data
Chiral SFC analysis

| | | | Co-solvent | Flow | | |
|---|---|---|---|---|---|---|
| Cpd | Column Name | Co-solvent | % | [g/min] | RT [min] | Purity [%] |
| Cpd 005-En1 | (3) | MeOH | 40 | 4 | 3.42 | 99.7 |
| Cpd 005-En2 | (3) | MeOH | 40 | 4 | 5.18 | 99 |
| Cpd 006 _ En1 | (1) | MeOH | 40 | 3 | 2.13 | 99.6 |
| Cpd 006_En2 | (1) | MeOH | 40 | 3 | 4.48 | 99.9 |
| Cpd 009-En1 | (2) | MeOH | 40 | 3 | 2.81 | 99.2 |
| Cpd 009-En2 | (2) | MeOH | 40 | 3 | 6.6 | 98.7 |
| Cpd 010-En1 | (1) | MeOH | 40 | 3 | 3.29 | 99.9 |
| Cpd 010-En2 | (1) | MeOH | 40 | 3 | 6.69 | 99.9 |
| Cpd 013-En1 | (1) | MeOH | 40 | 3 | 3.26 | 99.8 |
| Cpd 013-En2 | (1) | MeOH | 40 | 3 | 4.91 | 98.7 |
| Cpd 014-En1 | (1) | MeOH | 40 | 3 | 3.24 | 99.8 |
| Cpd 014-En2 | (1) | MeOH | 40 | 3 | 6.1 | 96.1 |
| Cpd 017-En1 | (3) | MeOH | 30 | 4 | 7.06 | 99.7 |
| Cpd 017-En2 | (3) | MeOH | 30 | 4 | 9.37 | 99.8 |

Chiralpak AD-3(4.6*150 mm)3 μm = (1); Chiralpak IG-3(4.6*150 mm)3 μm = (2); CHIRALCEL OX-H (250 × 4.6 mm), 5 μm = (3).

### Part B

### Monitoring the TRPM3 Ion Channel Driven Ca²⁺ Uptake.

In order to monitor the inhibition of the mouse TRPM3α2 (mTRPM3) ion channel by the compounds of the invention, a cellular system making use of an mTRPM3alpha2 or hTRPM3 overexpressing cell line (flip-in HEK293) was used. The TRPM3 channel was stimulated/opened with Pregnenolone sulfate (PS) (50 μM) which results in Ca²⁺ influx.

For mTRPM3, the intracellular Ca²⁺ was measured with a Calcium responsive dye, Fluor-4 AMV ester (Invitrogen). Cells were cultured until a confluence of 80-90%, washed with Versene (Invitrogen) and detached from the surface by a short incubation with 0.05% Trypsin (Invitrogen). The trypsination process was stopped by the addition of complete cell culture medium (DMEM, glutamax, 10% FCS, NEAA, Pen-Strep). Cells were collected and resuspended in Krebs buffer without Calcium at RT.

Prior the cell seeding (±2000 cells/well into a black, 384 well plate (Greiner)) the diluted compound was added in the assay plate, together with the PS dissolved in Krebs buffer containing Calcium. This resulted in a 2.4mMv Ca²⁺ assay solution. Directly after cell addition the plates were read on an Envision fluorescence reader (Perkin Elmer) by an Excitation of 485 nM and emission at 535 nM.

Channel inhibition was calculated compared to a non-PS stimulated control versus a condition stimulated with PS (50 μM) with vehicle. The ability of the compounds of the invention to inhibit this activity was determined as: Percentage inhibition=[1-((RFU determined for sample with test compound present−RFU determined for sample with positive control inhibitor) divided by (RFU determined in the presence of vehicle−RFU determined for sample with positive control inhibitor))]*100.

The activities of the Example compounds tested are depicted in the table below. The activity ranges A, B and C refer to IC50 values in the Fluo-4 AM assay as follows: “A”: IC₅₀<1 μM; “B”: 1 μM≤IC₅₀≤20 μM and “C”: IC₅₀>20 μM

| Cpd | IC50 |
|---|---|
| Cpd 001 | B |
| Cpd 002 | A |
| Cpd 003 | B |
| Cpd 004 | A |
| Cpd 005-En1 | B |
| Cpd 005-En2 | C |
| Cpd 006 _ En1 | B |
| Cpd 006 _ En2 | B |
| Cpd 007 | B |
| Cpd 008-En1 | B |
| Cpd 008-En2 | A |
| Cpd 009-En1 | B |
| Cpd 009-En2 | B |
| Cpd 010-Enl | B |
| Cpd 010-En2 | B |
| Cpd 011 | B |
| Cpd 012 | B |
| Cpd 013-En1 | C |
| Cpd 013-En2 | A |
| Cpd 014-En1 | B |
| Cpd 014-En2 | A |
| Cpd 015-En1 | C |
| Cpd 015-En2 | A |
| Cpd 016 | C |
| Cpd 017-En1 | B |
| Cpd 017-En2 | B |
| Cpd 018 | B |
| Cpd 019 | C |
| Cpd 020 | B |
| Cpd 021-En1 | A |
| Cpd 021-En2 | C |
| Cpd 022-En1 | B |
| Cpd 023-En1 | B |
| Cpd 024 | B |
| Cpd 025 | A |
| Cpd 026 | B |
| Cpd 027 | B |
| Cpd 028 | B |
| Cpd 029 | B |
| Cpd 030 | C |
| Cpd 031 | C |
| Cpd 032 | C |
| Cpd 033 | B |
| Cpd 034 | C |
| Cpd 035 | B |

1. A compound of formula (I), a stereo-isomeric form, a physiologically acceptable salt, solvate and/or polymorph thereof 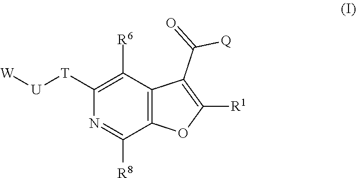
wherein
R¹ represents —F, —Cl, —Br, —I, —CN, —R^{w}, —OR^{w}, —OC(═O)R^{w}, —NR^{w}R^{x}, —NR^{w}C(═O)R^{x}, —SR^{w}, —S(═O)R^{w}, —S(═O)₂R^{w}, —C(═O)R^{w}, —C(═O)OR^{w}, or —C(═O)NR^{w}R^{x};
Q represents —NR³R⁴;
R³ represents —OH or —R^{Y1};
R⁴ represents —R² or —S(═O)₂R^{Y3};
or R³ and R⁴ together form a 4, 5, 6, 7 or 8 membered heterocycle containing 1 to 3 heteroatoms selected from N, O and S, saturated or unsaturated, unsubstituted or mono- or polysubstituted;
T represents —O— and U represents —(CR⁵R^{5′})ₙ—; or T represents —(CR⁵R⁵′)ₙ— and U represents —O—;
n is an integer selected from 1, 2, 3, 4, or 5;
R⁵ and R^{5′} independently of one another represent —R^{Y4};
R⁶, and R⁸ independently of one another represent —F, —Cl, —Br, —I, —CN, —NO₂, —SF₅, —R^{w}, —OR^{w}, —OC(═O)R^{w}, —NR^{w}R^{x}, —NR^{w}C(═O)R^{x}, —SR^{w}, —S(═O)R^{w}, —S(═O)₂R^{w}, —C(═O)R^{w}, —C(═O)OR^{w}, or —C(═O)NR^{w}R^{x};
W represents
3-14-membered cycloalkyl, saturated or non-aromatic unsaturated; unsubstituted, mono- or polysubstituted;
6-14-membered aryl, unsubstituted, mono- or polysubstituted;
3-14-membered heterocycloalkyl, saturated or unsaturated, mono- or polysubstituted;
5-14-membered heteroaryl, mono- or polysubstituted; or
—C₁-C₆-alkyl, —C₂-C₆-alkenyl, —C₂-C₆-alkynyl; in each case unsubstituted, mono- or polysubstituted;
wherein
R^{w} and R^{x} independently of one another in each case independently represent
—H;
C₁-C₆-alkyl, saturated or unsaturated, unsubstituted, mono- or polysubstituted;
C₁-C₆-heteroalkyl, saturated or unsaturated, unsubstituted, mono- or poly substituted;
3-14-membered cycloalkyl, saturated or non-aromatic unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted; or
3-14-membered heterocycloalkyl, saturated or unsaturated, unsubstituted, mono- or polysubstituted;
wherein said 3-14-membered heterocycloalkyl is optionally connected through —C₁-C₆-alkylene- or —C₁-C₆-heteroalkylene-, in each case saturated or unsaturated, unsubstituted, mono- or polysubstituted;
R¹, R², R³, and R⁴ independently of one another in each case independently represent
—H;
—C₁-C₆-alkyl, saturated or unsaturated, unsubstituted, mono- or polysubstituted;
—C₁-C₆-heteroalkyl, saturated or unsaturated, unsubstituted, mono- or poly substituted;
3-14-membered cycloalkyl, saturated or non-aromatic unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted;
3-14-membered heterocycloalkyl, saturated or unsaturated, unsubstituted, mono- or polysubstituted;
wherein said 3-14-membered heterocycloalkyl is optionally connected through —C₁-C₆-alkylene- or —C₁-C₆-heteroalkylene-, in each case saturated or unsaturated, unsubstituted, mono- or polysubstituted;
6-14-membered aryl, unsubstituted, mono- or polysubstituted; wherein said 6-14-membered aryl is optionally connected through —C₁-C₆-alkylene- or —C₁-C₆-heteroalkylene-, in each case saturated or unsaturated, unsubstituted, mono- or polysubstituted; or
5-14-membered heteroaryl, unsubstituted, mono- or polysubstituted; wherein said 5-14-membered heteroaryl is optionally connected through —C₁-C₆-alkylene- or —C₁-C₆-heteroalkylene-, in each case saturated or unsaturated, unsubstituted, mono- or polysubstituted;
and wherein “mono- or polysubstituted” in each case independently means substituted with one or more substituents independently of one another selected from —F, —Cl, —Br, —I, —CN, —C₁₋₆-alkyl, —CF₃, —CF₂H, —CFH₂, —CF₂Cl, —CFCl₂, —C₁₋₆-alkylene-CF₃, —C₁₋₆-alkylene-CF₂H, —C₁₋₆-alkylene-CFH₂, —C₁₋₆-alkylene-NH—C₁₋₆-alkylene-CF₃, —C₁₋₆-alkylene-N(C₁₋₆-alkyl)-C₁₋₆-alkylene-CF₃, —C(═O)—C₁₋₆-alkyl, —C₁₋₆-alkylene-C(═O)—C₁₋₆-alkyl, —C(═O)OH, —C₁₋₆-alkylene-C(═O)—OH, —C(═O)—OC₁₋₆-alkyl, —C₁₋₆-alkylene-C(═O)—OC₁₋₆-alkyl, —C(═O)O—C₁₋₆-alkylene-CF₃, —C(═O)—NH₂, —C₁₋₆-alkylene-C(═O)—NH₂, —C(═O)—NH(C₁₋₆-alkyl), —C₁₋₆-alkylene-C(═O)—NH(C₁₋₆-alkyl), —C(═O)—N(C₁₋₆-alkyl)₂, —C₁₋₆-alkylene-C(═O)—N(C₁₋₆-alkyl)₂, —C(═O)—NH(OH), —C₁₋₆-alkylene-C(═O)—NH(OH), —OH, —C₁₋₆-alkylene-OH, ═O, —OCF₃, —OCF₂H, —OCFH₂, —OCF₂Cl, —OCFCl₂, —O—C₁₋₆-alkyl, —C₁₋₆-alkylene-O—C₁₋₆-alkyl, —O—C₁₋₆-alkylene-O—C₁₋₆-alkyl, —O—C₁₋₆-alkylene-NH₂, —O—C₁₋₆-alkylene-NH—C₁₋₆-alkyl, —O—C₁₋₆-alkylene-N(C₁₋₆-alkyl)₂, —O—C(═O)—C₁₋₆-alkyl, —C₁₋₆-alkylene-O—C(═O)—C₁₋₆-alkyl, —O—C(═O)—O—C₁₋₆-alkyl, —C₁₋₆-alkylene-O—C(═O)—O—C₁₋₆-alkyl, —O—C(═O)—NH(C₁₋₆-alkyl), —C₁₋₆-alkylene-O—C(═O)—NH(C₁₋₆-alkyl), —O—C(═O)—N(C₁₋₆-alkyl)₂, —C₁₋₆-alkylene-O—C(═O)—N(C₁₋₆-alkyl)₂, —O—S(═O)₂—NH₂, —C₁₋₆-alkylene-O—S(═O)₂—NH₂, —O—S(═O)₂—NH(C₁₋₆-alkyl), —C₁₋₆-alkylene-O—S(═O)₂—NH(C₁₋₆-alkyl), —O—S(═O)₂—N(C₁₋₆-alkyl)₂, —C₁₋₆-alkylene-O—S(═O)₂—N(C₁₋₆-alkyl)₂, —NH₂, —NO, —NO₂, —C₁₋₆-alkylene-NH₂, —NH(C₁₋₆-alkyl), —C₁₋₆-alkylene-NH(C₁₋₆-alkyl), —N(C₁₋₆-alkyl)₂, —C₁₋₆-alkylene-N(C₁₋₆-alkyl)₂, —NH—C(═O)—C₁₋₆-alkyl, —C₁₋₆-alkylene-NH—C(═O)—C₁₋₆-alkyl, —NH—C(═O)—O—C₁₋₆-alkyl, —C₁₋₆-alkylene-NH—C(═O)—O—C₁₋₆-alkyl, —NH—C(═O)—NH₂, —C₁₋₆-alkylene-NH—C(═O)—NH₂, —NH—C(═O)—NH(C₁₋₆-alkyl), —C₁₋₆-alkylene-NH—C(═O)—NH(C₁₋₆-alkyl), —NH—C(═O)—N(C₁₋₆-alkyl)₂, —C₁₋₆-alkylene-NH—C(═O)—N(C₁₋₆-alkyl)₂, —N(C₁₋₆-alkyl)-C(═O)—C₁₋₆-alkyl, —C₁₋₆-alkylene-N(C₁₋₆-alkyl)-C(═O)—C₁₋₆-alkyl, —N(C₁₋₆-alkyl)-C(═O)—O—C₁₋₆-alkyl, —C₁₋₆-alkylene-N(C₁₋₆-alkyl)-C(═O)—O—C₁₋₆-alkyl, —N(C₁₋₆-alkyl)-C(═O)—NH₂, —C₁₋₆-alkylene-N(C₁₋₆-alkyl)-C(═O)—NH₂, —N(C₁₋₆-alkyl)-C(═O)—NH(C₁₋₆-alkyl), —C₁₋₆-alkylene-N(C₁₋₆-alkyl)-C(═O)—NH(C₁₋₆-alkyl), —N(C₁₋₆-alkyl)-C(═O)—N(C₁₋₆-alkyl)₂, —C₁₋₆-alkylene-N(C₁₋₆-alkyl)-C(═O)—N(C₁₋₆-alkyl)₂, —NH—S(═O)₂OH, —C₁₋₆-alkylene-NH—S(═O)₂OH, —NH—S(═O)₂—C₁₋₆-alkyl, —C₁₋₆-alkylene-NH—S(═O)₂—C₁₋₆-alkyl, —NH—S(═O)₂—O—C₁₋₆-alkyl, —C₁₋₆-alkylene-NH—S(═O)₂—O—C₁₋₆-alkyl, —NH—S(═O)₂—NH₂, —C₁₋₆-alkylene-NH—S(═O)₂—NH₂, —NH—S(═O)₂—NH(C₁₋₆-alkyl), —C₁₋₆-alkylene-NH—S(═O)₂—NH(C₁₋₆-alkyl), —NH—S(═O)₂N(C₁₋₆-alkyl)₂, —C₁₋₆-alkylene-NH—S(═O)₂N(C₁₋₆-alkyl)₂, —N(C₁₋₆-alkyl)-S(═O)₂—OH, —C₁₋₆-alkylene-N(C₁₋₆-alkyl)-S(═O)₂—OH, —N(C₁₋₆-alkyl)-S(═O)₂—C₁₋₆-alkyl, —C₁₋₆-alkylene-N(C₁₋₆-alkyl)-S(═O)₂—C₁₋₆-alkyl, —N(C₁₋₆-alkyl)-S(═O)₂—O—C₁₋₆-alkyl, —C₁₋₆-alkylene-N(C₁₋₆-alkyl)-S(═O)₂—O—C₁₋₆-alkyl, —N(C₁₋₆-alkyl)-S(═O)₂—NH₂, —C₁₋₆-alkylene-N(C₁₋₆-alkyl)-S(═O)₂—NH₂, —N(C₁₋₆-alkyl)-S(═O)₂—NH(C₁₋₆-alkyl), —C₁₋₆-alkylene-N(C₁₋₆-alkyl)-S(═O)₂—NH(C₁₋₆-alkyl), —N(C₁₋₆-alkyl)-S(═O)₂—N(C₁₋₆-alkyl)₂, —C₁₋₆-alkylene-N(C₁₋₆-alkyl)-S(═O)₂—N(C₁₋₆-alkyl)₂, —SH, ═S, —SF₅, —SCF₃, —SCF₂H, —SCFH₂, —S—C₁₋₆-alkyl, —C₁₋₆-alkylene-S—C₁₋₆-alkyl, —S(═O)—C₁₋₆-alkyl, —C₁₋₆-alkylene-S(═O)—C₁₋₆-alkyl, —S(═O)₂—C₁₋₆-alkyl, —C₁₋₆-alkylene-S(═O)₂—C₁₋₆-alkyl, —S(═O)₂—OH, —C₁₋₆-alkylene-S(═O)₂—OH, —S(═O)₂—O—C₁₋₆-alkyl, —C₁₋₆-alkylene-S(═O)₂—O—C₁₋₆-alkyl, —S(═O)₂—NH₂, —C₁₋₆-alkylene-S(═O)₂—NH₂, —S(═O)₂—NH(C₁₋₆-alkyl), —C₁₋₆-alkylene-S(═O)₂—NH(C₁₋₆-alkyl), —S(═O)₂—N(C₁₋₆-alkyl)₂, —C₁₋₆-alkylene-S(═O)₂—N(C₁₋₆-alkyl)₂, 3-14-membered cycloalkyl, —C₁₋₆-alkylene-(3-14-membered cycloalkyl), 3 to 14-membered heterocycloalkyl, —C₁₋₆-alkylene-(3 to 14-membered heterocycloalkyl), -phenyl, —C₁₋₆-alkylene-phenyl, 5 to 14-membered heteroaryl, —C₁₋₆-alkylene-(5 to 14-membered heteroaryl), —O-(3-14-membered cycloalkyl), —O-(3 to 14-membered heterocycloalkyl), —O-phenyl, —O-(5 to 14-membered heteroaryl), —C(═O)-(3-14-membered cycloalkyl), —C(═O)-(3 to 14-membered heterocycloalkyl), —C(═O)-phenyl, —C(═O)-(5 to 14-membered heteroaryl), —S(═O)₂-(3-14-membered cycloalkyl), —S(═O)₂-(3 to 14-membered heterocycloalkyl), —S(═O)₂-phenyl, or —S(═O)₂-(5 to 14-membered heteroaryl).

2. The compound according to claim 1, wherein T represents —O— and U represents —(CR⁵R⁵′)ₙ—.

3. The compound according to any one of claims 1 or 2, wherein R³ represents —H, —OH, —C₁₋₆-alkyl, —C₁₋₆-alkylene-OH, —C₁₋₆-alkylene-O—C₁₋₆-alkyl, —C₁₋₆-alkylene-NH₂, —C₁₋₆-alkylene-NH(C₁₋₆-alkyl), —C₁₋₆-alkylene-N(C₁₋₆-alkyl)₂, —CF₃, —CF₂H, —CFH₂, —CF₂Cl, —CFCl₂, —C₁₋₆-alkylene-CF₃, —C₁₋₆-alkylene-CF₂H, —C₁₋₆-alkylene-CFH₂, —C₁₋₆-alkylene-NH—C₁₋₆-alkylene-CF₃, or —C₁₋₆-alkylene-N(C₁₋₆-alkyl)-C₁₋₆-alkylene-CF₃.

4. The compound according to any one of claims 1 to 3, wherein R⁴ represents
—H;
—S(═O)₂C₁₋₆-alkyl, saturated, unsubstituted, monosubstituted or polysubstituted;
—S(═O)₂(3-14-membered cycloalkyl), saturated, monosubstituted or polysubstituted;
—C₁-C₆-alkyl, saturated or unsaturated, unsubstituted, mono- or poly substituted;
—C₁-C₆-heteroalkyl, saturated or unsaturated, unsubstituted, mono- or polysubstituted;
3-14-membered cycloalkyl, saturated or non-aromatic unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted;
3-14-membered heterocycloalkyl, saturated or unsaturated, unsubstituted, mono- or polysubstituted;
wherein said 3-14-membered heterocycloalkyl is optionally connected through —C₁-C₆-alkylene- or —C₁-C₆-heteroalkylene-, in each case saturated or unsaturated, unsubstituted, mono- or polysubstituted;
—C₁₋₆-alkylene-phenyl unsubstituted, mono- or polysubstituted;
-phenyl unsubstituted, mono- or polysubstituted; or
5-14-membered heteroaryl, unsubstituted, mono- or polysubstituted; wherein said 5-14-membered heteroaryl is optionally connected through —C₁-C₆-alkylene- or —C₁-C₆-heteroalkylene-, in each case saturated or unsaturated, unsubstituted, mono- or polysubstituted.

5. The compound according to any one of claims 1 to 4, wherein R⁴ represents
—H;
—S(═O)₂C₁₋₆-alkyl, saturated, unsubstituted, monosubstituted or polysubstituted with —F;
—S(═O)₂(3-14-membered cycloalkyl), saturated, unsubstituted;
—C₁₋₆-alkyl, saturated, unsubstituted, mono- or polysubstituted with substituents independently of one another selected from the group consisting of —F, —Cl, —Br, —I, —CF₃, —CF₂H, —CFH₂, —CF₂Cl, —CFCl₂, —C₁₋₆-alkylene-CF₃, —C(═O)—C₁₋₆-alkyl, —C(═O)OH, —C(═O)—OC₁₋₆-alkyl, —C(═O)O—C₁₋₆-alkylene-CF₃, —OH, ═O, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C₁₋₆-alkylene-OH, —C₁₋₆-alkylene-O—C₁₋₆-alkyl, —C₁₋₆-alkylene-NHC(═O)O—C₁₋₆-alkyl, —NHC(═O)O—C₁₋₆-alkyl; —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂, 3-14-membered cycloalkyl, -phenyl unsubstituted, oxetanyl, or pyrimidinyl;
3-14-membered cycloalkyl or —C₁₋₆-alkylene-(3-14-membered cycloalkyl), wherein —C₁₋₆-alkylene- is unsubstituted or mono- or polysubstituted with —F, —Cl, —Br, —I, —OH, wherein said 3-14-membered cycloalkyl is saturated, unsubstituted, monosubstituted or disubstituted with substituents independently of one another selected from the group consisting of —F, —Cl, —Br, —I, —CF₃, —CF₂H, —CFH₂, —CF₂Cl, —CFCl₂, —C₁₋₆-alkylene-CF₃, —C(═O)—C₁₋₆-alkyl, —C(═O)OH, —C(═O)—OC₁₋₆-alkyl, —C(═O)O—C₁₋₆-alkylene-CF₃, —OH, ═O, -OC₁₋₆-alkyl, —NH₂, —NHC₁₋₆-alkyl, —N(C₁₋₆-alkyl)₂, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C₁₋₆-alkylene-OH, —C₁₋₆-alkylene-O—C₁₋₆-alkyl, —C₁₋₆-alkylene-NHC(═O)O—C₁₋₆-alkyl, —NHC(═O)O—C₁₋₆-alkyl, —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂, 3-14-membered cycloalkyl, -phenyl unsubstituted, oxetanyl, or pyrimidinyl;
3-14-membered heterocycloalkyl or —C₁₋₆-alkylene-(3-14-membered heterocycloalkyl), wherein —C₁₋₆-alkylene- is unsubstituted or mono- or polysubstituted with —F, —Cl, —Br, —I, —OH, wherein said 3-14-membered heterocycloalkyl in each case is selected from azetane, 1,4-oxazepane, pyrrolidine, piperidine, azepane, diazepane, tetrahydrofuran, tetrahydropyran, oxetane, morpholine, piperazine, hexahydrocyclopenta[c]pyrrole, octahydrocyclopenta[c]pyrrole, octahydropyrrolo[1,2-a]pyrazin, 8-azabicyclo[3.2.1]octane, 9-azabicyclo[3.3.1]nonane, quinuclidine, hexahydro-1H-pyrrolizine, 2-oxaspiro[3.3]heptane, 2-azaspiro[3.3]heptane, 7-azaspiro[3.5]nonane, 1,1-dioxothiacyclohexane, in each case unsubstituted, mono- or polysubstituted with substituents independently of one another selected from the group consisting of —F, —Cl, —Br, —I, —C₁₋₆-alkyl, —CF₃, —CF₂H, —CFH₂, —CF₂Cl, —CFCl₂, —C₁₋₆-alkylene-CF₃, —C(═O)—C₁₋₆-alkyl, —C(═O)OH, —C(═O)—OC₁₋₆-alkyl, —C(═O)O—C₁₋₆-alkylene-CF₃, —OH, ═O, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C₁₋₆-alkylene-OH, —C₁₋₆-alkylene-O—C₁₋₆-alkyl, —C₁₋₆-alkylene-NHC(═O)O—C₁₋₆-alkyl, —NHC(═O)O—C₁₋₆-alkyl, —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂, 3-14-membered heterocycloalkyl, -phenyl unsubstituted, 5 to 14-membered heteroaryl;
5-14-membered heteroaryl or —C₁₋₆-alkylene-(5-14-membered heteroaryl), wherein —C₁₋₆-alkylene- is unsubstituted or monosubstituted with —F, —Cl, —Br, —I, —OH, wherein said 5-14-membered heteroaryl in each case is selected from the group consisting of pyridine, pyridazine, pyrazine, pyrazole, isoxazole, triazole, and [1,2,4]triazolo[4,3-a]pyrimidine, in each case unsubstituted, monosubstituted or disubstituted with substituents independently of one another selected from the group consisting of —F, —Cl, —Br, —I, —C₁₋₆-alkyl, —CF₃, —CF₂H, —CFH₂, —CF₂Cl, —CFCl₂, —C₁₋₆-alkylene-CF₃, —C(═O)—C₁₋₆-alkyl, —C(═O)OH, —C(═O)—OC₁₋₆-alkyl, —C(═O)O—C₁₋₆-alkylene-CF₃, —OH, ═O, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C₁₋₆-alkylene-OH, —C₁₋₆-alkylene-O—C₁₋₆-alkyl, —C₁₋₆-alkylene-NHC(═O)O—C₁₋₆-alkyl, —NHC(═O)O—C₁₋₆-alkyl, —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂, 3-14-membered cycloalkyl, 3-14-membered heterocycloalkyl, -phenyl unsubstituted, 5 to 14-membered heteroaryl.

6. The compound according to any one of claims 1 to 5, wherein
R³ represent —H; and
R⁴ represents —H;
S(═O)₂C₁₋₆-alkyl, saturated, unsubstituted, monosubstituted or polysubstituted with —F;
—S(═O)₂(3-14-membered cycloalkyl), saturated, unsubstituted;
—C₁₋₆-alkyl, saturated, unsubstituted, monosubstituted or disubstituted with substituents independently of one another selected from the group consisting of —OH, ═O, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-NH—C₁₋₆-alkyl, —C(═O)NH₂, —C(═O)—NH—C₁₋₃-alkyl, —C(═O)—N(C₁₋₃-alkyl)₂, or -phenyl unsubstituted;
3-14-membered cycloalkyl or —C₁₋₆-alkylene-(3-14-membered cycloalkyl), wherein —C₁₋₆-alkylene- is unsubstituted or monosubstituted with —OH, wherein said 3-14-membered cycloalkyl is saturated, unsubstituted, monosubstituted or disubstituted with substituents independently of one another selected from the group consisting of —C₁₋₆-alkyl, —C₁₋₆-alkylene-NH₂, —C₁₋₆-alkylene-NH—C₁₋₆-alkylene-CF₃, —C₁₋₆-alkylene-OH, —C₁₋₆-alkylene-NHC(═O)O—C₁₋₆-alkyl, —OH, —OC₁₋₆-alkyl, —NH₂, —N(C₁₋₆-alkyl)₂, —NHC(═O)O—C₁₋₆-alkyl;
3-14-membered heterocycloalkyl or —C₁₋₆-alkylene-(3-14-membered heterocycloalkyl), wherein —C₁₋₆-alkylene- is unsubstituted or monosubstituted with —OH, wherein said 3-14-membered heterocycloalkyl in each case is selected from azetane, 1,4-oxazepane, pyrrolidine, piperidine, azepane, diazepane, tetrahydrofuran, tetrahydropyran, oxetane, morpholine, piperazine, hexahydrocyclopenta[c]pyrrole, octahydrocyclopenta[c]pyrrole, octahydropyrrolo[1,2-a]pyrazin, 8-azabicyclo[3.2.1]octane, 9-azabicyclo-[3.3.1]nonane, quinuclidine, hexahydro-1H-pyrrolizine, 2-oxaspiro[3.3]heptane, 2-azaspiro[3.3]heptane, 7-azaspiro[3.5]nonane, 1,1-dioxothiacyclohexane, in each case unsubstituted, mono- or polysubstituted with substituents independently of one another selected from the group consisting of —F, —OH, ═O, —C₁₋₆-alkyl, —C₁₋₆-alkylene-CF₃, —C₁₋₆-alkylene-OH, —C₁₋₆-alkylene-O—C₁₋₆-alkyl, —NH₂, —N(C₁₋₆-alkyl)₂, —C₁₋₆-alkylene-NH₂, —C₁₋₆-alkylene-N(C₁₋₆-alkyl)₂, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —S(═O)₂C₁₋₆-alkyl, oxetanyl, pyrimidinyl, —C₁₋₆-alkylene-phenyl;
-phenyl unsubstituted;
5-14-membered heteroaryl or —C₁₋₆-alkylene-(5-14-membered heteroaryl), wherein —C₁₋₆-alkylene- is unsubstituted or monosubstituted with —OH, wherein said 5-14-membered heteroaryl in each case is selected from the group consisting of pyridine, pyridazine, pyrazine, pyrazole, isoxazole, triazole, and [1,2,4]triazolo[4,3-a]pyrimidine, in each case unsubstituted, monosubstituted or disubstituted with substituents independently of one another selected from the group consisting of —C₁₋₆-alkyl, —OH.

7. The compound according to any one of claims 1 to 6, wherein W represents
3-14-membered cycloalkyl, saturated or non-aromatic unsaturated, unsubstituted, mono- or polysubstituted with substituents independently selected from —F, —Cl, —Br, —I, —C₁₋₆-alkyl, —CF₃, —CF₂H, —CFH₂, —CF₂Cl, —CFCl₂, —C₁₋₆-alkylene-CF₃, —C(═O)—C₁₋₆-alkyl, —C(═O)OH, —C(═O)—OC₁₋₆-alkyl, —C(═O)O—C₁₋₆-alkylene-CF₃, —OH, ═O, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C₁₋₆-alkylene-OH, —C₁₋₆-alkylene-O—C₁₋₆-alkyl, —C₁₋₆-alkylene-NHC(═O)O—C₁₋₆-alkyl, —NHC(═O)O—C₁₋₆-alkyl, —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂, 3-14-membered cycloalkyl, 3-14-membered heterocycloalkyl, -phenyl unsubstituted, 5 to 14-membered heteroaryl;
6-14-membered aryl, unsubstituted, mono- or polysubstituted independently selected from —F, —Cl, —Br, —I, —C₁₋₆-alkyl, —CF₃, —CF₂H, —CFH₂, —CF₂Cl, —CFCl₂, —C₁₋₆-alkylene-CF₃, —C(═O)—C₁₋₆-alkyl, —C(═O)OH, —C(═O)—OC₁₋₆-alkyl, —C(═O)O—C₁₋₆-alkylene-CF₃, —OH, ═O, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C₁₋₆-alkylene-OH, —C₁₋₆-alkylene-O—C₁₋₆-alkyl, —C₁₋₆-alkylene-NHC(═O)O—C₁₋₆-alkyl, —NHC(═O)O—C₁₋₆-alkyl, —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂, 3-14-membered cycloalkyl, 3-14-membered heterocycloalkyl, -phenyl unsubstituted, 5 to 14-membered heteroaryl;
3-14-membered heterocycloalkyl, saturated or unsaturated, unsubstituted, mono- or polysubstituted independently selected from —F, —Cl, —Br, —I, —C₁₋₆-alkyl, —CF₃, —CF₂H, —CFH₂, —CF₂Cl, —CFCl₂, —C₁₋₆-alkylene-CF₃, —C(═O)—C₁₋₆-alkyl, —C(═O)OH, —C(═O)—OC₁₋₆-alkyl, —C(═O)O—C₁₋₆-alkylene-CF₃, —OH, ═O, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C₁₋₆-alkylene-OH, —C₁₋₆-alkylene-O—C₁₋₆-alkyl, —C₁₋₆-alkylene-NHC(═O)O—C₁₋₆-alkyl, —NHC(═O)O—C₁₋₆-alkyl, —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂, 3-14-membered cycloalkyl, 3-14-membered heterocycloalkyl, -phenyl unsubstituted, 5 to 14-membered heteroaryl;
5-14-membered heteroaryl, unsubstituted, mono- or polysubstituted independently selected from —F, —Cl, —Br, —I, —C₁₋₆-alkyl, —CF₃, —CF₂H, —CFH₂, —CF₂Cl, —CFCl₂, —C₁₋₆-alkylene-CF₃, —C(═O)—C₁₋₆-alkyl, —C(═O)OH, —C(═O)—OC₁₋₆-alkyl, —C(═O)O—C₁₋₆-alkylene-CF₃, —OH, ═O, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C₁₋₆-alkylene-OH, —C₁₋₆-alkylene-O—C₁₋₆-alkyl, —C₁₋₆-alkylene-NHC(═O)O—C₁₋₆-alkyl, —NHC(═O)O—C₁₋₆-alkyl, —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂, 3-14-membered cycloalkyl, 3-14-membered heterocycloalkyl, -phenyl unsubstituted, 5 to 14-membered heteroaryl; or
—C₁-C₆-alkyl, —C₂-C₆-alkenyl, —C₂-C₆-alkynyl; in each case unsubstituted, mono- or polysubstituted with substituents independently selected from —F, —Cl, —Br, —I, —C₁₋₆-alkyl, —CF₃, —CF₂H, —CFH₂, —CF₂Cl, —CFCl₂, —C₁₋₆-alkylene-CF₃, —C(═O)—C₁₋₆-alkyl, —C(═O)OH, —C(═O)—OC₁₋₆-alkyl, —C(═O)O—C₁₋₆-alkylene-CF₃, —OH, ═O, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C₁₋₆-alkylene-OH, —C₁₋₆-alkylene-O—C₁₋₆-alkyl, —C₁₋₆-alkylene-NHC(═O)O—C₁₋₆-alkyl, —NHC(═O)O—C₁₋₆-alkyl, —C(═O)NH₂, —C(═O)—NH—C₁₋₆-alkyl, —C(═O)—N(C₁₋₆-alkyl)₂, —S(═O)₂C₁₋₆-alkyl, —S(═O)₂—NH₂, —S(═O)₂—NH(C₁₋₆-alkyl), —S(═O)₂—N(C₁₋₆-alkyl)₂, 3-14-membered cycloalkyl, 3-14-membered heterocycloalkyl, -phenyl unsubstituted, 5 to 14-membered heteroaryl.

8. The compound according to any one of claims 1 to 7, wherein R¹ represents —H, —F, —Cl, —Br, —I, —CN, —C₁₋₆-alkyl, —O—C₁₋₆-alkyl, —C₁₋₆-alkylene-O—C₁₋₆-alkyl, —C₁₋₆-alkylene-NH(C₁₋₆-alkyl), —C₁₋₆-alkylene-N(C₁₋₆-alkyl)₂, —CF₃, —CF₂H, —CFH₂, —CF₂Cl, —CFCl₂, —C₁₋₆-alkylene-CF₃, —C₁₋₆-alkylene-CF₂H, —C₁₋₆-alkylene-CFH₂, —C₁₋₆-alkylene-NH—C₁₋₆-alkylene-CF₃, —C₁₋₆-alkylene-N(C₁₋₆-alkyl)-C₁₋₆-alkylene-CF₃, —C(═O)C₁₋₆-alkyl, —C(═O)OC₁₋₆-alkyl, —C(═O)NHC₁₋₆-alkyl, —C(═O)NH₂, —C(═O)N(C₁₋₆-alkyl)₂, —S(═O)—C₁₋₆-alkyl, —S(═O)₂—C₁₋₆-alkyl, —O—C₁₋₆-alkyl, -cyclopropyl unsubstituted, cyclobutyl unsubstituted, cyclopentyl unsubstituted or cyclohexyl unsubstituted.

9. The compound according to any one of claims 1 to 8, wherein R⁵ and R^{5′} independently of one another represent
—H;
—C₁-C₆-alkyl, saturated or unsaturated, unsubstituted, mono- or poly substituted;
—C₁-C₆-heteroalkyl, saturated or unsaturated, unsubstituted, mono- or poly substituted;
3-14-membered cycloalkyl, saturated or non-aromatic unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted.

10. The compound according to any one of claims 1 to 9, wherein R⁶, and R⁸ independently of one another represent
—H, —F, —Cl, —Br, —I, —OH, —SH, —SF₅, —CN, —NO₂, —C(═O)OH, —NH₂,
—C₁₋₆-alkyl, —CF₃, —CHF₂, —CH₂F,
—O—C₁₋₆-alkyl, —OCF₃, —OCHF₂, —OCH₂F,
—NHC₁₋₆-alkyl unsubstituted or substituted with one or more substituents independently of one another selected from —OH, ═O, —F, —Cl, —Br, —I, —SH, ═S, —CN, —CF₃, —CHF₂, —CH₂F, —OCF₃, —OCHF₂, —OCH₂F, SF₅, —NO₂, —C(═O)OH, —NH₂, and —C(═O)NH₂;
—N(C₁₋₆-alkyl)₂ unsubstituted or substituted with one or more substituents independently of one another selected from —OH, ═O, —F, —Cl, —Br, —I, —SH, ═S, —CN, —CF₃, —CHF₂, —CH₂F, —OCF₃, —OCHF₂, —OCH₂F, SF₅, —NO₂, —C(═O)OH, —NH₂, and —C(═O)NH₂;
—C(═O)OC₁₋₆-alkyl unsubstituted or substituted with one or more substituents independently of one another selected from —OH, ═O, —F, —Cl, —Br, —I, —SH, ═S, —CN, —CF₃, —CHF₂, —CH₂F, —OCF₃, —OCHF₂, —OCH₂F, SF₅, —NO₂, —C(═O)OH, —NH₂, and —C(═O)NH₂;
—OC(═O)C₁₋₆-alkyl unsubstituted or substituted with one or more substituents independently of one another selected from —OH, ═O, —F, —Cl, —Br, —I, —SH, ═S, —CN, —CF₃, —CHF₂, —CH₂F, —OCF₃, —OCHF₂, —OCH₂F, SF₅, —NO₂, —C(═O)OH, —NH₂, and —C(═O)NH₂; or
—C₁₋₆-heteroalkyl unsubstituted or substituted with one or more substituents independently of one another selected from —OH, ═O, —F, —Cl, —Br, —I, —SH, ═S, —CN, —CF₃, —CHF₂, —CH₂F, —OCF₃, —OCHF₂, —OCH₂F, SF₅, —NO₂, —C(═O)OH, —NH₂, and —C(═O)NH₂.

11. The compound according to any one of claims 1 to 10, which is selected from the group consisting of
Cpd 001—(2S)-3-hydroxy-2-({2-methyl-5-[(2-methyl-1,3-thiazol-5-yl)methoxy]furo[2,3-c]pyridin-3-yl}formamido)propanamide;
Cpd 002—(2S,3R)-2-({5-[(2-fluorophenyl)methoxy]-2-methylfuro[2,3-c]pyridin-3-yl}formamido)-3-hydroxybutanamide;
Cpd 003—(2S,3R)-3-hydroxy-2-({2-methyl-5-[(2-methyl-1,3-thiazol-5-yl)methoxy]furo[2,3-c]pyridin-3-yl}formamido)butanamide;
Cpd 004—(2S,3R)-3-hydroxy-2-({2-methyl-5-[(4-methyl-1,3-thiazol-5-yl)methoxy]furo[2,3-c]pyridin-3-yl}formamido)butanamide;
Cpd 005—2-({2-methyl-5-[(4-methyl-1,3-thiazol-5-yl)methoxy]furo[2,3-c]pyridin-3-yl}formamido)propanamide;
Cpd 006—2-({2-methyl-5-[(pyridin-2-yl)methoxy]furo[2,3-c]pyridin-3-yl}formamido)propanamide;
Cpd 007—2-({5-[(2-fluorophenyl)methoxy]-2-methylfuro[2,3-c]pyridin-3-yl}formamido)-2-methylpropanamide;
Cpd 008—2-({5-[(2-fluorophenyl)methoxy]-2-methylfuro[2,3-c]pyridin-3-yl}formamido)-3-hydroxypropanamide;
Cpd 009—2-({5-[(2-fluorophenyl)methoxy]-2-methylfuro[2,3-c]pyridin-3-yl}formamido)propanamide;
Cpd 010—2-{[5-(benzyloxy)-2-methylfuro[2,3-c]pyridin-3-yl]formamido}propanamide;
Cpd 011—2-methyl-2-({2-methyl-5-[(4-methyl-1,3-thiazol-5-yl)methoxy]furo[2,3-c]pyridin-3-yl}formamido)propanamide;
Cpd 012—2-methyl-2-({2-methyl-5-[(pyridin-2-yl)methoxy]furo[2,3-c]pyridin-3-yl}formamido)propanamide;
Cpd 013—3-hydroxy-2-({2-methyl-5-[(4-methyl-1,3-thiazol-5-yl)methoxy]furo[2,3-c]pyridin-3-yl}formamido)propanamide;
Cpd 014—3-hydroxy-2-({2-methyl-5-[(pyridin-2-yl)methoxy]furo[2,3-c]pyridin-3-yl}formamido)propanamide;
Cpd 015—5-(benzyloxy)-N-(4,4-difluoropyrrolidin-3-yl)-2-methylfuro[2,3-c]pyridine-3-carboxamide;
Cpd 016—5-(benzyloxy)-N-[4-(hydroxymethyl)oxan-4-yl]-2-methylfuro[2,3-c]pyridine-3-carboxamide;
Cpd 017—5-[(2-fluorophenyl)methoxy]-N-[1-(2-hydroxyethyl)-2-oxopyrrolidin-3-yl]-2-methylfuro[2,3-c]pyridine-3-carboxamide;
Cpd 018—5-[(2-fluorophenyl)methoxy]-N-[1-(hydroxymethyl)cyclopropyl]-2-methylfuro[2,3-c]pyridine-3-carboxamide;
Cpd 019—N-(3-carbamoyloxetan-3-yl)-2-methyl-5-[(4-methyl-1,3-thiazol-5-yl)methoxy]furo[2,3-c]pyridine-3-carboxamide;
Cpd 020—N-(3-carbamoyloxetan-3-yl)-2-methyl-5-[(pyridin-2-yl)methoxy]furo[2,3-c]pyridine-3-carboxamide;
Cpd 021—N-(4,4-difluoropyrrolidin-3-yl)-2-methyl-5-[(4-methyl-1,3-thiazol-5-yl)methoxy]furo[2,3-c]pyridine-3-carboxamide;
Cpd 022—N-(4,4-difluoropyrrolidin-3-yl)-2-methyl-5-(2-phenylethoxy)furo[2,3-c]pyridine-3-carboxamide;
Cpd 023—N-(4,4-difluoropyrrolidin-3-yl)-2-methyl-5-[2-(4-methyl-1,3-thiazol-5-yl)ethoxy]furo[2,3-c]pyridine-3-carboxamide;
Cpd 024—N-(4-carbamoyloxan-4-yl)-2-methyl-5-[(pyridin-2-yl)methoxy]furo[2,3-c]pyridine-3-carboxamide;
Cpd 025—N-[(4R)-3,3-difluoropiperidin-4-yl]-2-methyl-5-{[2-(trifluoromethyl)pyridin-3-yl]methoxy}furo[2,3-c]pyridine-3-carboxamide;
Cpd 026—N-[(4S)-3,3-difluoropiperidin-4-yl]-2-methyl-5-{[2-(trifluoromethyl)pyridin-3-yl]methoxy}furo[2,3-c]pyridine-3-carboxamide;
Cpd 027—N-[1-(2-hydroxyethyl)-2-oxopyrrolidin-3-yl]-2-methyl-5-[(pyridin-2-yl)methoxy]furo[2,3-c]pyridine-3-carboxamide;
Cpd 028—N-[1-(hydroxymethyl)cyclobutyl]-2-methyl-5-[(4-methyl-1,3-thiazol-5-yl)methoxy]furo[2,3-c]pyridine-3-carboxamide;
Cpd 029—N-[1-(hydroxymethyl)cyclobutyl]-2-methyl-5-[(pyridin-2-yl)methoxy]furo[2,3-c]pyridine-3-carboxamide;
Cpd 030—N-[1-(hydroxymethyl)cyclopropyl]-2-methyl-5-[(4-methyl-1,3-thiazol-5-yl)methoxy]furo[2,3-c]pyridine-3-carboxamide;
Cpd 031—N-[1-(hydroxymethyl)cyclopropyl]-2-methyl-5-[(pyridin-2-yl)methoxy]furo[2,3-c]pyridine-3-carboxamide;
Cpd 032—N-[1-(hydroxymethyl)cyclopropyl]-5-[(2-methoxypyridin-3-yl)methoxy]-2-methylfuro[2,3-c]pyridine-3-carboxamide;
Cpd 033—N-[3-(hydroxymethyl)oxetan-3-yl]-2-methyl-5-[(4-methyl-1,3-thiazol-5-yl)methoxy]furo[2,3-c]pyridine-3-carboxamide;
Cpd 034—N-[3-(hydroxymethyl)oxetan-3-yl]-2-methyl-5-[(pyridin-2-yl)methoxy]furo[2,3-c]pyridine-3-carboxamide;
Cpd 035—N-[4-(hydroxymethyl)oxan-4-yl]-2-methyl-5-[(4-methyl-1,3-thiazol-5-yl)methoxy]furo[2,3-c]pyridine-3-carboxamide.

12. A pharmaceutical composition comprising a compound according to any one of claims 1 to 11.

13. A compound according to any one of claims 1 to 11 or the pharmaceutical composition according to claim 12, for use as a medicament.

14. A compound according to any one of claims 1 to 11 or the pharmaceutical composition according to claim 12, for use in the treatment of pain or epilepsy.

15. The compound for use or the pharmaceutical composition for use according to claim 14, wherein the pain is selected from nociceptive pain, inflammatory pain, and neuropathic pain; preferably post-operative pain.
